(19) United States
(12) Patent Application Publication
(10) Pub. No.: US 2026/0102518 A1
(43) Pub. Date: Apr. 16, 2026

(54) Gene Therapy for Genetic and Acquired Vasculopathies

(71) Applicant: The General Hospital Corporation, Boston, MA (US)

(72) Inventors: Patricia L. Musolino, Boston, MA (US); Mark Evan Lindsay, Winchester, MA (US); Benjamin P. Kleinstiver, Boston, MA (US); Casey A. Maguire, Arlington, MA (US); David Young Chung, Boston, MA (US); Christian Lacks Lino Cardenas, Boston, MA (US); Rajeev Malhotra, Boston, MA (US)

(21) Appl. No.: 19/116,185

(22) PCT Filed: Sep. 29, 2023
(86) PCT No.: PCT/US2023/075589

§ 371 Date: Mar. 27, 2025

Related U.S. Application Data
(60) Provisional application No. 63/411,367, filed on Sep. 29, 2022.
(60) Provisional application No. 63/459,463, filed on Apr. 14, 2023.

(51) Int. Cl. A61K 48/00; C12N 9/22; C12N 15/11; C12N 15/86

(52) U.S. Cl. CPC A61K 48/0058; C12N 9/22; C12N 15/11; C12N 15/86; C12N 2310/20; C12N 2750/14143; C12N 2750/14145

(57) ABSTRACT

Described herein are gene-targeted therapies and compositions that can include a vessel-specific viral vector, preferably in combination with a HDAC9-derived promoter to transduce SMC and deliver a base editor that corrects mutant alleles or a Cas nuclease that knocks out the mutant allele.

### CLAIM OF PRIORITY

This application claims the benefit of U.S. Provisional Patent Application Ser. Nos. 63/411,367, filed on Sep. 29, 2022, and 63/459,463, filed on Apr. 14, 2023. The entire contents of the foregoing are hereby incorporated by reference.

### FEDERALLY SPONSORED RESEARCH OR DEVELOPMENT

This invention was made with Government support under Grant Nos. NS117575, HL142809, NS065743, NS066225, and NS094683 awarded by the National Institutes of Health. The Government has certain rights in the invention.

### SEQUENCE LISTING

This application contains a Sequence Listing that has been submitted electronically as an XML file named “29539-0616WO1_SL_ST26.XML.” The XML file, created on Sep. 28, 2023, is 85,394 bytes in size. The material in the XML file is hereby incorporated by reference in its entirety.

### TECHNICAL FIELD

Described herein are gene-targeted therapies and compositions that can include a vessel-specific viral vector, preferably in combination with a HDAC9-derived promoter to transfect SMC and deliver a base editor that selectively corrects mutant alleles in SMCs or a Cas nuclease for selective knockout of the mutant allele.

### BACKGROUND

Genetic vasculopathies cause up to 10% of childhood strokes (10-20/100,000, over 8,000 cases per year). The majority affect smooth muscle cell (SMC) function and are caused by missense mutations. Pathogenic missense mutations in the actin alpha 2, smooth muscle (ACTA2) gene at arginine 179 (most commonly replaced by histidine-referred here also as ACTA2 R179H) cause a severe syndrome termed multisystemic smooth muscle dysfunction syndrome (MSMDS) characterized by systemic smooth muscle cell (SMC) dysfunction, hypotension, aortic aneurysms and devastating cerebrovascular disease that leads to death in the first 3 decades of life.

### SUMMARY

This Summary is provided to introduce a selection of concepts in a simplified form that are further described below in the Detailed Description. Other features, details, utilities, and advantages of the claimed subject matter will be apparent from the following written Detailed Description including those aspects illustrated in the accompanying drawings and defined in the appended claims.

Provided herein are isolated nucleic acids comprising an HDAC9-derived promoter sequence that has at least 90% identity to SEQ ID NO: 4 and optionally contains at least one substitution modification relative to SEQ ID NO: 4, or a promoter sequence that has at least 90% identity to SEQ ID NO: 5, and optionally contains at least one substitution modification relative to SEQ ID NO: 5, or a promoter sequence that has at least 90% identity to SEQ ID NO: 6 and optionally contains at least one substitution modification relative to SEQ ID NO: 6, or a promoter sequence that has at least 90% identity to SEQ ID NO: 85 and optionally contains at least one substitution modification relative to SEQ ID NO: 85, or a promoter sequence that has at least 90% identity to SEQ ID NO: 86 and optionally contains at least one substitution modification relative to SEQ ID NO: 86, or a promoter sequence that has at least 90% identity to SEQ ID NO: 87 and optionally contains at least one substitution modification relative to SEQ ID NO: 87. In some embodiments, the sequence is not a naturally occurring sequence, e.g., does not include any naturally occurring SNPs.

Also provided herein are isolated nucleic acids comprising a heterologous (i.e., other than HDAC9) transgene and an HDAC9-derived promoter sequence that has at least 90% identity to SEQ ID NO: 4, 5, 6, 85, 86, or 87, preferably wherein the promoter sequence is operably linked to the heterologous transgene, to drive expression of the heterologous transgene.

In some embodiments, transcription by the promoter sequence is inducible by actin depolymerization agents within a cell. In some embodiments, the promoter drives transcription of the heterologous coding sequence in vascular smooth muscle cells.

Also provided are vectors comprising the HDAC9-derived promoters described herein, and optionally a heterologous nucleic acid sequence (e.g., a transgene), optionally wherein the vector is an AAV vector or is encapsulated by an adeno-associated virus (AAV) capsid. In some instances, the heterologous nucleic acid sequence (e.g., transgene, e.g., a genome editing agent) is split between two or more vectors, and the two or more vectors are encapsulated by two or more AAVs. E.g., a first vector encoding a portion of a heterologous nucleic acid sequence is encapsulated by a first AAV, a second vector encoding a second portion of the heterologous nucleic acid sequence is encapsulated by a second AAV, etc.

Additionally provided herein are adeno-associated virus (AAV) comprising (i) a peptide sequence PRPPSTH (SEQ ID NO:1), MAEPGAR (SEQ ID NO:25), MLYADNT (SEQ ID NO: 26), or SQDPSTL (SEQ ID NO: 27), in its capsid (i.e., inserted into the capsid protein), and (ii) a nucleic acid sequence comprising a smooth muscle cell-specific promoter or active portion thereof encapsulated by the capsid. In some embodiments, the smooth muscle cell-specific promoter is an HDAC9-derived promoter. In some embodiments, the HDAC9-derived promoter comprises a sequence that has at least 90% identity to SEQ ID NO: 4, 5, 6, 85, 86, or 87.

In some embodiments, the smooth muscle promoter, e.g., HDAC9-derived promoter, is operably linked to a heterologous transgene. In some embodiments, the transgene encodes a genome editor or an intein-split construct thereof, optionally selected from CRISPR nucleases, cytosine base editors (CBEs), adenine base editors (ABEs), and CRISPR prime editors (PEs). In some embodiments, the transgene encodes:
a. a CRISPR/Cas base editor or an intein-split construct thereof, and/or an Arg179His mutant-allele specific guide RNA directing a base editor to the mutation, and/or an Arg179Cys mutant-allele specific guide RNA directing a base editor to the mutation, or
b. a CRISPR/Cas genome editor or an intein-split construct thereof, and/or an Arg179His mutant-allele specific guide RNA directing a genome editor to the mutation, and/or an Arg179Cys mutant-allele specific guide RNA directing a base editor to the mutation.

In some embodiments, the sequence PRPPSTH (SEQ ID NO: 1) is inserted into VP1 protein of the AAV vector, e.g., in a position corresponding to amino acids 588 and 589 of SEQ ID NO: 2.

In some embodiments, the AAV serotype is AAV-9 or AAV-2.

Additionally provided herein are compositions comprising the vectors described herein, e.g., the AV vectors described herein.

Further, provided herein are recombinant episomes comprising a nucleic acid sequence that encodes an adeno-associated virus (AAV) capsid having one or more of peptides selected from the group consisting of PRPPSTH (SEQ ID NO: 1), MAEPGAR (SEQ ID NO: 25), MLYADNT (SEQ ID NO: 26), and SQDPSTL (SEQ ID NO: 27) on its capsid, and a nucleic acid sequence comprising a smooth muscle specific promoter or active portion thereof encapsulated by the capsid.

In some embodiments, at least two copies of the one or more of peptides selected from the group consisting of PRPPSTH (SEQ ID NO:1), MAEPGAR (SEQ ID NO:25), MLYADNT (SEQ ID NO:26), and SQDPSTL (SEQ ID NO:27) are inserted into the capsid of the AAV vector. In some embodiments, the at least two copies are of a same peptide. In some embodiments, the at least two copies are of a different peptide. In some embodiments, the one or more of peptides selected from the group consisting of PRPPSTH (SEQ ID NO:1), MAEPGAR (SEQ ID NO:25), MLYADNT (SEQ ID NO:26), and SQDPSTL (SEQ ID NO: 27) are inserted into the VP1 protein of the AAV vector. In some embodiments, the one or more of peptides selected from the group consisting of PRPPSTH (SEQ ID NO:1), MAEPGAR (SEQ ID NO:25), MLYADNT (SEQ ID NO: 26), and SQDPSTL (SEQ ID NO: 27) are inserted into the VP1 protein of the AAV vector in a position corresponding to amino acids 588 and 589 of SEQ ID NO: 2.

In some embodiments, the AAV serotype is AAV-9 or AAV-2.

Also provided are compositions comprising the AAV vectors encoded by the recombinant episomes described herein.

Further, provided herein are methods of correcting an Arg179His mutation or an Arg179Cys mutation in the genome of a cell. The methods comprise delivering to the cell: (i) a CRISPR/Cas base editor or an intein-split construct thereof, and an Arg179His or an Arg179Cys mutant-allele specific guide RNA directing the base editor to the mutation, or (ii) a CRISPR/Cas genome editor or an intein-split construct thereof, and an Arg179His or an Arg179Cys mutant-allele specific guide RNA directing the genome editor to the mutation.

In some embodiments, the base editor or intein-split construct thereof, genome editor or intein-split construct thereof, and/or guide RNA are listed in Table 1 and/or Table 2 (comprising SEQ ID NOs: 55-79), optionally comprising SEQ ID NO:60.

In some embodiments, the Arg179His or the Arg179Cys mutant-allele specific CRISPR/Cas base editor is an adenine base editor or an intein-split construct thereof comprising the wild-type SpCas9, or D1135L/S1136W/G1218K/E1219Q/R1335Q/T1337R (SpG), A61R/L1111R/D1135L/S1136W/G1218K/E1219Q/N1317R/A1322R/R1333P/R1335 Q/T1337R (SpRY), D10T(optional)/1322V/S409I/E427G/R654L/R753G/R1114G/D1135N/V1139A/D1180G/E1 219V/Q1221H/A1320V/R1333K (SpCas9-NRRH), D10T (optional)/1322V/S409I/E427G/R654L/R753G/R1114G/D1135N/E1219V/D1332N/R1 335Q/T1337N/S1338T/H1349R (SpCas9-NRCH), D1135M/S1136Q/G1218K/E1219S/R1335E/T1337R (MQSKER), D1135V/G1218R/R1335Q/T1337R (VRQR), or S55R/D1135V/G1218R/R1335Q/T1337R (VRQR(S55R)) variants of Streptococcus pyogenes Cas9 protein (SpCas9). In some embodiments, the spacer sequence of the guide RNA targets the sequence TGCATCTGGATCTGGCTGGC (SEQ ID NO:28) (HES1208-A4 gRNA) with a CGA PAM and the target adenine in position 4 of the spacer, e.g., optionally with ABE8e-SpCas9-VRQR. ABE8e-SpCas9-VRQR(S55R), ABE8e-SpG, ABE8e-SpRY, or ABE8e-SpCas9-NRRH (Table 1) or an intein-split construct thereof; or the sequence TCATGCATCTGGATCTGGCT (SEQ ID NO: 24, HES1210-A7 gRNA) with a GGC PAM and the target adenine in position A7 of the spacer, optionally with ABE8e-SpG, ABE8e-SpRY, ABE8e-SpCas9-NRCH, ABE8e-MQSKER (Table 1) or an intein-split construct thereof; or the sequence ATCATGCATCTGGATCTGGC (SEQ ID NO:23, HES1212-A8 gRNA) with a TGG PAM and the target adenine in position A8 of the spacer, optionally with ABE8e-SpCas9, ABE8e-SpG, ABE8e-SpRY, or ABE8e-SpCas9-NRRH (Table 1) or an intein-split construct thereof.

In some embodiments, the genome editor is a wild-type SpCas9 nuclease or an intein-split construct thereof, and in some embodiments, the spacer sequence of the guide RNA targets the sequence TGCCATCATGCATCTGGATC (HES1235, SEQ ID NO:83) or AGCCAGATCCAGATGCATGA (HES1236, SEQ ID NO:84).

In some embodiments, the base editor or intein-split construct thereof and guide RNA, or the genome editor or intein-split construct thereof and guide RNA, are delivered to the cell using an adeno-associated virus (AAV) vectors, preferably an AAV comprising the sequence PRPPSTH (SEQ ID NO: 1) inserted into the VP1 protein, in a position corresponding to between amino acids 588 and 589 of SEQ ID NO:2, and preferably comprising an HDAC9 promoter or active portion thereof.

In some embodiments, the HDAC9 promoter comprises a sequence that has at least 90% identity to SEQ ID NO: 4, 5, 6, 85, 86, or 87.

In some embodiments, the cell is in a living subject. In some embodiments, the living subject is a human with: (i) an Arg179His mutation in an allele of ACTA2, and the method comprises delivering a base editor or an intein-split construct thereof and/or guide RNA, or (ii) an Arg179Cys mutation in an allele of ACTA2, and the method comprises delivering a genome editor or an intein-split construct thereof and/or guide RNA.

Additionally provided herein are methods for treating a vasculopathy, optionally multisystem smooth muscle dysfunction syndrome (MSMDS), in a subject who has an ACTA2 R179H mutation. The methods comprise delivering to the vasculature of the subject a therapeutically effective amount of a gene therapy agent comprising: a CRISPR/Cas base editor or an intein-split construct thereof, and a guide RNA directing the base editor to the Arg179His mutation; or a CRISPR/Cas genome editor or an intein-split construct thereof, and a guide RNA directing the genome editor to the Arg179His mutation. In some embodiments, the base editor or intein-split construct thereof and/or guide RNA are listed in Table 1 and/or Table 2 (comprising SEQ ID NOs: 55-79), optionally comprising SEQ ID NO:60.

In some embodiments, the Arg179His or the Arg179Cys mutant-allele specific CRISPR/Cas base editor is an adenine base editor or an intein-split construct thereof comprising the wild-type SpCas9, SpG, SpRY, VRQR, VRQR(S55R), NRCH, NRRH, or MQKSER variants of Streptococcus pyogenes Cas9 protein (SpCas9). In some embodiments, the spacer sequence of the guide RNA targets the sequence TGCATCTGGATCTGGCTGGC (SEQ ID NO:28) (HES1208-A4 gRNA) with a CGA PAM and the target adenine in position 4 of the spacer, e.g., in combination with or optionally with ABE8e-SpCas9-VRQR, ABE80-SpCas9-VRQR(S55R), ABE8e-SpG, ABE8e-SpRY, or ABE8e-SpCas9-NRRH (Table 1) or an intein-split construct thereof; or the sequence TCATGCATCTGGATCTGGCT (SEQ ID NO: 24, HES1210-A7 gRNA) with a GGC PAM and the target adenine in position A7 of the spacer, optionally with ABE8e-SpG, ABE8e-SpRY, ABE8e-SpCas9-NRCH, ABE8e-MQSKER (Table 1) or an intein-split construct thereof; or the sequence ATCATGCATCTGGATCTGGC (SEQ ID NO:23, HES1212-A8 gRNA) with a TGG PAM and the target adenine in position A8 of the spacer, optionally with ABE8e-SpCas9, ABE8e-SpG, ABE8e-SpRY, or ABE8e-SpCas9-NRRH (Table 1) or an intein-split construct thereof.

In some embodiments, the genome editor or intein-split construct thereof and/or guide RNA are listed in Table 1 and/or Table 2 (comprising SEQ ID NOs: 55-79), optionally comprising SEQ ID NO:60.

In some embodiments, the genome editor is a wild-type SpCas9 or an intein-split construct thereof. In some embodiments, the spacer sequence of the guide RNA directing the genome editor to the mutation comprises the sequence TGCCATCATGCATCTGGATC (HES1235, SEQ ID NO:83) or AGCCAGATCCAGATGCATGA (HES1236, SEQ ID NO: 84).

In some embodiments, the base editor or genome editor or intein-split construct thereof and guide RNA are delivered to the cell using adeno-associated virus (AAV) vectors, preferably AAV comprising the sequence PRPPSTH (SEQ ID NO: 1) inserted into the VP1 protein, in a position corresponding to between amino acids 588 and 589 of SEQ ID NO:2, and preferably comprising an HDAC9 promoter or active portion thereof.

In some embodiments, the HDAC9 promoter comprises a sequence that has at least 90% identity to SEQ ID NO: 4, 5, 6, 85, 86, or 87.

In some embodiments, the gene therapy agent is administered to the subject by local or systemic delivery. In some embodiments, the gene therapy agent is administered systemically by intravenous or intra-arterial delivery:

Other features and advantages of the invention will be apparent from the following detailed description and figures, and from the claims.

### DESCRIPTION OF DRAWINGS

The foregoing and other features and advantages of the present invention will be more fully understood from the following detailed description of illustrative embodiments taken in conjunction with the accompanying drawings in which:

FIGS. 1A-H: collectively illustrate development and characterization of a mouse model of MSMDS. FIG. 1A depicts an Acta2R179H mouse model allele structure (Allele name Acta2^{R179Hfl}). Cre excision results in the replacement of normal coding sequence with a new exon 6 coding for Histidine substitution at position 179. FIG. 1B is a graph illustrating survival function of Myh11-Cre:Acta2R179Hfl/+, Wnt1-Cre:Acta2R179Hfl/+, and Acta2R179Hfl/+ mice reveals early spontaneous mortality. Log rank test p<0.001 for both, Myh11 and Wnt1-Cre mutant mice compared to controls. FIG. 1C is a picture of three mice in a cage and a line graph illustrating that Myh11-Cre:Acta2R179Hfl/+ mice are smaller and gain weight slower than Wnt1 Cre:Acta2^{R179Hfl/+} or Acta2R179Hfl/+ mice. P values: ** p<0.01: *** p<0.001. FIG. 1D is a picture of the abdominal wall of a control and a Myh11-Cre:Acta2R179Hfl/+ mice, illustrating that the Myh11-Cre:Acta2R179Hfl/+ is thin and commonly exhibits clearly visible distension. Mice are 4 months of age at the time of the picture. FIG. 1E depicts a histopathology examination of pulmonary parenchyma revealing increased alveolar area, with decreased numbers of type 2 pneumocytes (as assessed by SPTFC and RAGE staining) in mutant mice compared to control mice. FIG. 1F depicts intestinal distension and stool clustering in Myh11-Cre:Acta2R179Hfl/+ mice. FIG. 1G depicts kidney hypertrophy and hydronephrosis. FIG. 1H depicts bladder distension in Myh11-Cre:Acta2R179Hfl/+ mice.

FIGS. 2A-C collectively illustrate ex vivo characterization of Smooth Muscle Cells from Acta2R179H mice. FIG. 2A depicts cultured vascular smooth muscle cells (VSMCs) from Myh11-Cre:Acta2R179Hfl/+ and Acta2R179Hfl/+ mice aortas stained with rhodamine-labeled phalloidin (F-actin), DAPI (nuclei) and GFP (Myh11-cre expression). FIG. 2B depicts cultured VSMCs from Myh11Cre:Acta2R179Hfl/+ and Acta2R179Hfl/+ stained for F-actin, DAPI (nuclei) and Myh11 and ACTA2 Smooth Muscle Actin (SMA) after treatment with DMSO (control) or TGFbeta1 at 10 ng/cc. FIG. 2C depicts cultured high resolution imaging of the VSMC cytoskeleton with F-actin and SMA with overlay. Lack of colocalization is quantified (right).

FIGS. 3A-E collectively illustrate large Vessel Characterization in Acta2R179H mice. FIG. 3A depicts an ultrasound still image and post latex injection photography of the ascending aorta and carotid arteries in Wnt1-Cre:Acta2R179Hfl/+, Myh11-Cre:Acta2R17 9Hfl/+, and Acta2R179Hfl/+ mice. FIG. 3B is a chart depicting quantification of ultrasound diameters. n=7, 7, 7 respectively (Root=aortic root, Asc=ascending aorta, Cad=common carotid artery). Error bars are standard deviations. FIG. 3C depicts hematoxylin and eosin staining and Masson's trichrome staining of ascending aortas from in Wnt1-Cre:Acta2R179Hfl/+, Myh11-Cre:Acta2R179Hfl/+, and Acta2R179Hfl/+ mice demonstrates cellular disarray and increased collagen deposition in aortas from mutant mice. FIG. 3D depicts immunofluorescence of aortic tissue from Wnt1-Cre:Acta2R179Hfl/+, Myh11-Cre:Acta2R179Hfl/+, and Acta2R179Hfl/+ mice demonstrates loss of filamentous actin (F-actin) and SM22 staining in regions expressing cre recombinase. FIG. 3E depicts systolic, diastolic and mean arterial invasive blood pressure (SBP, DBP and MAP respectively) measurements of 6 Acta2R179Hfl/+ control mice and 5 Myh11-Cre:Acta2R179Hfl/+ mutant mice under isoflurane anesthesia. Myh11-Cre:Acta2R179Hfl/+ mutant mouse showed a trend towards lower SBP and Pulse Pressure (SBP-DBP) compared to controls.

FIGS. 4A-C collectively depict the results of behavioral testing in Acta2R179H mice from open field test (OFT) and Y-maze in Acta2R179Hfl/+ control mice and Wnt1-Cre:Acta2R179Hfl/+ mutant mice. FIG. 4A depicts representative OFT tracks showing decreased distance traveled in the Wnt1-Cre:Acta2R179Hfl/+ mutant mice compared to Acta2R179Hfl/+ control mice over 5 minutes. The thick lines delineate an outside track to calculate thigmotaxis (time spent in the outer track/total time). Distance traveled, average speed, and thigmotaxis are shown before cranial windows are placed (pre) and at different week intervals. FIG. 4B is a chart depicting percent alternation and number of entries into each arm of the Y-maze. n=4 control and 3 Wnt1-Cre:Acta2R179Hfl/+ mice. FIG. 4C is a chart depicting rotarod performance of 12 control and 8 Wnt1-Cre:Acta2R179Hfl/+ mice (*p=0.0024). Error bars are standard deviations.

FIGS. 5A-G collectively depict the characterization of cerebral vasculopathy in Acta2R179H mice. In FIG. 5A, latex injection in a 4-month-old Wnt1-Cre:Acta2R179Hfl/+ mice demonstrates narrowing of the terminal ICA (arrows) and straightening of MCA branching on the lateral view. Histological examination of the Myh11-Cre:Acta2R179Hfl/+ mice (FIGS. 5 B-G) depicted steno-occlusive phenotype as early as 8 weeks of age and confirmed development of luminal narrowing at expense of VSMC proliferation in the vessel wall in H&E (light arrows in FIGS. 5 B-G), abnormal elastin architecture in Congo Red (dark arrow in FIG. 5F) with loss of post-mortem contraction artifact (FIG. 5F) and increased collagen revealed by Trichrome staining (FIGS. 5 D-G) in cerebral arteries (FIGS. 5 B-E) and small vessels (FIG. 5F) compared to representative controls. Bars=50 μm (FIG. 5A), 100 μm (FIG. 5B), 75 μm (FIGS. C-D) and 20 μm (FIGS. E-G).

FIGS. 6A-D collectively depict histopathology of neurodegeneration in Acta2R179H mice. FIG. 6A depicts severe white matter loss with vacuolar rarefication, which is most severe in the Myh11-Cre:Acta2R179Hfl/+ mice when compared to control mice on quantification of myelin basic protein staining (MBP) (FIG. 6B). FIG. 6C is a pair of images depicting intracytoplasmic neuronal inclusions akin to neurofibrillary tangles (arrows) in Acta2R179Hfl/+ (left) and Myh11-Cre:Acta2R179Hfl/+ (right) mice, with insets showing thioflavin positive-immunofluorescence in motor cortex of mutant mice. FIG. 6D depicts quantification of neuronal loss in the dentate gyrus of Myh11-Cre:Acta2R179Hfl/+ mice.

FIGS. 7A-C collectively depict neurovascular connectivity in Acta2R179H mice. FIG. 7A is an illustration of OIS set up with LED illuminating the surface of the mouse brain and its raw in vivo image captured through the glass window. Left visual cortex functional hyperemia and timelapse D[HbT] maps showing delayed activation after right visual field alternating checkerboard stimulus in the Wnt1-Cre:Acta2R179Hfl/+ mouse. FIG. 7B depicts resting state functional connectivity (RSFC) maps denoting lower amplitude of hemodynamic fluctuations in the BOLD frequency range (0.01-0.1 Hz) from 12 minutes record and global connectivity-measured by average correlation coefficient of each pixel to every other pixel in the brain-in the Wnt1-Cre:Acta2R179Hfl/+. Black circles: Acta2R179Hfl/+ control and grey triangles: Wnt1-Cre:Acta2R179Hfl/+ mice. FIG. 7C depicts an averaged RSFC global connectivity map denoting seeds placement and corresponding motor, sensory, retrosplenial, and visual area connectivity at different time points denote lower connectivity (most severe motor) in the Wnt1-Cre:Acta2R179Hfl/+ mouse compared to controls.

FIGS. 8A-C collectively depict induced ischemic stroke through unilateral carotid occlusion. FIG. 8A depicts histological and immunofluorescence of ischemic injury 3 days after unilateral ICA ligation. Evidence of lack of blood flow (CBF) redistribution between contralateral and ipsilateral hemispheres in the Wnt1-Cre:Acta2R179Hfl/+ mice (n=3) is shown by ipsilateral multifocal infarctions with increased cellularity, loss of neurons and massively dilated vessels in trichrome staining and upregulation of Hypoxia Inducible Factor 1-alpha (HIF1a) and total Tau (most severe ipsilaterally). No significant ischemia was observed in the Acta2R179Hfl/+ control mice (n=3). Bars=100 μm (trichrome), 250 μm and 25 μm for IF. FIG. 8B depicts a long axis section of carotid artery distal to surgical occlusion in left carotid artery. As seen in FIG. 8B, Myh11-Cre:Acta2R179Hfl/+ mice exhibit exaggerated neointimal formation when compared to Acta2R179Hfl/+ control littermates. FIG. 8C are pictures of mice after carotid occlusion Myh11-Cre:Acta2R179Hfl/+ mice exhibit left eye sclerosis (white arrow) and necrosis of the left ear (grey arrow) presumably from lack of left sided external carotid arterial blood flow, unlike Acta2R179Hfl/+ control mice who tolerate unilateral carotid occlusion.

FIG. 9 shows a western blot and bar graph quantifying levels of globular (left band or G) and polymerized (right band or F) actin in human skin fibroblasts from a patient with an ACTA2 R179H mutation, a patient with ACTA2 R179C mutation, and a control (unaffected). Cells expressing the mutant ACTA2 protein had a decreased amount of polymerized (F-actin).

FIGS. 10A-B collectively show images of scratch migration assay experiments in which human skin fibroblasts were grown from a patient with an ACTA2 R179H mutation, a patient with ACTA2R179C mutation, and two controls (unaffected). Cells were plated on cell chambers that contain an insert of consistent width that prevents cellular growth in that area. The insert was removed and cells were photographed at time zero and at 24 hours. Cells migrated into the space previously occupied by the insert. FIG. 10A shows that cells from patients with ACTA2 R179H and ACTA2 R179C migrate into the space faster than control cells. FIG. 10B shows that TGF-beta treatment was found to slow the migration of both control and patient cells.

FIGS. 11A-F collectively show that HDAC9 expression in the animal model of MSMDS (Acta2 R179H) is increased and the Hdac9 promoter region shows transcriptional activation in response to environmental or genetic disruption of vascular smooth muscle homeostasis modeling the human disease state. FIG. 11A is a western blot from total lysates of ascending aortas in Acta2R179Hfl/+ control and Myh11-Cre:Acta2R179Hfl/+ mutant mice. Mutant Myh11-Cre:Acta2R179Hfl/+ aortas show increased levels of Hdac9 which associates with low levels of contractile proteins including Cnn1. FIG. 11B depicts bioinformatic identification of transcription factor binding site motifs on the human HDAC9 promoter region involving 2500 base pairs upstream of the start (ATG) codon. The HDAC9 promoter was divided into 3 different fragments to assay its bioactivity in vitro and in vivo. FIG. 11C is a schematic representation of the construct used to investigate HDAC9 promoter bioactivity: Red fluorescent protein (RFP) expression was driven by the HDAC9 promoter. FIG. 11D, left panel, shows transfection of the HDAC9-RFP plasmid in human skin fibroblasts of healthy (ACTA2 R179) or ACTA2 R179H (ACTA2 H179) patient. Microscopy analysis of the RFP expression shows activation of the 3 HDAC9 promoter fragments especially Fragment P2 in fibroblasts from the patient carrying the ACTA2 R179H mutation. Right panel, healthy human aortic smooth muscle cells were transfected with the HDAC9-promoter fragments and treated with latrunculin (3 uM). HDAC9 fragment P2 shows higher transcriptional activation in response of the cellular stressor latrunculin. FIG. 11E shows a western blot analysis of HDAC9-promoter fragment P2 driving RFP expression from total lysates of wild-type (ACTA2 R179) or mutant acta2 (ACTA2 H179) HEK cells. RFP expression shows increased activity of the HDAC9-promoter fragment P2 in the disease state. FIG. 11F shows a western blot analysis of total lysates from human aortic smooth muscle cells transfected with HDAC9-promoter fragment P2. RFP expression shows increased activity of the HDAC9-promoter fragment P2 in response to the actin depolymerizing agent, latrunculin.

FIGS. 12A-D collectively show specific activation of the HDAC9 promoter in the context of smooth muscle cell-associated disease in vivo. FIG. 12A depicts a histological analysis of brains from Acta2R179Hfl/+ control and Myh11-Cre:Acta2R179Hfl/+ mutant mice that were injected with AAV8 containing RFP downstream of the HDAC9 promoter fragment P2. Higher RFP expression is detected in arterioles of brains from Myh11-Cre:Acta2R179Hfl/+ mutant mice. Similarly, aortas (FIG. 12B) and kidneys (FIG. 12C) show higher expression of RFP in smooth muscle cells from Myh11-Cre:Acta2R179Hfl/+ mutant mice compared Acta2R179Hfl/+ control mice. FIG. 12D shows low expression of the RFP protein in livers from both Acta2R179Hfl/+ control and Myh11-Cre:Acta2R179Hfl/+ mutant mice. SMCs were identified as cells positive for SMA.

FIGS. 13A-D collectively show activation of the AAV8-based HDAC9 promoter-RFP fragment P2 in multiple animal models of vascular-associated disease in vivo, including: brain (FIG. 13A), heart (FIG. 13B), kidney (FIG. 13C), bladder (FIG. 13D).

FIGS. 14A-G collectively show the generation of ACTA2 R179H cell models. FIG. 14A is a schematic of how CRISPR-Cas genome editing can be deployed to generate and revert the ACTA2 R179H mutation (steps 1 and 2, respectively); shown are SEQ ID NO: 29 (GCCATCATGCGTCTGGATCTG) and SEQ ID NO: 30 (GCCATCATGCATCTGGATCTG). FIG. 14B demonstrates an efficiency of generating a pure ACTA2 R179H edit in HEK 293T cells via prime editing. Properties that can be varied to alter editing efficiency include the reverse transcriptase template (RTT) length and the primer binding site (PBS) length of the pegRNA, as well as the PE3 nicking site. FIGS. 14C-E depict visualizations of the genotypes of three separate clonal cell lines bearing some proportion of the ACTA2 R179H (CAT codon) allele. The CGT allele is the R179 allele. Shown are SEQ ID NOs: 31-32 (14C), 33-35 (14B), and 33, 34, and 36 (14E), respectively. FIGS. 14F-14G summarize molecular characterization results of different generated ACTA2 R179H cell lines using western blot and bar graph quantifying levels of globular (G) and polymerized (F) actin. On average, cells expressing the mutant ACTA2 protein showed decreased amount of polymerized (F-actin) and higher G/F ratios than HEK 293T cells with wild type ACTA2.

FIGS. 15A-I collectively show CRISPR base editing approaches to correct ACTA2 R179H. FIG. 15A is a schematic of A-to-G base editors (ABEs) to correct the mutation causing R179H. FIGS. B-E are depictions of the ABE target sites accessible using different CRISPR-Cas variants, which position the intended adenine base that must be edited in different phases of the edit windows of the ABEs; shown are SEQ ID NO: 30 (GCCATCATGCATCTGGATCTG) and SEQ ID NO: 37 (AIMHLDLAGRD). FIGS. 15F and 15G depict the results of experiments using R179H line 1 (see FIG. 14C), various ABE and gRNA constructs were delivered to assess the ability to convert R179H back to R179. The proportion of R179H alleles that remain following editing (FIG. 15F), or the fraction of R179H alleles that are corrected to R179 (FIG. 15G), are shown. In FIG. 15F, the horizontal dashed line represents the average proportion of NGS reads harboring the R179H allele in control unedited samples (grey bar). FIG. 15H depicts a separate experiment using the R179H line 2 (see FIG. 14D). FIG. 15H illustrates the proportion of R179H alleles that are perfectly reverted to R179 (via H179R change), that have both H179R and M178V changes, or just M178V bystander editing. All experiments were performed in biological triplicate, with individual data points plotted and the mean and s.e.m. shown. FIG. 15I depicts preliminary western blot results of G/F actin ratio in HEK ACTA2 R179H line 5 before and after base editing with ABE8e-SpCas9-VRQR demonstrating restoration of actin polymerization and reduction of G/F actin to control wild type levels.

FIGS. 16A-C collectively depict allele-selective knockout of ACTA2 R179H. FIG. 16A is a schematic depicting the use of a CRISPR-Cas enzyme to selectively target and knockout the mutant R179H allele, while leaving the wild-type R179 allele intact. FIG. 16B illustrates results of experiments using R179H line 1 (see FIG. 14C), various CRISPR-Cas nuclease and gRNA constructs were delivered to assess the ability to knockout the R179H allele. The horizontal dashed line represents the average proportion of NGS reads harboring the R179H allele in control unedited samples (black bar). FIG. 16C is a graph depicting a selection of the data from panel B, the fraction of R179H alleles that are knocked out are shown. All experiments were performed in biological triplicate, with individual data points plotted and the mean and s.e.m. shown.

FIGS. 17A-D collectively demonstrate that AAV-PR-CBA-Cre mediates vasculature-tropic transduction in transgenic Ai9 mice (CAG-floxed-STOP-tdTomato). Mice were injected systemically with AAV-PR-CBA-Cre and sacrificed three weeks later. FIG. 17A is a whole-hemisphere image of tdTomato immunofluorescence displaying vasculature transduction by AAV-PR. FIG. 17B is an image of the cortical region displaying transduced vasculature and DAPI. FIGS. 17C-D are high magnification image of the boxed region from (FIG. 17B) showing transduced vasculature.

FIGS. 18A-B collectively depict the evaluation of additional A-to-G base editor (ABE) constructs to correct ACTA2 R179H. FIG. 18A shows that in experiments using a homozygous ACTA2 R179H HEK 293T cell line (line 4), various ABE and gRNA constructs were delivered to assess the ability to convert R179H back to R179. The proportion of A-to-G editing in the R179 CAT codon is shown. Datapoints are shown as dots for n=3 independent biological replicates (transfections on separate days), with mean and s.e.m. shown. The gRNA target sequences (with spacer and PAM) are shown below the graph, SEQ ID NOs: 38-40, respectively. FIG. 18B shows analysis of bystander editing as approximate proportions of total A-to-G editing in the R179 CAT codon (data reanalyzed from panel A). The intended edit of a single H179R alteration is shown as the bottom section of each bar. Mean and s.e.m. shown for n=3.

FIGS. 19A-C collectively depict the evaluation of split ABE constructs to correct ACTA2 R179H. FIG. 19A is a schematic of the two plasmids used for most HEK 293T transfections. The first plasmid expresses the ABE8e construct (with various modifications to alter PAM preference and other properties), while the second plasmid is utilized to express the gRNA. Elements not drawn to scale. FIG. 19B is a schematic of plasmids encoding the dual-AAV constructs. The first plasmid encodes the ABE8e domain with the N-terminal portion of Cas9 (with various modifications to alter PAM preference and other properties), while the second plasmid encodes the C-terminal portion of Cas9 (with various modifications to alter PAM preference and other properties) along with the gRNA expression cassette. The two halves of the ABE8e construct are rejoined post-translationally via the Npu intein. Elements not drawn to scale. FIG. 19C is a comparison of R179H-to-H179R editing with constructs including the canonical single ABE8e plasmids co-transfected along with a separate gRNA expression plasmid encoding the A4 NGA PAM gRNA (treatments 1-4), or dual vectors encoding halves of the ABE8e-SpCas9-VRQR(S55R) construct with the A4 NGA PAM gRNA (treatment 5). Experiments were performed using a homozygous ACTA2 R179H HEK 293T cell line (line 4), where various ABE8e constructs were delivered to assess the ability to convert R179H back to R179. Datapoints for A-to-G editing in the CAT codon are shown as dots for n=3 independent biological replicates (transfections on separate days), with mean and s.e.m.

FIGS. 20A-D collectively depict in vivo editing of Acta R179H Mouse Model. FIG. 20A is a schematic representation of the ABE editor, AAV-dual intein strategy and table with doses use of each intein vector in 2 different cohorts (2 and 6-week-old). Retroorbital (FIG. 20B) and intravenous tail vein (FIG. 20C) injections were performed in Acta2R179Hfl/+ control and Myh11-Cre:Acta2R179Hfl/+ mutant mice. A non-manipulated group of mice for each cohort and each AAV vector (AAV9 and AAV-PR) was used as untreated controls. FIG. 20D depicts a summary table with characteristics and interventions of all mice used in this study.

FIGS. 21A-B collectively depict the results of the overall assessment of in vivo editing in mouse models of ACTA2 R179H. FIGS. 21A-B are summaries of injections of a 1:1 ratio of N-terminal to C-terminal AAVs were performed with 6 e11 vg of each AAV per mouse (1.2 e12 vg/mouse total) via tail vein injection into Acta2R179Hfl/+ control mice at 6 weeks of age (cohort #1). FIGS. 21A-B show experiments performed using untreated mice and those treated with AAV9 or AAV-PR. Quantification of the proportion of amplicon sequencing reads with the R179 codon (CGT) (FIG. 21A), and levels of A-to-G editing (converting CAT to CGT) versus control samples (calculated as described in the methods; FIG. 21B). Dots for n=2 mice (biological replicates), with mean and s.e.m. shown.

FIGS. 21C-D are summaries of injections of a 1:1 ratio of N-terminal to C-terminal AAVs were performed with 8 e10 vg of each AAV per mouse (1.6 e11 vg/mouse total) via retroorbital injection into Acta2R179Hfl/+ control mice at 2-3 weeks of age (cohort #2). FIGS. 21C-D show experiments performed using untreated mice and those treated with AAV9 or AAV-PR. Quantification of the proportion of amplicon sequencing reads with the R179 codon (CGT) (FIG. 21C), and levels of A-to-G editing (converting CAT to CGT) versus control samples (calculated as described in the methods; FIG. 21D). Dots for n=2 mice (biological replicates), with mean and s.e.m. shown.

FIGS. 21E-F are summaries of injections of a 1:1 ratio of N-terminal to C-terminal AAVs were performed with 6 e11 vg of each AAV (1.2 e12 vg/mouse total) via tail vein injection into Myh11-Cre:Acta2R179Hfl/+ mutant mice at 6 weeks of age (cohort #1). Here shown experiments performed using untreated mice and those treated with AAV9 or AAV-PR. Quantification of the proportion of amplicon sequencing reads with the R179 codon (CGT) (FIG. 21E), and levels of A-to-G editing (converting CAT to CGT) versus control samples (calculated as described in the methods; FIG. 21F). Dots for n=1 or 2 mice (biological replicates), with mean and s.e.m. shown.

FIGS. 21G-H are summaries of injections of a 1:1 ratio of N-terminal to C-terminal AAVs were performed with 8 e10 vg of each AAV (1.6 e11 vg/mouse total) via retroorbital injection into Myh11-Cre:Acta2R179Hfl/+ mutant mice at 2-3 weeks of age (cohort #2). Here shown experiments performed using untreated mice and those treated with AAV9 or AAV-PR. Quantification of the proportion of amplicon sequencing reads with the R179 codon (CGT) (FIG. 21G), and levels of A-to-G editing (converting CAT to CGT) versus control samples (calculated as described in the methods; FIG. 21H). Dots for n=2 or 3 mice (biological replicates), with mean and s.e.m. shown.

FIGS. 22A-G collectively summarize quantification of AAV genomes in mouse models of Acta2 R179H. FIGS. 22A-22G show the AAV genome copy number relative to genomic copies of the mouse GAPDH gene (quantified by ddPCR), using tissues extracted from untreated mice or those treated with AAV9 or AAV-PR (encoding the split ABE8e-SpCas9-VRQR(S55R) base editor along with the A4 NGA PAM gRNA). The AAV genome copy number was quantified in liver (FIG. 22A), brain vasculature (FIG. 22B), lungs (FIG. 22C), kidney (FIG. 22D), bladder (FIG. 22E) heart (FIG. 22F), or ascending aorta (FIG. 22G). Mice from cohort 1 were injected using a 1:1 ratio of N-terminal to C-terminal AAVs at a 6 e11 vg of each AAV per mouse (1.2 e12 vg/mouse total) via tail vein injection at 6 weeks of age; mice from cohort 2 were injected using a 1:1 ratio of N-terminal to C-terminal AAVs at a 8 e10 vg of each AAV per mouse (1.6 e11 vg/mouse total) via retro-orbital injection into Acta2R179Hfl/+ control and Myh11-Cre:Acta2R179Hfl/+ mutant mice mice at 2-3 weeks of age.

FIGS. 23A-B collectively show selected tissues assessment of in vivo editing in mouse models of Acta2 R179H. Mice from cohort 2 were injected using a 1:1 ratio of N-terminal to C-terminal AAVs at a 8 e10 vg of each AAV per mouse (1.6 e11 vg/mouse total) via retro-orbital injection into Acta2R179Hfl/+ control and Myh11-Cre:Acta2R179Hfl/+ mutant mice at 2-3 weeks of age. FIG. 23A shows averaged AAV genome copy number relative to genomic copies of GAPDH gene quantified by ddPCR, using tissues extracted from untreated mice or those treated with AAV9 or AAV-PR (encoding the split ABE8e-SpCas9-VRQR(S55R) base editor along with the A4 NGA PAM gRNA) in the liver, brain vasculature and kidney. FIG. 23B shows averaged levels of A-to-G editing in mutant versus control treated samples (calculated as described in the methods). Untreated animals showed no AAV genomes or A-to-G editing. Dots denote each mouse (biological replicates), with mean and s.e.m. shown.

FIGS. 24A-B collectively show ex vivo vascular expression of Cas9 in 2 week-old treated mice. FIG. 24A shows mRNA expression quantified using qPCR of liver and aorta tissues 2 weeks after injections of AAV9 (mouse ID #34 & 49) or AAV-PR (mouse ID #40 & 52) dual split ABE8e-SpCas9-VRQR(S55R) or untreated (mouse ID #32 & 37) mice of cohort #2 (injected at 2-week-old). FIG. 24B shows representative photographs of liver immunofluorescence of Cas9 protein and ACTA2 depicting higher levels of expression of the enzyme in the vasculature (veins, arteries and sinusoidal capillaries) in AAV-PR vs AAV9 in Acta2R179Hfl/+ control (upper row) and Myh11-Cre:Acta2R179Hfl/+ mutant (lower row) retro-orbitally-injected at 2-3 weeks of age. The lack of hepatocyte expression is likely due to high level of replication in the liver at this young age.

FIGS. 25A-C show schematics of vector payload and dosing (A) and schedule of events (B) on mouse treatment trial, and (C) shows genotype and characteristics of P3 efficacy study mice cohort.

FIG. 26 shows Kaplan-Meier survival function of Myh11-Cre:Acta2R179Hfl/+ mutant mice (total n=11; 6 male, 5 female; 6 AAVPR packaged, 5 AAV9 packaged) injected with total 1.6 e11 vg/mouse (1:1 ratio of N-terminal to C-terminal AAVs) at day 3 of life (P3) with dual-AAV split ABE8e-SpCas9-VRQR(S55R) editor or untreated. All surviving mice were sacrificed at 8 weeks of life.

FIG. 27 shows rotarod performance of Myh11-Cre:Acta2R179Hfl/+ mutant mice injected with total 1.6 e11 vg/mouse at day 3 of life (P3) with dual-AAV split ABE8e-SpCas9-VRQR(S55R) editor (1:1 ratio of N-terminal to C-terminal AAVs) (total n=11; 6 AAVPR packaged, 5 AAV9 packaged) compared to Acta2R179Hfl/+ control mice or untreated Myh11-Cre:Acta2R179Hfl/+ mutant untreated mice. Mutant mice treated with split ABE8e-SpCas9-VRQR(S55R) performed motor tasks as well as Acta2R179Hfl/+ control mice.

FIG. 28 shows transthoracic echocardiogram measurement of the aortic root and ascending aortic diameter at 8 weeks of life from Myh11-Cre:Acta2R179Hfl/+ mutant mice injected with dual-AAV split ABE8e-SpCas9-VRQR(S55R) editor at P3 when compared to Acta2R179Hfl/+ control mice or untreated Myh11-Cre:Acta2R179Hfl/+ mutant mice. Injected mice showed significantly smaller aortic root and ascending aortic dimensions.

FIGS. 29A-B show weekly weight measurements of dual-AAV split ABE8e-SpCas9-VRQR(S55R) editor P3 injected Myh11-Cre:Acta2R179Hfl/+ mutant mice compared to Myh11-cre control mice or untreated Myh11-Cre:Acta2R179Hfl/+ mutant mice. Male mice (29A; 6 Treated) and females (29B; 5 treated) shown separately. Injected mice restored normal weight gain when compared to untreated Myh11-Cre:Acta2R179Hfl/+ mutant mice.

FIG. 30A shows results of open field behavioral testing of dual-AAV split ABE8e-SpCas9-VRQR(S55R) editor P3 injected Myh11-Cre:Acta2R179Hfl/+ mutant mice compared to untreated Myh11-Cre:Acta2R179Hfl/+ mutant mice. While untreated mice demonstrated remarkably decreased distance traveled starting at 6 weeks of life, injected Myh11-Cre:Acta2R179Hfl/+ mutant mice maintained previous behavior.

FIG. 30B shows bright field microphotographs of coronal brain 8-micron sections stained with ACTA2 (SMA) where control mice denote classic wrinkled interna elastica layer of muscular arteries at the base of the brain (Middle and Posterior Cerebral Arteries) and in the parenchyma (hippocampal arteries) in control, mutant untreated and treated representative mice. Scale bars=20 microns.

FIGS. 31A-I. In vivo editing of ACTA2-R179H via AAV9 delivery at P3, cohort 3. A-to-G editing in humanized mouse models of ACTA2 R179H following delivery of the split ABE8e-SpCas9-VRQR(S55R) base editor along with the A4 NGA PAM gRNA via AAV9. Mice from cohort 3 were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜2.510 vg/mouse total) at 3 days of age. Tissues were collected at 8 weeks of age. Editing was assessed by amplicon sequencing with quantification of the percentage of A-to-G editing at R179H (converting CAT to CGT) versus control samples. Dots for n=1, editing for each individual mouse assessed across various tissues including liver (panel A), brain vasculature (panel B), ascending aorta (panel C), heart (panel D), lungs (panel E), kidney (panel F), bladder (panel G), small intestine (panel H), or large intestine (panel I).

FIGS. 32A-I. In vivo editing of ACTA2-R179H via AAV-PR delivery at P3, cohort 3. A-to-G editing in humanized mouse models of ACTA2 R179H following delivery of the split ABE8e-SpCas9-VRQR(S55R) base editor along with the A4 NGA PAM gRNA via AAV-PR. Mice from cohort 3 were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜2.5 e10 vg/mouse total) at 3 days of age. Tissues were collected at 8 weeks of age. Editing was assessed by amplicon sequencing with quantification of the percentage of A-to-G editing at R179H (converting CAT to CGT) versus control samples. Dots for n=1, editing for each individual mouse assessed across various tissues including liver (A), brain vasculature (B), ascending aorta (C), heart (D), lungs (E), kidney (F), bladder (G), small intestine (H), or large intestine (I). Data point in A (ID 175) was not collected due to a technical issue with sample collection.

FIGS. 33A-B. Purity of base editing outcomes. (A) Schematic illustrating the theoretical edit window of a base editor (BE). BEs edit most efficiently within a sequence window at the PAM distal end of the spacer sequence, with ideal placement of the target adenine to be edited within the edit window. Co-occurrence of other non-target bases within the edit window may lead to unwanted editing of nearby bases (so-called bystander editing). (B) Consequences of bystander edits near ACTA2 R179H. Schematic of the R179H locus highlighting the consequence of bystander base editing of nearby adenines or cytosines, which could cause M178V, L180M, L180V, L180L (silent) or D181G edits; shown are SEQ ID NOs: 30, 37, and 41-50).

FIGS. 34A-I. Bystander editing of ACTA2-M178V via AAV9 delivery at P3, cohort 3. A-to-G editing at A-1 to create ACTA2-M178V mutations in humanized mouse models of ACTA2 R179H following delivery of the split ABE8e-SpCas9-VRQR(S55R) base editor along with the A4 NGA PAM gRNA via AAV9. Mice from cohort 3 were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜2.5 e10 vg/mouse total) at 3 days of age. Tissues were collected at 8 weeks of age. Editing was assessed by amplicon sequencing with quantification of the percentage of A-to-G editing at M178V (converting ATG to GTG) versus control samples. Dots for n=1, editing for each individual mouse assessed across various tissues including liver (A), brain vasculature (B), ascending aorta (C), heart (D), lungs (E), kidney (F), bladder (G), small intestine (H), or large intestine (I).

FIGS. 35A-I. Bystander editing of ACTA2-M178V via AAV-PR delivery at P3, cohort 3. A-to-G editing at A-1 to create ACTA2-M178V mutations in humanized mouse models of ACTA2 R179H following delivery of the split ABE8e-SpCas9-VRQR(S55R) base editor along with the A4 NGA PAM gRNA via AAV-PR. Mice from cohort 3 were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜2.5 e10 vg/mouse total) at 3 days of age. Tissues were collected at 8 weeks of age. Editing was assessed by amplicon sequencing with quantification of the percentage of A-to-G editing at M178V (converting ATG to GTG) versus control samples. Dots for n=1, editing for each individual mouse assessed across various tissues including liver (A), brain vasculature (B), ascending aorta (C), heart (D), lungs (E), kidney (F), bladder (G), small intestine (H), or large intestine (I).

FIGS. 36A-I. Bystander editing of ACTA2-D181G via AAV9 delivery at P3, cohort 3. A-to-G editing at A10 to create ACTA2-D181G mutations in humanized mouse models of ACTA2 R179H following delivery of the split ABE8e-SpCas9-VRQR(S55R) base editor along with the A4 NGA PAM gRNA via AAV9. Mice from cohort 3 were injected via retro-orbital injection into Acta2R179Hfl/+ control and Myh11-Cre:Acta2R 179Hfl/+ mutant mice using a 1:1 ratio of N-terminal to C-terminal AAVs at a dose of 1.3 e13 vg/kg of each AAV per mouse (˜2.5 e10 vg/mouse total) at 3 days of age. Tissues were collected at 8 weeks of age. Editing was assessed by amplicon sequencing with quantification of the percentage of A-to-G editing at D181G (converting GAT to GGT) versus control samples. Dots for n=1, editing for each individual mouse assessed across various tissues including liver (A), brain vasculature (B), ascending aorta (C), heart (D), lungs (E), kidney (F), bladder (G), small intestine (H), or large intestine (I).

FIGS. 37A-I. Bystander editing of ACTA2-D181G via AAV-PR delivery at P3, cohort 3. A-to-G editing at A10 to create ACTA2-D181G mutations in humanized mouse models of ACTA2 R179H following delivery of the split ABE8e-SpCas9-VRQR(S55R) base editor along with the A4 NGA PAM gRNA via AAV-PR. Mice from cohort 3 were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜2.5 e10 vg/mouse total) at 3 days of age. Tissues were collected at 8 weeks of age. Editing was assessed by amplicon sequencing with quantification of the percentage of A-to-G editing at D181G (converting GAT to GGT) versus control samples. Dots for n=1, editing for each individual mouse assessed across various tissues including liver (A), brain vasculature (B), ascending aorta (C), heart (D), lungs (E), kidney (F), bladder (G), small intestine (H), or large intestine (I).

FIGS. 38A-I. Bystander editing of ACTA2-L180M/V/L via AAV9 delivery at P3, cohort 3. C-to-A, C-to-G, or C-to-T editing at C6 to create ACTA2-L180M, -L180V, or a silent -L180L mutation in humanized mouse models of ACTA2 R179H following delivery of the split ABE8-SpCas9-VRQR(S55R) base editor along with the A4 NGA PAM gRNA via AAV9. Mice from cohort 3 were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜2.5 e10 vg/mouse total) at 3 days of age. Tissues were collected at 8 weeks of age. Editing was assessed by amplicon sequencing with quantification of the percentage of C-to-A/G/T editing at L180 (converting CTG to ATG/GTG/TTG, respectively) versus control samples. Dots for n=1, editing for each individual mouse assessed across various tissues including liver (A), brain vasculature (B), ascending aorta (C), heart (D), lungs (E), kidney (F), bladder (G), small intestine (H), or large intestine (I).

FIGS. 39A-I. Bystander editing of ACTA2-L180M/V/L via AAV-PR delivery at P3, cohort 3. C-to-A, C-to-G, or C-to-T editing at C6 to create ACTA2-L180M, -L180V, or a silent -L180L mutation in humanized mouse models of ACTA2 R179H following delivery of the split ABE8e-SpCas9-VRQR(S55R) base editor along with the A4 NGA PAM gRNA via AAV-PR. Mice from cohort 3 were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜2.5 e10 vg/mouse total) at 3 days of age. Tissues were collected at 8 weeks of age. Editing was assessed by amplicon sequencing with quantification of the percentage of C-to-A/G/T editing at L180 (converting CTG to ATG/GTG/TTG, respectively) versus control samples. Dots for n=1, editing for each individual mouse assessed across various tissues including liver (A), brain vasculature (B), ascending aorta (C), heart (D), lungs (E), kidney (F), bladder (G), small intestine (H), or large intestine (I).

FIGS. 40A-I. Insertions and deletion mutations via AAV9 delivery at P3, cohort 3. Levels of insertion or deletion mutations (indels) observed in humanized mouse models of ACTA2 R179H following delivery of the split ABE8e-SpCas9-VRQR(S55R) base editor along with the A4 NGA PAM gRNA via AAV9. Mice from cohort 3 were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜2.5 e10 vg/mouse total) at 3 days of age. Tissues were collected at 8 weeks of age. Editing was assessed by amplicon sequencing with quantification of the percentage of indels versus control samples. Dots for n=1, editing for each individual mouse assessed across various tissues including liver (A), brain vasculature (B), ascending aorta (C), heart (D), lungs (E), kidney (F), bladder (G), small intestine (H), or large intestine (I).

FIGS. 41A-I. Insertions and deletion mutations via AAV-PR delivery at P3, cohort 3. Levels of insertion or deletion mutations (indels) observed in humanized mouse models of ACTA2 R179H following delivery of the split ABE8e-SpCas9-VRQR(S55R) base editor along with the A4 NGA PAM gRNA via AAV-PR. Mice from cohort 3 were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜2.5 e10 vg/mouse total) at 3 days of age. Tissues were collected at 8 weeks of age. Editing was assessed by amplicon sequencing with quantification of the percentage of indels versus control samples. Dots for n=1, editing for each individual mouse assessed across various tissues including liver (A), brain vasculature (B), ascending aorta (C), heart (D), lungs (E), kidney (F), bladder (G), small intestine (H), or large intestine (I).

FIGS. 42A-F. Quantification of AAV transduction in mouse tissues from cohort 3. (A-B) The AAV genome copy number relative to genomic copies of the mouse GAPDH gene was quantified from genomic DNA via ddPCR, using tissues extracted from untreated mice or those treated with AAV-PR or AAV9 (panels A and B, respectively) encoding the split ABE8e-SpCas9-VRQR(S55R) base editor along with the A4 NGA PAM gRNA. AAV genome copy number was quantified from selected mice in tissue samples including liver, brain, aorta, and heart. (C-D) Expression of the mRNA Cas9 transcript relative to GAPDH transcript levels was quantified from a cDNA library via ddPCR, using tissues extracted from untreated mice or those treated with AAV-PR (N- or C-term AAV in panels C and D, respectively) or with AAV9 (N- or C-term AAV in panels E and F, respectively). Mice from cohort 3 were injected via retro-orbital injection into Acta2R179Hfl/+ control and Myh11-Cre:Acta2R179Hfl/+ mutant mice using a 1:1 ratio of N-terminal to C-terminal AAVs at a dose of 2.6 e13 vg/kg of each AAV per mouse at 3 days of age. Tissues were collected at 8 weeks of age. Dots for n=1.

FIGS. 43A-J. In vivo editing of ACTA2-R179H via AAV9 delivery at P14, cohort 3. A-to-G editing in humanized mouse models of ACTA2 R179H following delivery of the split ABE8e-SpCas9-VRQR(S55R) base editor along with the A4 NGA PAM gRNA via AAV9. Mice from cohort 3 were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜8 e10 vg/mouse total) at 14 days of age. Tissues were collected at 8 weeks of age. Editing was assessed by amplicon sequencing with quantification of the percentage of A-to-G editing at R179H (converting CAT to CGT) versus control samples. Dots for n=1, editing for each individual mouse assessed across various tissues including liver (A), brain vasculature (B), ascending aorta (C), descending aorta (D), heart (E), lungs (F), kidney (G), bladder (H), small intestine (I), or large intestine (J).

FIGS. 44A-J. Bystander editing of ACTA2-M178V via AAV9 delivery at P14, cohort 3. A-to-G editing at A-1 to create ACTA2-M178V mutations in humanized mouse models of ACTA2 R179H following delivery of the split ABE8e-SpCas9-VRQR(S55R) base editor along with the A4 NGA PAM gRNA via AAV9. Mice from this cohort were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜2.5 e10 vg/mouse total) at 14 days of age. Tissues were collected at 8 weeks of age. Editing was assessed by amplicon sequencing with quantification of the percentage of A-to-G editing at M178V (converting ATG to GTG) versus control samples. Dots for n=1, editing for each individual mouse assessed across various tissues including liver (A), brain vasculature (B), ascending aorta (C), descending aorta (D), heart (E), lungs (F), kidney (G), bladder (H), small intestine (I), or large intestine (J).

FIGS. 45A-J. Bystander editing of ACTA2-D181G via AAV9 delivery at P14, cohort 3. A-to-G editing at A10 to create ACTA2-D181G mutations in humanized mouse models of ACTA2 R179H following delivery of the split ABE8e-SpCas9-VRQR(S55R) base editor along with the A4 NGA PAM gRNA via AAV9. Mice from this cohort were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜2.5 e10 vg/mouse total) at 14 days of age. Tissues were collected at 8 weeks of age. Editing was assessed by amplicon sequencing with quantification of the percentage of A-to-G editing at D181G (converting GAT to GGT) versus control samples. Dots for n=1, editing for each individual mouse assessed across various tissues including liver (A), brain vasculature (B), ascending aorta (C), descending aorta (D), heart (E), lungs (F), kidney (G), bladder (H), small intestine (I), or large intestine (J).

FIGS. 46A-J. Bystander editing of ACTA2-L180M/V/L via AAV9 delivery at P14, cohort 3. C-to-A, C-to-G, or C-to-T editing at C6 to create ACTA2-L180M, -L180V, or a silent-L180L mutation in humanized mouse models of ACTA2 R179H following delivery of the split ABE8e-SpCas9-VRQR(S55R) base editor along with the A4 NGA PAM gRNA via AAV9. Mice from cohort 3 were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜2.50 e10 vg/mouse total) at 14 days of age. Tissues were collected at 8 weeks of age. Editing was assessed by amplicon sequencing with quantification of the percentage of C-to-A/G/T editing at L180 (converting CTG to ATG/GTG/TTG, respectively) versus control samples. Dots for n=1, editing for each individual mouse assessed across various tissues including liver (A), brain vasculature (B), ascending aorta (C), descending aorta (D), heart (E), lungs (F), kidney (G), bladder (H) small intestine (I), and large intestine (J).

FIGS. 47A-J. Insertions and deletions mutations via AAV9 delivery at P14, cohort 3. Levels of insertion or deletion mutations (indels) observed in humanized mouse models of ACTA2 R179H following delivery of the split ABE8e-SpCas9-VRQR(S55R) base editor along with the A4 NGA PAM gRNA via AAV9. Mice from this cohort were injected via retro-orbital injection into Acta2R179Hfl/+ control and Myh11-Cre:Acta2R179Hfl/+ mutant mice using a 1:1 ratio of N-terminal to C-terminal AAVs at a dose of 1.3 e13 vg/kg of each AAV per mouse (˜2.5 e10 vg/mouse total) at 14 days of age. Tissues were collected at 8 weeks of age. Editing was assessed by amplicon sequencing with quantification of the percentage of indels versus control samples. Dots for n=1, editing for each individual mouse assessed across various tissues including liver (A), brain vasculature (B), ascending aorta (C), descending aorta (D), heart (E), lungs (F), kidney (G), bladder (H), small intestine (I), or large intestine (J).

FIG. 48. A schematic illustration of methods described herein for modeling and correcting the ACTA2 R179H mutation via genome editing. First, a model cell line harboring an ACTA2 R179H mutation was created using prime editing. Shown are SEQ ID NOs. 29 (GCCATCATGCGTCTGGATCTG): 50 (AIMRLDL); 30 (GCCATCATGCATCTGGATCTG); and 51 (AIMHLDL.

FIG. 49. Schematic showing the sequence region of ACTA2 exon 5 including the R179H mutation, with two potential target sites that can be bound by versions of Cas9 (VRQR and WT SpCas9) that can be used to make the intended edit, with potential bystander edit M178V; shown are SEQ ID NOs: 53 and 54. To the right is a graph showing percent A-to-G editing to correct ACTA2 R179H, using ABE8e-SpCas9 (“WT”), ABE8e-SpCas9-VRQR (“VRQR”), or ABE8e-SpCas9-VRQR(S55R) (“VRQR+”), demonstrating improved A-to-G editing efficiency and minimized M178V bystander editing with VRQR+ in plasmid transfections of our ACTA2 R179H HEK 293T cell line. The gRNAs used for each base editor are indicated below the x-axis, where the A4 gRNA positions the target adenine in position 4 of the target site spacer (counting from the PAM distal end): the A8 gRNA positions the target adenine in position 8 of the spacer.

FIG. 50. Schematic representation of the human HDAC9 promoter and identified Transcription factor binding sites (TFBs). HDAC9 fragment 2 (P2) containing ˜1200 bp was further divided into 3 new fragments of about ˜330 bp (P2.1, P2.2 and P2.3).

FIG. 51. Analysis of RFP expression by microscopy in wild-type HEK cells: 2 ug each of plasmids containing P2.1, P2.2 and P2.3 driving expression of red fluorescent protein (RFP) were transfected in wild-type HEK cells. 72 hrs. post transfection, cells were fixed and analyzed on a fluorescent microscope. Results show promoter activity for P2.1 and P2.2. as observed by the red color intensity. P2.3 showed lower activity. Ctrl group represents untransfected cells.

FIGS. 52A-B. Analysis of RFP expression by western blot in HEK cells expressing the MSMDS-causing mutation: 2 ug of each plasmids containing P2.1, P2.2 and P2.3 were transfected in wild-type and mutant (R179H) HEK cells. 72 hrs. post transfection cells total lysates were used to blot against RFP. In correlation with the microscopy results, P2.1 and P2.2 showed higher promoter activity.

It should be understood that the drawings and pictures are not necessarily to scale.

### DEFINITIONS AND ABBREVIATIONS

Unless otherwise defined, all technical and scientific terms used herein have the same meaning as commonly understood by one of ordinary skill in the art to which this invention belongs. Methods and materials are described herein for use in the present invention: other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.
MSMDS, as used herein, is an abbreviation for multisystemic smooth muscle dysfunction syndrome.
MFS, as used herein, is an abbreviation for Marfan syndrome.
SCAD, as used herein, is an abbreviation for Spontaneous Coronary Artery Dissection.
MS, as used herein, is an abbreviation for Myhre syndrome.

As used herein, the following are abbreviations for the Animal model genotypes of the aforementioned conditions: MSMDS: Myh11-Cre:Acta2R179Hfl/+. Marfan Syndrome: Fbn1^{C1039G/+}. SCAD: Col3a1^{+/-}:Col5a1^{+/-}. Myhre Syndrome: Myh11-Cre:Smad4V499Ifl/+.
SMC, as used herein, is an abbreviation for smooth muscle cell.
CMV, as used herein, is an abbreviation for cytomegalovirus.
BPNLS, as used herein, is an abbreviation for bipartite SV40 nuclear localization signal.
ABE8e, as used herein, is an abbreviation for adenosine deaminase domain version 8e.
EGFP, as used herein, is an abbreviation for enhanced green fluorescent protein;
bGH pA, as used herein, is an abbreviation for bovine growth hormone polyadenylation signal;
ITR, as used herein, is an abbreviation for inverted terminal repeat;
CBh, as used herein, is an abbreviation for chicken β-actin hybrid promoter;
NpuN and NpuC, as used herein, are abbreviations for the N- and C-terminal domains of the split alpha subunit of the DNA polymerase III (DnaE) intein from Nostoc punctiforme PCC73102 (Npu); woodchuck hepatitis virus posttranscriptional regulatory element (WPRE).

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH₂). The term “C-terminus” relates to the end of an amino acid chain (protein or polypeptide), terminated by a free carboxyl group (—COOH).

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using the GAP program using the default parameters, e.g., a Blossum 62 scoring matrix with a gap penalty of 12, a gap extend penalty of 4, and a frameshift gap penalty of 5; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “in vitro” includes artificial environments and to processes or reactions that occur within an artificial environment (e.g., a test tube). The term “in vivo” includes natural environments (e.g., a cell or organism or body) and to processes or reactions that occur within a natural environment. The term “ex vivo” includes cells that have been removed from the body of an individual (mouse or human) and to processes or reactions that occur within such cells.

Unless otherwise apparent from the context, the term “about” encompasses values within a standard margin of error of measurement (e.g., SEM) of a stated value.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

### DETAILED DESCRIPTION

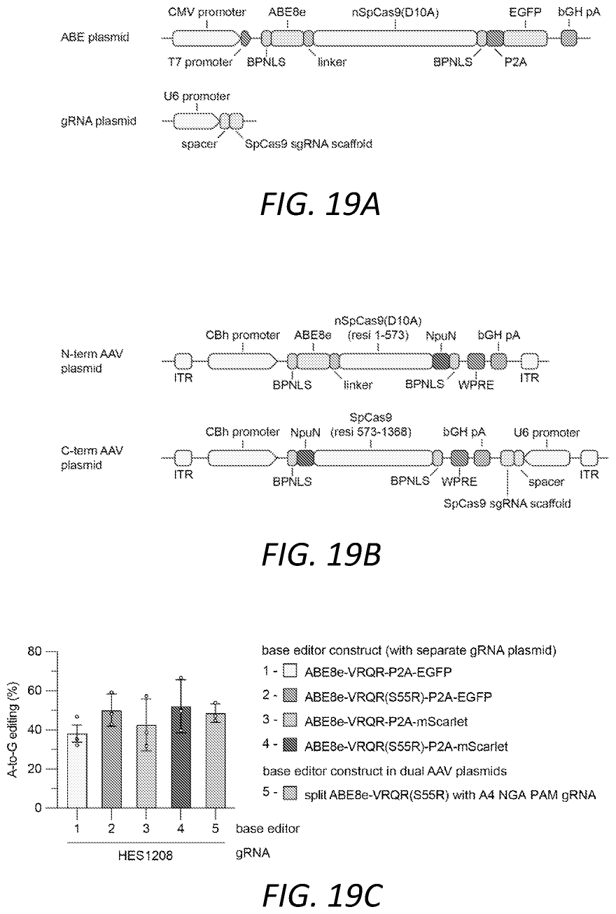

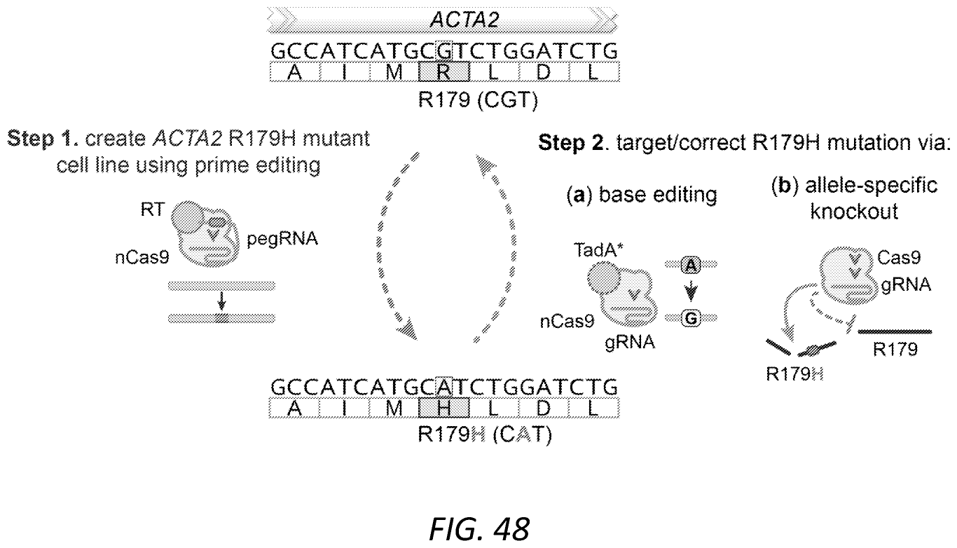

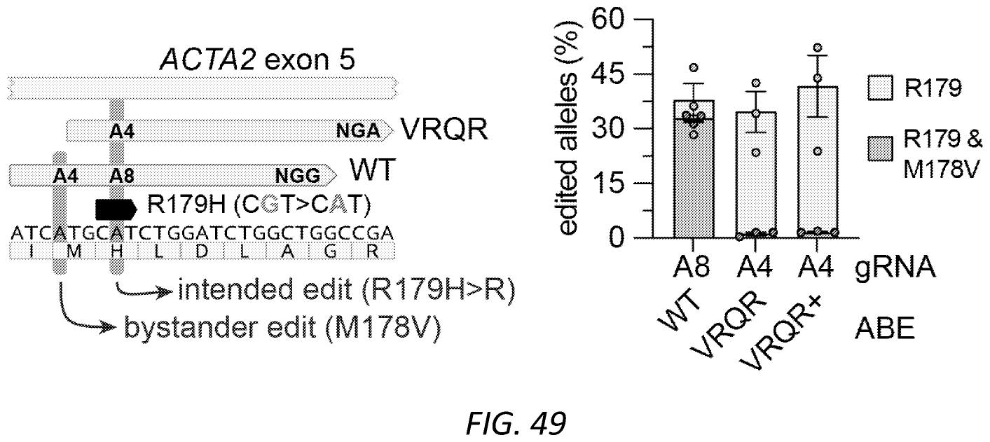

Multisystemic smooth muscle dysfunction syndrome (MSMDS, OMIM #613834) is an ultrarare genetic smooth muscle myopathy with major dysfunction in the vascular, respiratory, enteric, and genitourinary systems. Cases described to date have been monogenic, associated with the missense variation at arginine 179 of the ACTA2 gene, most commonly creating a missense variant (R179H). Since this disease is caused by a single base G-to-A mutation, it is a potential target for gene knockout or correction via CRISPR technologies. To scalably evaluate effective genome editing approaches to correct the ACTA2^{R179H} mutation, we first developed a clonal HEK 293T cell line bearing the R179H substitution (FIG. 48). We designed and tested various combinations of prime editors and pegRNAs, selecting the most optimal pair to generate several ACTA2^{R179H} cell lines of varying zygosity. Using heterozygous or homozygous cell lines, we evaluated dozens of CRISPR-Cas strategies to knockout or correct the mutant allele, via nucleases and base editors, respectively (FIG. 48). With adenine base editors (ABEs), we achieved high levels of A-to-G correction of the disease-causing G-to-A mutation (as illustrated in FIG. 49), when paired with different gRNAs that position the edit window of the ABE deaminase domain over the mutated nucleotide (relying on either WT SpCas9 or our engineered PAM variants). Sequencing from transfections in the ACTA2^{R179H} cell line revealed that although ABE8e-SpCas9 could correct the mutation, the intended edit was accompanied by very high levels of unwanted editing of nearby ‘bystander’ adenines. However, an SpCas9 PAM variant paired with an alternate gRNA (shifting the edit window of the base editor) enabled efficient and specific correction of the target base with minimal bystander editing. To investigate the translatability of our base editing approach in vivo, we created a knock in murine model of MSMDS with an exon advancement strategy that allows for controlled cre-inducible mutant allele expression (Acta2^{R179H/fl}). The mutant allele in heterozygosity was activated using Cre recombinase driven by the smooth muscle cell (SMC) specific Myh11 promoter. Myh11-Cre:Acta2^{R179Hfl/+} mice exhibit multiple phenotypes consistent with MSMDS including neurovascular dysfunction with neurodegeneration, aortic enlargement, intestinal dysmotility, and premature death. For in vivo delivery of the ABE, we cloned an intein-split construct compatible with dual adeno-associated virus (AAV) delivery (illustrated in FIGS. 19A-B). The constructs were packaged in two AAV serotypes, including AAV9 and our novel AAV capsid optimized for murine vascular delivery. Analysis of Myh11-Cre:Acta2^{R179Hfl/+} mice retro-orbitally injected at P3 with dual AAVs revealed highly efficient and specific genomic correction of the R179H causing mutation in multiple tissues (˜60% in hepatocytes). Importantly, the P3 injected mice exhibited a dramatic phenotypic recovery, including improved weight gain, rotarod and open field performance, reduced aortic diameters, and vastly extended lifespan. Described herein is an efficient therapeutic approach to improve clinical outcomes in MSMDS via in vivo base editing.

### I. Overview

All of the functionalities described in connection with one embodiment of the methods, compositions, or formulations described herein are intended to be applicable to the additional embodiments of the methods, compositions, or formulations described herein except where expressly stated or where the feature or function is incompatible with the additional embodiments. For example, where a given feature or function of component is expressly described in connection with one embodiment but not expressly mentioned in connection with an alternative embodiment, it should be understood that the feature or component may be deployed, utilized, or implemented in connection with the alternative embodiment unless the feature or component is incompatible with the alternative embodiment.

Provided herein are nucleic acid sequences comprising a HDAC-9 derived promoter for driving expression of a transgene in a diseased cell. In some cases, such nucleic acid sequences comprising the HDAC9-derived promoters are encapsulated by an AAV vector that further facilitates delivery of such nucleic acids to a target cell.

Provided herein are gene-targeted therapies that use a viral vector, e.g., a smooth muscle cell-specific viral vector (which preferentially transduces smooth muscle cells), preferably in combination with a HDAC9-derived promoter to transfect SMC and deliver: 1) a base editor that selectively corrects mutant alleles in SMCs; or 2) a Cas-driven selective mutant allele deletion. An in vivo selection of an AAV9 capsid-based peptide library that traffics to brain after intravenous (iv) delivery yielded AAV-PR as a candidate for selective transfection of endothelium and SMC. The present disclosure provides a class of AAV9 capsid-based peptide modified virus, including AAV-PR, that selectively transfects endothelium, pericytes and SMC after intravenous (iv) delivery. Such AAV's can also use an HDAC9-derived promoter as described herein.

### II. Vessel-Targeted Adeno-Associated Viral Gene Delivery Vector

Viral vectors for use in the present methods and compositions include recombinant retroviruses, adenovirus, adeno-associated virus, alphavirus, and lentivirus, comprising the targeting peptides described herein and optionally a transgene for expression in a target tissue.

A preferred viral vector system useful for delivery of nucleic acids in the present methods is the adeno-associated virus (AAV). AAV is a tiny non-enveloped virus having a 25 nm capsid. No disease is known or has been shown to be associated with the wild-type virus. AAV has a single-stranded DNA (ssDNA) genome. AAV has been shown to exhibit long-term episomal transgene expression, and AAV has demonstrated excellent transgene expression in the brain, particularly in neurons. Space for exogenous DNA in AAV is generally limited to an amount of nucleic acid that can physically fit inside the particle. For example, AAV types 1-5 can package up to 6 kb DNA, and in some reports AAV5 has been shown to package up to 8.9 kb DNA. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985): Wondisford et al., Mol. Endocrinol. 2:32-39 (1988): Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. The present disclosure contemplates uses of peptides that can be incorporated into an AAV capsid-thus providing capsid modified AAVs, e.g., AAV-PR—for selectively transfecting endothelium, pericytes and SMC after delivery to a subject. Such AAV's can also be used for delivery of a nucleic acid comprising an HDAC9-derived promoter as described herein. In some embodiments, an AAV suitable for use with a nucleic acid or a targeting peptide of the disclosure is AAV1, AAV2, AAV3, AAV4, AAV5, AAV6, AV6.2, AAV7, AAV8, rh.8, AAV9, rh.10, rh.39, rh.43 or CSp3; for CNS use, in some embodiments the AAV is AAV1, AAV2, AAV4, AAV5, AAV6, AAV8, or AAV9.

In preferred embodiments, the present methods use vessel-specific viral vectors that have been shown to transduce cerebral vasculature in large mammals and humans, including AAV such as AAV2 or 9, as well as capsid-modified AAVs that have improved specificity, transient expression, and/or higher transduction efficiency for SMCs including AAV9-PR, a modified version of AAV9 described herein and in WO2022232327 (which is incorporated by reference herein in its entirety). AAV-PR comprises the sequence PRPPSTH (SEQ ID NO: 1) in the capsid (i.e., inserted into the VP1 protein in a position corresponding to amino acids 588 and 589 of SEQ ID NO:2); alternatively, AAV-MA (comprising the sequence MAEPGAR (SEQ ID NO:25)), AAV-ML (comprising the sequence MLYADNT (SEQ ID NO:26), or AAV-SQ (comprising the sequence SQDPSTL (SEQ ID NO:27) inserted into the VP1 protein in a position corresponding to amino acids 588 and 589 of SEQ ID NO:2). AAV-PR was shown to traffic to brain after intravenous (iv) delivery, with highly efficient transduction of endothelium, pericytes and only sparsely astrocytes, and no transduction of glial or neuronal cells. AAV-PR transduced the intima of capillaries, perforating arterioles and subarachnoid cerebral arteries (GFP) and vascular smooth muscle cells of cerebral arteries (SMCs).

An exemplary wild type AAV9 capsid protein VP1 (Q6JC40-1) sequence is as follows:

```
(SEQ ID NO: 2)
                10 20 30 40 50
MAADGYLPDW LEDNLSEGIR EWWALKPGAP QPKANQQHOD NARGLVLPGY
                60 70 80 90 100
KYLGPGNGLD KGEPVNAADA AALEHDKAYD QQLKAGDNPY LKYNHADAEF
              110 120 130 140 150
QERLKEDTSF GGNLGRAVFQ AKKRLLEPLG LVEEAAKTAP GKKRPVEQSP
              160 170 180 190 200
QEPDSSAGIG KSGAQPAKKR LNFGQTGDTE SVPDPQPIGE PPAAPSGVGS
              210 220 230 240 250
LTMASGGGAP VADNNEGADG VGSSSGNWHC DSQWLGDRVI TTSTRTWALP
              260 270 280 290 300
TYNNHLYKQI SNSTSGGSSN DNAYFGYSTP WGYFDFNRFH CHFSPRDWQR
              310 320 330 340 350
LINNNWGFRP KRLNFKLFNI QVKEVTDNNG VKTIANNLTS TVQVFTDSDY
              360 370 380 390 400
QLPYVLGSAH EGCLPPFPAD VEMIPQYGYL TLNDGSQAVG RSSFYCLEYF
              410 420 430 440 450
PSQMLRTGNN FQFSYEFENV PFHSSYAHSQ SLDRLMNPLI DQYLYYLSKT
              460 470 480 490 500
INGSGQNQQT LKFSVAGPSN MAVQGRNYIP GPSYRQQRVS TTVTQNNNSE
              510 520 530 540 550
FAWPGASSWA LNGRNSLMNP GPAMASHKEG EDRFFPLSGS LIFGKQGTGR
              560 570 580 590 600
DNVDADKVMI TNEEEIKTTN PVATESYGQV ATNHQSAQAQ AQTGWVQNQG
              610 620 630 640 650
ILPGMVWQDR DVYLQGPIWA KIPHTDGNFH PSPLMGGFGM KHPPPQILIK
              660 670 680 690 700
NTPVPADPPT AFNKDKLNSF ITQYSTGQVS VEIEWELQKE NSKRWNPEIQ
              710 720 730
YTSNYYKSNN VEFAVNTEGV YSEPRPIGTR YLTRNL
```

Thus provided herein are AAV that include a capsid protein comprising a targeting peptide sequence of SEQ ID NO:1, preferably wherein the targeting peptide sequence has been inserted into the sequence of AAV9 VP1, e.g., between amino acids 588 and 589.

An exemplary amino acid sequence of AAV9 VP1 comprising the AAV-PR targeting sequence (shown in bold, lower case) is as follows:

(SEQ ID NO: 3)
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQprppsthAQAQT
GWVQNQGILPGMVWQDRDVYLQGPIWAKIPHTDGNFHPSPLMGGFGMKHP
PPQILIKNTPVPADPPTAFNKDKLNSFITQYSTGQVSVEIEWELQKENSK
RWNPEIQYTSNYYKSNNVEFAVNTEGVYSEPRPIGTRYLTRNL

In some of the present methods and compositions, the AAV can include a transgene sequence encoding a gene editor, e.g., an ACTA2 editor as described herein (e.g., a base editor for correcting the R179H mutation, or a Clustered Regularly Interspaced Short Palindromic Repeats (CRISPR) Cas nuclease specifically targeting an R179H mutant allele) or as known in the art, or a reporter protein, e.g., a fluorescent protein, an enzyme that catalyzes a reaction yielding a detectable product, or a cell surface antigen. The transgene is preferably linked to sequences that promote/drive/regulate expression of the transgene in the target tissue during diseased states, e.g., an HDAC9 or minimal HDAC9 promoter as described herein.

The virus can also include one or more sequences that promote expression of a transgene, e.g., one or more promoter sequences: enhancer sequences, e.g., 5′ untranslated region (UTR) or a 3′ UTR: a polyadenylation site; and/or insulator sequences. In some embodiments, the promoter is a vascular endothelial cell-specific promoter, e.g., VE-cadherin promoter, fms-like tyrosine kinase-1 (FLT-1), intercellular adhesion molecule-2 (ICAM-2), a Claudin 5 (CLDN-5), a von Willebrand factor (vWF) promoter, a TIE2 promoter, or a synthetic EC-specific promoter (see, e.g., Dai et al., J Virol. 2004 June; 78(12): 6209-6221) or SMC-specific promoter as described herein. In some embodiments, the promoter is a pan-cell type promoter, e.g., a “ubiquitous” promoter that drives expression in most cell types, e.g., cytomegalovirus (CMV) promoter (optionally with the CMV enhancer), chicken beta-actin (CBA) promoter, Rous sarcoma virus (RSV) LTR promoter (optionally with the RSV enhancer), SV40 promoter, dihydrofolate reductase promoter, phosphoglycerol kinase promoter, phosphoglycerol kinase (PGK) promoter, EF1alpha promoter, Ubiquitin C (UBC), B-glucuronidase (GUSB), and CMV immediate/early gene enhancer/CBA promoter (CAG); or a steroid promoter or metallothionein promoter. The woodchuck hepatitis virus posttranscriptional response element (WPRE) can also be used.

Other AAV that can be used in the present methods and compositions include AAV2.5 (generated from the AAV2 capsid with five mutations from AAV1, see Bowles et al., Mol Ther. 2012 February; 20(2):443-55), or AAV2/5 (with portions from AAV2 (ITRs and Rep) and AAV5 (Cap), see Hildinger et al., J Virol. 2001 July; 75(13):6199-203).

### III. HDAC9-Derived Promoters

In gene therapy; delivered genetic payloads must both be delivered to the target cells and contain useful regulatory elements that control expression. For instance in gene replacement therapy, a gene product would be expressed in the cell type deficient for the product, but would also be expressed at normal physiologic levels. The ability to take advantage of innate transcriptional mechanisms will be an advantage to gene delivery therapies. For gene editing therapeutics the challenges are somewhat different. Delivery of DNA that encodes gene editing enzymes, such as CRISPR and their derivatives, may benefit from high transient expression in target cells with silencing after the genomic editing is accomplished. For this task, expression mechanisms that are specific to the disease (e.g., expression is increased in the disease state but quiescent in cells that have become normal after gene editing has occurred) could be advantageous to existing technologies.

To further the advancement of gene therapies in vascular disorders, the disclosure identifies and demonstrates that a disease-associated transcriptional control element, namely the promoter of the histone deacetylase 9 gene, HDAC9, and fragments thereof are effective in triggering expression of a transgene in smooth muscle cells: in some conditions, this occurs in a disease specific manner, e.g., preferentially in SMCs affected by vascular disease. The acetylation and deacetylation of histones are critical determinants of chromatin structure, gene transcription, and cellular phenotype and allow for the coupling of extracellular signals with genomic architecture.³⁰ Histone deacetylases remove acetyl groups from histones and consist of a superfamily of 11 enzymes that are further subdivided into 4 families (HDAC class I, IIa, IIb and IV).31 HDAC9 belongs to the class Ila HDAC family,² and has been implicated through human genetics in multiple human vascular diseases involving VSMC function, including thoracic aortic aneurysm, abdominal aortic calcification, hypertension, intracranial aneurysms, ischemic stroke, and myocardial infarction.³²⁻²⁶ In previous work, we demonstrated upregulation of the HDAC9 transcript, as well as HDAC9 protein during vascular disease induced by mutations in genes causing thoracic aortic aneurysm (TAA). Modeling two aggressive forms of hereditary TAA, by lentiviral expression in human aortic VSMCs, (TGFR2^{G357W}, a severe Loeys-Dietz syndrome allele and ACTA2^{R179H} which causes a severe smooth muscle cell dystrophy),³⁷⁻³⁸ we observed strong upregulation of HDAC9 transcript in the diseased state in addition to changes in cellular morphology and migration.^{36,39} Consistent with cellular morphologic changes, we found increased transcriptional activity at the HDAC9 locus was closely linked to the ability of TAA-inducing mutations to disrupt the actin cytoskeleton. VSMCs expressing disease-associated alleles showed a higher ratio of depolymerized (G-actin) to polymerized (F-actin) actin upon direct examination. Indeed, actin depolymerizing agents such as latrunculin were able to induce HDAC9 transcriptional activity; independently of gene variants.

As shown herein, we mapped several regions of the HDAC9 promoter, identifying smaller regions (also referred to herein as P2 and P3, as well as subfragments P2.1, P2.2, and P2.3) that can be used to drive transgene expression in VSMCs. In some embodiments, this promoter is inducible by actin depolymerizing agents. We then transduced this region into VSMCs in several genetic vascular disease models and demonstrated disease-induced reporter expression. The sequences of the full length HDAC9 promoter (prom 1 or P1), as well as two fragments P2 (prom2) and P3 (prom3), and subfragments P2.1. P2.2, and P2.3 are as follows:

prom1 (2541 bp)
(SEQ ID NO: 4)
ataaatgttttgtagaataaaaaaaaaaaaagttcttcaaaagaaatctcaaatctccaaatggaaacaggtaaa
agtggagctcccctggttccacggagaaccttttttgaggaaacttaggcaactcgcaggtaccttatgtcatga
gacagagtttgaaaactacaattgactatctctaaatttcctcccaggtctaaaatgtgatgatagttactactt
cagtacatcatccttaaggaaaattattaggtccacactgtttctatcctttgaattttacacataaattttgta
atcaaaagtttatttgtaatatcagatggaatcagataattgctttttgttttttccactgacaggaacataaga
ttttgttgtgtagcttaagtcaaacgcagtttggaatatatattttttaaaaattgtaacttacatatccaaata
caatttttcaagaagtagagtattcagtagaaattaatctgtgaaagaagaggaattcagcagtggcctatttga
tgaatgatttaacgtgcttatttcttccctttcatcaaaactctgtgtccccttgtttgccccctctgacttcat
actctggagttgaccaagatccctcttccatcggattgttctgggaattttgaaataatctgctttttcctctct
ttcccctgttgcttctgatgccttagaattacattttcctcgctgatttagtttagaaaagagaaaagagcttcc
atgactagtagattatcacttttgggtttgctcttggaagtgacaagatgctaggatccctctttggaatgtaaa
atttatctcttatatagaaaggatataaatgtagcaccagagactataaaactctgatactatctactgtactgt
atagctgaacgccacaatgtgtctggtaatctattgactatcataaatgctatttctacagaaaagttaggaggt
ccatatttcgggcaaccaatgtatagctgaatgcagaacagtcatagttgggtactaaccatatatatgatttat
ccatcaacaggtgcatatgctcagaaattctgtatccataagaaatcagactactttcttttccttttgcaagta
aattgaatttagcctgagaggctgaggggaaattttcacatataagccacggttttgtgttttgtgttttgtttt
gtttatagatatagtactaactggatggatgcgataaaattcataggtggtactaagatacaataggatttgtga
aatggacaattgtcttgcataaatagcaagtaaaaaatcaagcctgtccttcataaaaattttattctggggtgt
gcttgttttccaaaagtacctctgctaaatctcctgttagtcctgaaactagaaggcagaaaagcttctagtgct
acagccaactgcagtgtagcctgagaaacaggcaacaaaaatagaacaccaggattgctgtgcgtgggtgaggca
gaaaccacattatgagcaaaagcttccagtattattttagaaccaatacagagctctgtacttctctcctctctc
ttccaaaaacacatactacaaaaataaaatgaaatgaaatgtatgtgcatttgccctcttagaattatgattctt
aattttttttcttgccttcctttctttggaagcgaatcgccagtatggaaacacagtgtgtaaagcaagcttcga
gagaggaaagagttaattggttttaaggccctgcgatagagaattatggttggaaagatagaggctggacagctg
ggtttgctggggtatttttaaatgcattaatgcaggctccaatcactcggccatgcttgacctatttttggctca
ggccgaccattgttctatttctgtgcctgtgggccatgctgttgttgattcatatgcaaatggattatcactcgc
tttagccaacttgagctgagagagactgagaaagggggaagagaggcacagacacagataggagaagggcaccgg
ctggagccacttgcaggactgagggtttttgcaacaaaaccctagcagcctgaagaactctaagccaggtttaat
tggtttctttttctcgtgggtagacttaataattttctacgtattctgacaaagaaataaccccgaagcacgttc
ctatttcccacctgcttgtagtttccgggataacctaaactccagagagctatagcatccactctgtcctttctg
ctttgcacacaggttggtaacatgggaaaagtgtccaggtctttttaaaagtggatgcccatttgagcagaaagg
aaatcattgtcgaagttgatcctctgctgcttctcctcagggaggagggagaaccagcgagggtagctcctgggg
ccggtgcactgagcagtgatgaatgtttcatgtagctgaagtaagagtgactggaatatgctgcagacaatttac
gagagtgactcctgtttttcctcag
Prom2 (1350 bp)
(SEQ ID NO: 5)
ataaatgttttgtagaataaaaaaaaaaaaagttcttcaaaagaaatctcaaatctccaaatggaaacaggtaaa
agtggagctcccctggttccacggagaaccttttttgaggaaacttaggcaactcgcaggtaccttatgtcatga
gacagagtttgaaaactacaattgactatctctaaatttcctcccaggtctaaaatgtgatgatagttactactt
cagtacatcatccttaaggaaaattattaggtccacactgtttctatcctttgaattttacacataaattttgta
atcaaaagtttatttgtaatatcagatggaatcagataattgctttttgttttttccactgacaggaacataaga
ttttgttgtgtagcttaagtcaaacgcagtttggaatatatattttttaaaaattgtaacttacatatccaaata
caatttttcaagaagtagagtattcagtagaaattaatctgtgaaagaagaggaattcagcagtggcctatttga
tgaatgatttaacgtgcttatttcttccctttcatcaaaactctgtgtccccttgtttgccccctctgacttcat
actctggagttgaccaagatccctcttccatcggattgttctgggaattttgaaataatctgctttttcctctct
ttcccctgttgcttctgatgccttagaattacattttcctcgctgatttagtttagaaaagagaaaagagcttcc
atgactagtagattatcacttttgggtttgctcttggaagtgacaagatgctaggatccctctttggaatgtaaa
atttatctcttatatagaaaggatataaatgtagcaccagagactataaaactctgatactatctactgtactgt
atagctgaacgccacaatgtgtctggtaatctattgactatcataaatgctatttctacagaaaagttaggaggt
ccatatttcgggcaaccaatgtatagctgaatgcagaacagtcatagttgggtactaaccatatatatgatttat
ccatcaacaggtgcatatgctcagaaattctgtatccataagaaatcagactactttcttttccttttgcaagta
aattgaatttagcctgagaggctgaggggaaattttcacatataagccacggttttgtgttttgtgttttgtttt
gtttatagatatagtactaactggatggatgcgataaaattcataggtggtactaagatacaataggatttgtga
aatggacaattgtcttgcataaatagcaagtaaaaaatcaagcctgtccttcataaaaattttattctggggtgt
Prom3 (1191 bp)
(SEQ ID NO: 6)
gcttgttttccaaaagtacctctgctaaatctcctgttagtcctgaaactagaaggcagaaaagcttctagtgct
acagccaactgcagtgtagcctgagaaacaggcaacaaaaatagaacaccaggattgctgtgcgtgggtgaggca
gaaaccacattatgagcaaaagcttccagtattattttagaaccaatacagagctctgtacttctctcctctctc
ttccaaaaacacatactacaaaaataaaatgaaatgaaatgtatgtgcatttgccctcttagaattatgattctt
aattttttttcttgccttcctttctttggaagcgaatcgccagtatggaaacacagtgtgtaaagcaagcttcga
gagaggaaagagttaattggttttaaggccctgcgatagagaattatggttggaaagatagaggctggacagctg
ggtttgctggggtatttttaaatgcattaatgcaggctccaatcactcggccatgcttgacctatttttggctca
ggccgaccattgttctatttctgtgcctgtgggccatgctgttgttgattcatatgcaaatggattatcactcgc
tttagccaacttgagctgagagagactgagaaagggggaagagaggcacagacacagataggagaagggcaccgg
ctggagccacttgcaggactgagggtttttgcaacaaaaccctagcagcctgaagaactctaagccaggtttaat
tggtttctttttctcgtgggtagacttaataattttctacgtattctgacaaagaaataaccccgaagcacgttc
ctatttcccacctgcttgtagtttccgggataacctaaactccagagagctatagcatccactctgtcctttctg
ctttgcacacaggttggtaacatgggaaaagtgtccaggtctttttaaaagtggatgcccatttgagcagaaagg
aaatcattgtcgaagttgatcctctgctgcttctcctcagggaggagggagaaccagcgagggtagctcctgggg
ccggtgcactgagcagtgatgaatgtttcatgtagctgaagtaagagtgactggaatatgctgcagacaatttac
gagagtgactcctgtttttcctcag
H9P2.1-RFP
(SEQ ID NO: 85)
Ataaatgttttgtagaataaaaaaaaaaaaagttcttcaaaagaaatctcaaatctccaaat
ggaaacaggtaaaagtggagctcccctggttccacggagaaccttttttgaggaaacttagg
caactcgcaggtaccttatgtcatgagacagagtttgaaaactacaattgactatctctaaa
tttcctcccaggtctaaaatgtgatgatagttactacttcagtacatcatccttaaggaaaa
ttattaggtccacactgtttctatcctttgaattttacacataaattttgtaatcaaaagtt
tatttgtaatatcagatggaatcagataattgctttttgttttttccactgacaggaacata
agattttgttgtgtagcttaagtcaaacgcagtttggaatatatattttttaaaaattgtaa
H9P2.2-RFP
(SEQ ID NO: 86)
Cttacatatccaaatacaatttttcaagaagtagagtattcagtagaaattaatctgtgaaa
gaagaggaattcagcagtggcctatttgatgaatgatttaacgtgcttatttcttccctttc
atcaaaactctgtgtccccttgtttgccccctctgacttcatactctggagttgaccaagat
ccctcttccatcggattgttctgggaattttgaaataatctgctttttcctctctttcccct
gttgcttctgatgccttagaattacattttcctcgctgatttagtttagaaaagagaaaaga
gcttccatgactagtagattatcacttttgggtttgctcttggaagtgacaagatgctagga
tccctctttggaatgtaaaatttatctcttatatagaaaggatataaatgtagcaccagaga
ctataaaactctgatactatctactgtactgtatagctgaacgccaca
H9P2.3-RFP
(SEQ ID NO: 87)
Atgtgtctggtaatctattgactatcataaatgctatttctacagaaaagttaggaggtcca
tatttcgggcaaccaatgtatagctgaatgcagaacagtcatagttgggtactaaccatata
tatgatttatccatcaacaggtgcatatgctcagaaattctgtatccataagaaatcagact
actttcttttccttttgcaagtaaattgaatttagcctgagaggctgaggggaaattttcac
atataagccacggttttgtgttttgtgttttgttttgtttatagatatagtactaactggat
ggatgcgataaaattcataggtggtactaagatacaataggatttgtgaaatggacaattgt
cttgcataaatagcaagtaaaaaatcaagcctgtccttcataaaaattttattctggggtgt

Expression of a transgene, e.g., in an expression vector such as an AAV, e.g., an AAV as described herein, can be driven by these synthetic HDAC9-derived promoters developed based on an HDAC9 promoter, e.g., the HDAC9 P1, P2, or P3 promoter or a portion thereof described herein (e.g., SEQ ID NO:4, 5, 6, 85, 86, or 87), or a promoter that has at least 80, 85, 90, 85, or 99% identity to an HDAC9 P1, P2, or P3 promoter or a portion thereof as described herein (e.g., 80, 85, 90, 85, or 99% identity to SEQ ID NO:4, 5, 6, 85, 86, or 87), but still retains the ability to drive expression of a transgene in SMCs. In some embodiments the promoter comprises SEQ ID NO:4, 5, 6 (e.g., the full length of SEQ ID NO: 4, 5, 6, 85, 86, or 87), with 1, 2, 3, 4, 5, 6, 7, 8, 9, or 10, e.g., up to 10 or 20, mutations or deletions with respect the SEQ ID NO:4, 5, 6, 85, 86, or 87, so long as the promoter retains the ability to drive expression of a transgene in SMCs.

### ACTA2 Arg179—Multiystemic Smooth Muscle Dysfunction Syndrome (MSMDS)

Mutations in the actin alpha 2, smooth muscle (ACTA2) gene account for 16% of all cases of familial thoracic aortic aneurysms (560-1200 cases per year in the US), one of the most common autosomal dominant genetic smooth muscle cell (SMC) vasculopathies (Guo et al., Nat Genet. 2007 December; 39(12):1488-93). Pathogenic variants in the ACTA2 Arginine 179 (ACTA2 Arg179) cause the most severe form of the disease wherein multisystemic smooth muscle dysfunction (affecting the lungs, bladder, gut, and eyes) and a progressive vasculopathy lead to aortic dissections, strokes, and death in the first two decades of life (Milewicz et al., Am J Med Genet A. 2010 October; 152A(10):2437-43; Regalado et al., Genet Med. 2018 October; 20(10):1206-1215; Munot et al., Brain. 2012 August; 135(Pt 8):2506-14). While patients can be diagnosed at the bedside in the first week of life by congenital mydriasis in a newborn presenting with patent ductus arteriosus, aortic dissections and major strokes commonly occur after 3 years of age following significant relative stenosis of the terminal carotid artery (Lauer et al., Neurology. 2021 Jan. 26; 96 (4):e538-e552) and dilation of the aorta (Regalado et al., Genet Med. 2018 October; 20(10):1206-1215).

The involvement of brain vasculature and its onset during childhood distinguish ACTA2 Arg179 MSMDS from other ACTA2 mutations. The cerebrovascular disease is characterized by cerebral small vessel disease (cSVD), white matter injury, progressive steno-occlusive vasculopathy (Moyamoya-like disease) and recurrent arterial ischemic strokes. In ACTA2 R179H disease, ischemic strokes commonly occur during episodes of hypotension or anesthesia suggesting severe impairment of the cerebral autoregulation (CA) and neurovascular coupling (NC), mechanisms required to maintain cerebral blood flow (CBF) during changes in arterial blood pressure and cortical activity, respectively. The lack of collateral vessel neoformation (Munot et al., 2012) suggests that mutant SMC not only impair cerebrovascular autoregulation but also the vessel remodeling capacity.

At the molecular level, ACTA2 R179H acts in a dominant negative effect by disrupting fibrillar actin bundling (polymerized F-actin) and causing ineffective contractility of SMC. Mutant SMCs thus alter their transcriptional regulation switching to a proliferative and secretory phenotype that leads to: 1) narrowing and occlusion of muscular arteries: 2) dilatation of elastic arteries due to abnormal elastin and collagen deposition: 3) impaired vasoreactivity leading to low systemic blood pressure and inability to dynamically redistribute blood flow, and 4) microvascular and blood brain barrier (BBB) dysfunction (Georgescu et al., Acta Neuropathol Commun. 2015 Dec. 4; 3:81).

Currently available treatments cannot arrest vascular disease progression and most children with ACTA2 Arg179 mutations suffer strokes before 7 years of age and aortic dissection or death in the second decade of life. Moreover, only palliative treatments exist to decrease disabling symptoms caused by eye, lung, bladder and gut smooth muscle dysfunction.

### IV. Genome Editing Agents

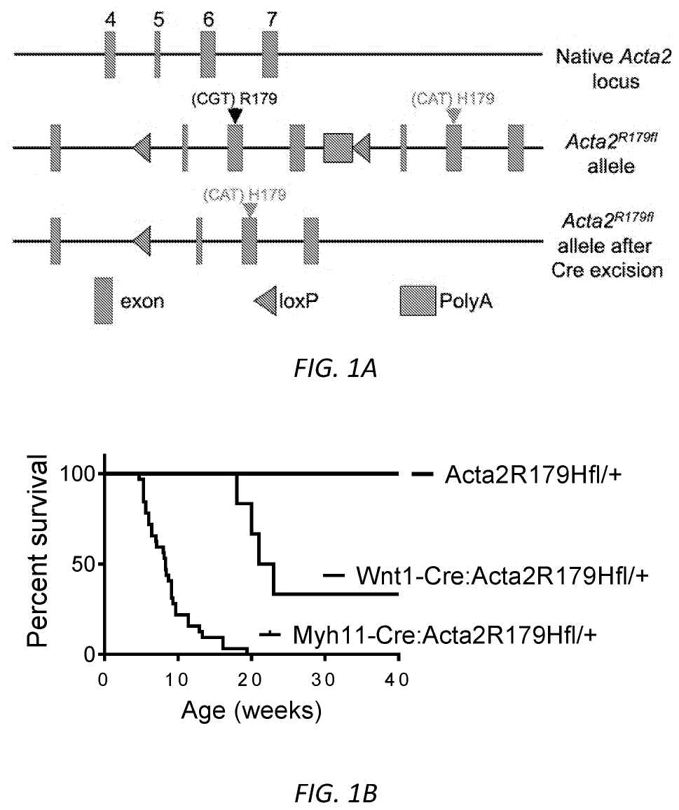

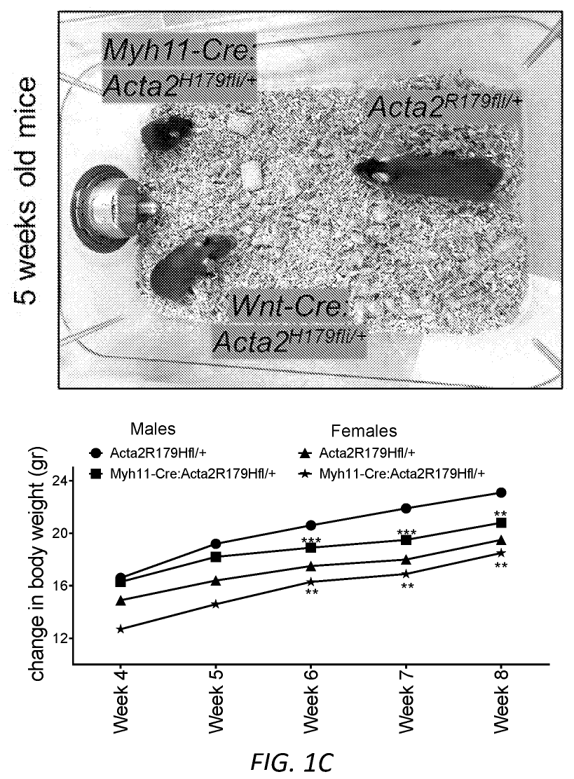

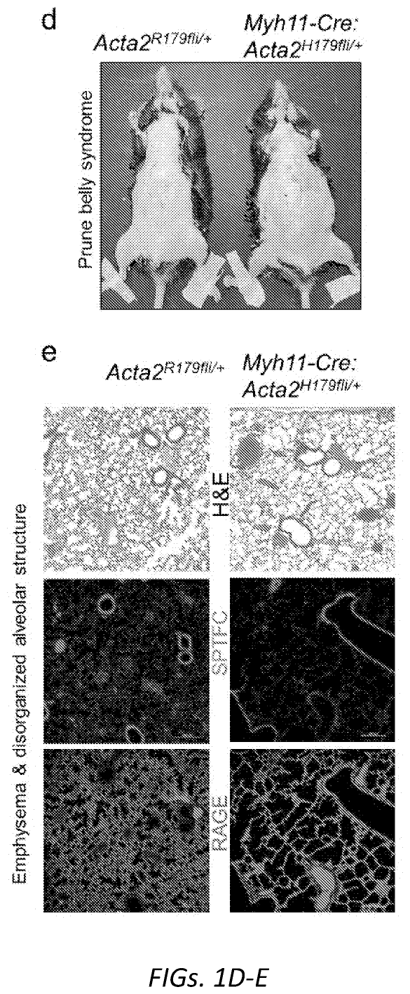

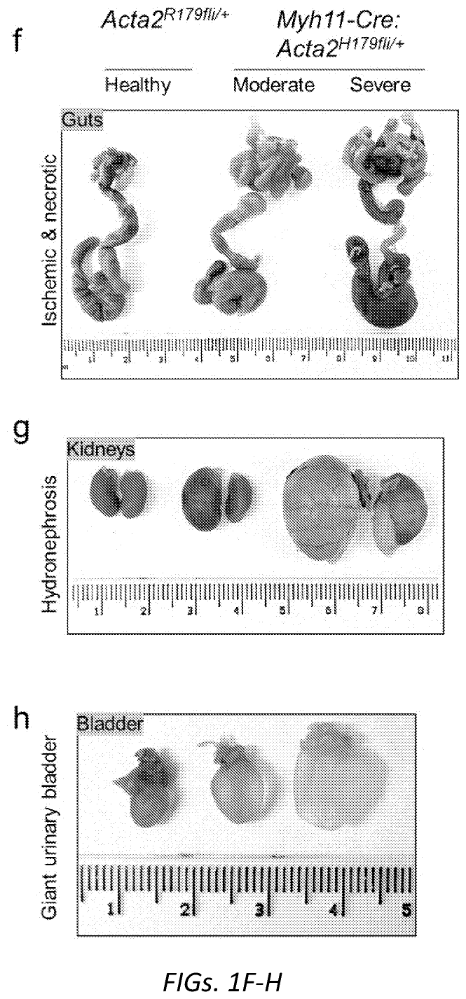

Genome editing technologies enable the permanent modification of DNA sequences in living cells. This process has been simplified by the discovery that CRISPR nucleases such as Cas9 can be readily programmed to edit DNA sites using a guide RNA (gRNA) 1.2. Once the Cas enzyme is complexed with the gRNA, the Cas9-gRNA ribonucleoprotein (RNP) molecule scans chromosomal sequences for a short protospacer-adjacent motif (PAM) directly beside the target site.³ The simplicity of re-targeting the Cas9 protein to new sites offers the ability to make precise changes to the genome. However, the requirement for the Cas9 protein to recognize a PAM fundamentally limits the breadth of editing. Recently developed CRISPR-Cas9 and -Cas 12a proteins with vastly expanded targeting ranges permit editing of previously inaccessible sequences can overcome this major limitation.⁴⁻⁸ Beyond traditional nuclease-based editing, cytosine base editors (CBEs) and adenine base editors (ABEs) have been recently developed that enable the introduction of precise C-to-T and A-to-G changes, respectively.⁹⁻¹² Additionally, CRISPR prime editors (PEs) permit the installation of custom changes into the genome by using a reverse transcriptase (RT) domain and a prime editor guide RNA (pegRNA; FIG. 1D).^{13,14} Thus, a number of options exist for using CRISPR-Cas enzymes for precise modelling or correction of disease-causing mutations. However, not all of these options are equally successful in generating specific changes with minimal off-target effects. Together, the continually expanding toolbox of CRISPR-Cas enzymes offers technologies that enable the precise modelling or correction of disease-causing mutations.

As shown herein, for correction of the R179H mutation an adenine base editor agent can include a base editor, e.g., a base editor comprising a catalytically dead Cas9 (dCas9) or a nickase Cas9 (nCas9) fused to a deaminase and guided by a single guide RNA (sgRNA) to a sequence of interest. In some embodiments, the deaminase comprises an engineered adenosine deaminase TadA monomer or dimer comprises a homodimeric or heterodimeric TadA domain from ABEmax, ABE7.10, or ABE8e: monomer or dimer TadA from ABE 0.1, 0.2, 1.1, 1.2, 2.1, 2.2, 2.3, 2.4, 2.5, 2.6, 2.7, 2.8, 2.9, 2.10, 2.11, 2.12, 3.1, 3.2, 3.3, 3.4, 3.5, 3.6, 3.7, 3.8, 4.1, 4.2, 4.3, 5.1, 5.2, 5.3, 5.4, 5.5, 5.6, 5.7, 5.8, 5.9, 5.10, 5.11, 5.12, 5.13, 5.14, 6.1, 6.2, 6.3, 6.4, 6.5, 6.6, 7.1, 7.2, 7.3, 7.4, 7.5, 7.6, 7.7, 7.8, 7.9, 7.10, ABEmax, ABE8.8, ABE8.13, ABE8.17, ABE8.20, ABE8e, ABE9, ABE9e, or K20A/R21A, V82G, or V106W variants thereof; E. coli TadA monomer, or homo- or heterodimers thereof fused to the N or C terminus, optionally comprising one or more mutations in either or both monomers, optionally TadA from miniABEmax-V82G, miniABEmax-K20A/R21A, miniABEmax-V106W, or another variant. In some embodiments, the Cas9 portion of the base editor comprises the D1135L/S1136W/G1218K/E1219Q/R1335Q/T1337R (SpG: see, e.g., WO 2021/151073), D1135V/G1218R/R1335Q/T1337R (VRQR: see, e.g., WO 2016/141224), S55R/D1135V/G1218R/R1335Q/T1337R (VRQR(S55R), also referred to as VRQR+: e.g. see Ref¹⁷), A61R/L1111R/D1135L/S1136W/G1218K/E1219Q/N1317R/A1322R/R1333P/R1335 Q/T1337R (SpRY: see, e.g., WO 2021/151073), D10T (optional)/1322V/S409I/E427G/R654L/R753G/R1114G/D1135N/V1139A/D1180G/E1 219V/Q1221H/A1320V/R1333K (SpCas9-NRRH: see, e.g., Ref¹⁸), D10T (optional)/1322V/S409I/E427G/R654L/R753G/R1114G/D1135N/E1219V/D1332N/R1 335Q/T1337N/S1338T/H1349R (SpCas9-NRCH: see, e.g., see, e.g., Ref¹⁸), D1135M/S1136Q/G1218K/E1219S/R1335E/T1337R (MQSKER: see, e.g., US20210261932) variants of SpCas9. In some embodiments, the methods use the HES1208-A4 gRNA (with an NGA PAM and the target adenine in position A4 of the spacer, optionally with ABE8e-SpCas9-VRQR, ABE8e-SpCas9-VRQR(S55R), ABE8e-SpG, ABE8e-SpRY, or ABE8e-SpCas9-NRRH; Table 1), or the HES1210-A7 gRNA (with a GGC PAM and the target adenine in position A7 of the spacer, optionally with ABE8e-SpG, ABE8e-SpRY, ABE8e-SpCas9-NRCH, ABE8e-MQSKER; Table 1); or HES1212-A8 (with a TGG PAM and the target adenine in position A8 of the spacer, optionally with ABE8e-SpCas9, ABE8e-SpG, ABE8e-SpRY, or ABE8e-SpCas9-NRRH; Table 1), provided an excellent combination of on-target editing activity without significant off-target or bystander base editing.

For allele-specific deletion of mutant alleles, genome editors, e.g., nucleases including those listed in Table 2, guided by a single guide RNA (sgRNA) to a sequence of interest, can be used. For example, wild-type SpCas9, optionally with the HES1236 or HES1235 gRNAs (Table 2), can be used to selectively knock-out the mutant R179H allele.

In any of the methods or compositions described herein, the base editor or gemome editor can be delivered as an intein-split construct, e.g., as described in Levy et al., Nat Biomed Eng 4, 97-110 (2020), WO2016112242, Truong et al., Nucleic Acids Res. 2015 Jul. 27; 43(13):6450-8; and Yuan et al., ACS Synth. Biol. 2022, 11, 7, 2513-2517.

TABLE 1
Target sites across ACTA2-R179H for base editing

| | Target site | | | Target | Target | | |
|---|---|---|---|---|---|---|---|
| Target site | with PAM (R1798 | SEQ ID | | nt: | site | | |
| name | bold) | NO: | gRNA ID | pos'n | length | PAM | Use with enzymes: |
| human-ACTA2- | CATCATGCATCTGGA | 55. | | A9 | 23 | CTG | ABE8e-SpRY |
| BE-NTG-SpCas9- | TCTGGCTG | | | | | | |
| A9 | | | | | | | |
| human-ACTA2- | ATCATGCATCTGGAT | 56. | HES1212 | A8 | 23 | TGG | ABE8e-SpCas9, ABE8e-SpG, ABE8e- |
| BE-NGG-SpCas9- | CTGGCTGG | | | | | | SpRY, ABE8e-SpCas9-NERH |
| A8 | | | | | | | |
| human-ACTA2- | TCATGCATCTGGATC | 57. | HES1210 | A7 | 23 | GGC | ABE8e-SpG, ASE8e-SpRY, ABE8e- |
| BE-NGC-SpCas9- | TGGCTGGC | | | | | | SpCas9-NRCH |
| A7 | | | | | | | |
| human-ACTA2- | CATGCATCTGGATCT | 58. | | A6 | 23 | GCC | ABE8e-SpRY |
| BE-NCC-Spcas9- | GGCTGGCC | | | | | | |
| A6 | | | | | | | |
| human-ACTA2- | ATGCATCTGGATCTG | 59. | | A5 | 23 | CCG | ABE8e-SpRY |
| BE-NCG-SpCas9 | GCTGGCCG | | | | | | |
| A5 | | | | | | | |
| human-ACTA2- | TGCATCTGGATCTGG | 60. | HES1208 | A4 | 23 | CGA | ABE8e-SpCas9-VRQR, ABE8e-SpCas9- |
| BE-NGA-SpCas9- | CTGGCCGA | | | | | | VRQR (S55R), ARB8e-SpG, ABE8e- |
| A4 | | | | | | | SpRY, ABE8e-SpCas9-NRRH |
| mouse-ACTA2- | TGCATCTGGACTTGG | 61. | HES1870 | A4 | 23 | CGA | ABESe-SpCas9-VRQR, ABE8e-SpCas9- |
| BE-NGA-SpCas9- | CTGGCCGA | | | | | | VRQR (S55R), ABE8e-SpG, ABBE8- |
| A4 | | | | | | | SpRY, ABE8e-SpCas9-NRRH |
| human-ACTA2- | TCATGCATCTGGATC | 62. | HEB1218 | A7 | 29 | CGA | ABE8e-SaCas9-KKH |
| BE-NNAGAT- | TGGCTGGCCGAGAT | | | | | GAT | |
| SaCas9-A7-23 nt | | | | | | | |
| human-ACTA2- | CATGCATCTGGATCT | 63. | HBS1216 | A6 | 28 | CGA | ABE8e-SaCas9-KKH |
| BE-NNAGAT- | GGTGGCCGAGAT | | | | | | |
| SaCas3-A6-22 nt | | | | | | | |
| human-ACTA2- | ATGCATCTGGATCTG | 64. | HES1214 | A5 | 27 | CGA | ABE8e-SaCas9-KKH |
| BE-WNAGAT- | GCTGGCCGAGAT | | | | | GAT | |
| Sacas9-A5-21 nt | | | | | | | |
| human-ACTA2- | TGCATCTGGATCTGG | 65. | | A4 | 26 | CGA | ABE8e-SaCas9-KKH |
| BE-NNAGAT- | CTGGCCGAGAT | | | | | GAT | |
| SaCas9-A4-20 nt | | | | | | | |

TABLE 2
Target sites across ACTA2-R179H for CRISPR-Cas nucleases

| | | SEQ | | target | target | | |
|---|---|---|---|---|---|---|---|
| | target site with PAM | ID | | nucleotide | site | | |
| target site name | (R179H bold) | NO: | QRNA ID | position | length | PAM | use with enzymes: |
| ACTA2-KO-NGG- | GCCCCATGCCATCATGCATCTGG | 66. | HES1234 | 3 | 23 | TGG | WT SpCas9, SpG |
| BpCas9-site1 | | | | | | | |
| ACTA2-KO-NGG- | TGCCATCATGCATCTGGATCTGG | 67. | HES1235 | 9 | 23 | TGG | WT SpCas9, SpG |
| SpCas9-site2 | | | | | | | |
| ACTA2-KO-NGG- | AGCCAGATCCAGATGCATGATGG | 68. | HES1236 | 7 | 23 | TGG | WT SpCas9, SpG |
| SpCas9-site3 | | | | | | | |
| ACTA2-KO-NGA- | CCCCATGCCATCATGCATCTGGA | 69. | HES1237 | 4 | 23 | GGA | VRQR, VRQR (S55R), |
| SpCas9-site4 | | | | | | | SpG, SpRY |
| ACTA2-KO-NGA- | GCCAGCCAGATCCAGATGCATGA | 70. | | 4 | 23 | TGA | VRQR, VRQR (S55R), |
| SpCas9-site5 | | | | | | | SpG, SpRY |
| ACTA2-KO-NGA- | GATCTCGGCCAGCCAGATCCAGA | 71. | | −4 | 24 | AGA | VRQR, VRQR (S55R) |
| SpCas9-site6 | T | | | | | T | |
| ACTA2-KO-NGC- | TGCCTTGCCCCATGCCATCATGC | 72. | | −4 | 24 | TGC | |
| SpCas9-site7 | A (NGCA > NGCG) | | | | | A | |
| ACTA2-KO-NGC- | GCCATCATGCATCTGGATGTGGC | 73. | | 10 | 23 | GGC | SpG |
| SpCas9-site8 | | | | | | | |
| ACTA2-KO-NGC- | GCCAGATCCAGATGCATGATGGC | 74. | | 8 | 23 | GGC | SpG |
| SpCass-site9 | | | | | | | |
| ACTA2-KO-NNGGAT- | CTTGCCCCATGCCATCATGCATC | 75. | HES1275 | 2 | 28 | CTG | Sacas9, SaCas9- |
| SaCas9-site10 | TGGAT | | | | | GAT | KKH |
| ACTA2-KO-NNTGAT- | CTCGGCCAGCCAGATCCAGATGC | 76. | HES1239 | 2 | 28 | CAT | SaCas9-KKH |
| SaCas9-KKH-site11 | ATGAT | | | | | GAT | |
| ACTA2-KO-NNAGAT- | GTGAGATCTCGGCCAGCCAGATC | 77. | HES1240 | −6 | 28 | CCA | SaCas9-KKH |
| SaCas9-KKA-site12 | CAGAT | | | | | GAT | |
| ACTAS-KO-TCCV- | TCCAGATGCATGATGGCATGGGG | 78. | HES1241 | 3 | 24 | TCC | enAsCas12a, |
| AsCas12s-site13 | C | | | | | A | AsCas12a |
| ACTA2-KO-TCCV- | TCCAGATGCATGATGGCATGGGG | 79. | HES1242 | 3 | 24 | TCC | LbCas12a |
| LbACas12a-site13 | C | | | | | A | |

### V. Methods of Treatment

Provided herein are methods of treating vasculopathies, including subjects who have genetic or acquired vasculopathies, by delivering a therapeutically effective amount of a gene therapy agent. The disclosure demonstrates effective delivery of a therapy comprising a base editor for correcting a mutation known to drive an exemplary genetic vasculopathy, and of a nuclease to delete mutant alleles. The gene therapy agent (e.g., a CRISPR Cas nuclease specifically targeting an R179H mutant allele without or with a repair template encoding a replacement nucleic acid, for treating vasculopathies in subjects who have an ACTA2 R179H mutation) can be delivered in a viral vector, e.g., an AAV as described herein, preferably AAV-PR, preferably wherein expression is driven by an HDAC9-derived promoter as described herein. The vector can be delivered by any suitable route, e.g., locally (e.g., by intraocular, intravesical, or intrathecal delivery) or systemically (e.g., by systemic intravenous delivery).

The present methods can be used to prevent (i.e., reduce the risk of) or delay progression of cerebral vasculopathy, white matter injury and strokes, e.g., in children without critical stenosis of the internal carotid artery (>70% narrowing between the petrous and clinoid ICA diameter). The present methods can also be used to prevent or delay dissection/aortic replacement surgery, e.g., in older children and young adults with high level of neurological function developing rapid progression of aortic dilatation. The present methods can also be used to treat multisystemic smooth muscle dysfunction syndrome (eye, lungs, bladder, gut) which can lead to life-threatening complications in this disease. In some embodiments, a treatment as described herein will lead to normalization of systemic blood pressure and positional orthostatic tachycardia.

In some embodiments, on the cellular level, the present methods lead to normalization of F/G actin ratio (e.g., detectable by western blot and cell immunofluorescence); of gene expression profile (e.g., detectable by mRNA sequencing or PCR); or of Migration, Contractility and Proliferation (e.g., detectable by assays described herein or known in the art).

### VI. Mouse and Cellular Models of ACTA2 R179H MSMDS

Also described herein are mouse models and several cellular models of MSMDS, including a conditional knock-in Acta2R179H mouse that when crossed with a Wnt1-Cre and Myh11-Cre mouse develops the characteristic SMC systemic dysregulation and early onset of aortic dilatation, cerebral steno-occlusive vasculopathy and BBB dysfunction. Also described is an ACTA2 R179H HEK 293T cell-line that permits evaluation of corrective genome editing strategies. These cellular models were used to demonstrate effective correction of the ACTA2 179 codon from the disease causing ‘CAT’ codon to the wild type ‘CGT,’ allele-specific knockout of the R179H allele using CRISPR-Cas nucleases. This strategy was optimize for effective viral-mediated neurovascular delivery of nucleic acids and selectively editing the CAT to CGT codon, mimicking the needed pharmacologic delivery strategy in patients.

Provided herein are conditional knock in ACTA2 Arg179His mouse models, the somatic and germ line cells of which include a mutant ACTA2 exon 6. The wild type allele is with a poly A terminator is surrounded by loxP sites. When the mice are crossed with a mouse expressing a Cre recombinase the wild type exon is removed and the mutant exon is spliced into the ACTA2 mRNA allele, resulting in expression of a mutant alpha smooth muscle actin within cell types expressing a Cre recombinase. For example, to activate the R179H allele in smooth muscle containing organs a cre recombinase driven by a smooth muscle cell myosin heavy chain promoter (Myh11-cre) or an HDAC9 promoter as described herein can be used. To express the mutant allele within the anterior cerebral circulation without other systemic effects, a neural crest specific cre allele (Wnt1-cre) 20 can be used.

Also provided herein are mammalian, e.g., human, cell lines that include an ACTA2 Arg179His or ACTA2 Arg179Cys mutant allele in their genomic DNA. Exemplary cell lines include HEK293, HekT293, CHO.

### EXAMPLES

The invention is further described in the following examples, which do not limit the scope of the invention described in the claims.

### Materials and Methods

The following materials and methods were used in examples described herein.

Generation of the knock-in Acta2^{R179H} mouse model: All mice were cared for under strict compliance with the Partners Institutional Animal Care and Use Committee (IACUC), regulated by the United States Department of Agriculture and the United States Public Health Service and Institutional Animal Care and Use Committee of Massachusetts General Hospital, MA, USA. The Acta2^{R179Hfl/+} mice were generated by subcontracting Cyagen company services (cyagen.com). Homozygous Acta2^{R179Hfl/R179Hfl} mice were crossed with either Myh11-cre (all smooth muscle) or Wnt1-cre (neural crest smooth muscle) for activation of the mutation. The B6.Cg-Tg(Myh11-cre, -EGFP)2Mik/J (cat #007742) and 129S4.Cg-E2f1Tg(Wnt1-cre)2Sor/J mice were purchased from Jackson laboratories. Both mouse lines were crossed to induce the expression of the Acta2 H179 mutant protein. For whole body histology mouse carcasses were imbedded in Buins fixative solution for >48 hrs and brain and organs dissected, paraphing mounted and processed for conventional staining techniques including Hematoxylin Eosin (H&E), Luxol Fast (modified Kluver's myelin sheath stanning), Trichrome and Cersyl Violet. In addition, for immunofluorescence analysis, fixed aortas and brains were cryopreserved with sucrose and embed in OCT or fixed with formalin 4%. Both male and female mice were included in all analysis (ratio 1:1).

Gross Organ images: For aortas and neurovascular circulation, latex was injected into the left ventricular apex under low pressure until it was visible in the femoral artery. Animals were then fixed in Formalin (10%) for 24 h before transfer to 70% ethanol for dissection, photography, and storage. Other organs were then resected.

Mouse aortic SMCs: SMCs were isolated by standard explant of the ascending section of the aortas from Acta2^{R179Hfl/+} or Myh11-Cre:Acta2^{R179Hfl/+} mice. In order to preserve cell identity, all experiments were carried out at passages 1-5. Human and murine SMCs were grown with SMC growth medium from Cell Applications Inc. (catalog 311-500).

Histology: Organs were then removed from the animals or dissected in situ for photography prior to paraffinization and sectioning (7 μM). Slides were produced for tissue staining or stained with standard stains including Elastin (Verhoeff-Van Gieson, Thermo Scientific, MI, USA) or F-actin (ActinGreen™ 488 ReadyProbes, ThermoFisher Scientific, USA) for quantitative analysis. For sequential fluorescence in situ hybridization and immunofluorescence microscopy aortas from human and mice were cryosectioned using OCT standard protocol.

Smooth muscle cell Immunofluorescence: Smooth muscle cell identity was assessed by immunofluorescence staining of contractile markers including Myh11 (Abcam, ab53219), a-SMA (Abcam, ab5694), Sm22α (Abcam, ab14106), Calponin1 (Abcam, ab46794), Smoothelin1 (Santa Cruz, sc-73042), and Vinculin1 (Abcam, ab18058). Cytoplasmic protein lysates were prepared using NE-PER Kit (Pierce, Rockford, IL, USA) and supplemented with 1 ul˜ of protease inhibitor cocktail (Roche) according to the manufacturer's instruction. Mice were euthanized and perfused with 1×PBS. Brains were dissected and snap frozen in liquid nitrogen. Brains were kept at −80° C. until ready to use. Coronal and sagittal brain sections (8 μm, 20 μm and 50 μm) starting at the olfactory bulb were prepared sequentially at using a Leica cryostat. Sections were left to dry for 15 minutes minimum before storage at −80° C. until needed for staining.

Cresyl Violet Staining: Brain sections were stained in 0.1% cresyl violet solution for 10 minutes. Following thus sections were rinsed quickly in distilled water. Differentiate in 95% ethyl alcohol for 2 minutes followed by dehydration in 100% alcohol twice for 5 mins each. Mount with permanent mounting medium and image.

Thioflavin Staining: Brain coronal sections were incubated in fresh filtered 1% Thioflavin-S solution for 8 minutes, at room temperature. Sections were washed twice for 3 mins each in 80% ethanol followed by a single wash in 95% ethanol for 3 min. Sections were washed with 3 exchanges of distilled water. Cover slipping was performed with mounting media and allowed to dry overnight. Following day the slides with coverslip was sealed with clear nail polish and imaged within a week.

Ultrasounds: Nair hair removal cream was used on all mice the day prior to ultrasounds. All ultrasounds were performed on awake, unsedated mice using the Visualsonics Vevo660 imaging system and a 30 MHz transducer. The aorta was imaged using a standard parasternal long axis view. Dimensions from each animal represent averages of measurements made on still frames in systole of the maximal internal diameter of the aortic valve annulus, aortic sinuses, sinotubular junction, or ascending aorta by a cardiologist blinded to genotype.

Neuroimaging Window Placement: A chronic glass coverslip was placed as previously described.⁴² In brief, the surface of the skull was cleared of any residual blood products or debris. A glass coverslip (12 mm diameter, Electron Microscopy Sciences, emsdiasum.com, Cat #72196-12) was cut with a diamond pen to approximate the shape of the dorsal skull surface. C&B Metabond cement was mixed using one scoop of Clear L-Powder, six drops of Quick Base, and one drop of catalyst (Product numbers S399, S398, and S371, Parkell, Edgewood, NY, USA) in a ceramic mixing dish pre-chilled on ice. The cement was used to adhere the glass coverslip to the exposed skull. Additional cement was applied to fill any remaining gaps between the skull and coverslip. The cement was cured for an additional 15 min and the animal allowed to recover.

Resting state functional connectivity (RSFC): RSFC was performed at indicated time points. For each imaging session, mice were anesthetized with 2,2,2-tribromoethanol (TBE), also known as Avertin, which provides excellent RSFC signals.⁴³ Injectable TBE was prepared by mixing 0.5 mL stock solution (10 g 2,2,2-tribromoethanol in 6.25 mL tert-amyl alcohol; Sigma-Aldrich, T48402-25G, and Fisher Scientific, A730-1, respectively) with 39.5 mL 0.9% normal saline, stored at 4° C. for no longer than one month, protecting from light at all stages. To ensure a smooth induction, TBE was injected in two to three divided doses intraperitoneally (0.3-0.4 mL initial dose, 0.1 mL at approximately 8 min, and 0.1 mL at approximately 12 min). More TBE was injected if the mouse did not achieve an adequate level of anesthesia to suppress withdrawal to toe pinch. The animal was placed on a homeothermic heating pad (37.0±0.1° C.) and head-fixed in a stereotaxic frame. The glass coverslip was cleaned with cotton-tipped applicators and diluted ethanol solution. Image acquisition typically began at 18-20 min after the initial dose of TBE.

The acquisition and processing of single wavelength functional optical intrinsic signal imaging have been described previously.^{44,45} The imaging surface was illuminated with a quartz tungsten halogen lamp (Techniquip R150, Capra Optical, Natick, MA) filtered at 570±10 nm and directed with a fiber optic cable. μManager software was used for image acquisition.⁴⁶ Images were acquired with a Cascade 512 F camera (Photometrics) at 512×512 pixel resolution, 11.1 FPS, and an exposure time of 50 ms for 12 min. A custom script was written in MATLAB (Math Works, Natick, MA, USA) for image processing. The image was down-sampled to 256×256 pixels. Based on measurements from the Paxinos and Franklin mouse atlas, the final scale is 47 μm/pixel. The optical density of each pixel over time was calculated and the density maps filtered between 0.008 Hz and 0.09 Hz. The signal was downsampled to 1 FPS. A brain mask was selected. The global signal was determined by taking the mean optical density for each frame. This global signal was then regressed from the optical density maps frame-by-frame over time. Imaging data were analyzed in a blinded manner. Seed-based connectivity maps were created by mapping the correlation coefficients between seeds placed in motor, somatosensory, retrosplenial, and visual cortex and the rest of the brain. The seed locations were primarily chosen for their anatomical distribution rather than a presumed functional role. Seed coordinates were guided by the Paxinos and Franklin mouse atlas and an atlas overlay adapted by White et al. Seed-to-seed connection matrices, global connectivity maps, and interhemispheric homotopic connectivity maps and indices were calculated as described previously.⁴³ The connection matrix data were also organized as topological circle plots.

Blood pressure, blood gas and CBF measurement: Blood pressures (BPs) were measured via femoral artery catheter (MacLab; ADInstruments, Colorado Springs, MO) under general anesthesia. During the procedure, mice were anesthetized under isoflurane 3% induction and 1-1.5% maintenance in 70% N₂O and 30% O₂. Mice temperature was maintained at 37° C. using rectal temperature and a heating pad. The left femoral artery was catheterized with heparinized PE10 tubing. After cannulation, the animals were waiting for stabilize BPs for minutes. After stabilization, arterial pressure (mmHg) was recorded (PowerLab, ADInstruments, Colorado Springs, CO, USA), and an arterial blood samples were drawn to measure pH, partial pressure oxygen (pO₂), partial pressure of carbon dioxide (pCO₂) at the same time that diffuse correlation spectroscopy (DCS) measurements were done. Systolic BPs (SBPs), means BPs (MBPs) and dilated BPs (DBPs) were assessed using stabilized value (Buckley et al., J Cereb Blood Flow Metab. 2015 December; 35 (12): 1995-2000; f.hubspotusercontent10.net/hubfs/40716/Resource%20Center%20HubDB% 20Files/Mouse%20Femoral%20Pressure%20Measurement%20(SP-3-sp).pdf).

Behavior: AnyMaze software (ver. 8.42, Stoelting, Wood Dale, IL, USA) was used for tracking and analysis. For the Open field testing (OFT), mice were placed in a 28×18 cm open field. Distance traveled and speed were recorded for 30 min. For the Y-maze, we used a Y-shaped apparatus consisting of three 33-cm arms with 15-cm-high walls, and a 7.6×7.6×7.6-cm triangular intersection. Each arm was identified with a symbol (square, circle, star). Mice were allowed access to the three arms for a total of 5 min, and their movements were recorded by AnyMaze. The number of times the mouse entered all three arms without re-entering the previous arm (i.e. triplets of ABC, ACB, BCA, etc. vs. ABA, CBC, etc.) and the total number of arm entries were recorded. From this, a percent alternation was calculated. The apparatus was cleaned with 70% ethanol between trials. For the rotarod, five trials were performed each day for three days. Mice were placed on a rod at a starting rotation of 4 r/min which was constantly accelerated to 40 r/min over the course of 120 s. The latency of falling off of the rod and the rod RPMs were recorded.

Carotid Artery Ligations: Carotid ligations were performed in 10-week-old mice. Briefly, animals were anesthetized using intraperitoneal ketamine/xylazine (80 and 12 mg/kg, respectively) followed by a small incision in the neck to expose the carotid artery. Then the left carotid was ligated at the carotid bifurcation level using an 8-0 silk suture. At 21 days mice were sacrificed and carotids were collected for histological analysis. Approximately 98% of wild-type mice developed stenotic lesions. Mice that developed thrombosis of the ligated carotid were excluded from the study (˜1%-2%). All the above mice and wild-type mice (C57BL/6J) were purchased from The Jackson Laboratory. For tissue analysis, animals were euthanized through inhalational isoflurane (Sigma-Aldrich) prior to tissue collection. All carotid ligation (left carotid) procedures were performed at 10 weeks of age. All experiments were performed on male and female animals at a 1:1 ratio.

Carotid Histology. Left and right carotids were cryosectioned using a standard OCT protocol and sectioning. The right carotid artery was unligated and served as an internal control. Neointimal analysis was performed from H&E-stained horizontal cross sections embedded in OCT compound. Briefly, the distal 0.2 mm of carotid from the ligation suture site was discarded followed by generation of 20 slides (10 μm). Slides 1, 5, 10, and 15 were H&E stained for quantification of neointima. For quantification of the neointima, internal and external elastic lamina perimeters and medial thickness from 4 quadrants were measured and averaged using ImageJ software (NIH). Slides 2-4, 6-9, and 11-14 were used for immunofluorescence staining.

### Plasmid Constructs

Brief descriptions of plasmids can be found in Table 3. Oligonucleotide sequences for amplicon sequencing and digital droplet PCR (ddPCR) can be found in Table 4. All modifications to plasmids were generated through standard molecular cloning via restriction digest and ligation or isothermal assembly. SpCas9 nuclease human expression plasmids were generated by subcloning the open reading frames of different Cas enzymes into RTW3027 (pCMV-T7-SpCas9-BPNLS-3×FLAG-P2A-EGFP)⁸. Adenine base editor (ABE) variants were generated by modifying ABEmax (Addgene plasmid 112101)²³ to alter the Cas coding sequence or to modify the deaminase to the recently described ABE8.20m or ABE8e domains.^{15,16} Plasmids encoding the split base editors for packaging in dual-AAV vector constructs were generated by modifying Cbh-v5-AAV-ABE-N-term (Addgene 137177) and Cbh-v5-AAV-ABE-C-term (Addgene 137178).¹⁹ Briefly the N-terminal plasmid was modified to substitute the ABE7.10 domain for ABE8e, and an S55R mutation was added to the SpCas9 coding sequence: the C-terminal plasmid was modified to include the SpCas9-VRQR mutations (D1135V/G1218R/R1335Q/T1337R) and the gRNA spacer sequence targeting the ACTA2^{R179H} allele in the mouse genome (see Table 3). Human cell expression plasmids for U6 promoter-driven SpCas9 sgRNAs were generated by annealing and ligating duplexed oligonucleotides corresponding to spacer sequences into BsmBI-digested BPK1520;⁴ U6 promoter-driven SaCas9 sgRNAs were generated by annealing and ligating duplexed oligo nucleotides corresponding to spacer sequences into BsmBI-digested BPK2660 (Kleinstiver et al., Nature Biotechnology, 2015; PMID 26524662). U6 promoter-driven Cas 12a crRNAs were generated by annealing and ligating duplexed oligo nucleotides corresponding to spacer sequences into BsmBI-digested BPK3079 and BPK3082 for AsCas12a and LbCas12a, respectively (Kleinstiver et al., Nature Biotechnology, 2019; PMID 30742127). U6 promoter driven pegRNAs were generated by annealing and ligating duplexed oligonucleotides corresponding to the spacer, the sgRNA scaffold, and the RTT/PBS sequence into BsmBI-digested MNW320 (pUC19-U6-[BsmBI]-terminator).

TABLE 3
List of plasmids

| plasmid ID | construct type | plasmid description |
|---|---|---|
| RTW3027 | nuclease expression | pCMV-T7-SpCas9-BPNLS(SV40)-3xFLAG-P2A- |
| | | EGFP |
| RTW3027 | nuclease expression | pCMV-T7-SpCas9-BPNLS(SV40)-3xFLAG-P2A- |
| | | EGFP |
| RTW3161 | nuclease expression | pCMV-T7-SpCas9-VRQR-BPNLS(SV40)-3xFLAG- |
| | | P2A-EGFP |
| RTW4177 | nuclease expression | pCMV-T7-SpG-BPNLS(SV40)-3xFLAG-P2A-EGFP |
| SQT1659 | nuclease expression | pCAG-AsCas12a-NLS(nuc)-3xHA |
| AAS848 | nuclease expression | pCAG-enAsCas12a(E174R/S542R/K548R)- |
| | | NLS(nuc)-3xHA |
| SQT1665 | nuclease expression | pCAG-LbCas12a-NLS(nuc)-3xHA |
| MSP1830 | nuclease expression | pCAG-SaCas9-KKH-NLS(SV40)-3xFLAG |
| BPK2139 | nuclease expression | pCAG-SaCas9-NLS(SV40)-3xFLAG |
| RTW4504 | A-to-G base editor | pCMV-T7-ABE7.10(max)-SpCas9-P2A-EGFP |
| | expression | |
| KAC1158 | A-to-G base editor | pCMV-T7-ABE8.20m-SpCas9-P2A-EGFP |
| | expression | |
| KAC978 | A-to-G base editor | pCMV-T7-ABE8e-SpCas9-P2A-EGFP |
| | expression | |
| RTW4945 | A-to-G base editor | pCMV-T7-ABE7.10(max)-SpCas9-VRQR-P2A- |
| | expression | EGFP |
| KAC1166 | A-to-G base editor | pCMV-T7-ABE8.20m-SpCas9-VRQR-P2A-EGFP |
| | expression | |
| KAC1309 | A-to-G base editor | pCMV-T7-ABE8e-SpCas9-VRQR-P2A-EGFP |
| | expression | |
| LLH245 | A-to-G base editor | pCMV-T7-ABE8e-SpCas9-VRQR-P2A-mScarlet |
| | expression | |
| RTW4562 | A-to-G base editor | pCMV-T7-ABE7.10(max)-SpG-P2A-EGFP |
| | expression | |
| KAC1160 | A-to-G base editor | pCMV-T7-ABE8.20m-SpG-P2A-EGFP |
| | expression | |
| KAC984 | A-to-G base editor | pCMV-T7-ABE8e-SpG-P2A-EGFP |
| | expression | |
| HES1425 | A-to-G base editor | pCMV-T7-ABE8e-SpCas9-VRQR(S55R)-P2A-EGFP |
| | expression | |
| LLH247 | A-to-G base editor | pCMV-T7-ABE8e-SpCas9-VRQR(S55R)-P2A- |
| | expression | mScarlet |
| RMD51 | A-to-G base editor | pCMV-T7-ABE8e-SpCas9-NRRH-P2A-EGFP |
| | expression | |
| RMD57 | A-to-G base editor | pCMV-T7-ABE8e-SpCas9-NRCH-P2A-EGFP |
| | expression | |
| KAC1364 | A-to-G base editor | pCMV-T7-ABE8e-SaCas9-P2A-EGFP |
| | expression | |
| KAC1152 | A-to-G base editor | pCMV-T7-ABE8e-SaCas9-KKH-P2A-EGFP |
| | expression | |
| CA21 | A-to-G base editor | pAAV-[ITR]-pCBh-BPNLS(SV40)-TadA8e-gs- |
| | expression | nSpCas9(D10A; S55R)-[N-term]-NpuN- |
| | | BPNLS(SV40)-WPRE-bGH_PA-[ITR] |
| LLH301 | A-to-G base editor | pAAV-pCbh-BPNLS-NpuC-SpCas9-VRQR-BPNLS- |
| | expression | ACTA2_human_NGA_gRNA-pU6 |
| LLH310 | A-to-G base editor | pAAV-pCbh-BPNLS-NpuC-SpCas9-VRQR-BPNLS- |
| | expression | ACTA2_mouse_NGA_gRNA-pU6 |
| MNW95 | prime editor | pCMV-PE2 |
| | expression | |
| HES824 | prime editor | pCMV-PE2-SpG |
| | expression | |
| BPK1520 | gRNA cloning | pUC19-U6-[BsmBI]-SpCas9sgRNA |
| BPK2660 | gRNA cloning | pUC19-U6-[BsmBI]-SaCas9sgRNA |

TABLE 4
Oligonucleotides

| oligo ID | oligo description | oligo sequence |
|---|---|---|
| oHES007 | P5 NGS primer for | ACACTCTTTCCCTACACGACGCTCTT |
| | human ACTA2-R179 | CCGATCTGTGCTGGACTCTGGAGATG |
| | locus | GTGT (SEQ ID NO: 7) |
| oHES008 | P7 NGS primer for | GACTGGAGTTCAGACGTGTGCTCTTC |
| | human ACTA2-R179 | CGATCTGGATGGTCCTGGAAGTTACT |
| | locus | GAGCA (SEQ ID NO: 8) |
| oCA103 | P5 NGS primer for | ACACTCTTTCCCTACACGACGCTCTT |
| | mouse ACTA2-R179 | CCGATCTGATGGTGTGACTCACAACG |
| | locus | TGCC (SEQ ID NO: 9) |
| oCA102 | P7 NGS primer for | GACTGGAGTTCAGACGTGTGCTCTTC |
| | mouse ACTA2-R179 | CGATCTGCCCTCCTTCTGATAGGTTA |
| | locus | CAGTG (SEQ ID NO: 10) |
| oCA80 | forward primer targeted | AATCAACCTCTGGATTACAA (SEQ ID |
| | to rWPRE for ddPCR | NO: 11) |
| | AAV quantification | |
| oCA81 | reverse primer targeted | ATAGCATGATACAAAGGCAT (SEQ ID |
| | to rWPRE for ddPCR | NO: 12) |
| | AAV quantification | |
| oCA72 | forward primer targeted | GATGTCCTTGGTGCACACTC (SEQ ID |
| | to mouse GAPDH for | NO: 13) |
| | control for ddPCR from | |
| | genomic DNA | |
| oCA73 | reverse primer targeted | GAGCTGAGATTGCCCCGC (SEQ ID |
| | to mouse GAPDH for | NO: 14) |
| | control for ddPCR from | |
| | genomic DNA | |
| oCA106 | forward primer for N- | CGAGAAGAAGAATGGCCTGTTCGG |
| | terminal AAV genome, | (SEQ ID NO: 15) |
| | used for ddPCR or RT- | |
| | qPCR | |
| oCA107 | reverse primer N- | CGACATCCTGAGAGTGAACACCG |
| | terminal AAV genome, | (SEQ ID NO: 16) |
| | used for ddPCR or RT- | |
| | qPCR | |
| oCA108 | forward primer for C- | GATCACCCTGAAGTCCAAGCTGGTG |
| | terminal AAV genome, | (SEQ ID NO: 17) |
| | used for ddPCR or RT- | |
| | qPCR | |
| oCA109 | Reverse primer for C- | GGCGATCATCTTCCGCACGTCG (SEQ |
| | terminal AAV genome, | ID NO: 18) |
| | used for ddPCR or RT- | |
| | qPCR | |
| oAK1 | Forward primer for | GGAAAACCCCATCAACGCCAG (SEQ |
| | amplifying the SpCas9 | ID NO: 19) |
| | sequence contained in | |
| | N-terminal AAV | |
| | genome | |
| oAK2 | Reverse primer for | GCTCTTGAAGTTGGGGGTCAG (SEQ |
| | amplifying the SpCas9 | ID NO: 20) |
| | sequence contained in | |
| | N-terminal AAV | |
| | genome | |
| prAK4 | Probe for detecting the | 6FAM-GACGGCTGG-ZEN- |
| | cDNA of SpCas9 | AAAATCTGATCG-IBFQ (SEQ ID NO: |
| | contained in the N- | 21) |
| | terminal AAV genome | |
| prAK2 | Probe for detecting the | 6FAM-CCGCCCTGA-ZEN- |
| | cDNA of SpCas9 | TCAAAAAGTACCC-IBFQ (SEQ ID NO: |
| | contained in the C- | 22) |
| | terminal AAV genome | |

### Human Cell Culture and Transfections

Human HEK 293T cells (ATCC) were cultured at 37° C. and 5% CO₂ in Dulbecco's Modified Eagle Medium (DMEM) supplemented with 10% heat-inactivated FBS (HI-FBS) and 1% penicillin/streptomycin. All tissue culture reagents were purchased from ThermoFisher. The supernatant media from cell cultures was analyzed monthly for the presence of mycoplasma using MycoAlert PLUS (Lonza).

For HEK 293T cell experiments, ˜2×10⁴ HEK 293T cells were seeded per well in 96-well plates between 20 and 24 hours prior to transfection. For nuclease experiments, 29 ng of nuclease and 12.5 ng of sgRNA expression plasmids (unless otherwise indicated) were mixed with 0.3 μL of TransIT-X2 (Mirus) in a total volume of 15 μL Opti-MEM (Thermo Fisher Scientific), incubated for 15 minutes at room temperature, and added to HEK 293T cells. For base editor experiments, 70 ng of base-editor and 30 ng of sgRNA expression plasmids were mixed with 0.72 μL of TransIT-X2 in a total volume of 15 μL Opti-MEM, incubated for 15 minutes at room temperature, and added to HEK 293T cells. For prime editor experiments, 70 ng of base-editor, 30 ng of pegRNA, and 30 ng of nicking sgRNA expression plasmids were mixed with 0.93 μL of TransIT-X2 in a total volume of 15 μL Opti-MEM, incubated for 15 minutes at room temperature, and added to HEK 293T cells. Transfections were halted after 72 hours. Genomic DNA was collected by discarding the media, resuspending the cells in 100 μL of quick lysis buffer (20 mM Hepes pH 7.5, 100 mM KCl, 5 mM MgCl2, 5% glycerol, 25 mM DTT, 0.1% Triton X-100, and 60 ng/ul Proteinase K (NEB)), heating the lysate for 6 minutes at 65° C. then 2 minutes at 98° C., and then storing at −20° C.

Extraction of Genomic DNA from Mouse Tissues

Genomic DNA was extracted from mouse tissues using the Agencourt DNAdvance protocol (Beckman Coulter). Briefly, approximately 10 to 20 mg samples of frozen tissue were incubated at 37° C. for 30 min prior to treatment. Lysis reactions were performed using LBH lysis buffer, 1 M DTT, and proteinase K (40 mg/mL) in 200 μL reactions, incubated overnight (18 to 20 hr) at 55° C. with shaking at 100 RPM. Genomic DNA was purified from lysate using Bind BBE solution containing magnetic beads and performing three washes with 70% ethanol. DNA was eluted in 200 μL of Elution buffer EBA, and the approximate concentrations of genomic DNA were quantified by Nanodrop.

### Next-Generation Sequencing and Data Analysis

The genome modification efficiencies of nucleases, base editors, and prime editors were determined by next-generation sequencing using a 2-step PCR-based Illumina library construction method. Briefly, genomic loci were amplified from approximately 100 ng of genomic DNA using Q5 High-fidelity DNA Polymerase (NEB) and the primers listed in Table 4. PCR products were purified using paramagnetic beads prepared as previously described.^{7,24} Approximately 20 ng of purified PCR product was used as template for a second PCR to add Illumina barcodes and adapter sequences using Q5 and the primers described in Walton et al., Science, 2020.⁸ PCR products were purified prior to quantification via capillary electrophoresis (Qiagen QIAxcel), normalization, and pooling. Final libraries were quantified by qPCR (Illumina Library qPCR Quantification Kit, KAPA Biosystems) and sequenced on a MiSeq sequencer using a 300-cycle v2 kit (Illumina). On-target genome editing activities were determined from sequencing data using CRISPResso2.²⁵ Levels of editing from the in vivo experiments were calculated as: ([% CGT in the treated samples]−[% CGT in the control])/[% CAT in the control].

### Quantification of AAV Genomes in Mouse Tissues

ddPCR reactions to quantify the approximate number of AAV genomes in transduced tissues were performed using approximately 20 to 60 ng of genomic DNA, 250 nM of each primer and 900 nM probe (see Table 4), and ddPCR supermix for probes (no dUTP) (BioRad) in 20 μL reactions. Droplets were generated using a QX200 Automated Droplet Generator (BioRad). Thermal cycling conditions were: 1 cycle of (95° C. for 10 min), 40 cycles of (94° C. for 30 sec, 58° C. for 1 min), 1 cycle of (98° C. for 10 min), hold at 4° C. PCR products were analyzed using a QX200 Droplet Reader (BioRad) and absolute quantification of inserts was determined using QuantaSoft (v1.7.4).

Quantification of Cas9 mRNA

Total RNA was extracted from homogenized liver and Aorta using with TRI Reagent (Ambion) according to the manufacturer's instructions. RNA quality and quantity were assessed using nanodrop spectrophotometer (A260/280 ratio). High-Capacity cDNA Reverse Transcription Kit (thermofisher) was used for quantitative conversion of up to 1 μg of total RNA to single-stranded cDNA. Cas9 gene-specific primer sets (oCA106-oCA109; Table 4) were designed and real-time RT-PCR (qPCR) reactions were performed in a LightCycler (Roche) using SYBR Green to monitor cDNA amplification. Transcript levels of genes were normalized to the expression values of the actin gene. Two technical replicates were done for each combination of cDNA and primer pair, and the quality of the PCR reactions was determined by analysis of the dissociation and amplification curves.

In separate experiments to quantify mRNA expression of either the N- or C-terminal SpCas9 fragments, RNA was extracted using the RNeasy Plus Universal Kit (Qiagen, Hilden, Germany). RNA was reverse transcribed using the RT2 First Strand Kit (Qiagen, Hilden, Germany) and used in ddPCR reactions as described above for gDNA. For ddPCR reactions, cDNA was normalized to 2 ng/μL and each ddPCR reaction contained 12 ng of cDNA, 250 nM of each primer and 900 nM probe (see Table 4), and ddPCR supermix for probes (no dUTP) (BioRad).

### Cas9 Enzyme Immunofluorescence

Liver tissue was fixed in 4% PFA and embedded in OCT following by cryosectioning at 8 mm. Then antigen retrieval was performed on a pressure cooker using the Borg Decloaker buffer (Biocare medical, Cat #BD1000G1). Liver sections were blocked with donkey serum at 10% for 1 hour followed by incubation overnight at 4C with anti-mouse Cas9 (Cell signaling, Cat #14697S) at 1:100 dilution. Slides were washed with PBS-tween at 0.1% 3 times for 3 min followed by incubation with secondary antibody (1:400) (ThermoFisher Scientific, USA) for 1 hr at room temperature. Then slides were washed with PBS-tween at 0.1%, 4 times for 3 min each and slides were mounted with diamond mounting medium containing DAPI. Slides were visualized with the Leica TCS SP8 confocal microscopy station and micrographs were digitized with the Leica Application Suite X software.

### Example 1. Neurovascular Dysfunction and Neurodegeneration in a Mouse Model of Multisystemic Smooth Muscle Dysfunction Syndrome

To characterize the neurovascular and systemic features of MSMDS we created a knock in mouse expressing a mutant SMA with a missense mutation at position 179 replacing arginine with histidine (Acta2^{R179fl/+}) under the control of tissue-specific cre excision. Herein, we characterize the multisystemic features of the animal model and an extensive and neurovascular evaluation to gain insight into the human syndrome.

Creation of ACTA2^{R179fl/+} knock in mouse—To characterize the neurovascular features of MSMDS a Cre-inducible ACTA2^{R179H} knock in mouse was created. The mouse contains a mutant exon 6 downstream of a polyA terminator and surrounded by loxP sites. Cre mediated excision advances the mutant exon to be spliced into the ACTA2 mRNA allele, resulting in expression of a mutant alpha smooth muscle actin within cell types expressing a Cre recombinase (FIG. 1A). To activate the R179H allele in multiple smooth muscle containing organs we used cre recombinase driven by a smooth muscle cell myosin heavy chain cre (Myh11-cre) and to activate the allele within the anterior cerebral circulation without other systemic effects, we used the neural crest specific cre allele (Wnt1-cre) 20. Systemically, Myh11-Cre:Acta2^{R179fl/+} mice were smaller, gained weight slower, and had early mortality when compared to either Wnt1-Cre:Acta2^{R179fl/+} or Acta2^{R179fl/+} littermates. (FIG. 1B-C). Other phenotypes reminiscent of MSMDS were notable including abdominal distention with thinning of the abdominal wall suggestive of Prune-Belly syndrome (FIG. 1C) and a primary pulmonary emphysema, both of which have been seen in human patients. FIG. 1D is a picture of the abdominal wall of a control and a Myh11-Cre:Acta2R179Hfl/+ mice, illustrating that the Myh11-Cre:Acta2R179Hfl/+ is thin and commonly exhibits clearly visible distension. Mice are 4 months of age at the time of the picture. Gross examination of abdominal organs demonstrates striking abnormalities including dilated bowel, hydronephrosis, and distended bladder (FIG. 1F-H). Bladder dysfunction was evident through analysis of voiding patterns in Myh11-Cre:Acta2^{R179fl/+} mice. Necropsy of spontaneous death in Mhy11-Cre:Acta2^{R179Hfl/+} mice revealed no evidence of aortic rupture but frequently necrotic, distended bowel loops.

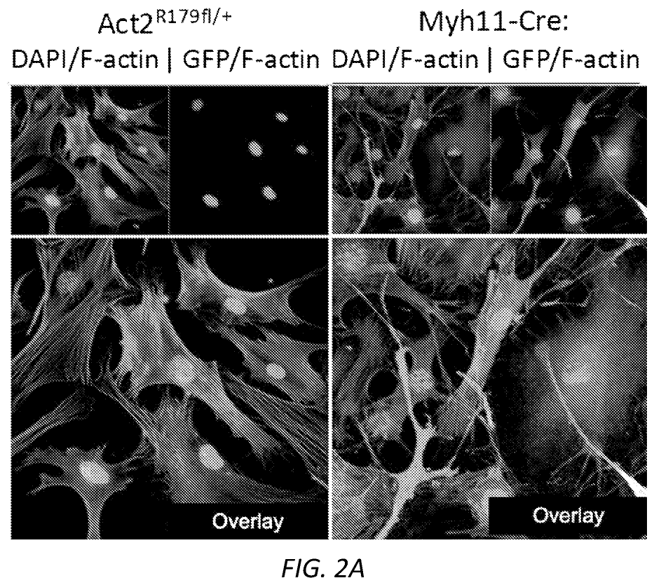

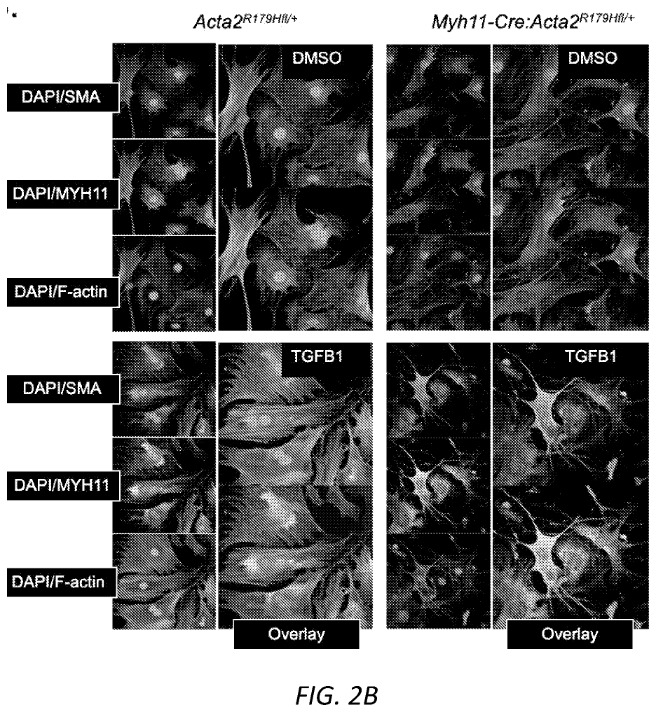

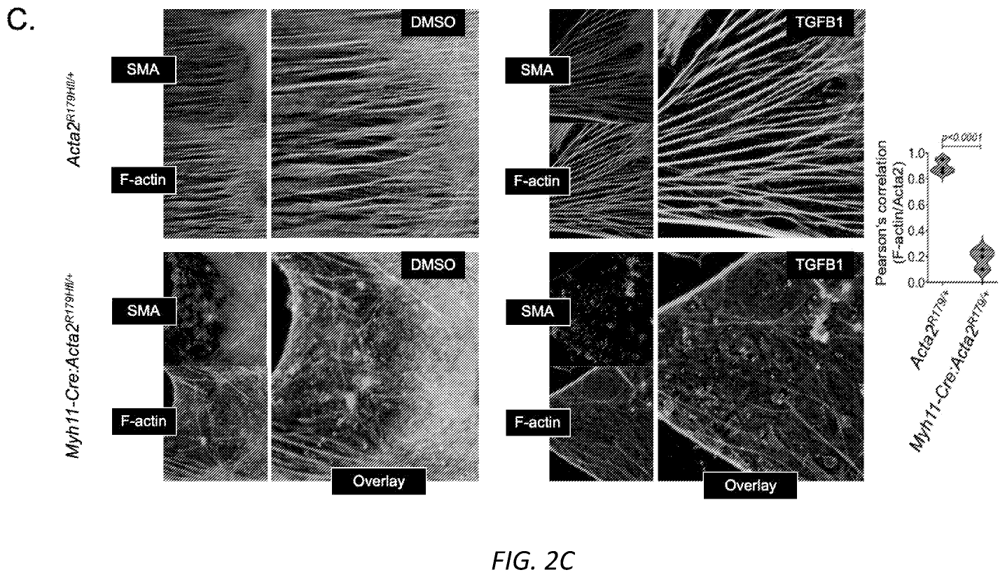

Characterization of smooth muscle cells in ACTA2^{R179fl/+} knock in mouse—We cultured vascular smooth muscle cells from the aortas of Myh11-Cre:Acta2^{R179fl/+} and control mice to determine cellular level changes in phenotype. Smooth muscle cells from Myh11-Cre:Acta2^{R179fl/+} mice were noted to adopt a less flattened cellular shape in comparison to wild type Acta2^{R179fl/+} control cells, although filopodial structures were maintained resulting in a more spindle-like appearance (FIG. 2A). The Myh11-Cre allele expressed a green fluorescent protein, allowing for verification of cellular identity. To examine the differences in cellular morphology we probed the structure of the filamentous actin cytoskeleton with rhodamine-labeled phalloidin. We also probed for the localization of SMA, the smooth muscle myosin (MYH11), and the actin cytoskeleton during stress fiber formation through the application of transforming growth factor beta (TGF-beta) (FIG. 2B). As previously described the ligand, TGF-beta, stimulated the formation of numerous thickened stress fibers in wild type Acta2^{R179fl/+} control cells, while Myh11-Cre:Acta2^{R179fl/+} cells could not form stress fibers (Park et al., Biochem Biophys Res Commun. 2017 Jan. 29; 483(1):129-134). At lower resolution, SMA staining often appeared diffuse and not arranged in filamentous structures in Myh11-Cre:Acta2^{R179fl/+} VSMCs. In fact, higher resolution magnification of the filamentous cytoskeleton in these cells also revealed both a lack of colocalization of SMA with F-actin and underdevelopment of stress fibers in Myh11-Cre:Acta2^{R179fl/+} cells (FIG. 2C).

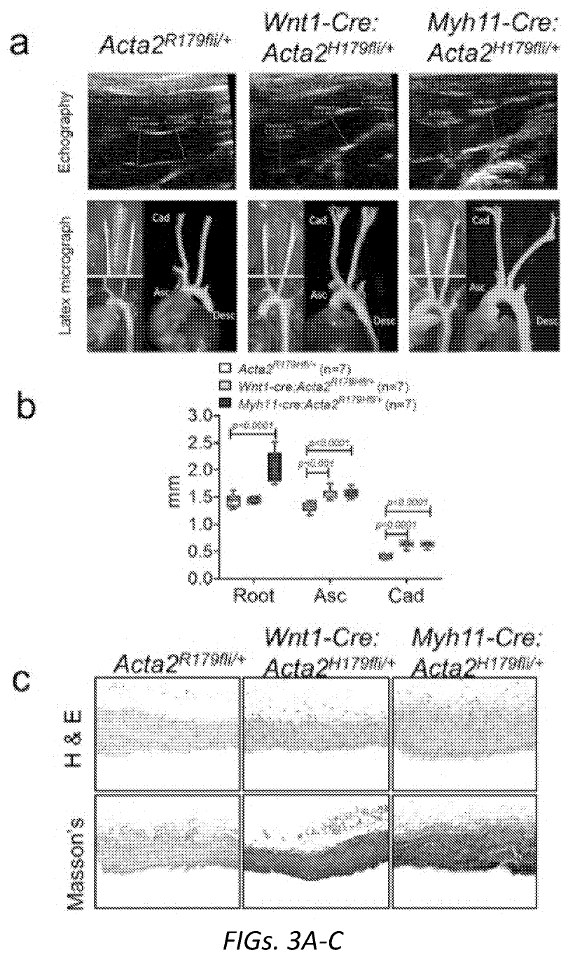

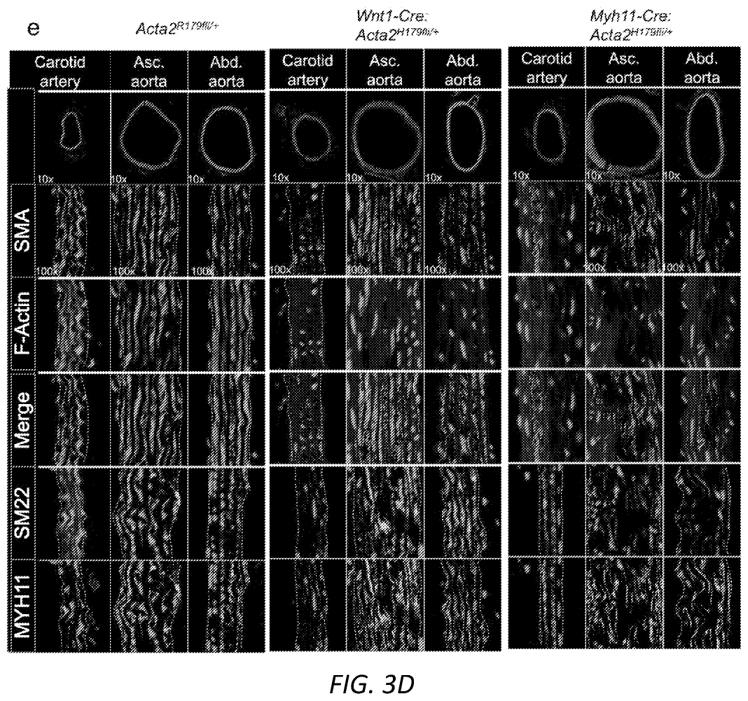

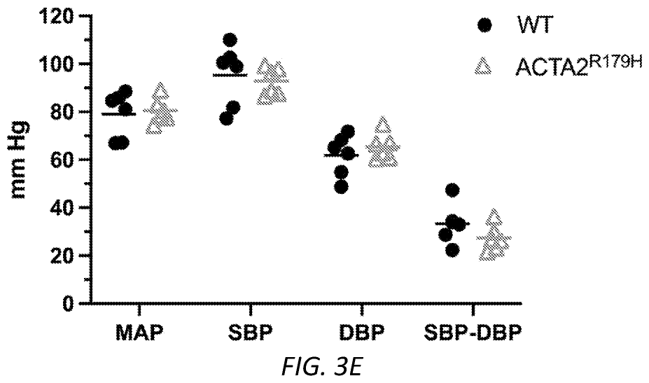

Cardiovascular features of ACTA2^{R179fl/+} knock in mouse—Conduit arterial failure is a common feature of MSMDS in human patients. The hearts of Myh11-Cre:ACTA2^{R179fl/+} and Wnt1-Cre:ACTA2^{R179fl/+} mice were normal in appearance (not shown), however there were multiple abnormalities of the aortas and carotid arteries of the mutant mice. Diameters of the ascending aorta and the proximal right brachiocephalic artery were significantly larger at two months of age in both Myh11-Cre:ACTA2^{R179fl/+} and Wnt1-Cre:ACTA2^{R179fl/+} mice when compared to wild type ACTA2^{R179fl/+} littermates via echocardiography or gross examination (FIG. 3A-B). Aortas from Myh11-Cre:ACTA2^{R179fli/+} and Wnt1-Cre:ACTA2^{R179fl/+} mice exhibited cellular disarray, a thickened wall, and increased collagen deposition in the adventitia (FIG. 3C). Immunofluorescence demonstrated characteristics similar to other models of vascular dysfunction, prominently including loss of filamentous actin staining in the vascular smooth muscle cells (VSMCs) (FIG. 3D). The contractile marker SM22a was depleted in diseased aortic tissue, while staining for both MYH11 and SMA itself were substantively unaffected (FIG. 3D). Physiological assessments of Myh11-Cre:Acta2R179Hfl/+ mutant mouse showed a trend towards lower systolic and pulse blood pressures when compared to controls (FIG. 3E).

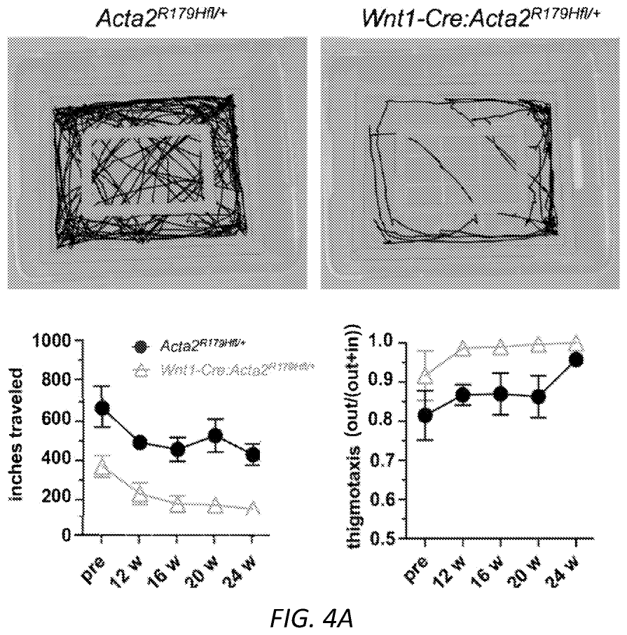

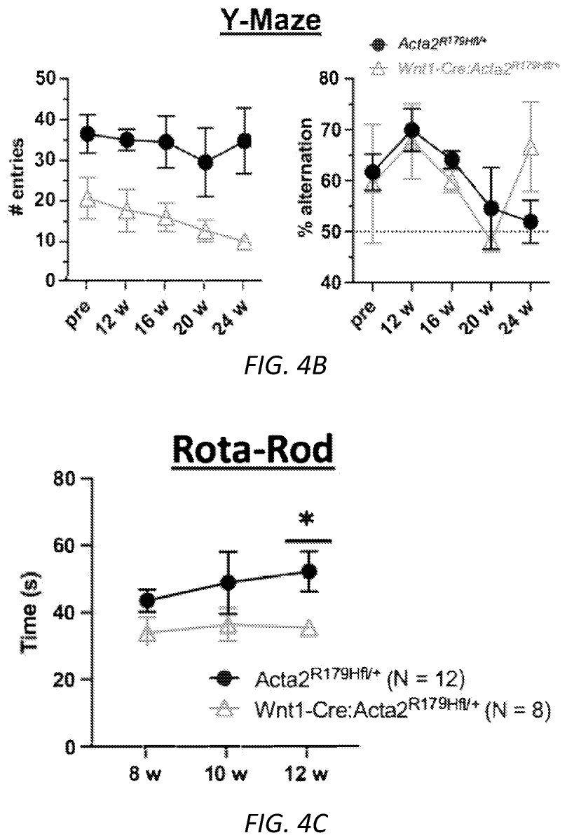

Neurobehavioral Features of the ACTA2^{R179H} knock in mouse—Patients with MSMDS suffer from fatigue, exercise intolerance and impairment of behavioral/cognitive ability (anxiety, executive and attention deficits). Up to a third of patients will motor disability (cerebral palsy).¹⁸ To assess whether our animal model demonstrates similar signs of this clinical disease we performed a series of behavioral assessments. Open field test (OFT) and Y-maze testing showed decreased distance traveled and average walking speed at baseline at 12, 16, 20 and 24 weeks of age in Wnt1-Cre:Acta2^{R179Hfl/+} mice compared to littermate controls (FIG. 4A-B). Furthermore, we found increased anxiety as evidenced by increased thigmotaxis in the Wnt1-Cre:Acta2^{R179Hfl/+} mouse (FIG. 4A). Y-maze testing suggests similar working spatial memory on the percent alternation assessment. However, the Wnt1-Cre:Acta2^{R179Hfl/+} mice had fewer entries into each arm of the Y-maze which suggests a lower level of motor activity, consistent with open field testing (FIG. 4B). There was a significant decrease in the latency of falling in rotarod testing in the Acta2^{R179Hfl/+} mice (FIG. 4C) without evidence of motor asymmetries or coordination issues as measured by the modified neurological severity scores (mNSS)⁵ which includes beam walk test.

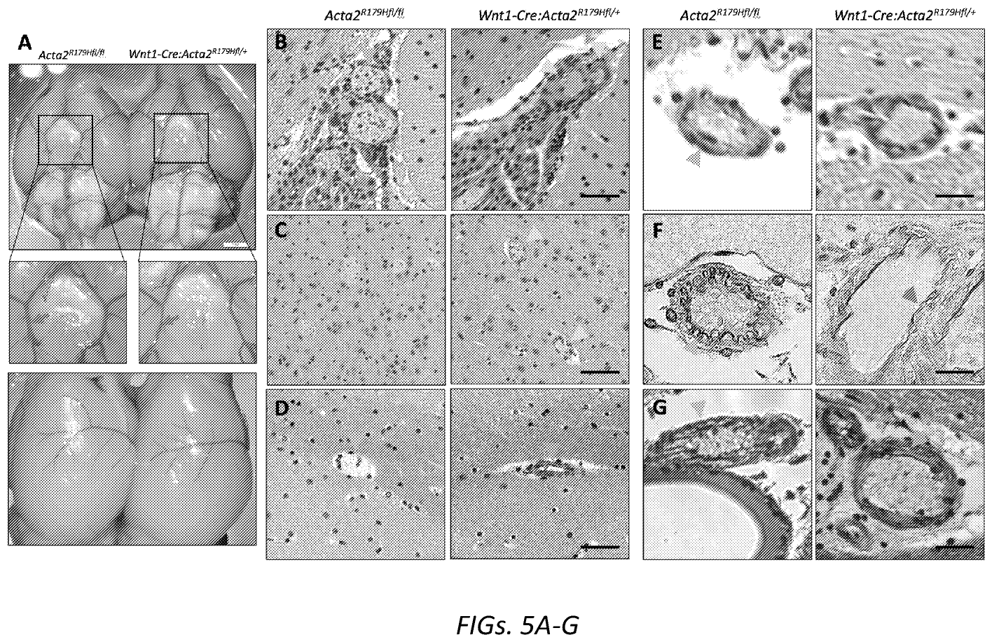

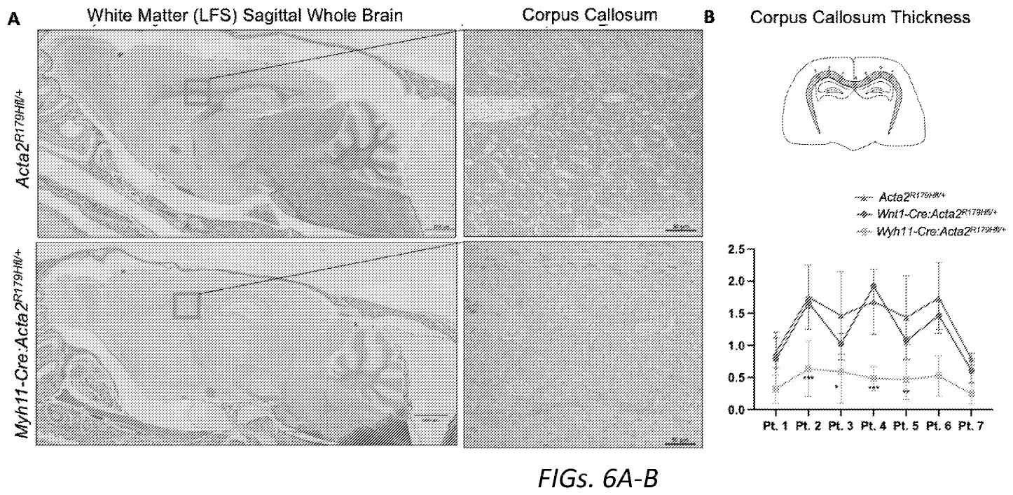

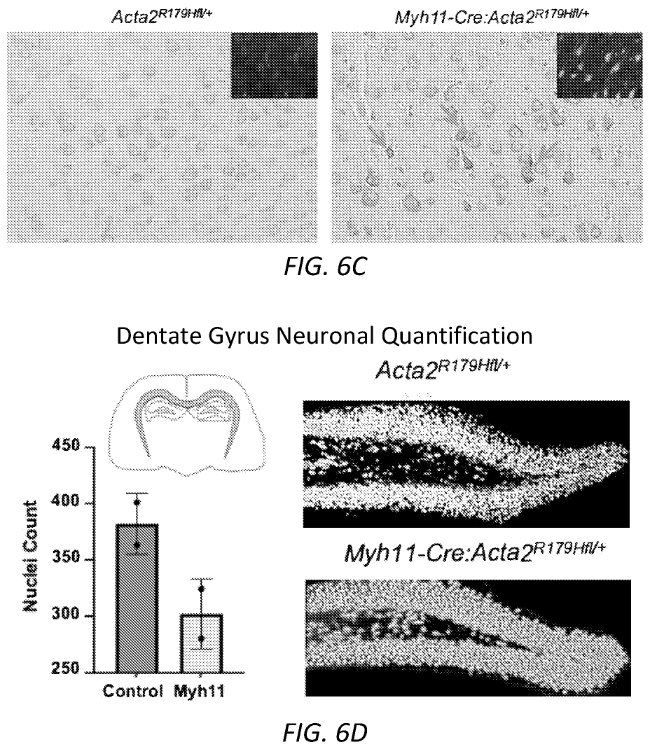

Neuropathologic and Cerebrovascular Features of the ACTA2^{R179H} knock in mouse—To seek pathologic correlates of these behavioral abnormalities we next examined the cerebral and cerebrovascular systems of the ACTA2^{R179H} knock in mouse. Wnt1-Cre:Acta2^{R179Hfl/+} mice showed narrowing of the intracranial ICA relative to its extracranial portion and pathognomonic straightening of its branching after latex injection (FIG. 5A). Histologic evaluation of intracranial arteries demonstrated disrupted elastin fibers, increased collagen deposition in the vessel wall, narrowing of the vessel lumen, and significant wall thickening. These features were seen earlier in the Myh11-Cre:Acta2^{R179Hfl/+} mice at 8 weeks, (FIG. 5B-G) compared to 16 weeks in the Wnt1-Cre:Acta2^{R179Hfl/+} (not shown). Cerebellar vessels were not affected in the Wnt1-Cre:Acta2^{R179Hfl/+} as expected given lack of Cre recombination in VSMCs in the vertebrobasilar system (derived from somatic mesoderm). At the parenchymal level both Wnt1-Cre:Acta2^{R179Hfl/+} and Myh11-Cre:Acta2^{R179Hfl/+} mice showed signs of ischemic neurodegeneration affecting both grey and white matter structures with an earlier and more severe phenotype in the Myh11-Cre:Acta2^{R179Hfl/+} mice. Significant white matter degeneration with loss of volume, vacuolation and enlarged peri-vascular spaces were seen in the corpus callosum, anterior commissure and internal capsule (plus cerebellar white matter in Myh11-Cre:Acta2R179Hfl: mice) (FIG. 6A-B). Cortical neurons with degenerative features-including red neurons, ghost neurons and neurons with swollen nuclei and intracytoplasmic neuronal inclusions akin neurofibrillary tangle-were observed in the motor/somatosensory cortices, caudoputamen and hippocampus (FIG. 6C). Quantification of dentate gyrus neurons in the hippocampus showed >50% count loss at 10 weeks of life (FIG. 6D). No significant difference in these indices were observed in a heterozygous Acta2^{+/-} mouse when compared to WT suggesting that a single copy of the gene is sufficient to prevent the cerebrovascular phenotype (data not shown).

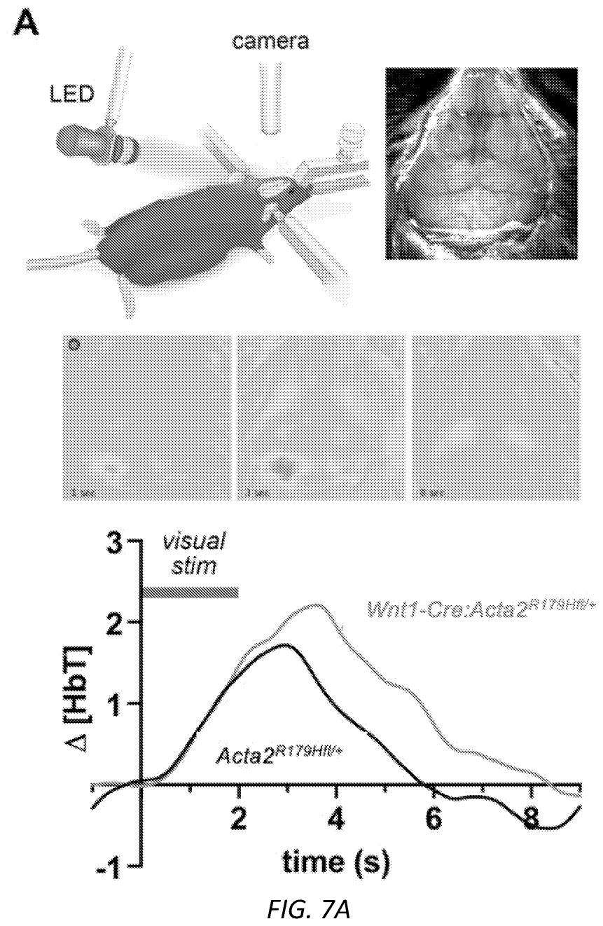

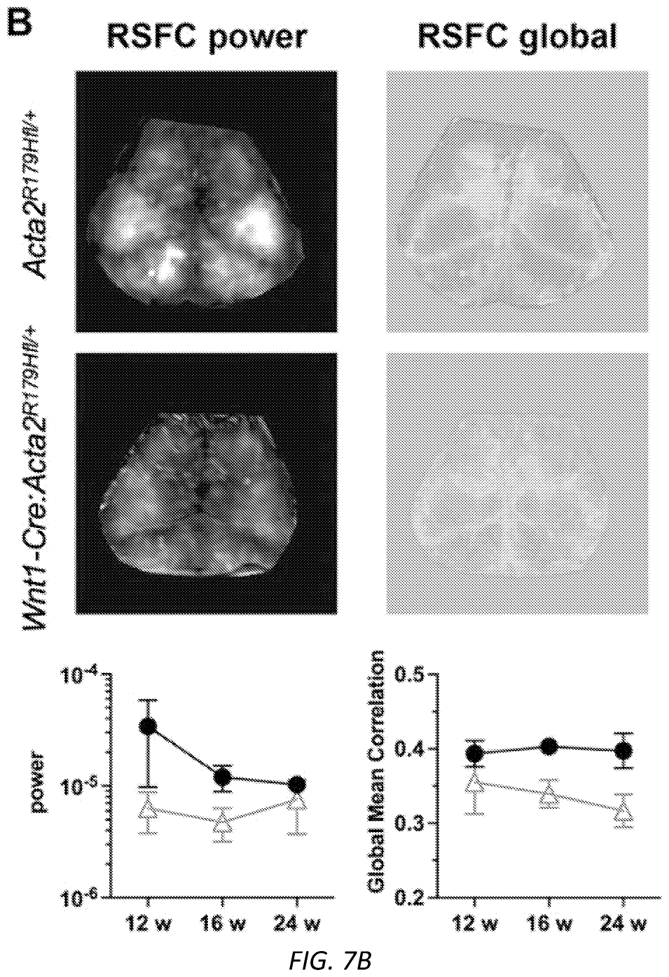

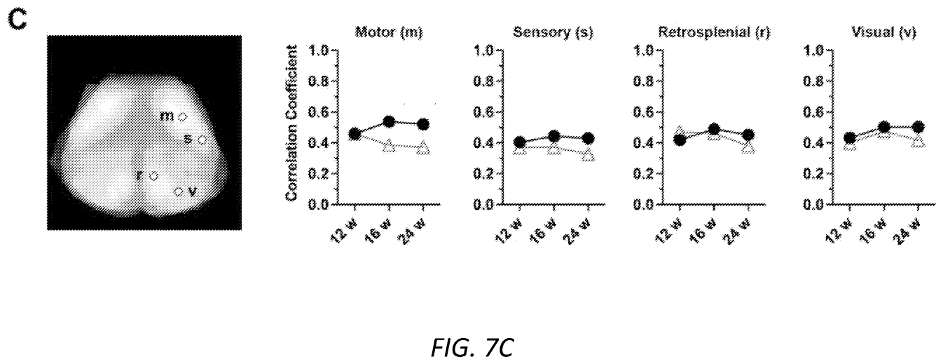

Neurovascular Connectivity—Neuronal activity creates a hemodynamic response that locally alters brain concentrations of deoxy- and oxy-hemoglobin producing a time-dependent signal representation of CBV that can be assessed by non-invasive functional optical intrinsic signal (OIS) imaging. This imaging can be performed over the dorsal surface of the mouse brain in vivo through chronic windows made by implanting a glass coverslip over an intact and unaltered mouse skull, as previously described²² (FIG. 7A). Analysis of the Wnt1-Cre:Acta2^{R179Hfl/+} mice at from 12 to 24 weeks of age showed significant alterations in neural connectivity (NC) affecting a measure of global resting state functional connectivity. Using Avertin anesthesia (which best preserves the hemodynamic response²³) we observed lower amplitude CBV fluctuations in the BOLD frequency range (0.01-0.1 Hz) early in the Wnt1-Cre:Acta2^{R179Hfl/+} mice while Acta2^{R179Hfl/+} controls reached these levels 3 months later (FIG. 7B). Acta2^{R179H} mice had a significant decrease in global and motor areas connectivity at first assessment followed by worsening and loss of connectivity in sensory, retrosplenial, and visual areas by 6 months of age (FIG. 7C). The severe and early motor connectivity loss and neurodegeneration found on histological studies of the Acta2^{R179Hfl/+} mice may explain our behavioral findings. Notably, loss of functional connectivity in the retrosplenial cortex, which heaviest projections originate in the hippocampal formation, predicts cognitive aging.²⁴

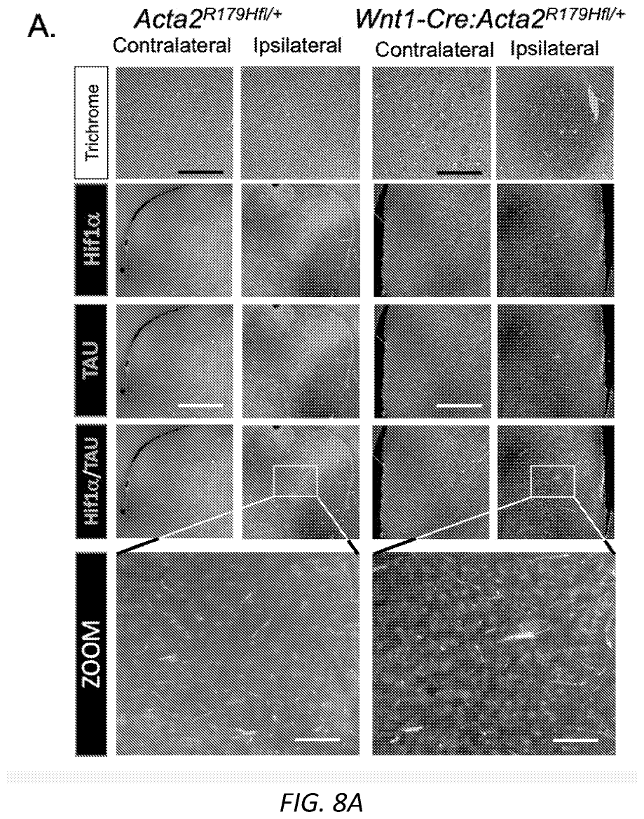

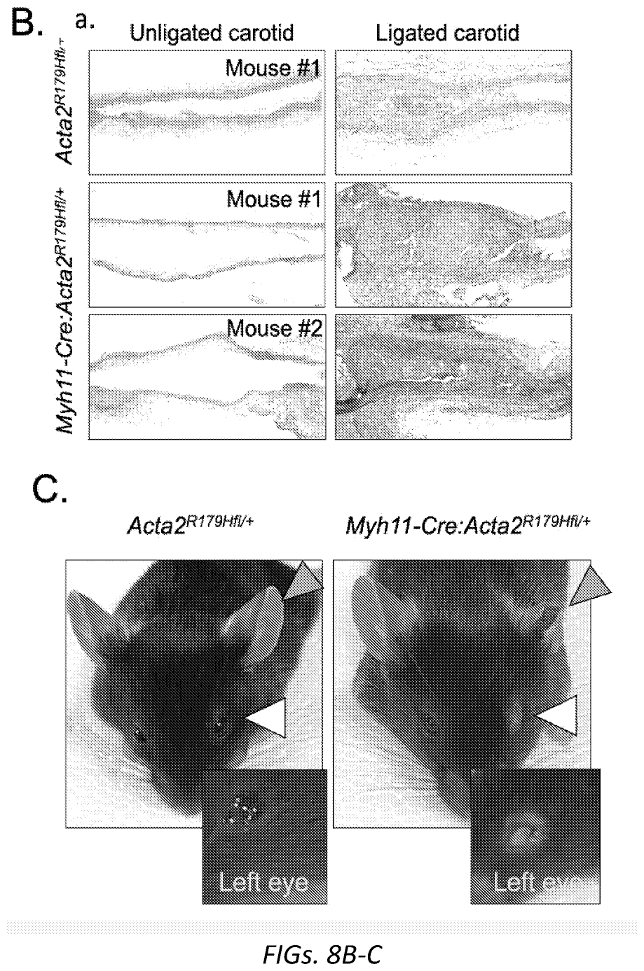

Induced Stroke in ACTA2^{R179H} mice—Mice expressing the ACTA2^{R179H} allele in the cerebral vasculature exhibit vascular abnormalities, ischemic neurodegeneration and subsequent connectivity and behavioral abnormalities. To simulate the hypoperfusion event-related ischemic injury observed in patients with MSMDS we induced cerebral hypoperfusion through unilateral surgical carotid arterial occlusion. In wild type mice, unilateral carotid artery occlusion is well tolerated, necessitating the creation of bilateral carotid artery occlusion models to achieve significant ischemic cerebral damage.^{25,26} In contrast, in our Wnt1-Cre:Acta2^{R179Hfl/+} and Myh11-Cre:Acta2^{R179Hfl/+} mouse models of disease, we noted significant mortality after unilateral carotid artery ligation, in contrast to control mice (Acta2^{R179Hfl/+}). To determine if this mortality was due to ischemic injury/stroke, we used histology to examine the brains of mice subjected to unilateral carotid artery occlusion (FIG. 8A). Immunofluorescent staining for hypoxia inducible factor 1-alpha (HIF1a), a marker of tissue ischemia, demonstrated strong signal in the Wnt1-Cre:Acta2^{R179Hfl/+} mice, at three days post ligation, accentuated on the side ipsilateral to the carotid ligation, but no significant HIF1a staining on either side of the brain in the ligated Acta2^{R179Hfl/+} littermate (FIG. 8A). H&E and Trichrome staining clearly showed evidence of ischemic tissue damage in the ipsilateral Wnt1-Cre:Acta2^{R179Hfl/+} mouse neural tissue (FIG. 8A, top row). Examination of carotid artery histology in the Myh11-Cre:Acta2^{R179Hfl/+} mice after carotid artery occlusion, revealed excessive neointimal proliferation in the ligated artery when compared to Acta2^{R179Hfl/+} mice (FIG. 8B), a finding noted in Acta2^{-/-} mice. Additionally, we noted necrosis of tissue supplied by the external carotid artery including the eye (cornea, conjunctiva) and the external auricle ipsilateral to common carotid occlusion highly suggestive of lack of ability to engage collateral blood flow from the contralateral external carotid via common anastomosis, another sing of SMC vessel dysfunction and lack of flow redistribution capacity (FIG. 8C).

### Example 2. Altered Actin and Cytoskeleton in Patients with ACTA2 R179 Mutations

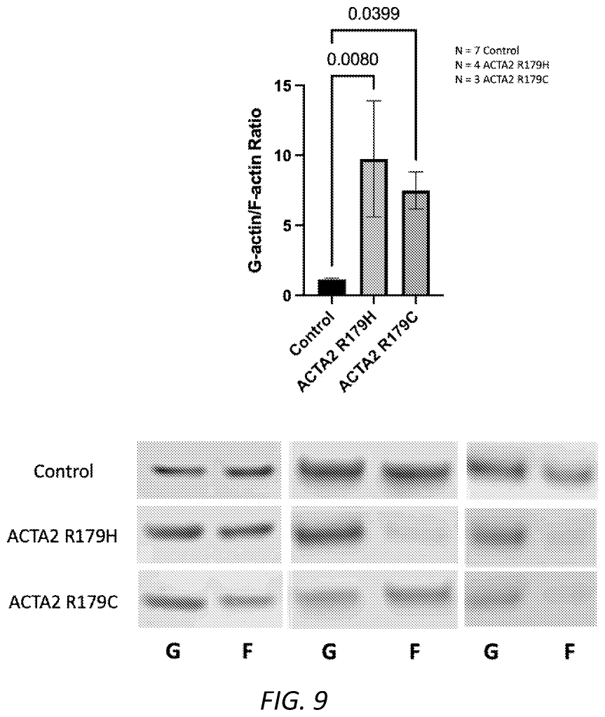

In this experiment, human skin fibroblasts were grown from a patient with an ACTA2 R179H mutation, a patient with ACTA2 R179C mutation, and a control (unaffected) subjects. Cell contents were fractionated through centrifugation to separate polymerized filamentous beta actin (F-actin) from globular beta actin (G-actin). Beta actin was identified via western blot with antibody directed against it (Cytoskeleton Inc., Clone 7A8.2.1). The results, shown in FIG. 9, demonstrate that cells expressing the mutant ACTA2 proteins have a decreased amount of polymerized (F-actin) and altered ratios of G/F actin in the affected subjects.

In addition, the same cells were stained for the presence of polymerized beta-actin (F-actin) with phalloidin-FITC and an α-smooth muscle actin antibody, and the cell nuclei were stained with DAPI. To induce a smooth muscle-like state the cells were incubated in the presence of TGF-beta (10 ng/mL) for 24 hrs. Stress fiber induction was less robust in R179H and R179C patient cells. In addition, in human cells with R179H or R179C ACTA2 mutations, α-smooth muscle actin failed to colocalize efficiently to stress fibers, as seen in wild type cells.

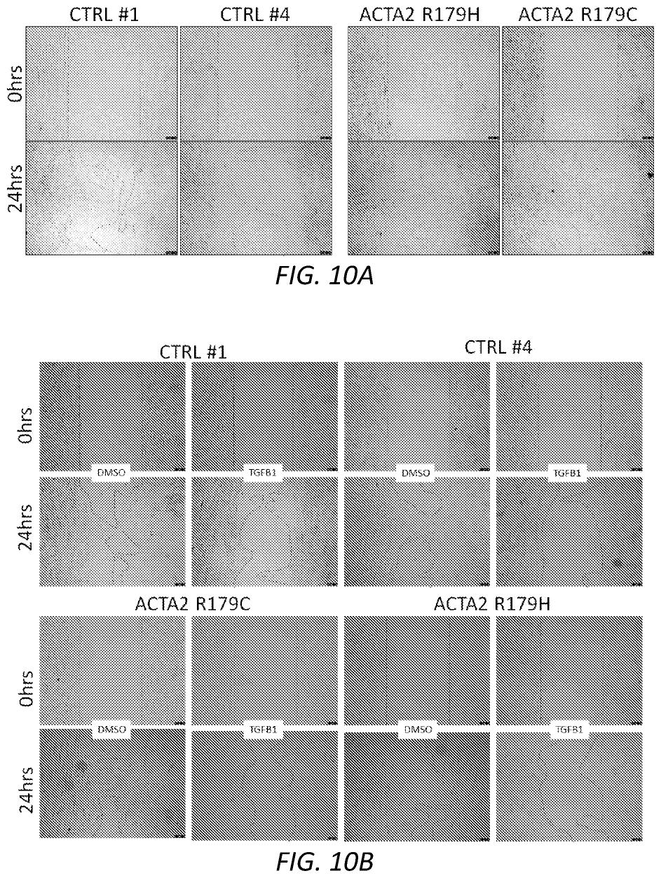

A scratch assay was also performed. In this experiment human skin fibroblasts were grown from a patient with an ACTA2 R179H mutation, a patient with ACTA2 R179C mutation, and two control (unaffected) subjects. The cells were plated on cell chambers that contained an insert of consistent width that prevented cellular growth in that area. The insert was removed, and the cells were photographed at time zero and at 24 hours later. Cells then migrated into the space previously occupied by the insert. The results, seen in FIG. 10A, showed that cells from patients with R179H and R179C migrate into the space faster than control cells. TGF-beta treatment was found to slow the migration of both control and patient cells (see FIG. 10B).

### Example 3. Minimal HDAC9 Promoter Directs Protein Expression in Dysfunctional VSMCs

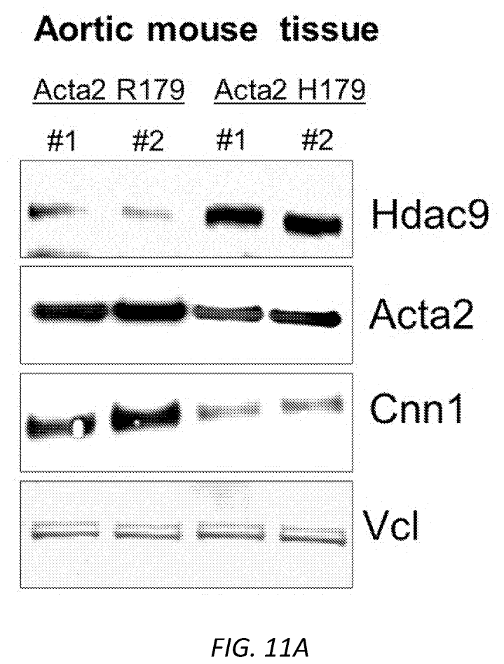

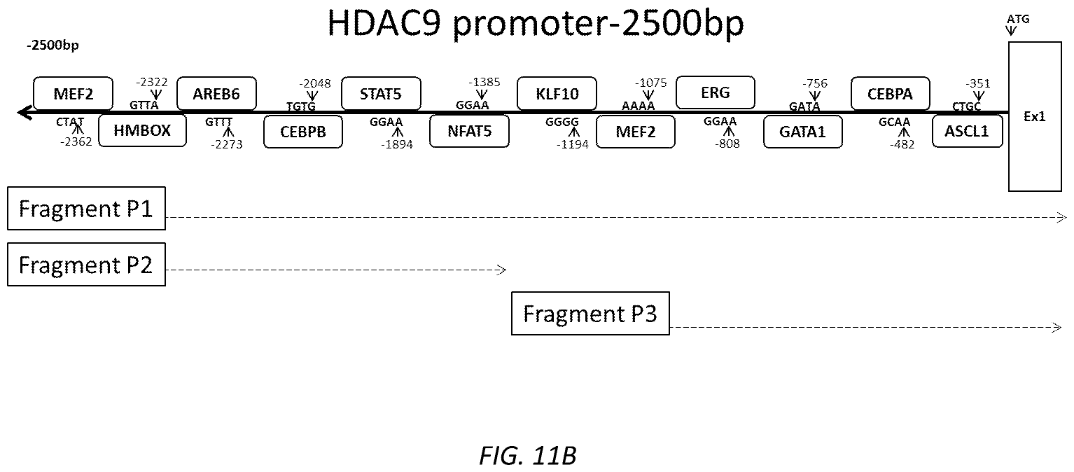

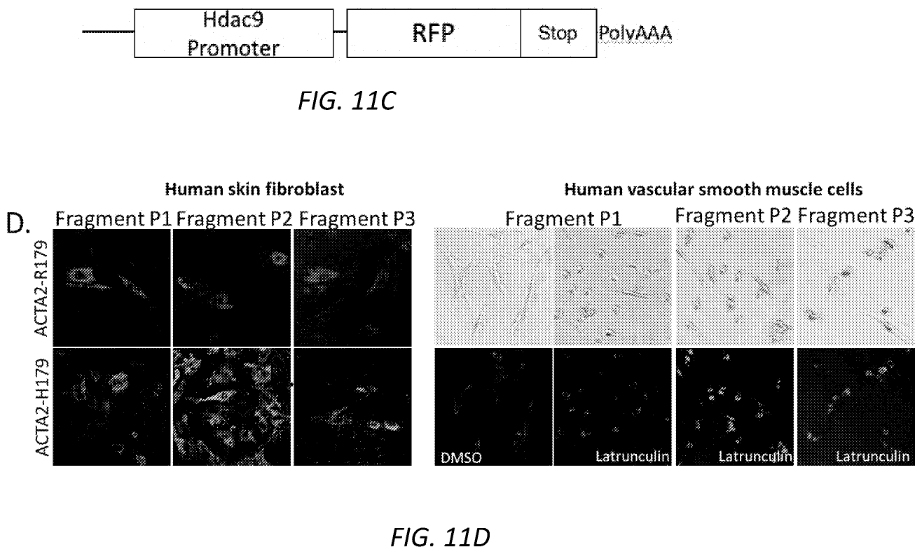

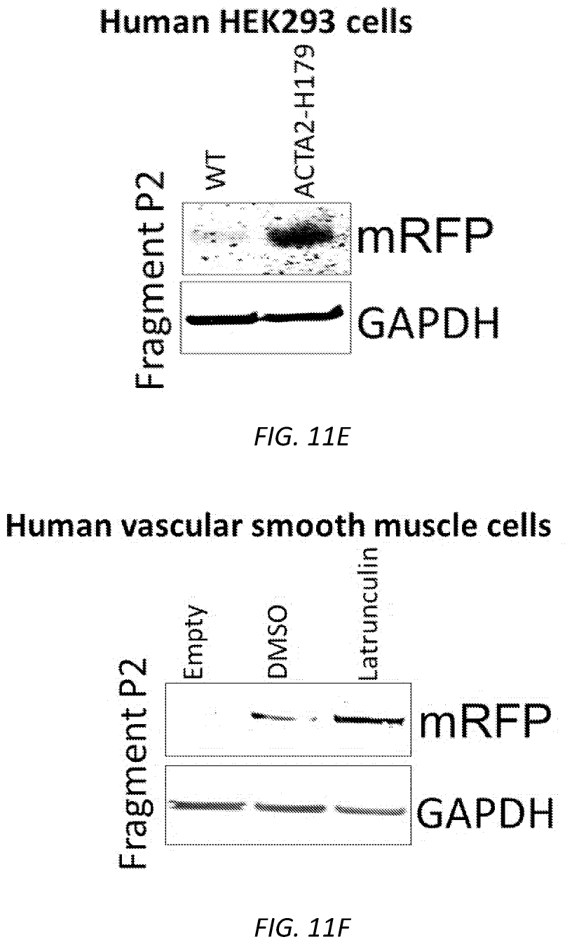

To investigate the feasibility of using the human HDAC9 promoter in gene therapeutic applications within VSMCs, we first examined the expression of the native Hdac9 protein within the murine aorta in an experimental model of multisystemic smooth muscle dysfunction syndrome (MSMDS). As compared to Acta2^{R179Hfl/+} mice (wild type), aortas from Myh11-Cre:Acta2^{R179Hf/+} (disease) mice demonstrated upregulation of the Hdac9 protein by immunoblotting, coincident with downregulation of alpha smooth muscle actin (ACTA2) and calponin (CNN1) (FIG. 11A). The promoter region of HDAC9 contains multiple identifiable trans-acting sites including sites for KLF factors, STAT proteins, NFAT factors, as well as MEF2 binding sites as previously described⁴⁰ (FIG. 11B). To find a smaller portion of the −2500 to +0 bp, herein named P1, promoter region with disease-induced upregulation activity we created smaller fragments (−2500-1300 bp, P2) and (−1300 to +0 bp, P3) to investigate individually (FIG. 11C). We fused each of these fragments to a pcDNA3-RFP reporter for in vitro expression in cells. The individual plasmids were transfected into two types of cells: skin fibroblasts isolated from patients with MSMDS (ACTA2 R179H mutation) or control fibroblasts as well as control human VSMCs to which we added the actin depolymerizing agent, latrunculin. In both cases, we noted induced expression of the HDAC9 promoter either comparing wild type fibroblasts to diseased (ACTA2 R179H mutation) fibroblasts, or in wild type VSMCs upon the application of latrunculin (FIGS. 11D-F). Also, in both cell types the highest expression was seen with the P2 fragment driving the expression of the RFP reporter.

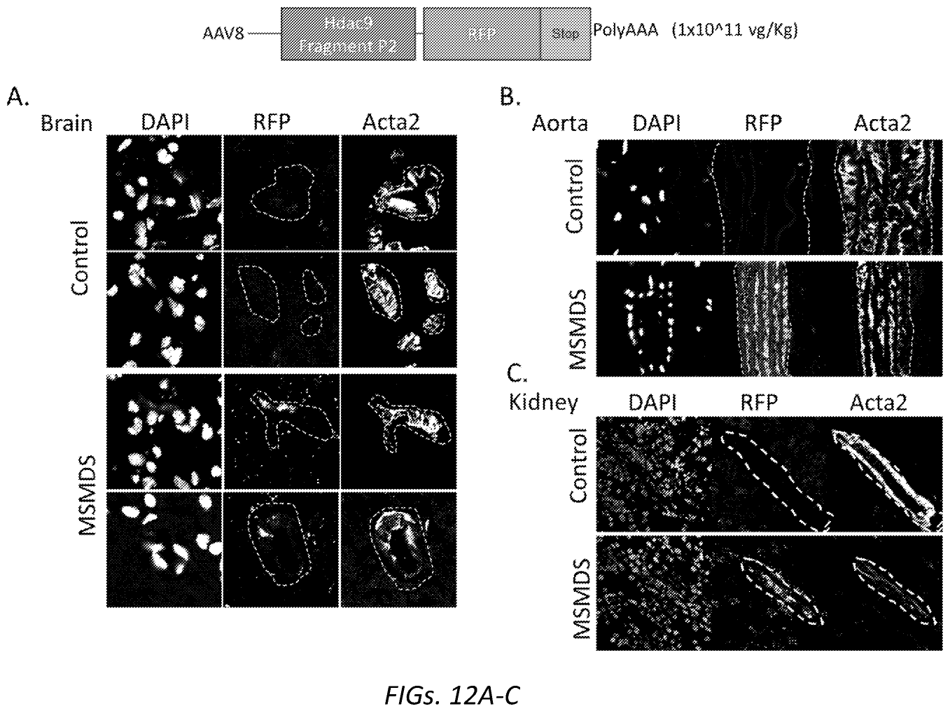

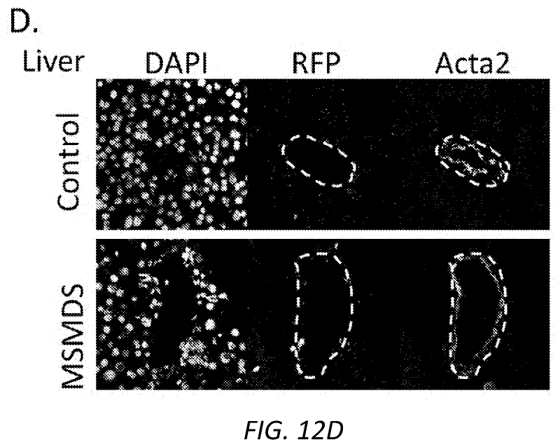

To determine the suitability of the P2 HDAC9 promoter fragment to direct disease-induced expression of proteins within VSMCs we created an Adeno-associated virus, serotype 8 (AAV8) that directs expression of RFP under the control of the P2 HDAC9 promoter fragment (AAV8-P2-RFP) (FIGS. 12A-D). After transfection of 1×10\hat{ }11 vp/kg into either Acta2^{R179Hfl/+} (control), or Myh11-Cre:Acta2^{R179Hlf+} (MSMDS) mice we evaluated multiple tissues for expression of RFP. As shown by colocalization with α-SMA, AAV8-P2-RFP directed RFP expression in the vascular compartment of the brain (FIG. 12A), aorta (FIG. 12B), and kidney (FIG. 12C) in the Myh11-Cre:Acta2^{R179Hfl} (MSMDS) but not Acta2^{R179Hfl/+} (control) mice. Expression in liver, which is highly transduced by AAV8 vectors (rational for serotype selection in this experiment), and its vasculature (FIG. 12D) was not enriched overall demonstrating improved VSCM selectivity and lower off-target liver transduction with this promoter gene therapy approach.

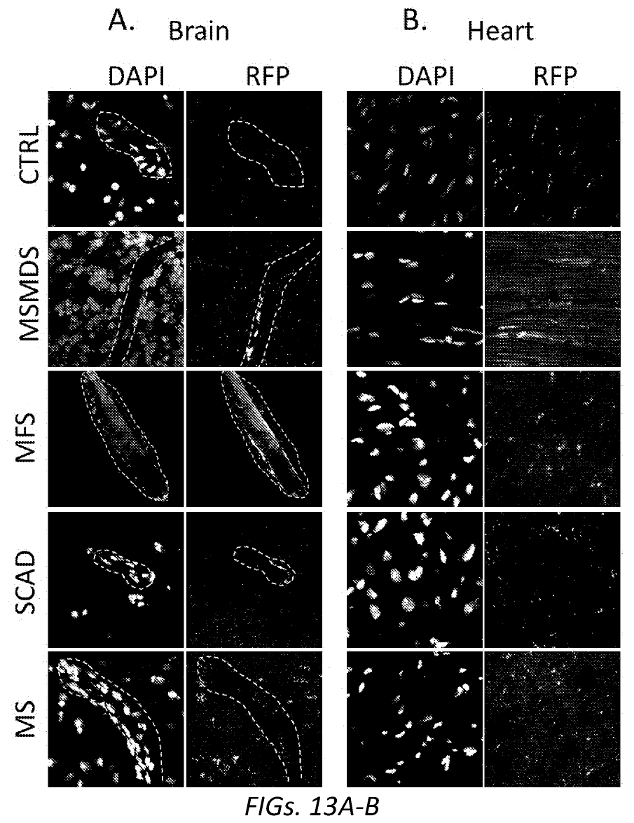

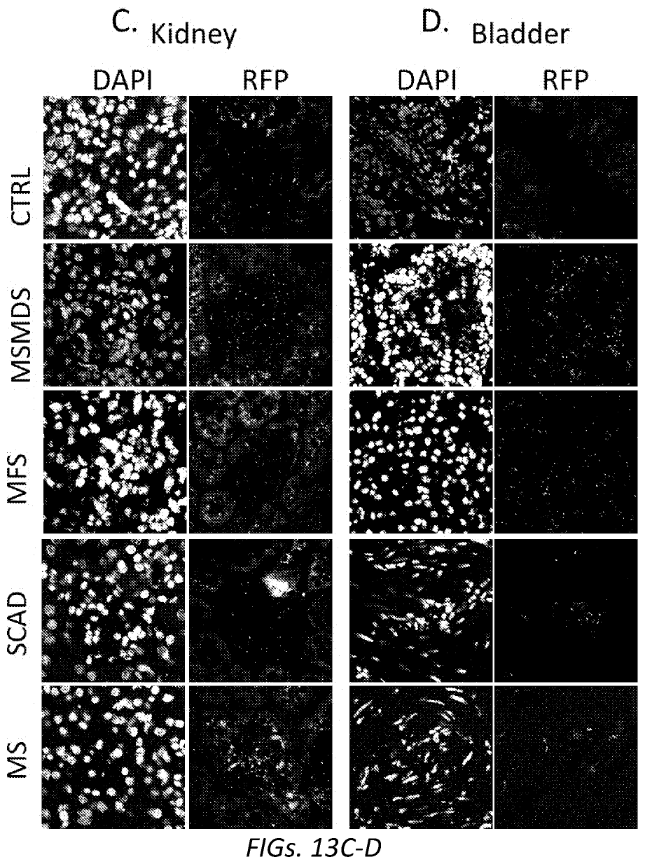

Finally, we tested the ability of the P2 HDAC9 promoter fragment to transactivate transcription in multiple vascular disease models including Myh11-Cre:Acta2^{R179Hfl} (MSMDS), Fbn1^{C1039G/+} (Marfan syndrome)⁴¹, Col3a1^{+/-}:Col5a^{+/-} (a model of coronary artery dissection, SCAD), and CMV-Cre:Smad4^{14991/fl/+} (Myhre syndrome). In these models we noted the expression of the RFP construct in vasculature and SMC of multiple organs including brain, heart, kidney, and bladder (FIGS. 13A-D). These data indicate that the HDAC9 P2 promoter fragment has general applicability to other vascular disease entities.

### Example 4. Generating ACTA2-R179H Cell Lines

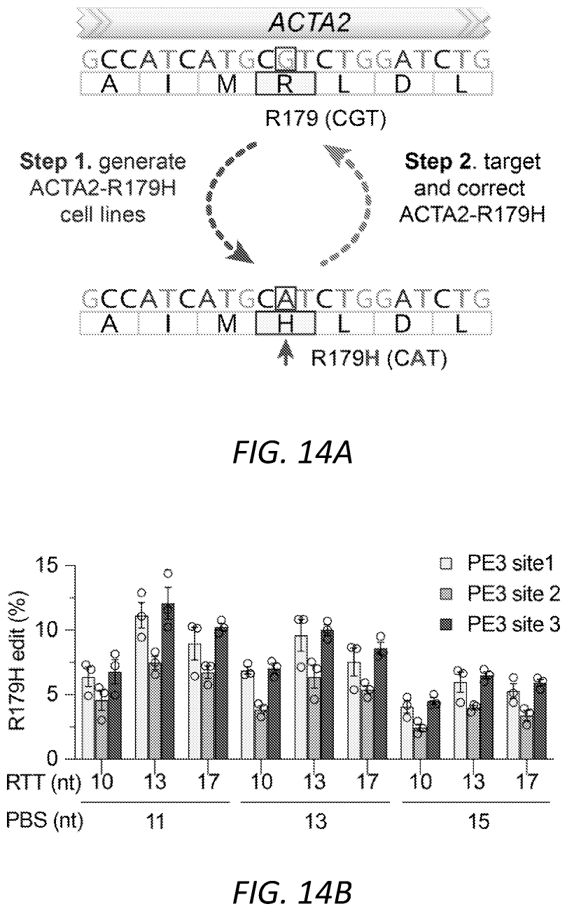

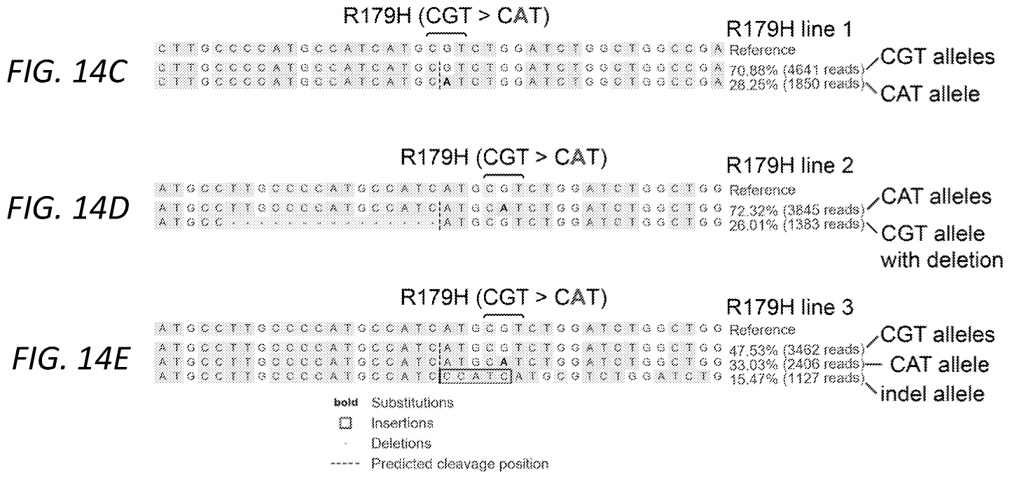

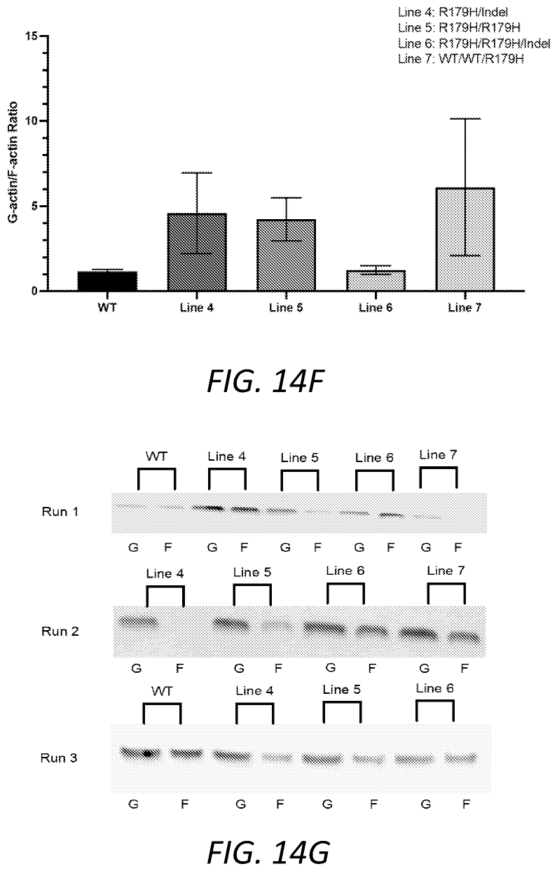

Here we investigated the potential of CRISPR-Cas editors to model and correct a substitution of arginine positioned at residue 179 for a histidine, in the ACTA2 gene, due to a single base missense mutation (CGT→CAT), referred to herein as the ACTA2 R179H mutation (FIG. 14a).

### Generation of Cell Lines

Cell lines containing the human ACTA2 R179H mutation were generated by transfecting HEK 293T cells with a prime editor expression plasmid encoding an EGFP reporter (pCMV-PE2-P2A-EGFP; HES1117) along with pegRNA and PE3 nicking sgRNA expression plasmids (HES902 and HES724, respectively). HES902 has a spacer sequence of GCCCCATGCCATCATGCGTC (SEQ ID NO:80), and a pegRNA sequence of GCCAGATCCAGATGCATGATGGCA (SEQ ID NO:81): HES724 has a spacer sequence of GGCCTCACCAGTAGTAACGA (SEQ ID NO:82). Approximately 72 hours post transfection, EGFP positive cells were sorted into a 96-well plates, grown for two passages, and then dilution plated into several 96-well plates. Individual clones were monitored by microscopy and genotypes were analyzed by extracting genomic DNA, performing PCR and sending for Sanger sequencing, or performing PCR and performing amplicon sequencing (as described below). A second round of clone dilution and selection was also performed to enrich for more stable single cell genotypes. Clones harboring ACTA2 R179H genotypes were expanded, and aliquots of ˜3×10⁶ cells were frozen in complete media with 10% extra FBS for storage in LiN2.

### Characterization of ACTA2-R179H Cell Lines

To evaluate safe and effective genome editing approaches to correct the ACTA2 R179H mutation, we first developed cell lines bearing the R179H substitution. We generated ACTA2 R179H mutation in HEK 293T cells, owing to their ease of transfection and culturing, which would permit high-throughput evaluation of dozens of CRISPR-Cas enzymes, base editors, and gRNA combinations. To generate the R179H cell line, we designed and tested three approaches: (1) nuclease-based homology-directed repair (HDR) using a single-stranded DNA donor (ssODN) template encoding the R179H mutation, (2) base editing to convert Arg179 to His, and (3) prime editing by various combinations of PE and pegRNA constructs (FIG. 14b). With the HDR approach, we achieved efficient R179H generation but that was also accompanies by high levels of insertion or deletion mutations (indels) in the population of cells, making it unlikely to identify a homozygous R179H or heterozygous R179(wild-type)/R179H clone. With base editing targeting the non-coding strand, depending on the gRNA and CBE constructs we observed R179H editing but also observed bystander edits of nearby bottom strand cytosines (which would additionally generate unwanted M1781 or D181K mutations). Finally, by selecting the most optimal permutations of PE and pegRNA combinations for prime editing, we achieved ˜12% ACTA2 R179H in bulk cell transfections.

Amongst the HDR and prime editing approaches to generate ACTA2 R179H lines, we cloned out three cell lines and genotyped them. The approximate genotypes of these cell lines, where the ACTA2 locus in HEK 293T cells appears to be triploid, were R179/R179/R179H (line 1), R179H/R179H/indel (15 bp deletion) (line 2), and R179/R179H/indel (5 bp insertion) (line 3) (FIGS. 14C-14E, respectively). Given the genotypes of these cell lines, we then dilution plated these cell lines in an attempt to get purer R179 or R179H allele balances. In doing so, we were able to derive four additional lines with cleaner genotypes, including R179H/indel(15 bp deletion) (line 4), and R179H/R179H (homozygous mutant) (line 5). Subsequent molecular characterization of ACTA2 protein expression patterns using western blot to quantify levels of globular (G) and polymerized (F) actin showed decreased amount of polymerized (F-actin) and higher G/F ratios than wild type HEK cells in mutant cells (FIGS. 14F-G). Together, these cell lines offer a range of R179H genotypes to test various corrective genome editing strategies.

### Example 5. Assessment of A-to-G Base Editing Approaches to Correct ACTA2 R179H

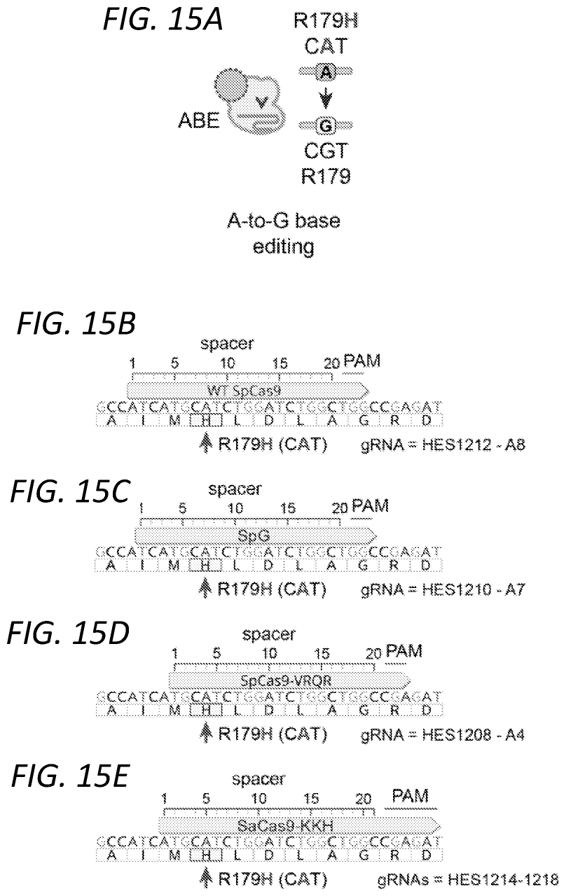

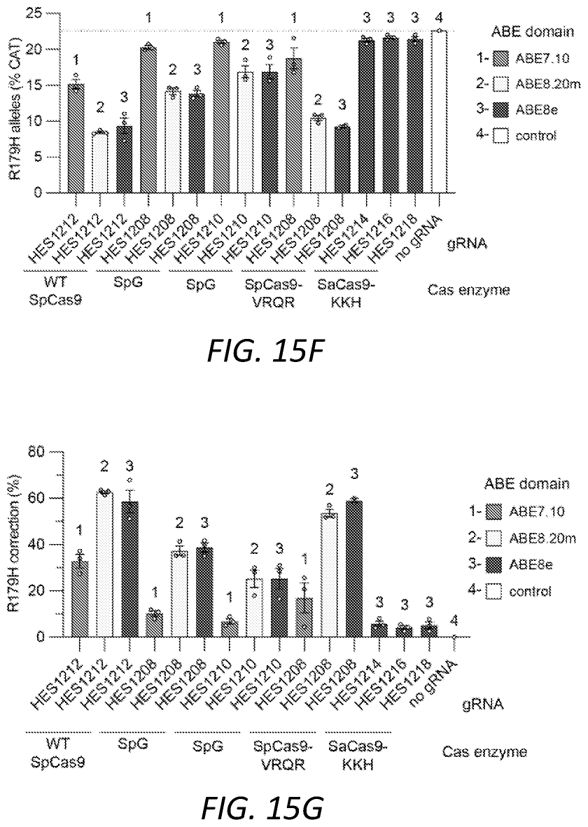

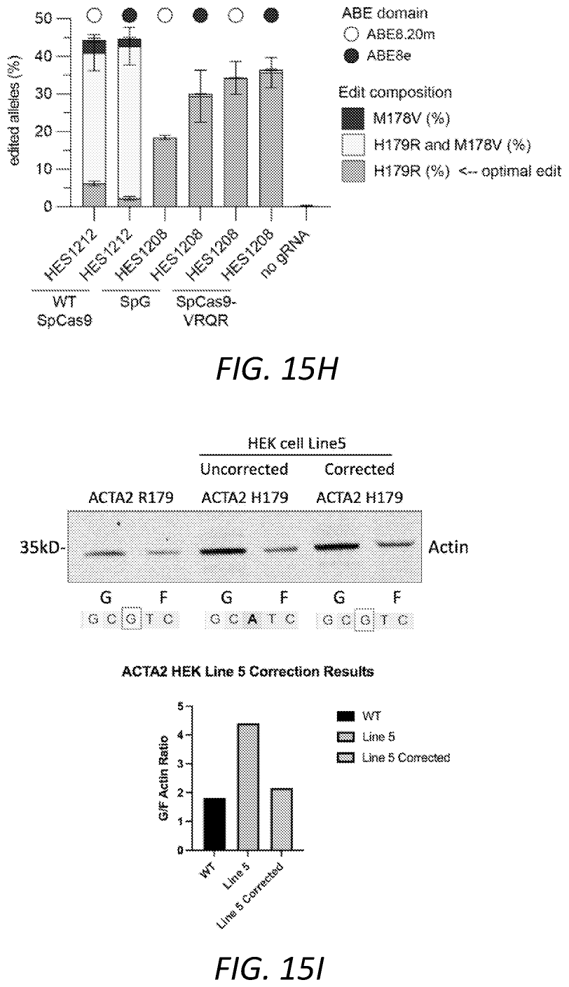

We initially selected one cell line bearing a heterozygous R179H mutation (line 1, FIG. 14C) to use as a model for examining approaches based on ABE-mediated sequence correction (FIG. 15A). To determine whether we could use ABEs to revert the disease-causing G-to-A change (CGT>CAT), we designed various gRNAs that would position the edit window of the ABE deaminase domain over the mutated nucleotide (relying on the WT SpCas9 that can target sites with NGG PAMs, the SpG variant for targeting sites with NGN PAMs⁸, the SpCas9-VRQR variant for targeting sites with NGA PAMs^{4.6}, and the SaCas9-KKH variant for targeting sites with NNGRRT PAMs⁵ (see FIGS. 15B-E, respectively). These target sites would position the target adenine for the R179H change in either the 8^{th}, 7^{th}, 4^{th}, or 5^{th} position of the spacer sequence, respectively (see FIGS. 15B-E). We also explored different deaminase components of the ABE, testing ABE7.10, ABE8.20m, and ABE8e domains^{10,15,16} (which exhibit varied A-to-G base editing efficiencies). Excitingly, next-generation sequencing (NGS) results from transfections using these ABEs and gRNAs in the ACTA2 R179H cell line 1 revealed that several enzymes could efficiently reduce the proportion of R179H (CAT) alleles in the population of cells (FIG. 15F). Focusing only on the R179H allele, many ABEs and gRNAs led to high levels of reversion to the wild-type R179 sequence (FIG. 15G). The selection of specific gRNAs, Cas variants, and deaminase domains had a major impact on the editing efficiency. These results establish the feasibility of using base editors to directly correct the ACTA2 R179H mutation. There are additional combinations of gRNAs (e.g., new target sites or varying the spacer length), Cas enzymes, and ABE domains that can be explored to improve editing potency and selectivity (Table 1).

Next we performed additional base editing experiments using a second cell line bearing the R179H/R179H/indel genotype (line 2, FIG. 14D). We selected a subset of the best-preforming base editors from our initial experiment and transfected them into R179H line 2. Once again, we observed very robust levels of R179H correction (FIG. 15H). However, this time we also analyzed the edit purity, detecting also editing of an adenine upstream that can cause an M178V mutation. By plotting the fraction of perfect H179R corrections, perfect H179R corrections with the M179V bystander edit, and M179V bystander edits only, our data revealed that the SpG and VRQR variants with the ABE8e or ABE8.20 domains and using the HES1208 gRNA (with an NGA PAM) offer the highest levels of ‘pure’ H179R correction (FIG. 15H).

### Example 6. Allele-Specific Editing Approaches to Knockout the ACTA2 R179H Allele

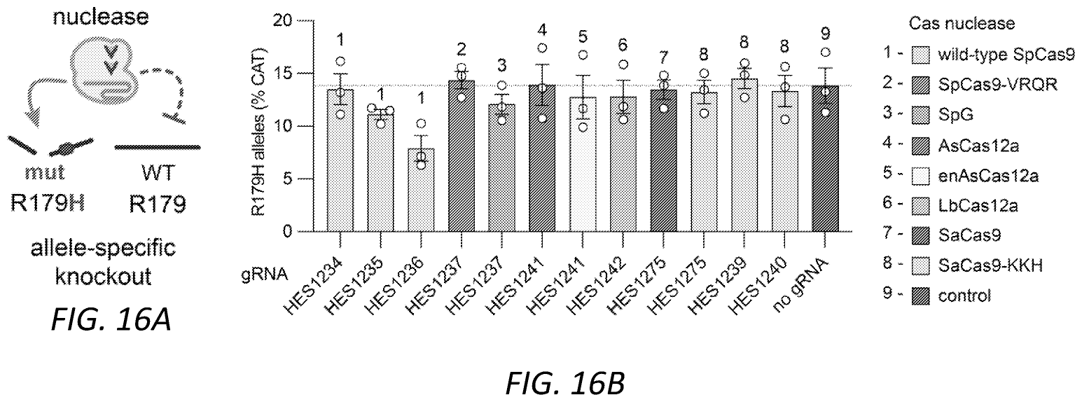

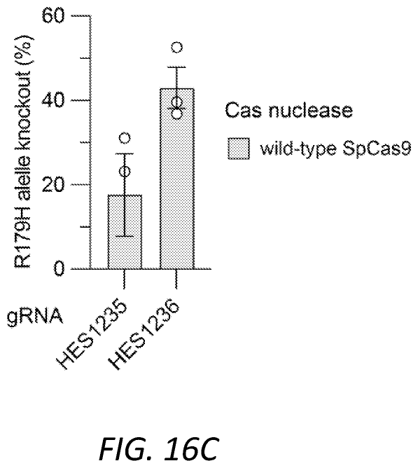

Further, we explored an allele-selective knockout approach to selectively destroy the R179H allele (FIG. 16A). Since the ACTA2 R179H variant is a dominant negative gain of function mutation, we hypothesized that knockout of the R179H allele without creating indels on the wild-type R179 allele might also be a viable therapeutic strategy. We selected the heterozygous R179H cell line (line 1, FIG. 14C) as a model for examining allele-selective editing since it harbors both wild-type and R179H sequences. Following transfection of the Cas nucleases and gRNAs into R179H line 1, three days later we extracted genomic DNA and performed targeted NGS to assess the levels of remaining R179H allele. Our sequencing revealed that several Cas enzymes are capable of knocking down the proportion of the R179H allele (FIG. 16B), with wild-type SpCas9 exhibiting the greatest levels of R179H allele reduction. When programmed with two of the gRNAs that we assayed, wild-type SpCas9 selectively knocked-out between 17.6-43.0% of the mutant R179H allele (FIG. 16C). There are additional combinations of gRNAs (e.g. new target sites or varying the spacer length) and Cas nucleases that can be explored to improve editing potency and selectivity (Table 2).

### Example 7. An Engineered Peptide Displaying AAV9 Capsid, AAV-PR, Mediates a Vasculature-Enriched Transduction Phenotype in Brain after Intravenous Delivery

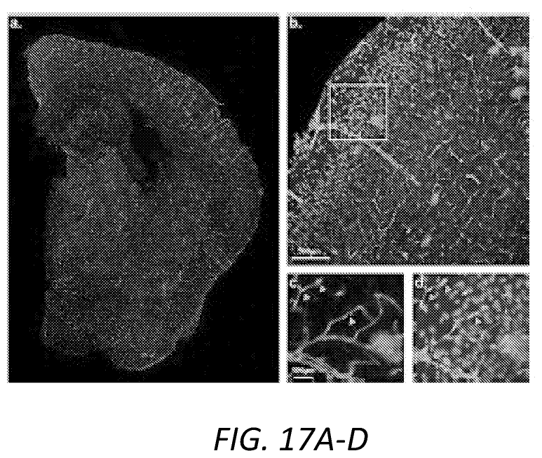

We packaged a single stranded AAV-CBA-Cre genome into capsids comprising the peptide, PRPPSTH (SEQ ID NO:1), named AAV-PR, and injected this vector (1×10¹² vg/mouse) into the tail vein of adult Ai9 mice that have a Cre-sensitive CAG-floxed-STOP-tdTomato reporter in all cells. Any cells that were successfully transduced by the AAV vector and have Cre-expressed should result in tdTomato expression. Mice were sacrificed three weeks post injection, brains sectioned, and tdTomato detected with immunofluorescence staining. In mice injected with AAV-PR, we observed a distinct vasculature immunostaining of tdTomato expression throughout the entire brain (FIGS. 17A-D). This profile is in stark contrast to the tropism of parental AAV9-CBA-Cre in adult Ai9 mice, which mediates transduction of mostly astrocytes and neurons.

### Example 8. Expression of Base Editors and CRISPR Targeting ACTA2 in SMCs In Vitro and In Vivo

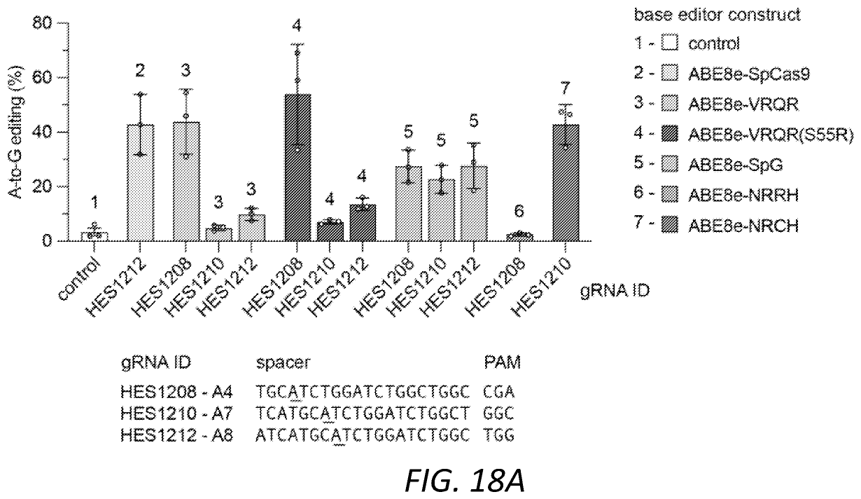

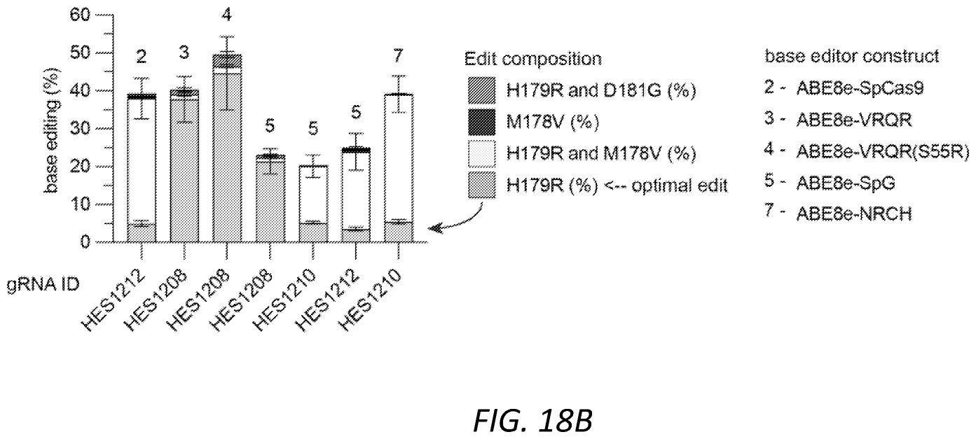

Given the promise of our base editor results, which demonstrated the feasibility of reverting the ACTA2 R179H CAT codon back to the canonical CGT codon, we explored the use of additional engineered ABEs in hope of achieving higher levels of correction. To do so, we utilized our homozygous HEK 293T ACTA2 R179H cell line to perform additional transfections. We assessed the editing efficiencies of ABE8 e16 versions of WT SpCas9, SpCas9-VRQR 4.6 (harboring D1135V/G1218R/R1335Q/T1337R mutations), SpCas9-VRQR harboring an activity-enhancing S55R mutation 17, SpG 8, and two PAM-relaxed variants SpCas9-NRRH and SpCas9-NRCH 18. We tested these enzymes using all or some of the sgRNAs targeting the three sites harboring NGA, NGC, or NGG PAMs that would appropriately place the target adenine in position A4, A7, or A8 of the edit window of the base editor (FIG. 18A). Approximately 72 hours post-transfection, genomic DNA was harvested, and we performed amplicon sequencing to analyze editing in the ACTA2 gene at or near codon R179 (FIG. 18A). Similar to our previous results, we observed high levels of intended CAT-to-CGT editing in the target ACTA R179H codon with ABE8e-SpCas9 using the A8 gRNA (FIG. 18A. With ABE8e-SpCas9-VRQR and ABE8e-SpCas9-VRQR(S55R), we observed high levels of editing only with the A4 gRNA, consistent with the preference of this SpCas9 variant to recognize sites with NGA PAMs. Given prior nuclease data showing the superiority of the SpCas9-VRQR(S55R) over SpCas9-VRQR 17, we observed a substantial improvement in A-to-G editing with ABE8e-SpCas9-VRQR(S55R) (FIG. 18A). Next, with ABE8e-SpG we observed modest levels of editing with any of the three sgRNAs (since all target sites harbor NGG PAMs), with ABE8e-SpCas9-NRRH we observed low levels of editing with the A4 NGA PAM gRNA, and with ABE8e-SpCas9-NRCH we observed high levels of editing with the A7 NGC PAM gRNA (FIG. 18A). However, closer inspection of the mutation profile across each allele revealed that many of the high efficiency ABE8e-gRNA combinations led to bystander edits of unknown consequences (FIG. 18B). In fact, only ABE8e-SpCas9-VRQR, ABE8e-SpCas9-VRQR(S55R), and ABE8e-SpG when paired with the A4 NGA PAM gRNA resulted in “clean” edits of the H179R reversion only. Use of ABE8e-SpCas9 or ABE8e-SpG with the A8 NGG PAM gRNA, and ABE8e-SpG or ABE8e-SpCas9-NRCH with the A7 NGC PAM gRNA led to dual H179R and M178V editing (FIG. 18B); we observed co-occurrence of H179R and D181 edits in a small fraction of reads ABE8e-SpCas9-VRQR, ABE8e-SpCas9-VRQR(S55R), and ABE8e-SpG when paired with the A4 NGA PAM gRNA. From these results, we proceeded with ABE8e-SpCas9-VRQR(S55R) and the A4 NGA PAM gRNA for in vivo mouse studies, given that we observed the most efficient editing with this construct and also the purest edit outcomes for H179R only.

Evaluation of Split Base Editors for Packaging into AAV Vectors

Thus far our assessment of editing efficiency had been performed using a typical two-plasmid method, where one plasmid encodes the ABE and a second encodes the gRNA (FIG. 19a). Towards assessment of in vivo editing, we therefore generated plasmids encoding these constructs in AAV vectors with the goal of producing AAVs. Due to the large sizes of SpCas9-based base editor coding sequences, we endeavored to split the ABE8e-SpCas9-VRQR(S55R) sequence into two parts, similar as to what has been done before for other ABE7.10 and CBE constructs. The first AAV construct encodes the ABE8e domain and an N-terminal portion of SpCas9 fused to the N-terminal end of the split alpha subunit of the DNA polymerase III (DnaE) intein from Nostoc punctiforme PCC73102 (Npu)²⁰⁻²² (FIG. 19b). The second AAV construct encodes the C-terminal Npu intein domain fused to the C-terminal domain of SpCas9 along with the SpCas9 gRNA expression cassette (FIG. 19b). Notably, the N-terminal SpCas9 construct includes the D10A nickase mutation (required for efficient base editing) and the activity-enhancing S55R mutation¹⁷, while the C-terminal SpCas9 construct encodes the VRQR mutations. We performed transfections in our homozygous ACTA2 R179H HEK 293T cell line to compare ABE8e-SpCas9-VRQR to ABE8e-SpCas9-VRQR(S55R) with P2A-EGFP or P2A-mScarlet reporters all in single plasmid architectures with a separate A4 NGA PAM gRNA plasmid (FIG. 19a), along with the dual-AAV split ABE8e-SpCas9-VRQR(S55R) architecture encoding the A4 NGA PAM gRNA in the second AAV construct (FIG. 19b). We observed comparable editing between each condition, suggesting that the dual AAV plasmid architecture could lead to high levels of correction of the R179H mutation (FIG. 19c).

### In Vivo Editing of Mouse ACTA2 R179H (Myh11-Cre:Acta2R179Hfl/+ Genotype)

Encouraged by these findings, we applied base editing in vivo to correct our mouse model of ACTA2 R179H (C57BL/6 Myh11-Cre:Acta2R179Hfl/+ mutant mice) which as shown herein recapitulates hallmark symptoms of the human brain, aorta and multisystemic pathological phenotypes. The Acta2R179Hfl allele contains 2 copies of exon 6 (FIG. 1A), one with a CGT codon (Wild type) and one with a CAT codon (mutant) at position 179, complicating interpretation of sequence based analysis of editing efficiency. After cre-mediated excision the wild type codon within the Acta2R179Hfl allele is excised (CGT codon). Therefore within Cre-expressing cells within the mouse, the Acta2R179Hfl allele only contains the mutant codon (CAT codon). Since it is useful to compare editing efficiency in animals that have an activated allele and are therefore phenotypically affected, this necessitates an unequal codon frequency in experimental animal tissues when compared to control tissues. The baseline codon frequency in the Myh11-Cre:Acta2R 179Hfl/+ mutant animals is a 1:1 ratio of the wild-type allele (one CGT codon and one CAT codon) within smooth muscle cells. In the same Myh11-Cre:Acta2R179Hfl/+ mutant animals, cells not expressing cre recombinase have a codon ratio of 2:1 (one CAT codon and two CGT codons). Similarly, in control animals with the genotype Acta2R179Hfl/+, all cells contain the 2:1 codon ratio (one CAT codon and two CGT codons). To account for this complexity, we designed sequencing primers that do not recognize the altered wildtype codon in the Acta2R179Hfl allele making sequencing interpretable as a baseline 1:1 CAT to CGT codon ratio within cre-expressing cells and 1:1 CGT to CGT codon ratio in cells that do not express cre recombinase (see below).

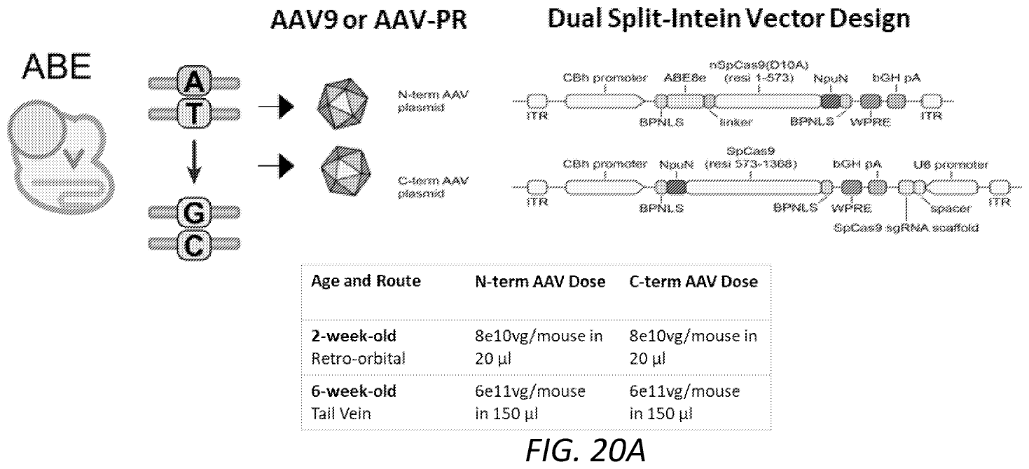

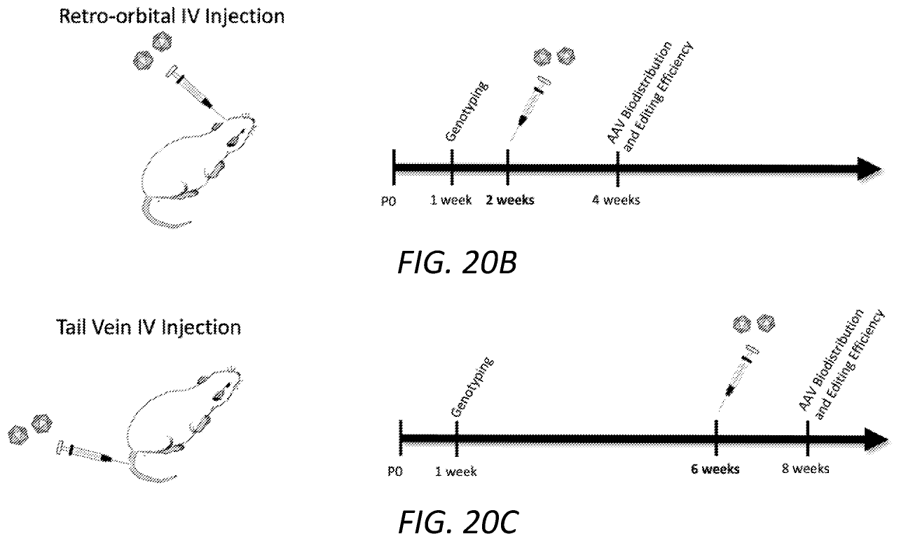

Using the above mentioned dual-AAV split ABE8e-SpCas9-VRQR(S55R) packaged into two different AAV viral vectors (AAV9 and our novel AAV-PR) we injected Acta2R179Hfl/+ control and Myh11-Cre:Acta2R179Hfl/+ mutant mice. To compare the effects of injection route and timing on in vivo editing we performed retro-orbital injection of 2-week (2-3-week-old mice) (n=9) and 6-week-old mice (n=7). Two week-old mice were injected with 1.3 e13 vg/kg of each AAV for a total of 2.7 e13 vg/kg AAV per mouse (1.6 e11 vg/6 grs mouse total AAV) and 6-week-old mice were injected with 4 e13 vg/kg of each AAV vector for a total injection of 8 e13 vg/kg AAV per mouse (1.2 e12 gc/15 grs mouse total AAV). Mice were euthanized at the age of six weeks and editing and Cas9 enzyme mRNA and protein expression were evaluated in various tissues following standard DNA, RNA and protein extraction protocols (see FIG. 20A-D).

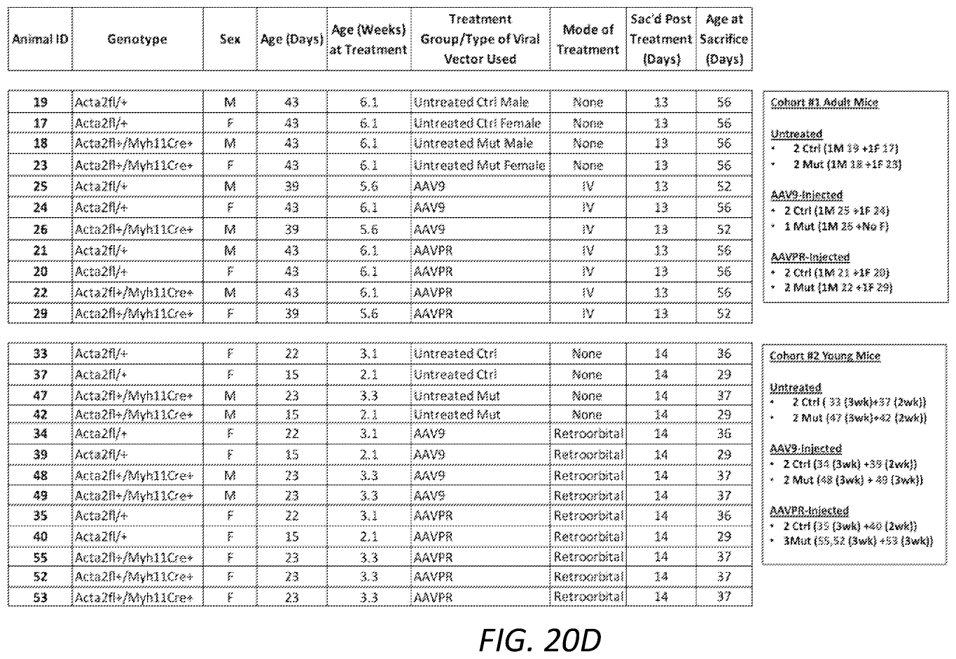

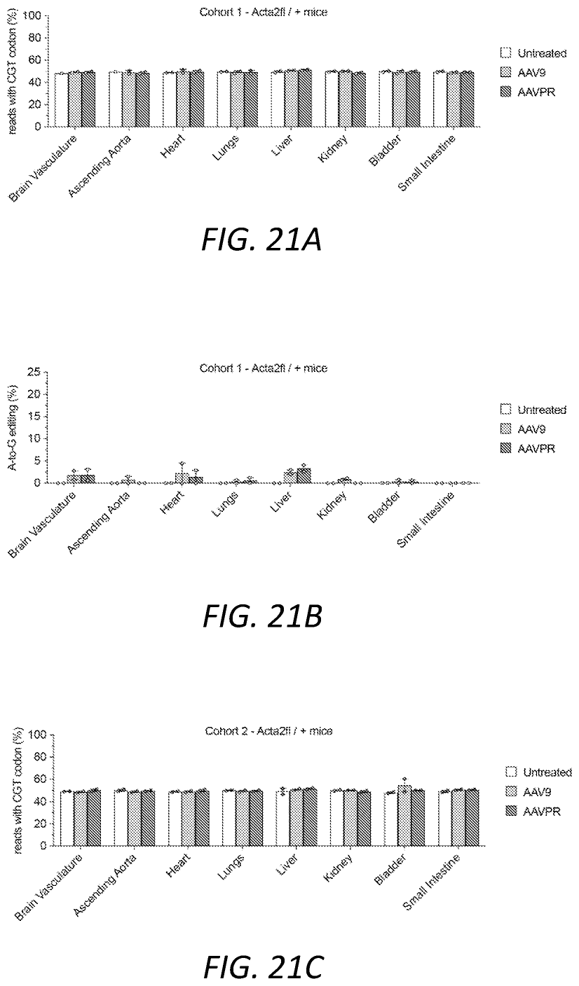

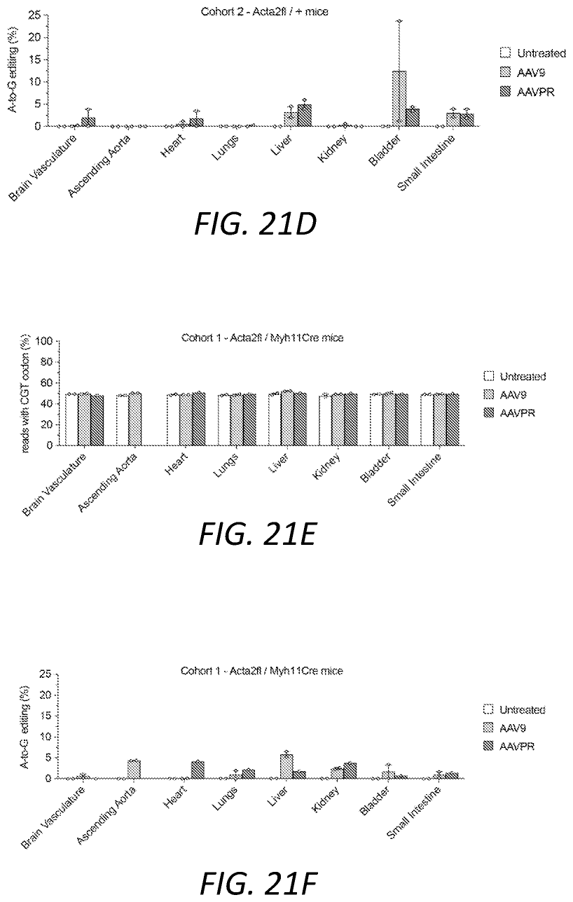

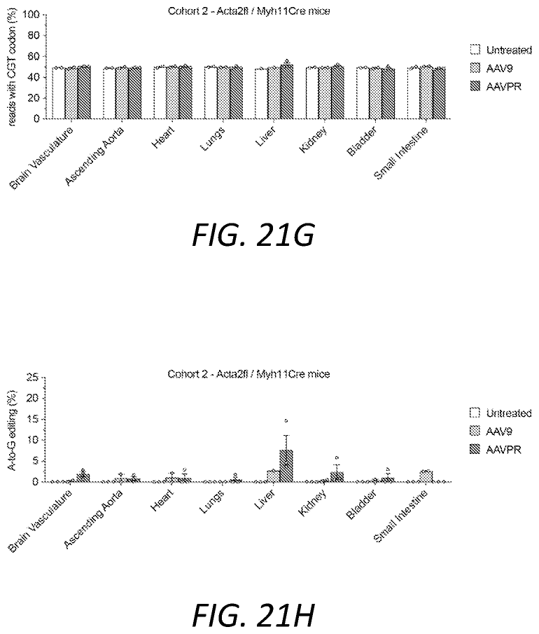

Extracted genomic DNA from both cohorts of the Acta2R179Hfl/+ control and Myh11-Cre:Acta2R179Hfl/+ mutant mice treated with AAV9, AAV-PR, or untreated was used to analyze editing at the Acta2 R179H locus, by performing amplicon sequencing. Irrespective of the actual genotype and cre recombination status of the animals in these injections, our sequencing approach amplifies a basal genotype of a 1:1 ratio of the wild-type allele (R179 with CGT codon) and mutant allele (R179H with CAT codon) given that our primers do not recognize the altered sequence of the wild-type exon 6 in the mutant allele before or after cre recombination. Thus, our primers only amplify the mutant copy on the mutant allele and the wild-type locus of a wild-type allele, and do not amplify or sequence the wild-type exon 6 encoded on the mutant allele. Amplification and sequencing of this locus led us to observe variable levels of editing in various tissues depending on the cohort of the animals and the serotype of AAV used in the injections (FIGS. 21A-H). Notably, the younger mice of cohort #2 (2-week-old) showed higher transduction and CAT to CGT editing levels in the brain vasculature when the dual-AAV split ABE8e-SpCas9-VRQR(S55R) was delivered with AAV-PR when compared to AAV9. Liver editing was also more efficient in younger mice when using AAV-PR vector despite a relative lower amount of AAV genome copies potentially suggesting higher levels of ABE expression following cell transduction. Untreated mice showed no transduction or editing in any tested tissues (FIGS. 21A-H).

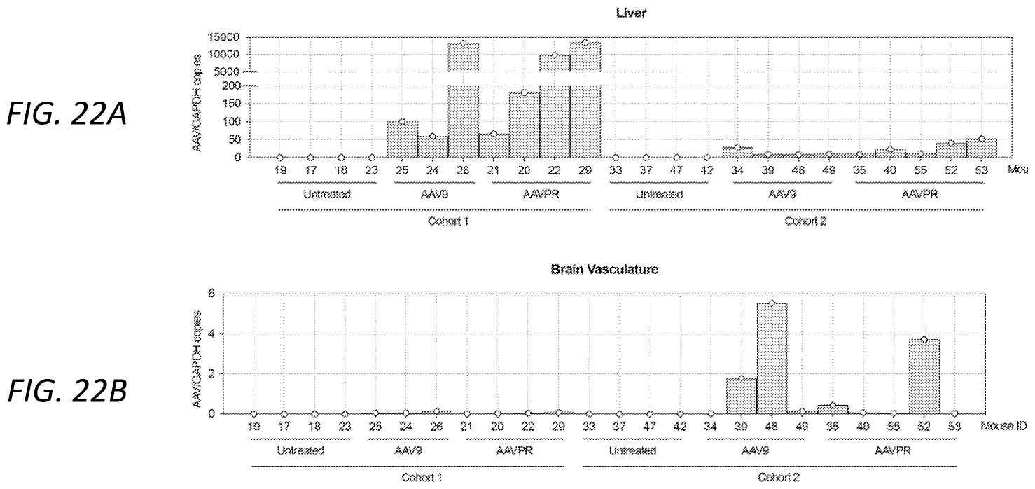

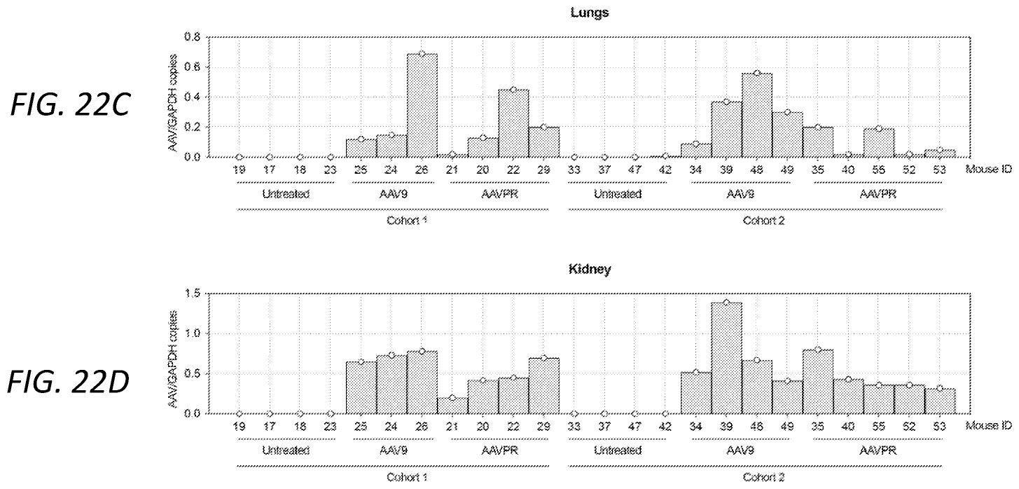

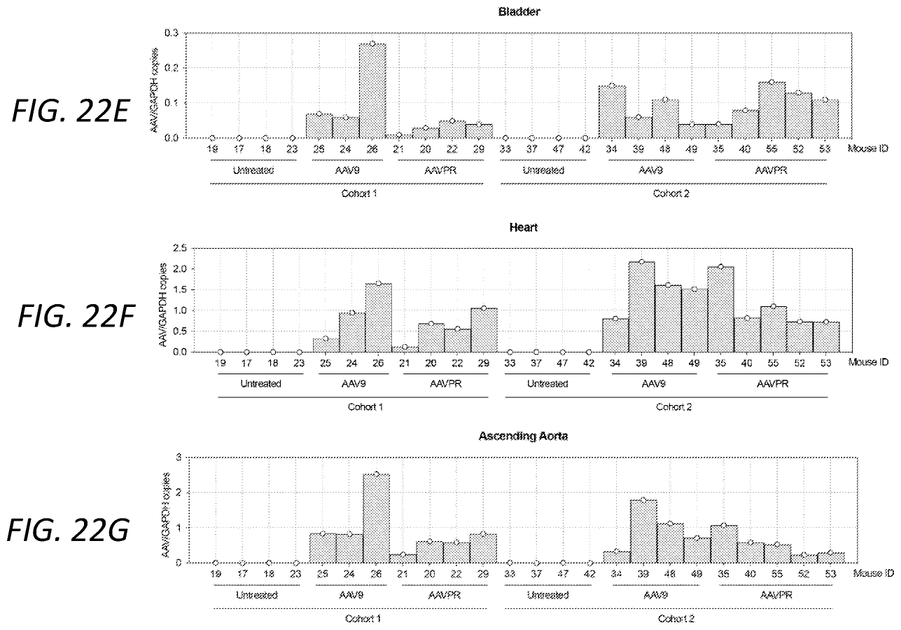

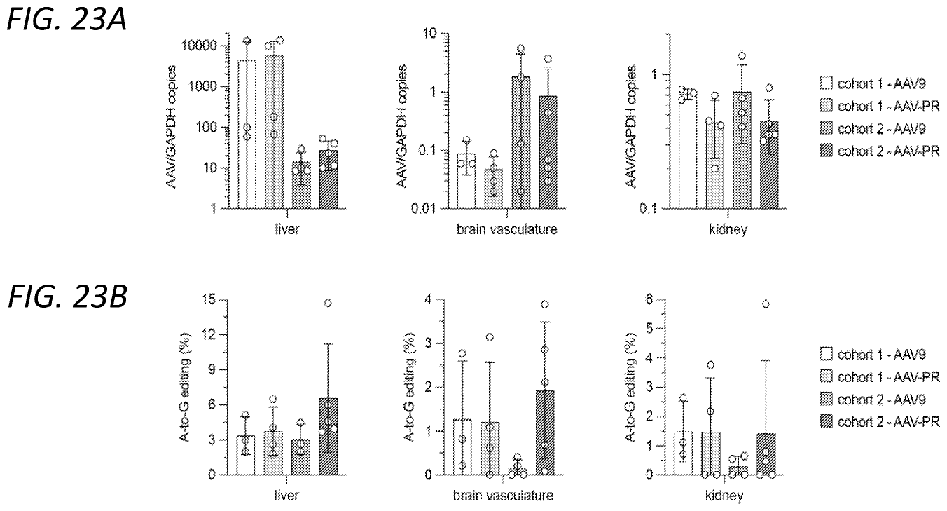

To analyze the levels of AAV transduction in each tissue and in each cohort of injections, we utilized the extracted genomic DNA (containing AAV genomes) to perform ddPCR (FIGS. 22A-G). As expected, for both AAV9 and AAV-PR, we observed the highest levels of AAV transduction in the livers of both cohorts of animals, though this observation was much more pronounced in cohort 1 where direct intravenous injection via tail vein was used and when AAV-PR was used as the vector (FIG. 20A). To determine Cas9 enzyme expression in liver and vasculature (aorta) Cas9 mRNA quantification using qPCR and Cas9 immunofluorescence were performed. Higher levels of Cas9 mRNA expression were found in the vasculature (aorta) of 2-week-old mice (cohort #2) treated with dual-AAV-PR when compared to AAV9 (FIG. 23A). Moreover, Cas9 immunofluorescence confirmed primarily vascular (arterial, vein and sinusoidal capillaries) Cas9 protein expression in the liver 2 weeks after injections (FIG. 23B). The lower expression in hepatocytes maybe due to the high mitotic rates during the liver growth of young mice, diluting the episomal Cas9 expression of subsequent hepatocyte generations. In contrast, the low mitotic rates of vascular cells (endothelium, pericytes and SMCs) contribute to the longer Cas9 expression.

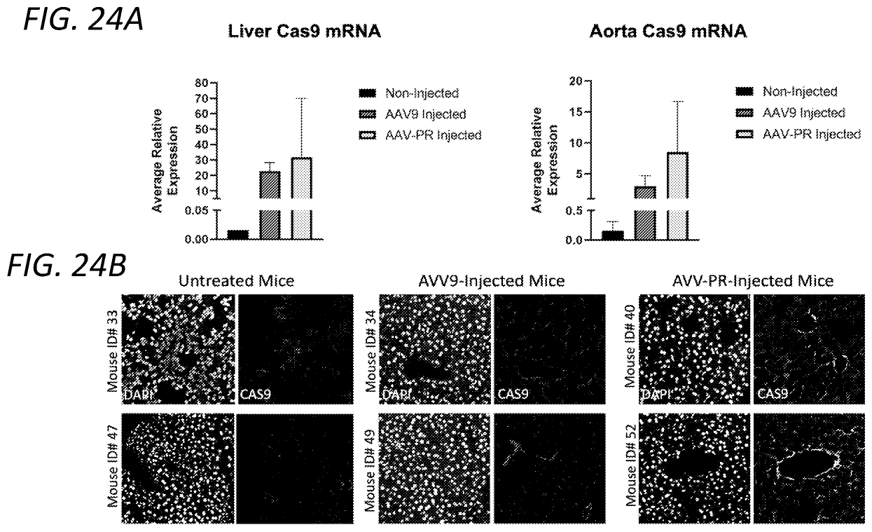

These data taken together show detectable levels of A-to-G editing in both control and mutant mice (both harboring a copy of the mutant allele R179H with CAT codon) that are encouraging particularly in light of the short term survival allotted to these mice. In young mice (cohort #2) a trend towards higher editing efficiency was observed when injected with dual-AAV-PR split ABE8e-SpCas9-VRQR(S55R) compared to the dual-AAV9 split ABE8e-SpCas9-VRQR(S55R) injected group (See FIGS. 24A and 24B).

### Example 9. In Vivo Editing of ACTA2 R179H (C57BL/6 Myh11-Cre:Acta2R179Hfl/+ Genotype) Mice at p3—Therapeutic Efficacy

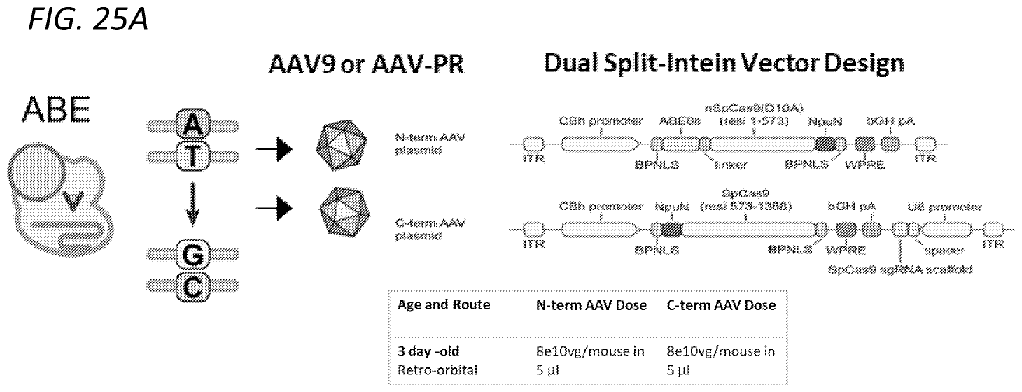

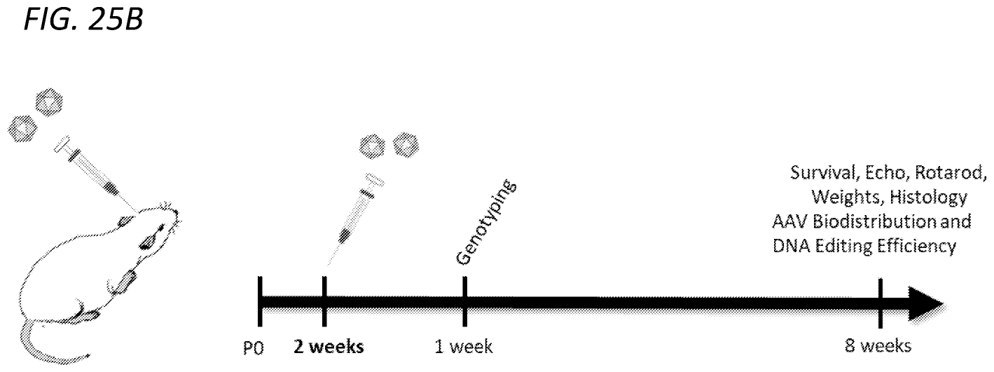

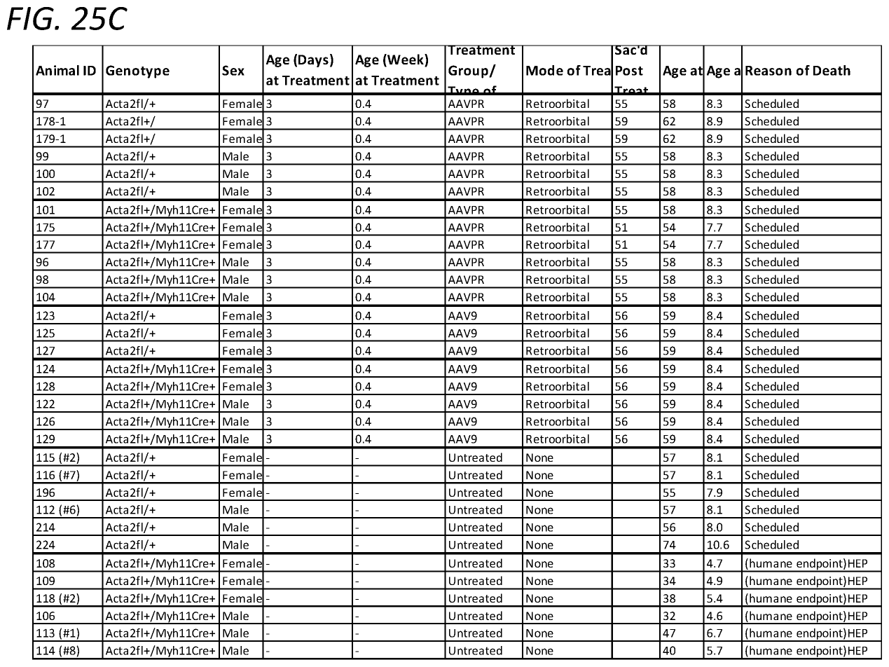

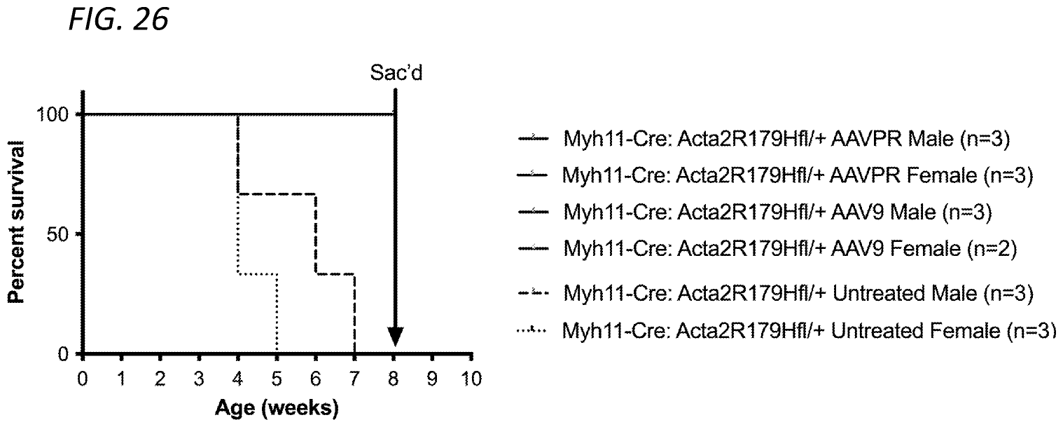

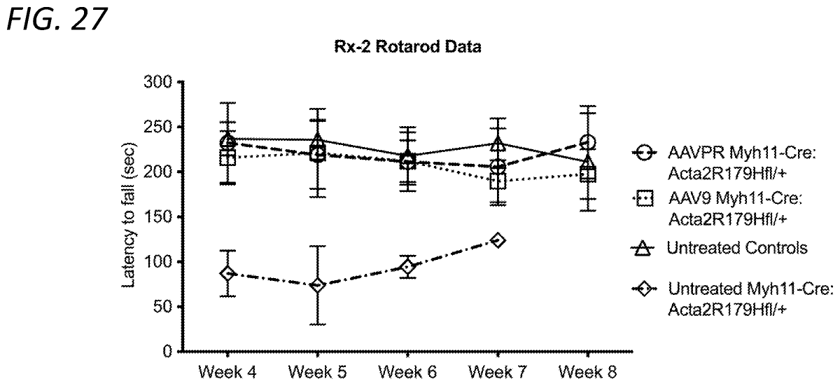

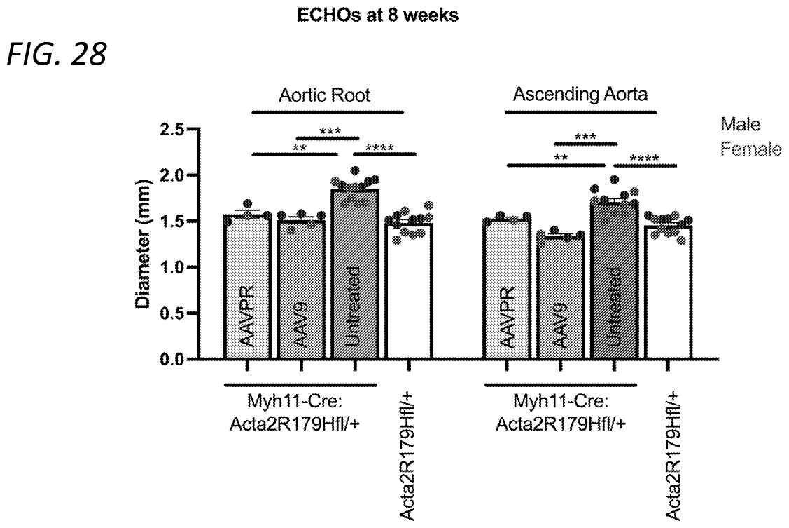

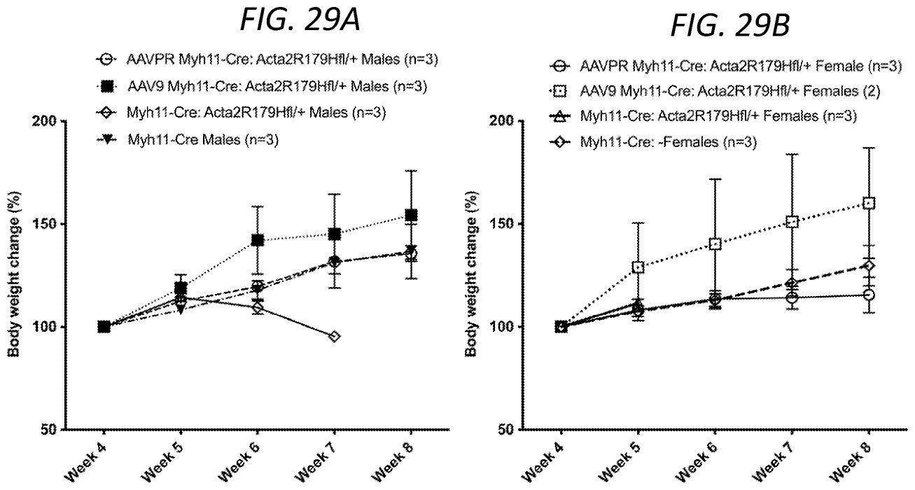

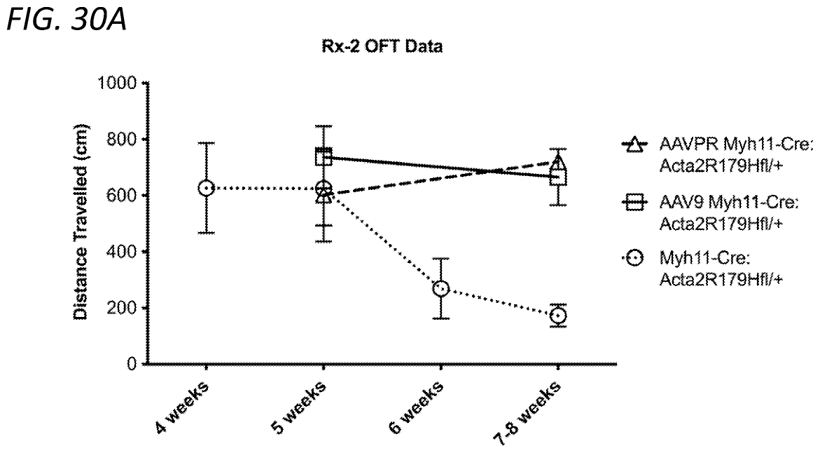

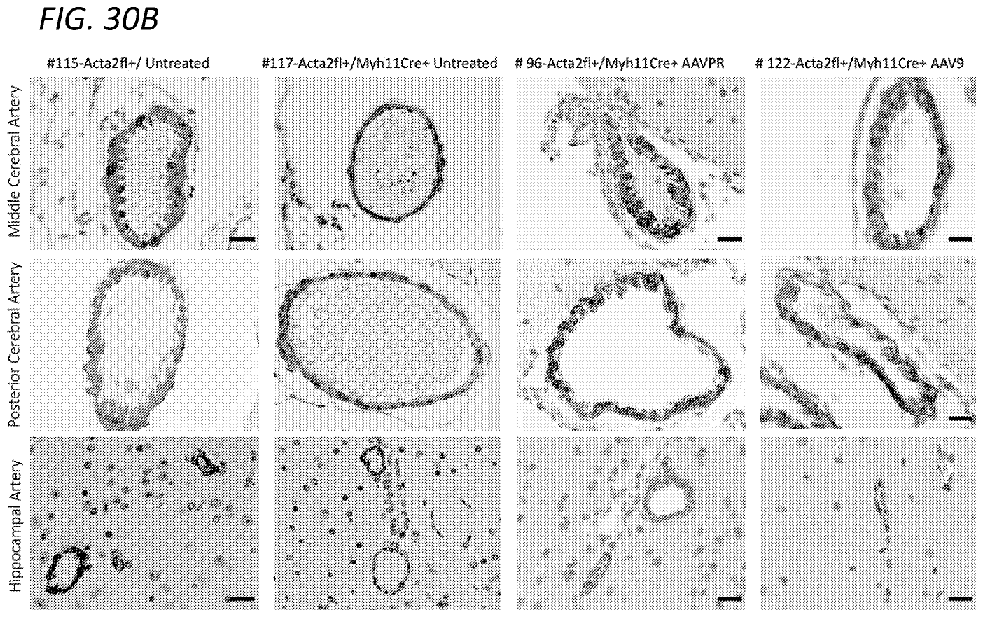

To determine therapeutic efficacy in vivo we proceeded to injected 3 day-old mice with the above mentioned dual-AAV split ABE8e-SpCas9-VRQR(S55R) packaged into two different AAV viral vectors (AAV9 and our novel AAV-PR) at 1.3 e13 vg/kg of each AAV for a total of 2.7 e13 vg/kg AAV per mouse (1.6 e11 vg/2 grs mouse total AAV) into Acta2R179Hfl/+ control and Myh11-Cre:Acta2R179Hfl/+ mutant mice via retro-orbital injection (n=6 control and 6 mutant mice for AAV-PR and n=6 controls and 5 mutant for AAV9) (FIGS. 25A-C). Mice were euthanized at the age of eight weeks and survival, rotarod test, aortic root diameter determined by echocardiography (ECHO), weights, open field test performance, histology, AAV genome biodistribution and A-G editing efficiency were evaluated in various tissues following standard DNA, RNA and protein extraction protocols. Treatment with dual-AAV split ABE8e-SpCas9-VRQR(S55R) editors resulted in increased survival and normalization of weight gain, aortic root diameter, rotarod and open field behavioral performances of Myh11-Cre:Acta2R179Hfl/+ mutant mice (see FIGS. 26, 27, 28, 29A-B, and 30A). Histological analyses of organs and brain vasculature showed normalization of vessel diameters, tortuosity and smooth muscle cell contractility in muscular arteries demonstrated by wrinkling of interna elastica layer and ACTA2 (SMA) staining is shown in FIG. 30B. No severe toxicity from the treatment was observed in all injected mice up to day of sacrifice.

These data taken together demonstrate in vivo that A-G editing leading to a significant prevention of disease phenotype in an animal model of the disease.

### Assessment of On-Target R179H Editing in a Cohort of P3 AAV-Injected Mice

To determine whether injections of the ABE-encoding AAV vectors earlier in the life of the animals may lead to increased correction of R179H, we injected an additional cohort (#3) of our mouse model of ACTA2 R179H (C57BL/6 Myh11-Cre:Acta2R179Hfl/+). We performed P3 injections with AAV9 or AAV-PR packaged dual AAV vectors encoding the split ABE8e-SpCas9-VRQR(S55R) construct with the A4 NGA PAM gRNA via retro-orbital injection using 1.3 e¹³ vg/kg of each N- and C-terminal vector for a total of 2.601013 vg/kg per mouse (with n=8 and n=14 mice for AAV9 and AAV-PR, respectively). Mice were euthanized after 8 weeks and genomic DNA was extracted from sections of various tissues.

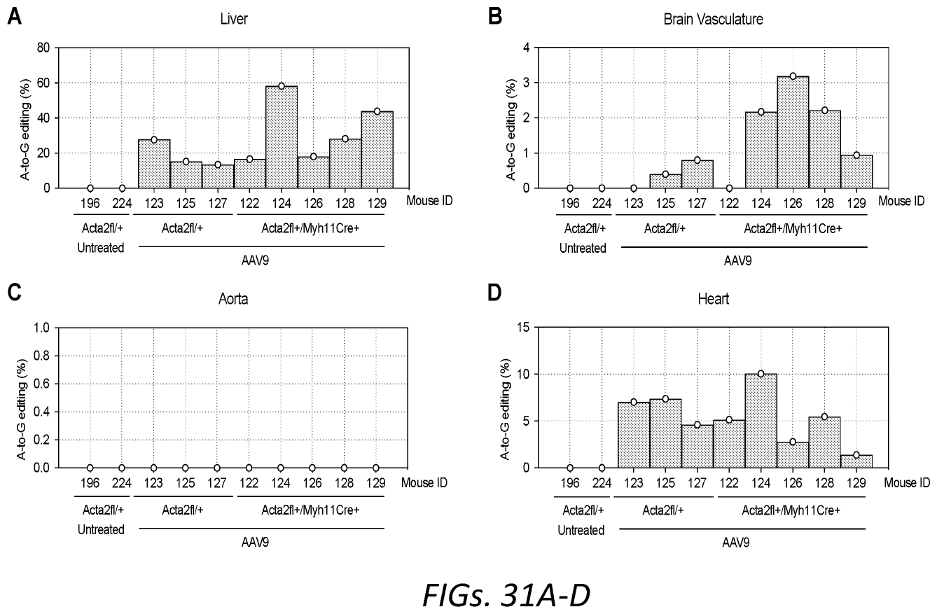

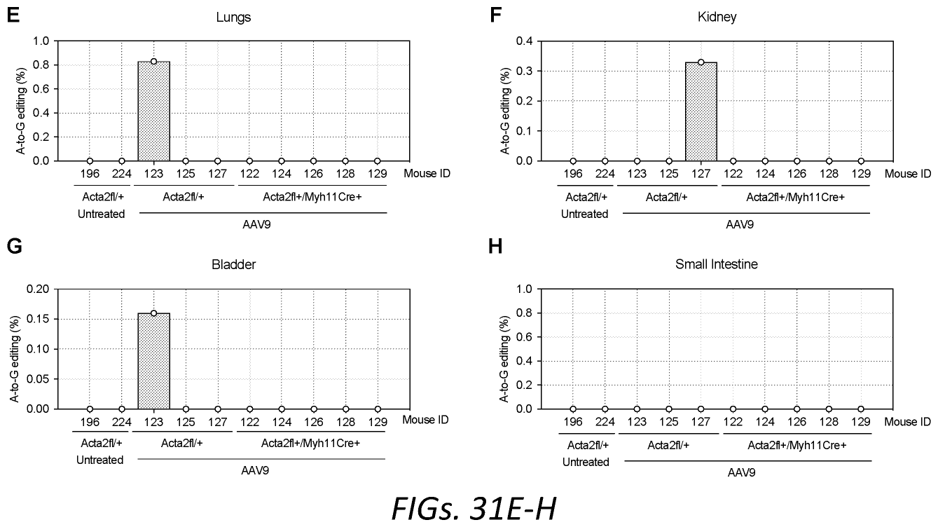

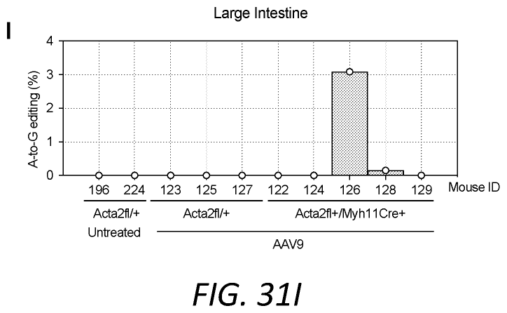

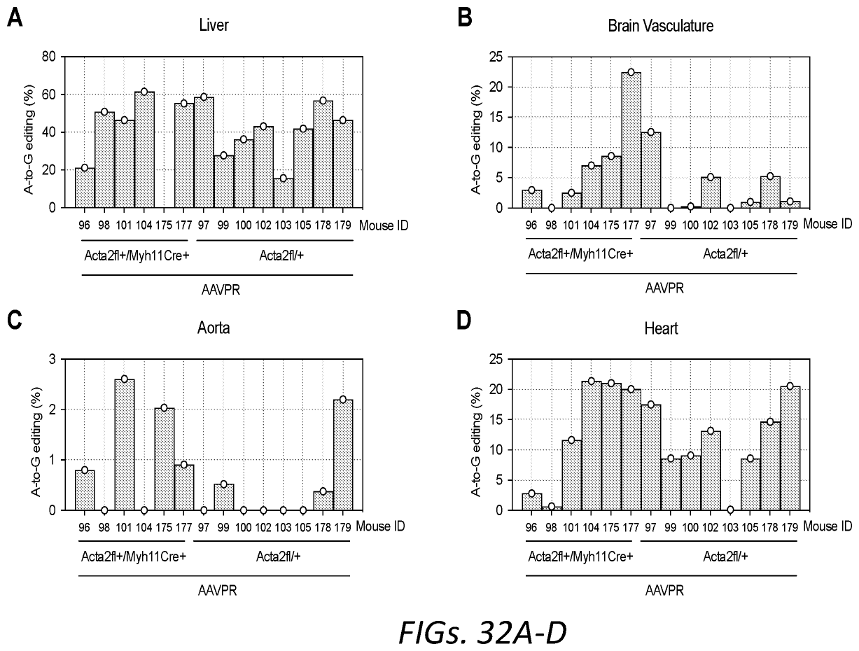

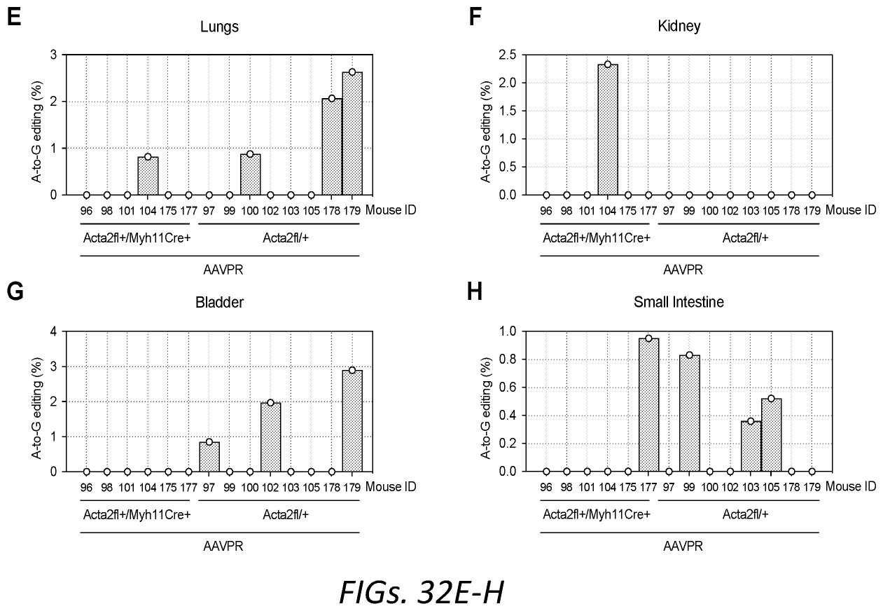

We analyzed editing at the humanized Acta2 R179H locus by amplicon sequencing in both Acta2R179Hfl/+ control and Myh11-Cre:Acta2R179Hfl/+ mutant mice. A-to-G editing to correct R179H was assessed in bulk tissue across all treatment groups without selection or sorting for transduced cells (Table 5). Consistent with the previous results demonstrating that injections earlier might lead to more efficient correction, we observed substantially higher correction of R179H when injecting at P3 compared to later injections in prior cohorts. When analyzing tissues from mice treated with dual AAV9 vectors encoding ABE8e-SpCas9-VRQR(S55R) with the A4 gRNA, in liver we observed up to 60% A-to-G editing at R179H>R (converting CAT to CGT) versus control samples (FIG. 31A). Across various other tissues including brain vasculature, ascending aorta, heart, lungs, kidney, bladder, small intestine, and large intestine (FIGS. 31B-I, respectively; Table 5), in many cases we detected appreciable levels of editing in bulk tissue (up to ˜3% in brain vasculature and ˜10% in heart, without sorting for transduced cells). We then analyzed editing in animals from the same cohort but treated with dual AAV-PR vectors encoding the same ABE and gRNA. In most cases, we observed higher levels of editing from AAV-PR treated mice compared to the AAV9 across nearly all tissues analyzed (FIGS. 32A-I and Table 5). For instance, we achieved higher average editing in liver reaching ˜43.3% with AAV-PR versus ˜27.7% mean editing with AAV9 (FIG. 31A vs. 32A). Similarly, we observed ˜4.9% and ˜1.2% mean editing in brain vasculature (FIG. 31B vs. 32B for AAV9 and AAV-PR, respectively) and ˜12.1% and ˜5.5% mean editing in heart (FIG. 31D vs. 32D for AAV9 and AAV-PR, respectively).

Together, these results reveal that our in vivo ABE8e-SpCas9-VRQR(S55R) with gRNA A4 editing approach resulted in high levels of ACTA2-R179H correction, especially when the AAV-encoded ABE constructs are delivered early in life.

### Analysis of Unintended Edits

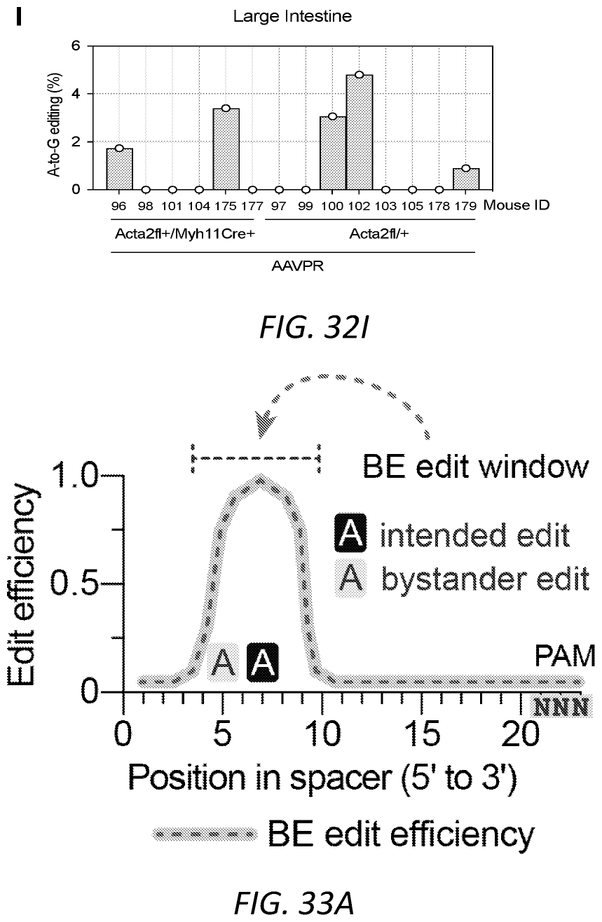

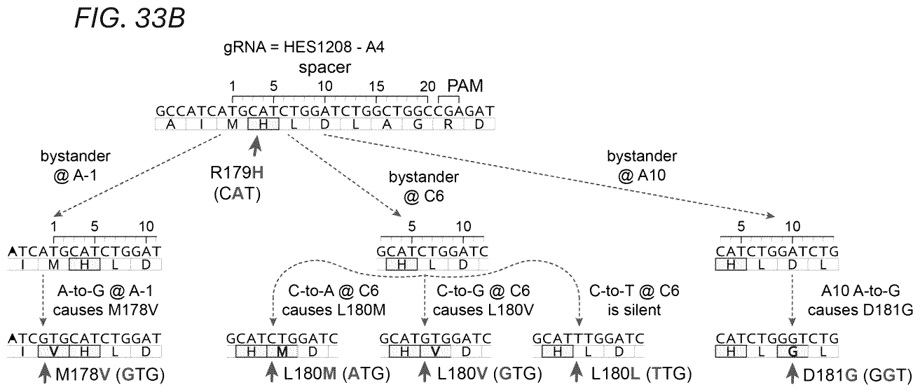

We also explored the degree to which this ABE strategy could create different types of unwanted edits at the on-target site when using AAV-delivered ABE8e-SpCas9-VRQR(S55R) with gRNA A4. We analyzed the level of bystander A-to-G editing of two nearby adenine bases that can cause ACTA2 M178V or D181G mutations (located in positions A-1 and A10, respectively, compared to the intended A4 edit; FIG. 33; Table 5). Since ABEs are known to also cause unwanted edits at cytosines located in position 6 (C6) of an ABE target site (when the cytosine is found in a thymine-cytosine (TC) sequence context; Kim et al., Nature Biotechnology, 2019, PMID: 31548727; Li et al., Nature Communications, 2020, PMID: 33203850; Jeong et al., Nature Biotechnology, 2021, PMID: 34211162), we also analyzed the propensity for our ABEs to generate bystander C6-to-A, C6-to-G, or C6-to-T edits, which cause amino acid changes L180M, L180V, or a silent L180L, respectively (FIG. 33B; Table 5).

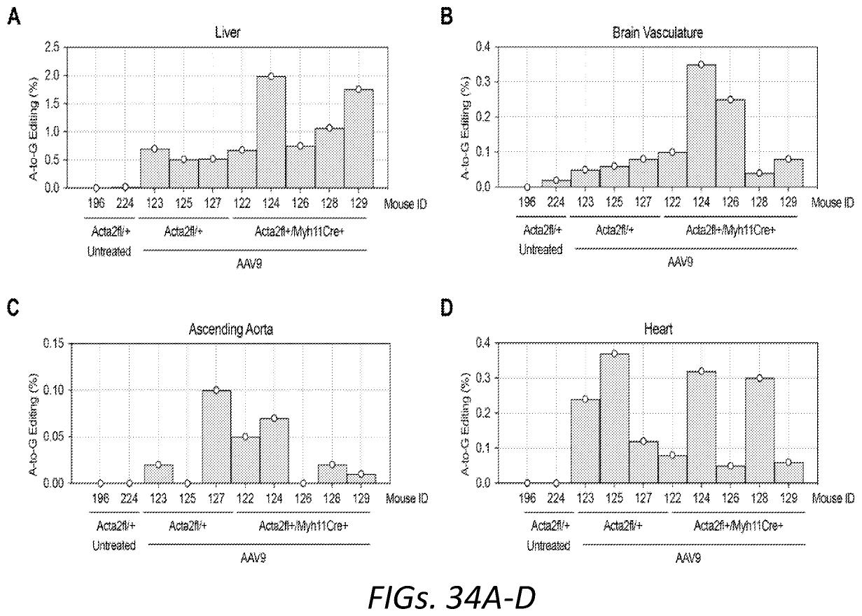

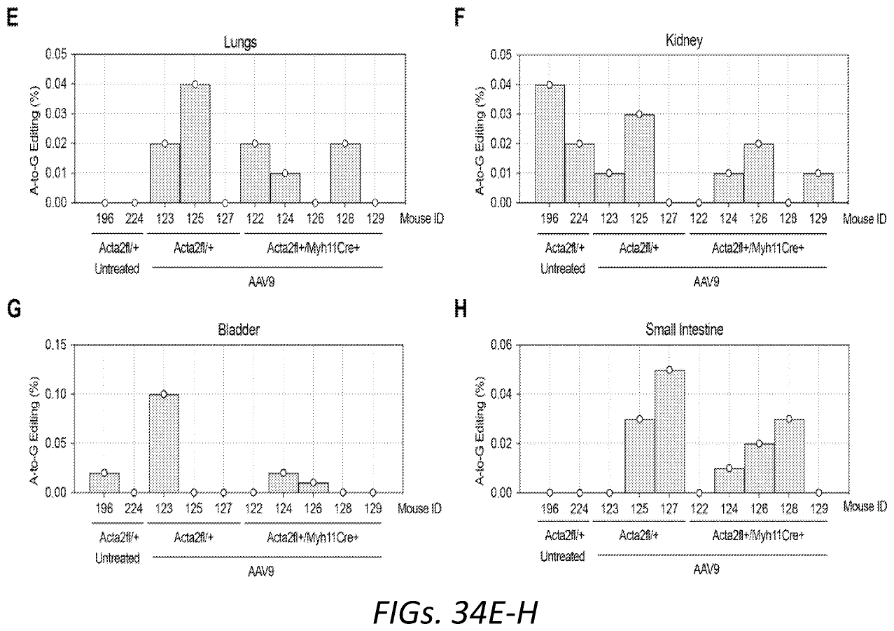

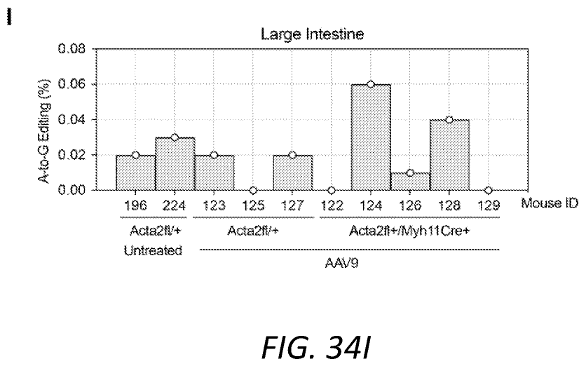

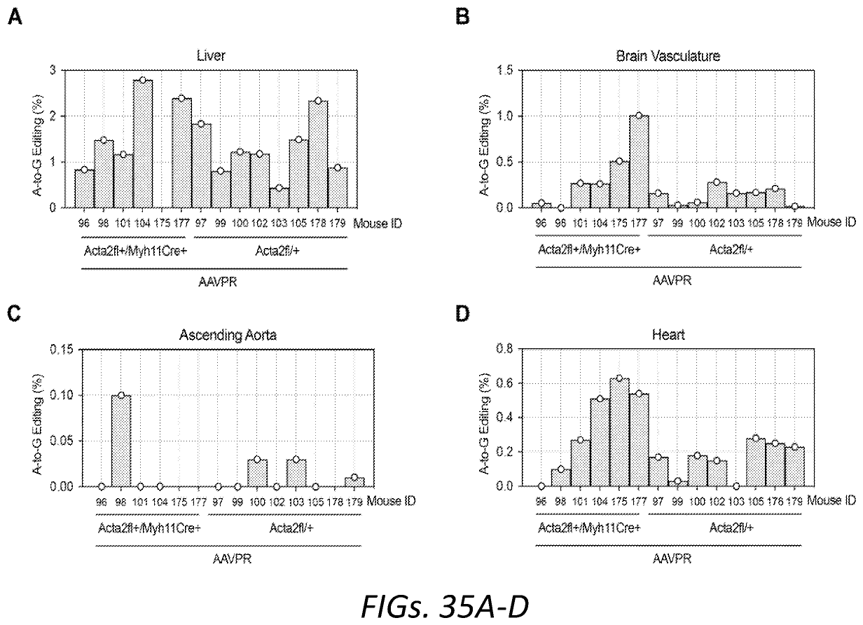

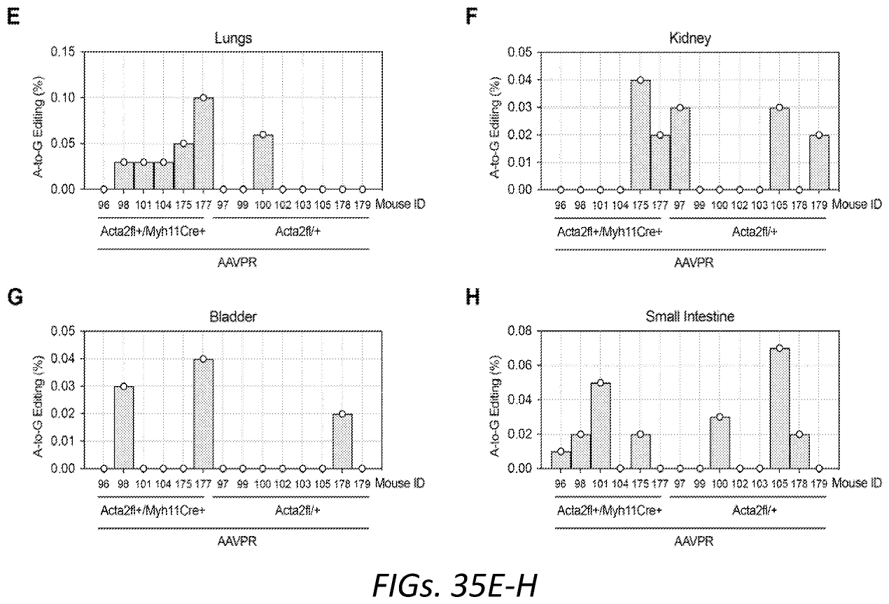

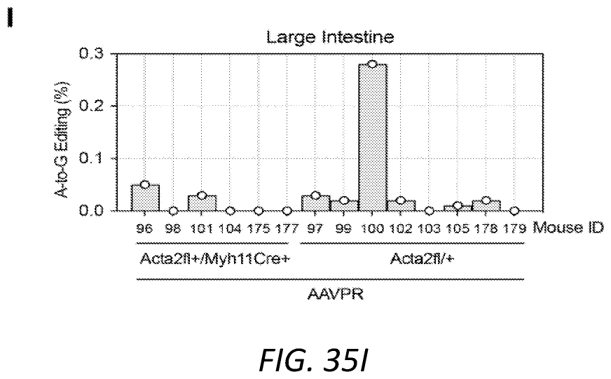

For A-1 bystander editing that causes ACTA2 M178V, for mice treated with dual AAV9 vectors, we observed a range of M178V bystander editing across each tissue (FIGS. 34A-I; Table 5). In liver we observed up to 2% M178V editing (FIG. 34A), with lower levels of bystander editing in brain vasculature (<0.4%; FIG. 34B), aorta (<0.1%; FIG. 34C), or heart (<0.4%; FIG. 34D). For most other tissues, we observed levels of bystander editing closer to the levels observed in untreated controls (FIGS. 34E-I). For mice treated with dual AAV-PR vectors (FIGS. 35A-I; Table 5), the trend of bystander editing stratified by tissue was similar to what we observed with AAV9 (FIG. 34A-I). However, the general levels of bystander editing per tissue with AAV-PR vectors (FIGS. 35A-I) were higher than what we observed in tissues treated with AAV9 (FIGS. 34A-I), consistent with our observation of higher levels of intended editing of the A4 base with AAV-PR.

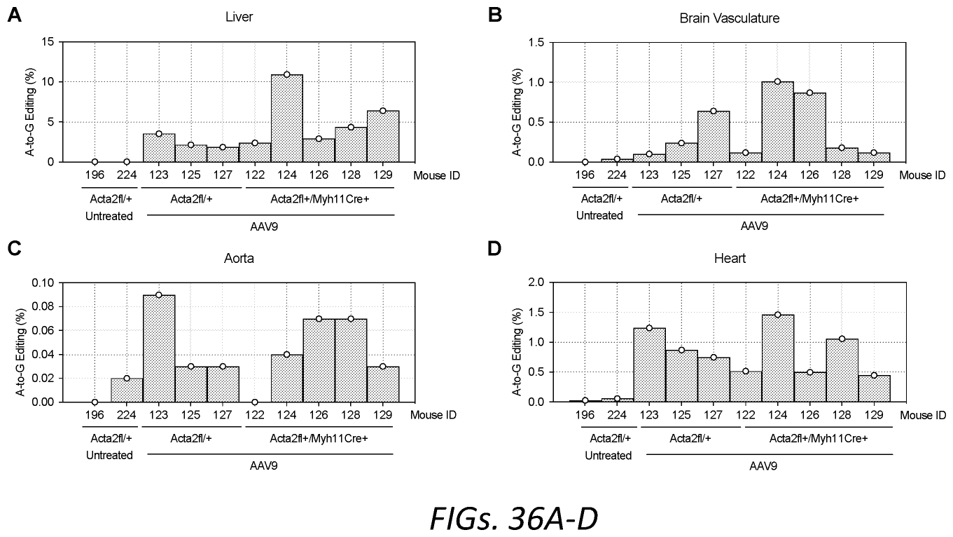

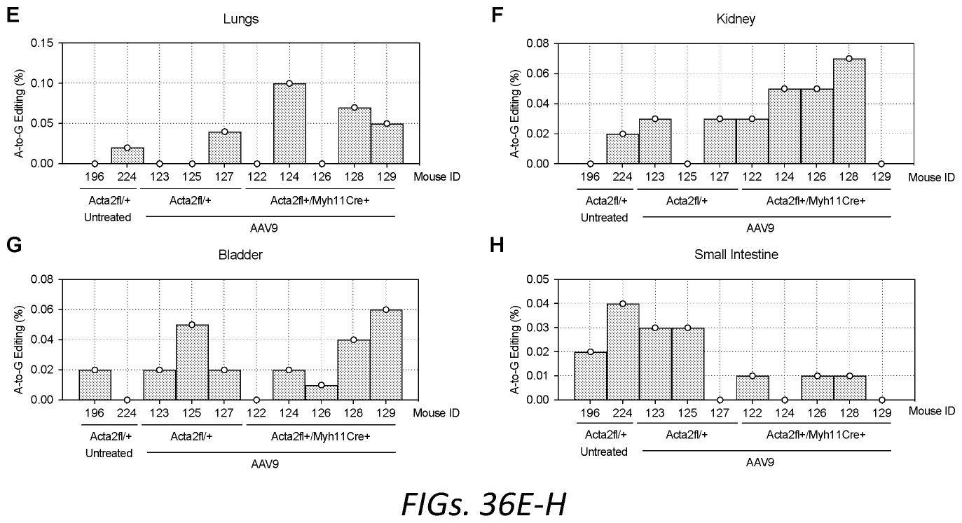

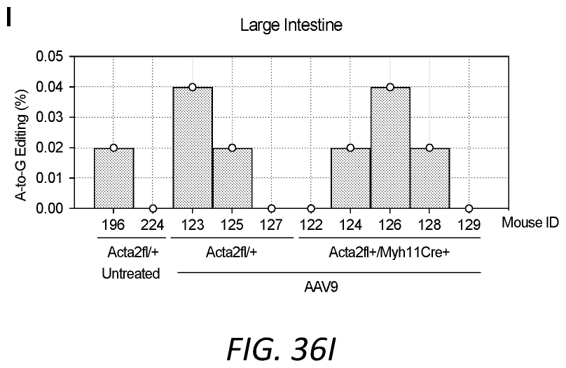

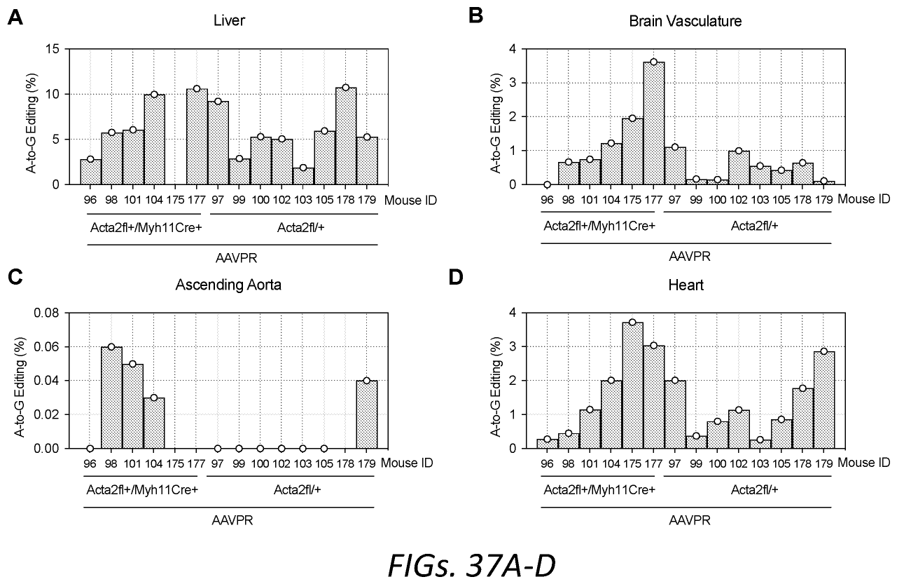

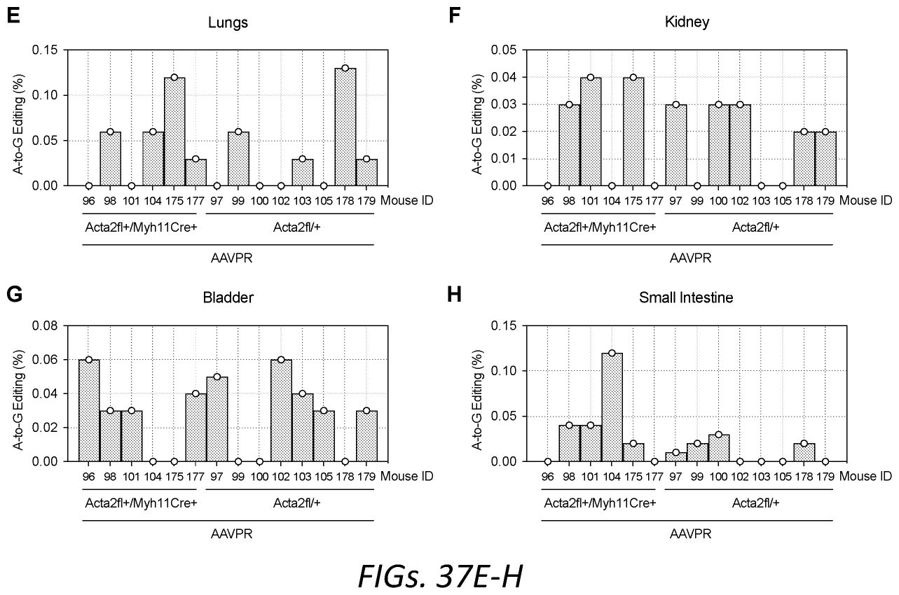

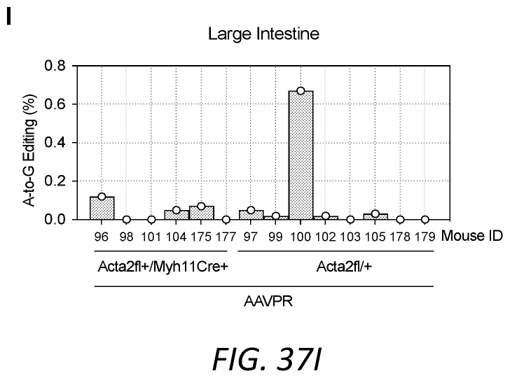

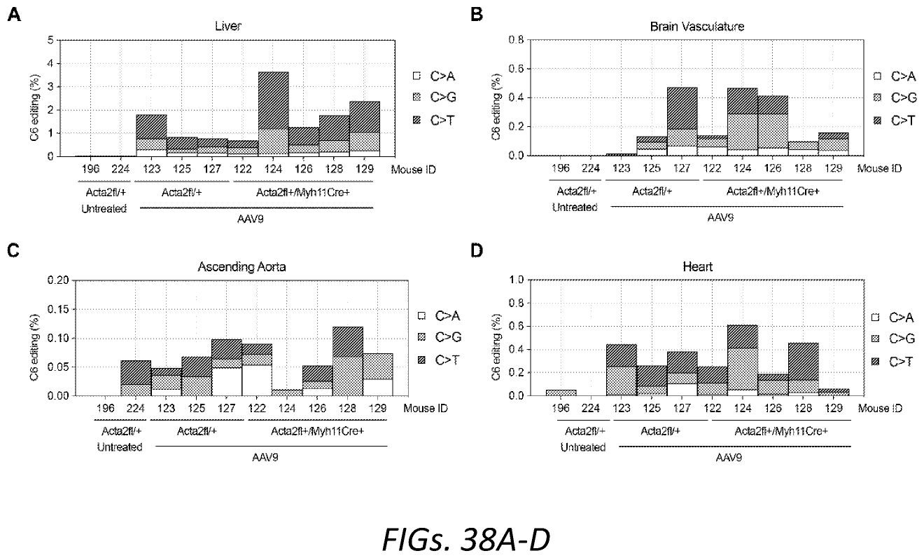

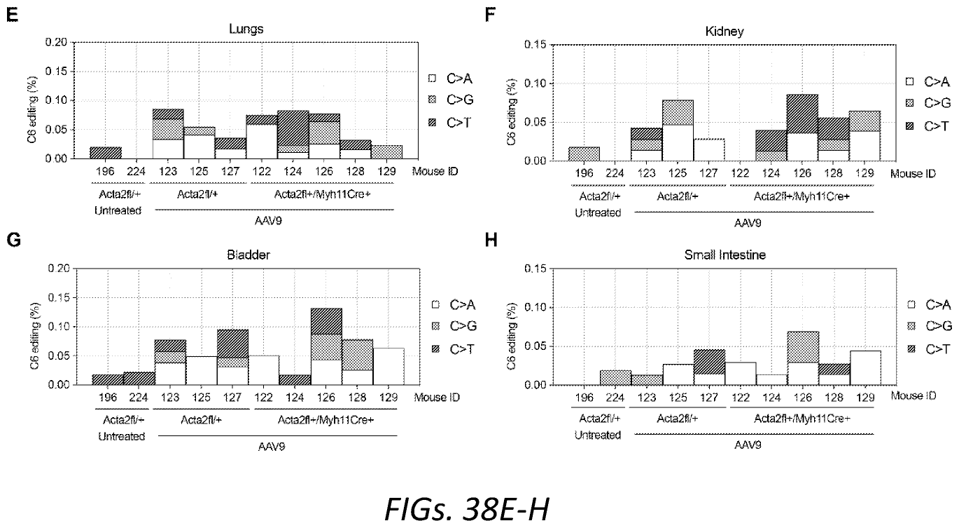

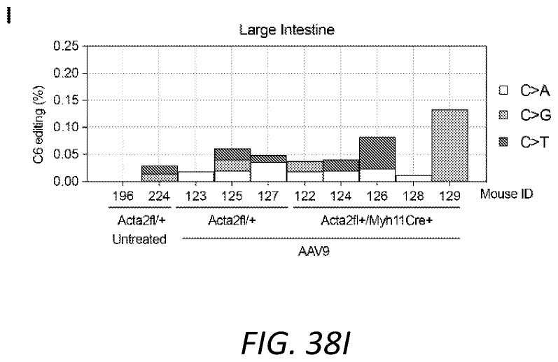

Next, we also analyzed A10 bystander editing that causes ACTA2 D181G (FIG. 33). For mice treated with dual AAV9 vectors, we observed a range of D181G bystander editing across each tissue (FIGS. 36A-I; Table 5). In liver we observed up to 10% D181G editing (FIG. 36A), with lower levels of bystander editing in brain vasculature (<1%; FIG. 36B), aorta (<0.1%; FIG. 36C), or heart (<1.5%; FIG. 36D). For most other tissues, we observed levels of bystander editing closer to the levels observed in untreated controls (FIGS. 36E-I). For mice treated with dual AAV-PR vectors (FIGS. 37A-I; Table 5), the trend of bystander editing stratified by tissue was similar to what we observed with AAV9 (FIG. 36A-I). However, the mean levels of bystander editing per tissue with AAV-PR vectors (FIGS. 38A-I) were higher than what we observed in tissues treated with AAV9 (FIGS. 37A-I), consistent with our observation of higher levels of intended editing of the A4 base with AAV-PR.

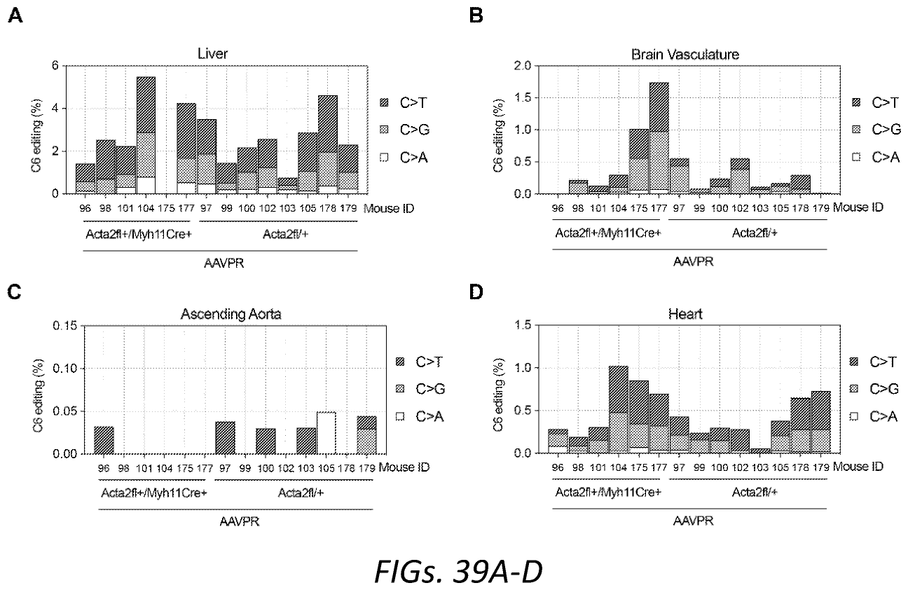

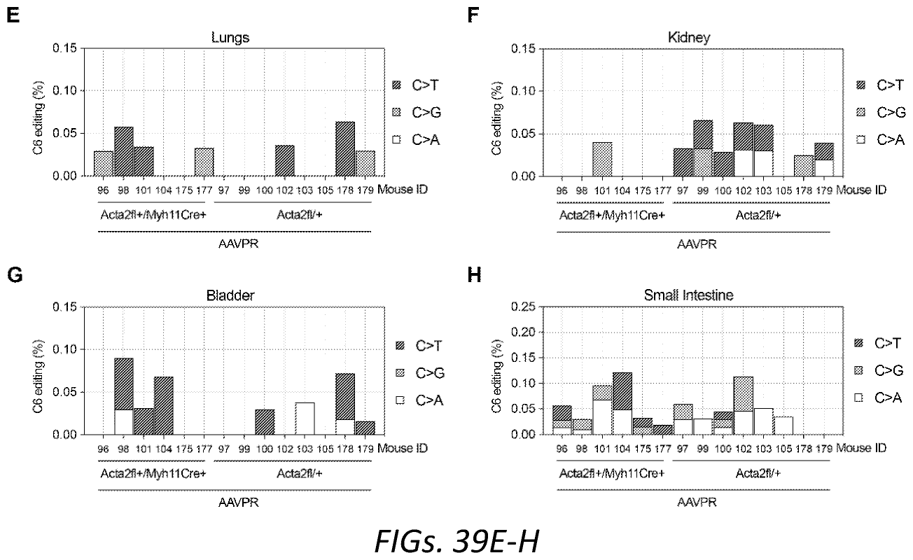

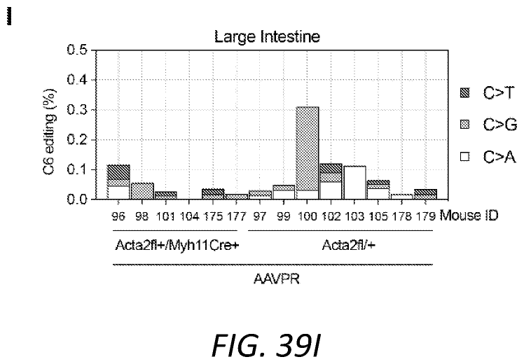

We also analyzed C6A, C6G, or C6T editing that would cause L180M, L180V, or a silent L180L, respectively (FIG. 33). For mice treated with dual AAV9 vectors, we observed a range of L180 bystander editing across each tissue (FIGS. 38A-I; Table 5). In liver we observed up to 3.6% total L180 editing (FIG. 38A), with lower levels of bystander editing in brain vasculature (<0.5%; FIG. 38B), aorta (<0.12%; FIG. 38C), or heart (<0.6%; FIG. 38D). For most other tissues, we observed levels of bystander editing closer to the levels observed in untreated controls (FIGS. 38E-I). For mice treated with dual AAV-PR vectors (FIGS. 39A-I; Table 5), the trend of bystander L180 editing stratified by tissue was similar to what we observed with AAV9 (FIG. 38A-I), though, once again, the mean levels of bystander editing per tissue with AAV-PR vectors were higher, consistent with our observation of higher levels of intended editing of the A4 base with AAV-PR. In general, the most prevalent C6 bystander edit was a C-to-T, which is a silent L180L substitution that we therefore anticipate should not impact the function of ACTA2. Furthermore, the second most common C6 substitution is a C-to-G that causes L180V, leading to a biochemically similar amino acid side chain (similar size and biophysical properties), which may lead to similar ACTA function. The proportion of total C6 bystander edits across each of the three classes of substitutions (C-to-A, C-to-G, and C-to-T) appeared to vary slightly by tissue, consistent with prior reports in different cell lines (Burnett et al., Frontiers in Genome Editing, 2022, PMID: 35910415).

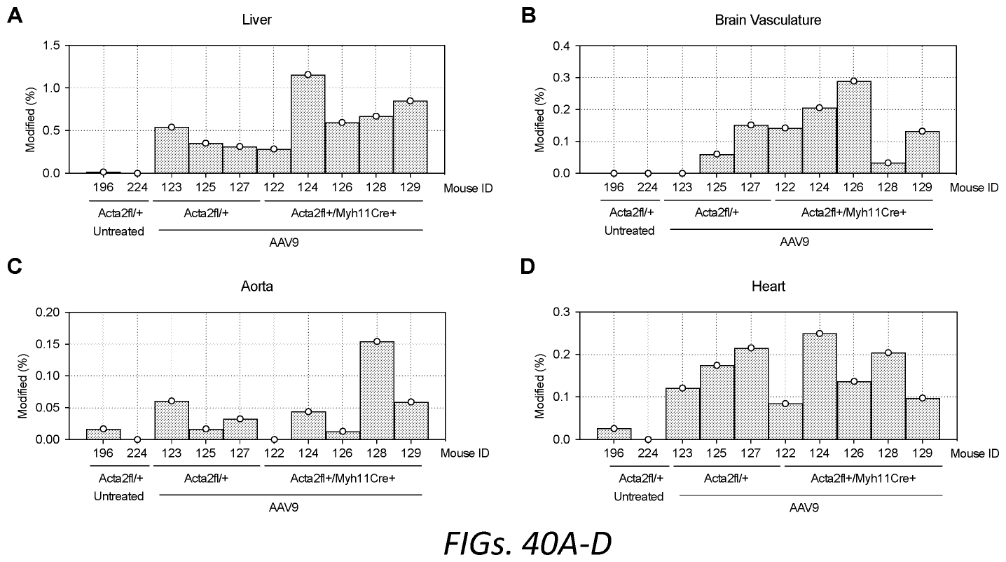

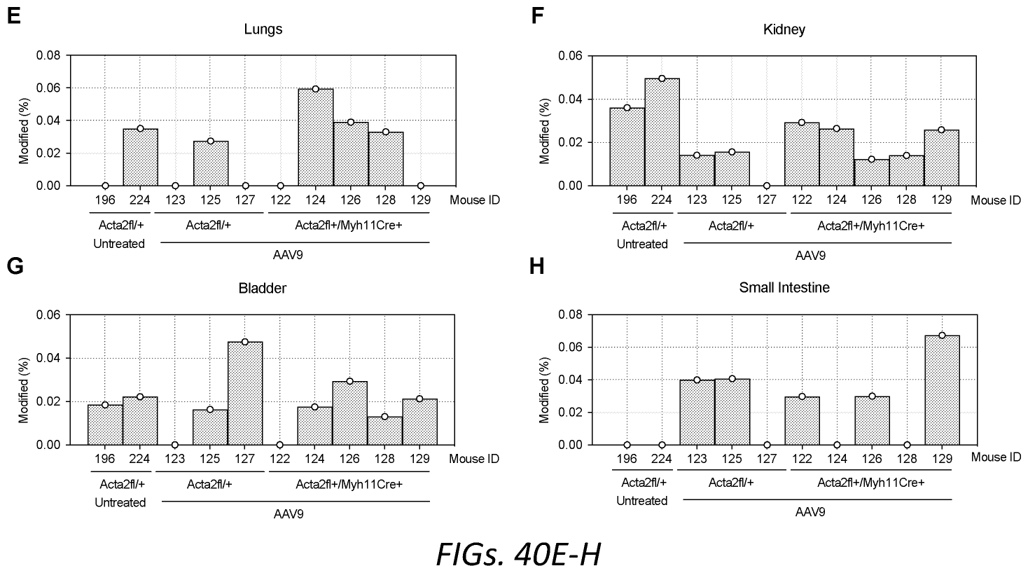

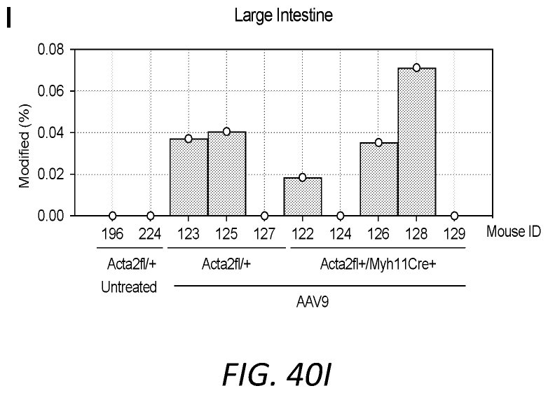

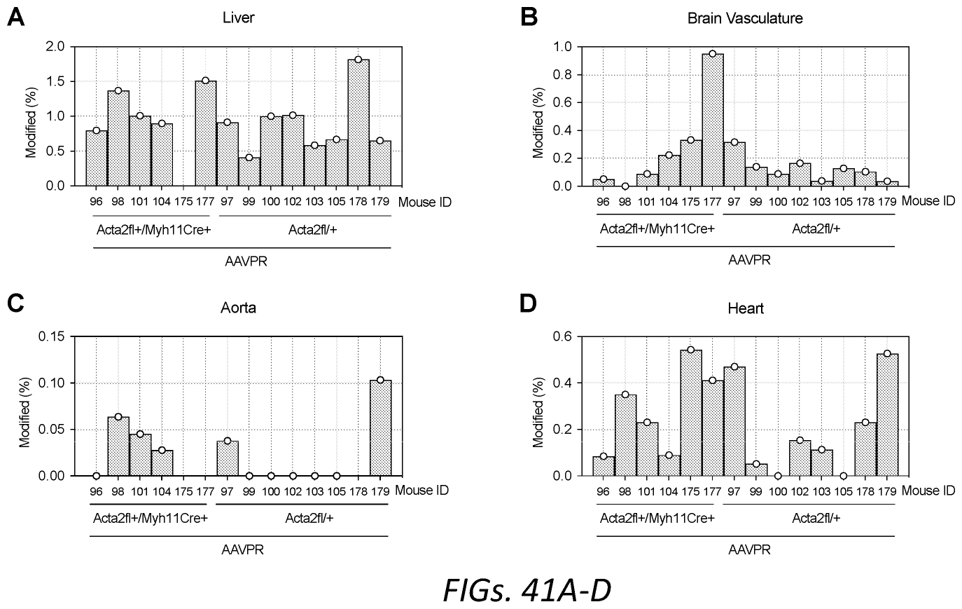

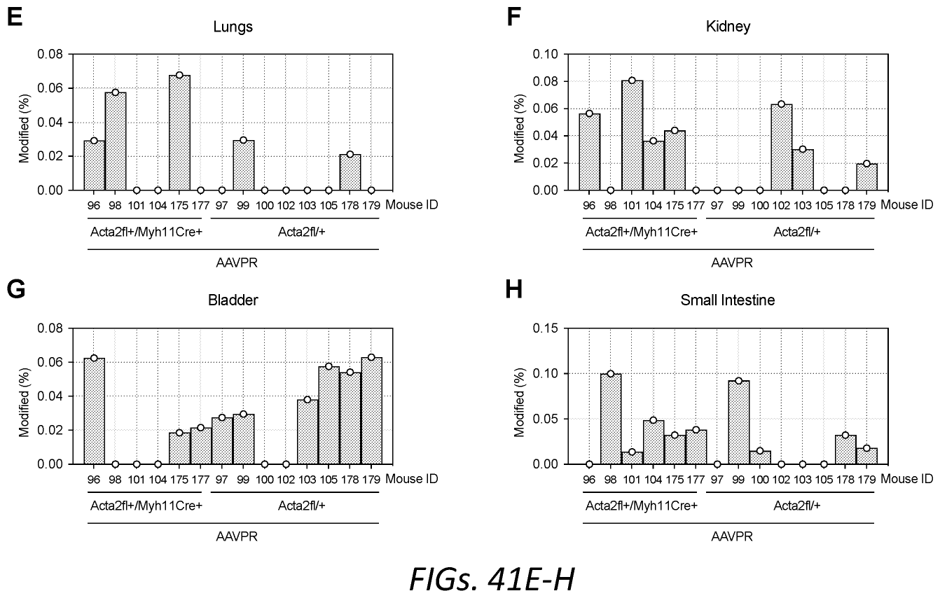

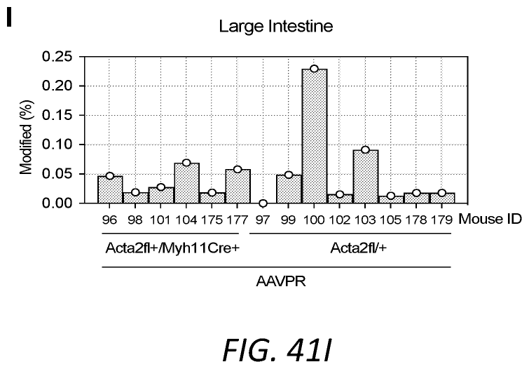

We then analyzed the propensity of our ABE approach to create unwanted insertion or deletion mutations (indels) at the A4 target site (Table 5). For AAV9-treated mice (FIGS. 40A-I), we sometimes observed indels in the 0.1-1% range for liver, brain vasculature, aorta, or heart (FIGS. 40A-D, respectively). For the other tissues, indels were typically <0.05% and in most cases near control levels (FIGS. 40E-I). In mice treated with AAV-PR vectors (FIGS. 41A-I), as expected due to higher levels of overall editing, we observed slightly elevated levels of indels compared to AAV9-treated mice (FIGS. 40A-I and FIGS. 41A-I, for AAV9 and AAV-PR, respectively).

Despite these collected levels of unwanted editing (bystander editing and/or indels), we observed much higher levels of on-target editing at ACTA2-R179H that led to dramatic phenotypic recovery in the Myh11-Cre:Acta2R179Hfl/+ mice. These observations suggest that the unwanted edits were either innocuous or have little functional consequence (the bystander edits), or occurred at levels that did not detract from the phenotypic recovery of the animals or do not impact their general viability.

### AAV Transduction and Transgene Expression

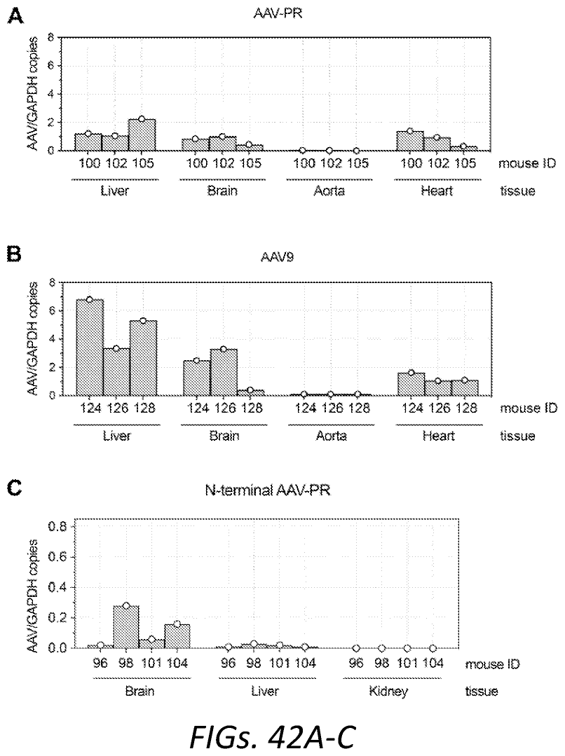

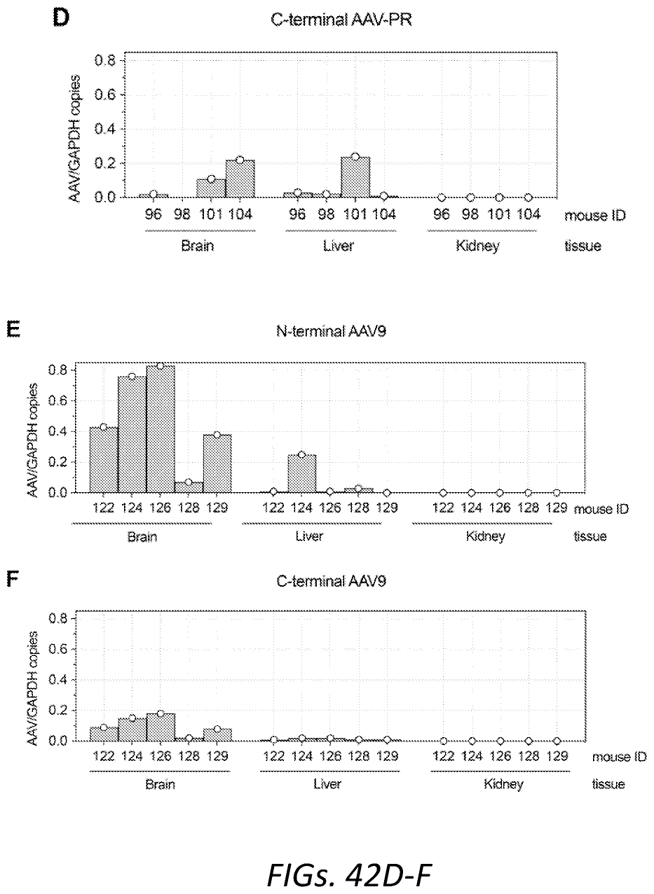

Next, we analyzed the levels of AAV transduction in selected tissues and animals from cohort 3. We initially quantified the approximate number of AAV genomes per haploid genome by performing ddPCR on extracted genomic DNA (containing AAV genomes) from both AAV-PR and AAV9 treated animals (FIGS. 42A and 42B, respectively). Samples treated with AAV9 appeared to exhibit slightly higher levels of AAV transduction in liver and brain compared to those treated with AAV-PR, with comparable transduction in the heart (FIGS. 42A and 42B). We then analyzed expression of either the N- or C-terminal mRNA from either AAV by extracting RNA, generating cDNA, and performing ddPCR. Depending on the tissue and AAV serotype, we generally observed comparable levels of expression from the N- and C-terminal AAV constructs (FIGS. 42C-F). Differences in AAV genome copy number or mRNA expression levels compared to A-to-G editing may be due to cell division and dilution of the AAV genome/transgene across the growth and lifespan of the animals.

### Assessment of On-Target R179H Editing in a Cohort of P14 AAV-Injected Mice

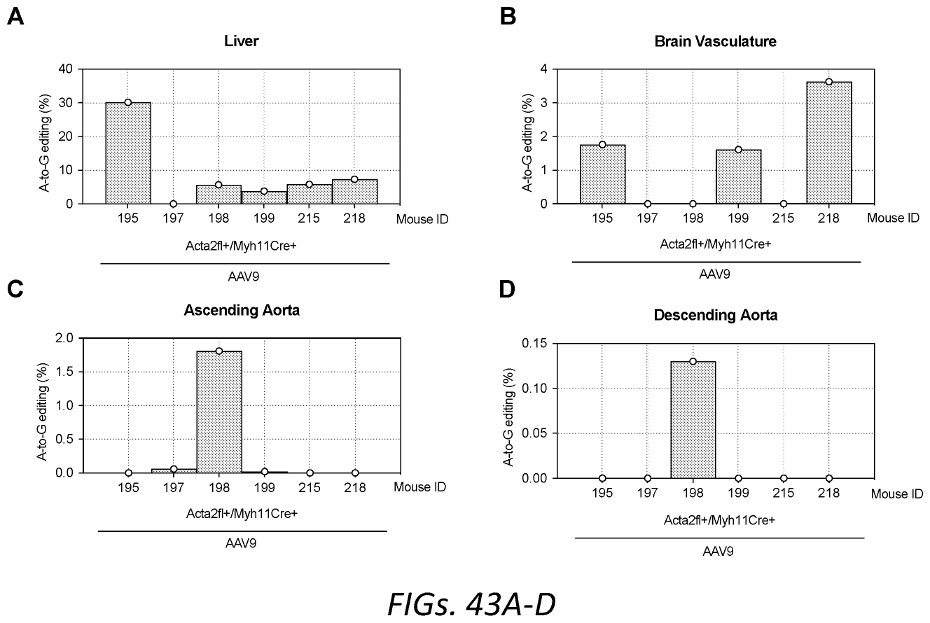

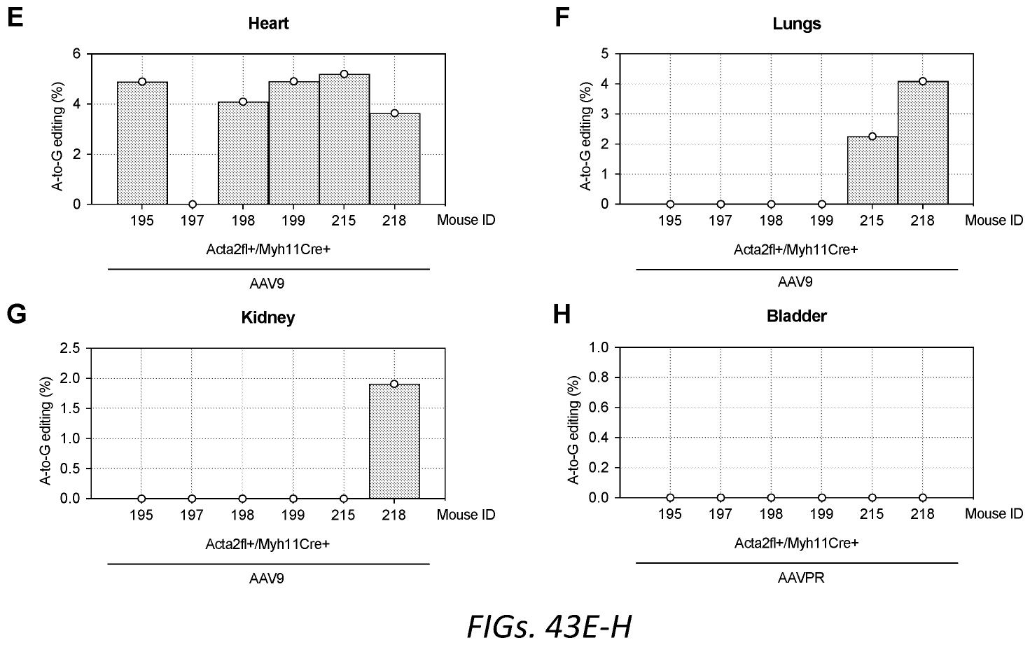

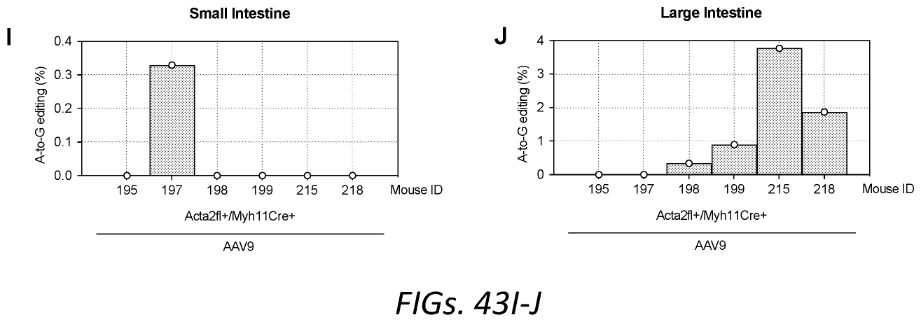

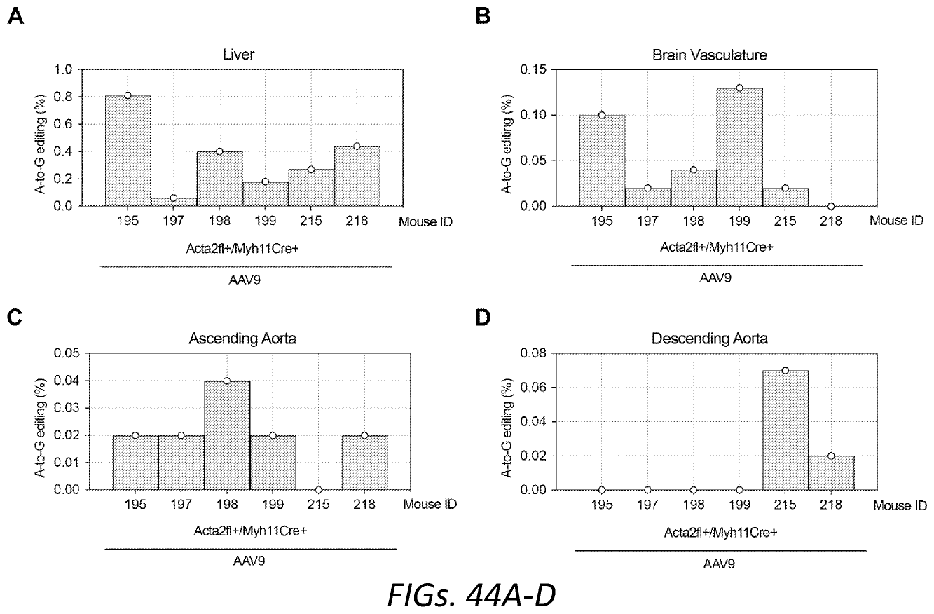

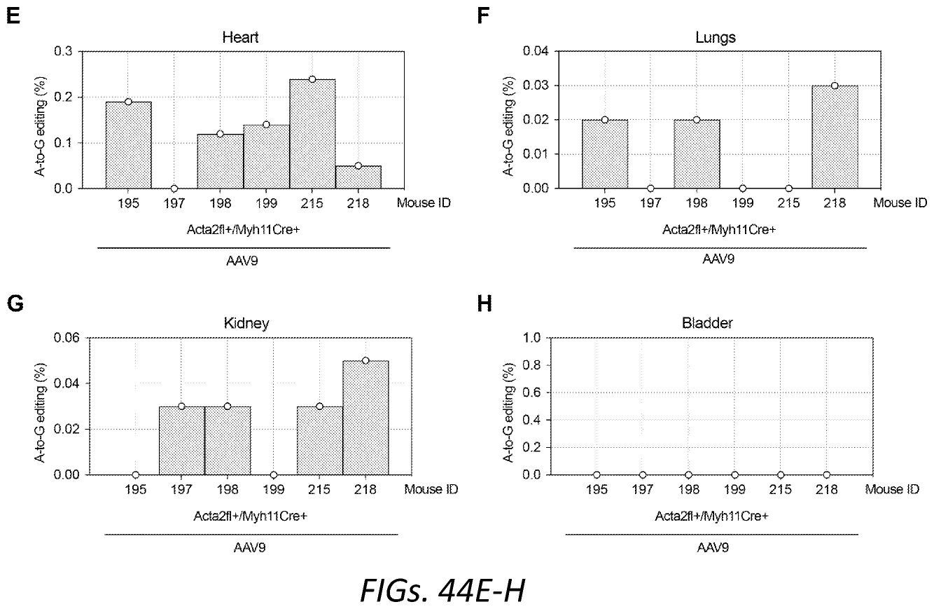

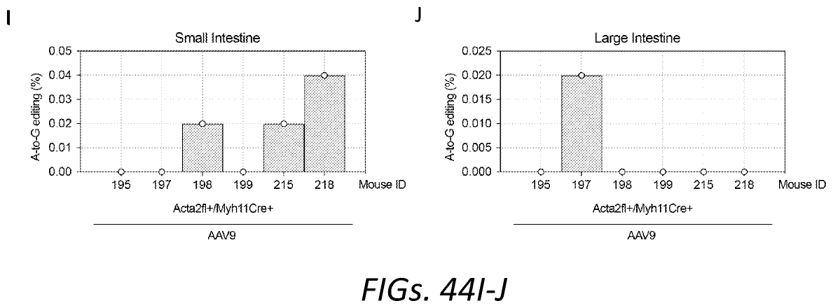

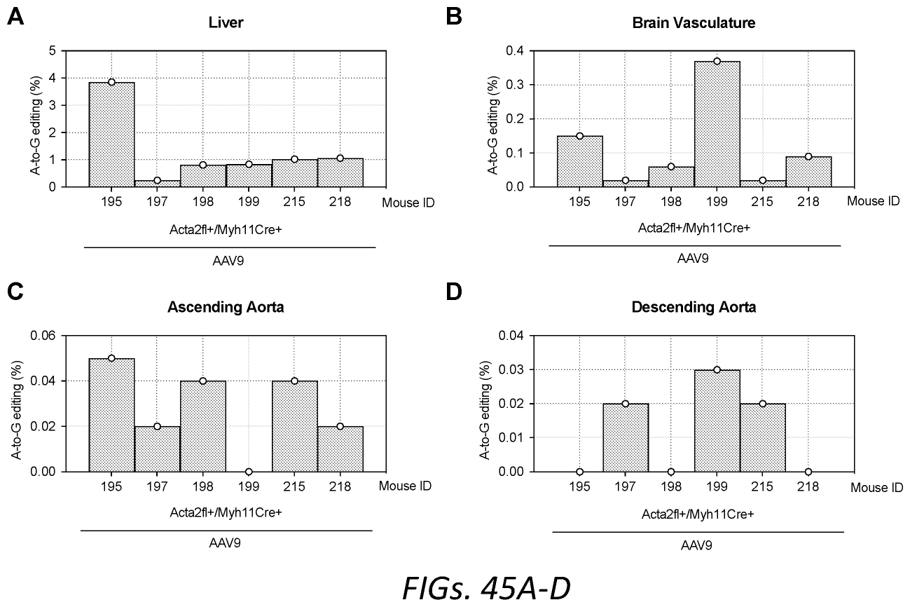

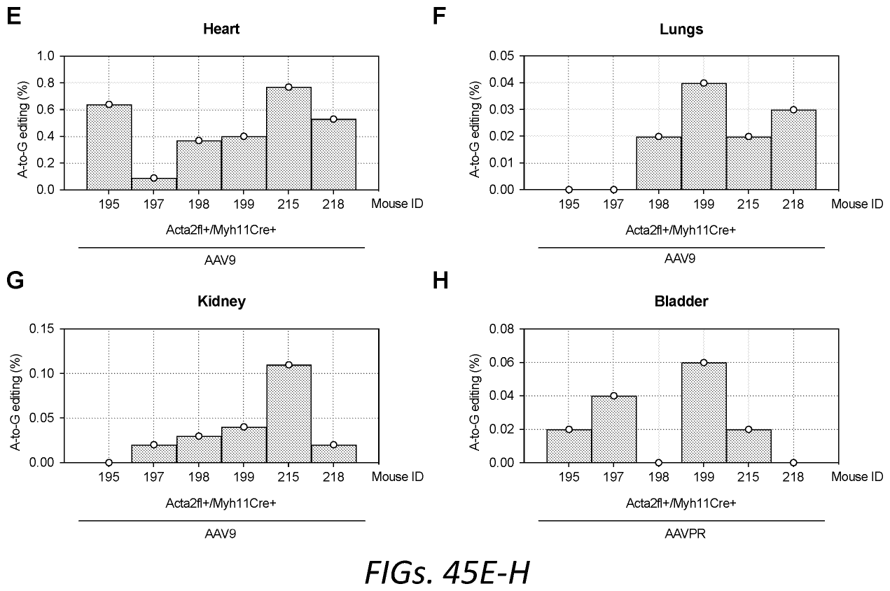

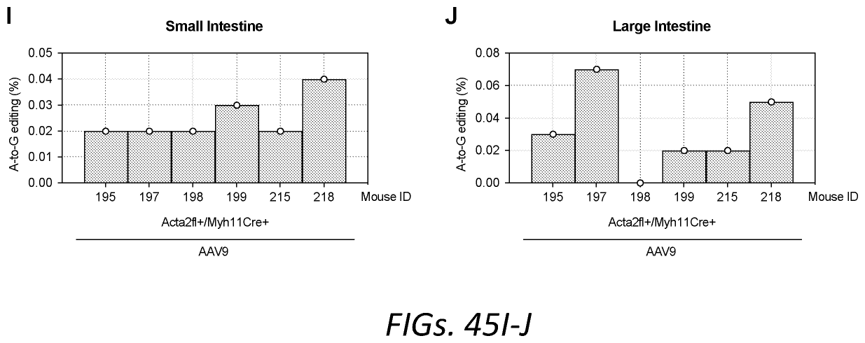

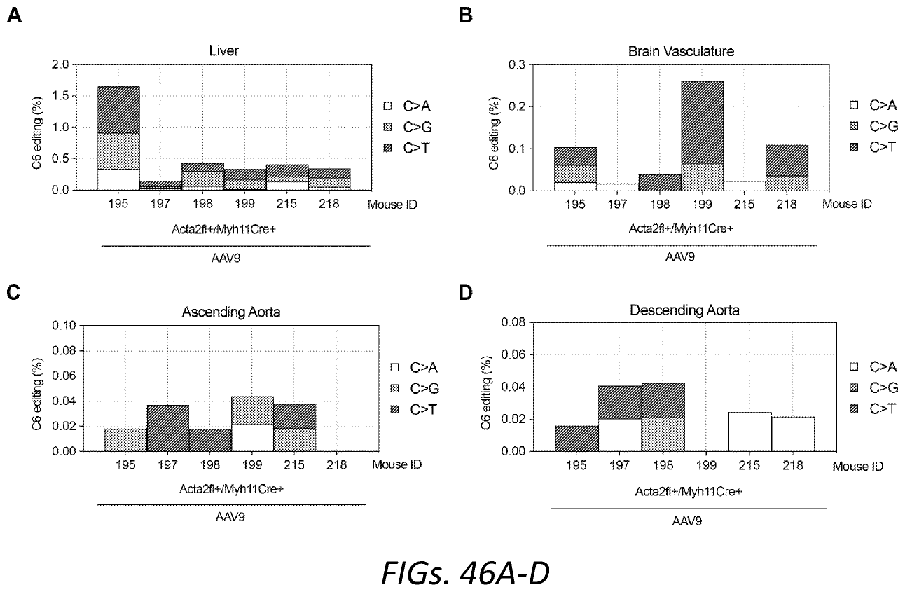

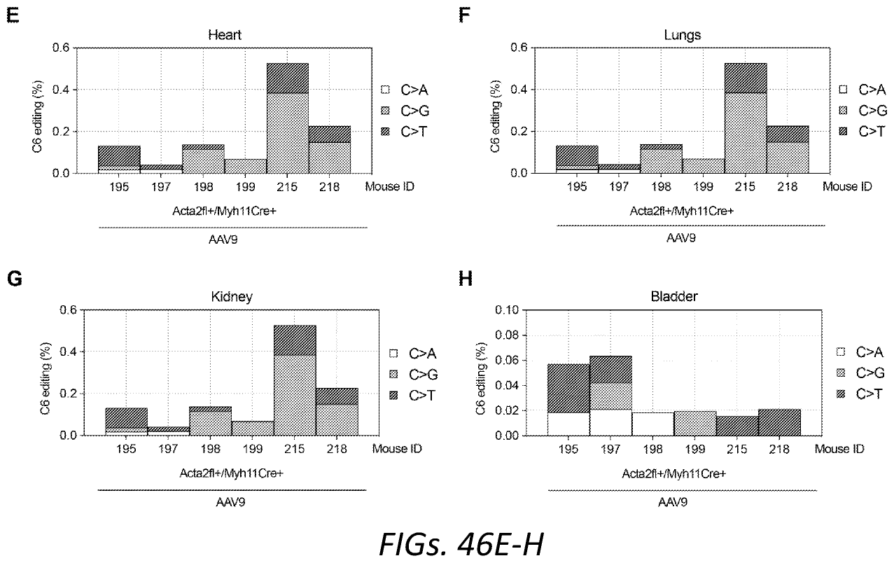

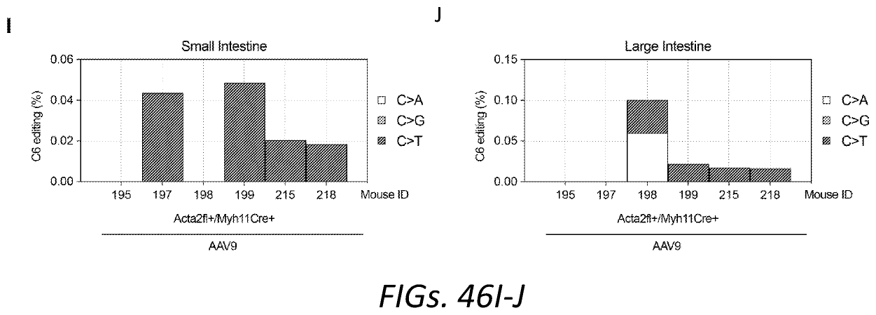

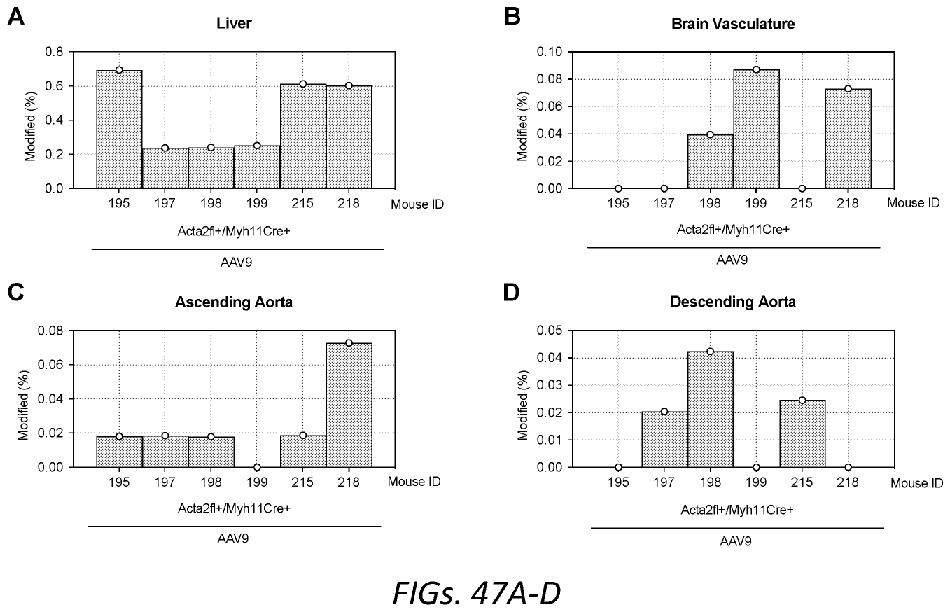

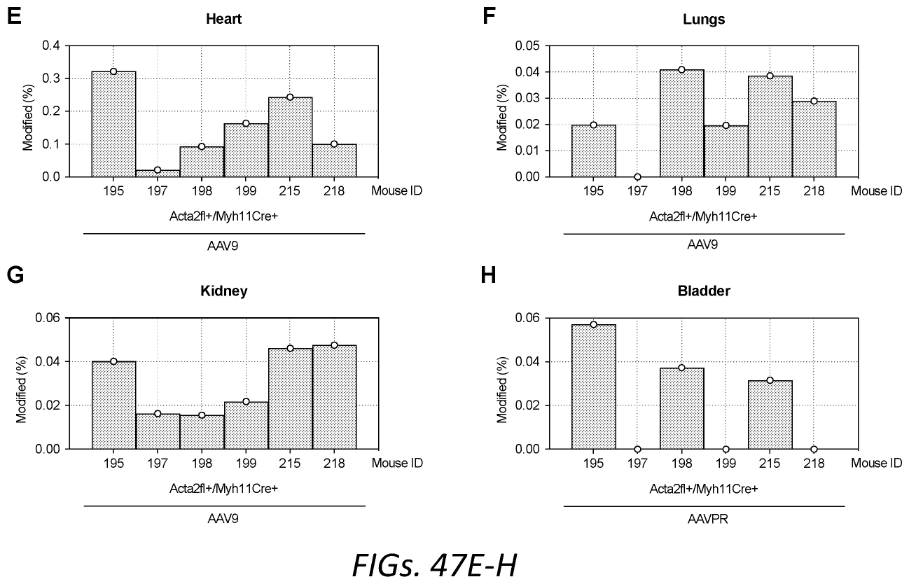

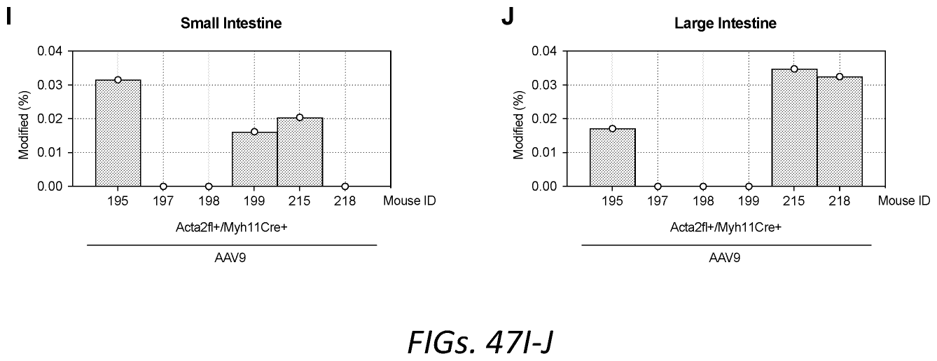

We also injected a smaller cohort of mice at P14 using AAV9 packaged dual AAV vectors encoding the split ABE8e-SpCas9-VRQR(S55R) construct with the A4 NGA PAM gRNA, as described above but at a later point in life (P14 instead of P3) (Table 5). Relative to our P3 injections, we observed lower levels of overall editing across various tissues and mice in this small cohort injected later in life (FIGS. 43A-J). Reduced on-target editing at A4 in this cohort was accompanied by lower overall levels of bystander editing of M178V, D181G, or L180M/V/L editing (FIGS. 44A-J, 45A-J, and 46A-J, respectively), along with reduced levels of indels (FIGS. 47A-J).

### SUMMARY

These additional data from Cohorts 3 demonstrate that injecting the mice with the AAV-encoded ABE and gRNA earlier in life can lead to much higher levels of correction of the ACTA2 R179H mutation, and that we can achieve high levels of intended reversion of ACTA2 R179H to R179 with low levels of unwanted bystander edits or indels. The striking phenotypic recovery that was observed in the mice indicates that the bystander edits (in addition to the R179H correction) might be functionally innocuous or tolerated by the ACTA2 protein (including the biochemically similar L180V substitution or the silent L180L change).

TABLE 5
Sequencing results from mouse studies

| | On- | | | | | | |
|---|---|---|---|---|---|---|---|
| | target | | | | | | |
| | editing | | | | | | |
| | (%) | Undesired editing (%) - bystander edits or indels | | | | | |
| Group_Tissue_MouseID | A4-to-G | A10-to-G | A-1-to-G | C6-to-A | C6-to-G | C6-to-T | Indels (%) |
| AAV9-P3_Liver_122 | 16.746% | 2.386% | 0.678% | 0.108% | 0.285% | 0.298% | 0.285% |
| AAV9-P3_Liver_123 | 27.609% | 3.543% | 0.702% | 0.303% | 0.495% | 1.005% | 0.543% |
| AAV9-P3_Liver_124 | 58.279% | 10.922% | 1.988% | 0.135% | 1.081% | 2.431% | 1.158% |
| AAV9-P3_Liver_125 | 15.223% | 2.151% | 0.507% | 0.172% | 0.163% | 0.497% | 0.354% |
| AAV9-P3_Liver_126 | 18.055% | 2.929% | 0.750% | 0.176% | 0.351% | 0.738% | 0.598% |
| AAV9-P3_Liver_127 | 13.539% | 1.860% | 0.520% | 0.164% | 0.260% | 0.356% | 0.315% |
| AAV9-P3_Liver_128 | 28.306% | 4.364% | 1.072% | 0.211% | 0.498% | 1.072% | 0.670% |
| AAV9-P3_Liver_129 | 43.849% | 6.415% | 1.757% | 0.249% | 0.806% | 1.333% | 0.849% |
| AAV9-P14_Liver_195 | 30.169% | 3.865% | 0.807% | 0.338% | 0.582% | 0.732% | 0.694% |
| AAV9-P14_Liver_197 | 0.000% | 0.252% | 0.059% | 0.030% | 0.030% | 0.089% | 0.237% |
| AAV9-P14_Liver_198 | 5.681% | 0.816% | 0.400% | 0.064% | 0.240% | 0.128% | 0.240% |
| AAV9-P14_Liver_199 | 3.802% | 0.837% | 0.184% | 0.017% | 0.151% | 0.167% | 0.251% |
| AAV9-P14_Liver_215 | 5.834% | 1.021% | 0.272% | 0.136% | 0.085% | 0.187% | 0.612% |
| AAV9-P14_Liver_218 | 7.363% | 1.060% | 0.439% | 0.055% | 0.146% | 0.146% | 0.603% |
| AAV9-P3_BrVasc_122 | 0.000% | 0.122% | 0.102% | 0.061% | 0.061% | 0.020% | 0.143% |
| AAV9-P3_BrVasc_123 | 0.000% | 0.101% | 0.051% | 0.000% | 0.000% | 0.013% | 0.000% |
| AAV9-P3_BrVasc_124 | 2.174% | 1.011% | 0.348% | 0.043% | 0.250% | 0.174% | 0.207% |
| AAV9-P3_BrVasc_125 | 0.398% | 0.241% | 0.060% | 0.048% | 0.048% | 0.036% | 0.060% |
| AAV9-P3_BrVasc_126 | 3.188% | 0.869% | 0.248% | 0.055% | 0.235% | 0.124% | 0.290% |
| AAV9-P3_BrVasc_127 | 0.795% | 0.643% | 0.085% | 0.068% | 0.118% | 0.288% | 0.152% |
| AAV9-P3_BrVasc_128 | 2.219% | 0.177% | 0.044% | 0.044% | 0.055% | 0.000% | 0.033% |
| AAV9-P3_BrVasc_129 | 0.943% | 0.120% | 0.080% | 0.040% | 0.080% | 0.040% | 0.133% |
| AAV9-P14_BrVasc_195 | 1.762% | 0.145% | 0.104% | 0.021% | 0.041% | 0.041% | 0.000% |
| AAV9-P14_BrVasc_197 | 0.000% | 0.017% | 0.017% | 0.017% | 0.000% | 0.000% | 0.000% |
| AAV9-P14_BrVasc_198 | 0.000% | 0.059% | 0.040% | 0.000% | 0.000% | 0.040% | 0.040% |
| AAV9-P14_BrVasc_199 | 1.614% | 0.371% | 0.131% | 0.000% | 0.065% | 0.196% | 0.087% |
| AAV9-P14_BrVasc_215 | 0.000% | 0.023% | 0.023% | 0.023% | 0.000% | 0.000% | 0.000% |
| AAV9-P14_BrVasc_218 | 3.631% | 0.091% | 0.000% | 0.000% | 0.036% | 0.073% | 0.073% |
| AAV9-P3_AsAort_122 | 0.000% | 0.000% | 0.054% | 0.054% | 0.018% | 0.018% | 0.000% |
| AAV9-P3_AsAort_123 | 0.000% | 0.085% | 0.024% | 0.012% | 0.024% | 0.012% | 0.061% |
| AAV9-P3_AsAort_124 | 0.000% | 0.044% | 0.066% | 0.000% | 0.011% | 0.000% | 0.044% |
| AAV9-P3_AsAort_125 | 0.000% | 0.034% | 0.000% | 0.000% | 0.034% | 0.034% | 0.017% |
| AAV9-P3_AsAort_126 | 0.000% | 0.066% | 0.000% | 0.013% | 0.013% | 0.026% | 0.013% |
| AAV9-P3_AsAort_127 | 0.000% | 0.033% | 0.098% | 0.049% | 0.016% | 0.033% | 0.033% |
| AAV9-P3_AsAort_128 | 0.000% | 0.069% | 0.017% | 0.000% | 0.069% | 0.051% | 0.154% |
| AAV9-P3_AsAort_129 | 0.000% | 0.030% | 0.015% | 0.030% | 0.044% | 0.000% | 0.059% |
| AAV9-P14_AsAort_195 | 0.000% | 0.054% | 0.018% | 0.000% | 0.018% | 0.000% | 0.018% |
| AAV9-P14_AsAort_197 | 0.056% | 0.019% | 0.019% | 0.000% | 0.000% | 0.037% | 0.019% |
| AAV9-P14_AsAort_198 | 1.811% | 0.036% | 0.036% | 0.000% | 0.000% | 0.018% | 0.018% |
| AAV9-P14_AsAort_199 | 0.022% | 0.000% | 0.022% | 0.022% | 0.022% | 0.000% | 0.000% |
| AAV9-P14_AsAort_215 | 0.000% | 0.038% | 0.000% | 0.000% | 0.019% | 0.019% | 0.019% |
| AAV9-P14_AsAort_218 | 0.000% | 0.024% | 0.024% | 0.000% | 0.000% | 0.000% | 0.073% |
| AAV9-P14_DeAort_195 | 0.000% | 0.000% | 0.000% | 0.000% | 0.000% | 0.016% | 0.000% |
| AAV9-P14_DeAort_197 | 0.000% | 0.020% | 0.000% | 0.020% | 0.000% | 0.020% | 2.043% |
| AAV9-P14_DeAort_198 | 0.127% | 0.000% | 0.000% | 0.000% | 0.021% | 0.021% | 4.239% |
| AAV9-P14_DeAort_199 | 0.000% | 0.026% | 0.000% | 0.000% | 0.000% | 0.000% | 0.000% |
| AAV9-P14_DeAort_215 | 0.000% | 0.025% | 0.074% | 0.025% | 0.000% | 0.000% | 2.453% |
| AAV9-P14_DeAort_218 | 0.000% | 0.000% | 0.022% | 0.022% | 0.000% | 0.000% | 0.000% |
| AAV9-P3_Heart_122 | 5.140% | 0.522% | 0.085% | 0.014% | 0.099% | 0.141% | 0.085% |
| AAV9-P3_Heart_123 | 7.021% | 1.242% | 0.243% | 0.014% | 0.243% | 0.189% | 0.122% |
| AAV9-P3_Heart_124 | 10.043% | 1.459% | 0.319% | 0.056% | 0.361% | 0.194% | 0.250% |
| AAV9-P3_Heart_125 | 7.366% | 0.874% | 0.372% | 0.022% | 0.066% | 0.175% | 0.175% |
| AAV9-P3_Heart_126 | 2.796% | 0.498% | 0.051% | 0.017% | 0.120% | 0.051% | 0.137% |
| AAV9-P3_Heart_127 | 4.634% | 0.754% | 0.123% | 0.108% | 0.092% | 0.185% | 0.216% |
| AAV9-P3_Heart_128 | 5.480% | 1.055% | 0.299% | 0.031% | 0.110% | 0.315% | 0.205% |
| AAV9-P3_Heart_129 | 1.390% | 0.451% | 0.061% | 0.000% | 0.037% | 0.024% | 0.098% |
| AAV9-P14_Heart_195 | 4.901% | 0.643% | 0.189% | 0.019% | 0.019% | 0.095% | 0.322% |
| AAV9-P14_Heart_197 | 0.000% | 0.086% | 0.000% | 0.021% | 0.000% | 0.021% | 0.021% |
| AAV9-P14_Heart_198 | 4.103% | 0.373% | 0.117% | 0.000% | 0.117% | 0.023% | 0.093% |
| AAV9-P14_Heart_199 | 4.907% | 0.397% | 0.140% | 0.000% | 0.070% | 0.000% | 0.164% |
| AAV9-P14_Heart_215 | 5.201% | 0.772% | 0.244% | 0.000% | 0.386% | 0.142% | 0.244% |
| AAV9-P14_Heart_218 | 3.642% | 0.531% | 0.051% | 0.000% | 0.152% | 0.076% | 0.101% |
| AAV9-P3_Lungs_122 | 0.000% | 0.000% | 0.015% | 0.060% | 0.000% | 0.015% | 0.000% |
| AAV9-P3_Lungs_123 | 0.825% | 0.000% | 0.017% | 0.034% | 0.034% | 0.017% | 0.000% |
| AAV9-P3_Lungs_124 | 0.000% | 0.095% | 0.012% | 0.012% | 0.012% | 0.059% | 0.059% |
| AAV9-P3_Lungs_125 | 0.000% | 0.000% | 0.041% | 0.041% | 0.014% | 0.000% | 0.028% |
| AAV9-P3_Lungs_126 | 0.000% | 0.000% | 0.000% | 0.026% | 0.039% | 0.013% | 0.039% |
| AAV9-P3_Lungs_127 | 0.000% | 0.036% | 0.000% | 0.018% | 0.000% | 0.018% | 0.000% |
| AAV9-P3_Lungs_128 | 0.000% | 0.066% | 0.017% | 0.017% | 0.000% | 0.017% | 0.033% |
| AAV9-P3_Lungs_129 | 0.000% | 0.048% | 0.000% | 0.000% | 0.024% | 0.000% | 0.000% |
| AAV9-P14_Lungs_195 | 0.000% | 0.000% | 0.020% | 0.000% | 0.000% | 0.000% | 0.020% |
| AAV9-P14_Lungs_197 | 0.000% | 0.000% | 0.000% | 0.000% | 0.000% | 0.000% | 0.000% |
| AAV9-P14_Lungs_198 | 0.000% | 0.020% | 0.020% | 0.000% | 0.000% | 0.000% | 0.041% |
| AAV9-P14_Lungs_199 | 0.000% | 0.039% | 0.000% | 0.020% | 0.020% | 0.000% | 0.020% |
| AAV9-P14_Lungs_215 | 2.256% | 0.019% | 0.000% | 0.000% | 0.000% | 0.019% | 0.039% |
| AAV9-P14_Lungs_218 | 4.088% | 0.029% | 0.029% | 0.000% | 0.029% | 0.029% | 0.029% |
| AAV9-P3_Kidney_122 | 0.000% | 0.029% | 0.000% | 0.000% | 0.000% | 0.000% | 0.029% |
| AAV9-P3_Kidney_123 | 0.000% | 0.028% | 0.014% | 0.014% | 0.014% | 0.014% | 0.014% |
| AAV9-P3_Kidney_124 | 0.000% | 0.053% | 0.013% | 0.000% | 0.013% | 0.027% | 0.027% |
| AAV9-P3_Kidney_125 | 0.000% | 0.000% | 0.031% | 0.047% | 0.031% | 0.000% | 0.016% |
| AAV9-P3_Kidney_126 | 0.000% | 0.049% | 0.025% | 0.037% | 0.000% | 0.049% | 0.012% |
| AAV9-P3_Kidney_127 | 0.329% | 0.029% | 0.000% | 0.029% | 0.000% | 0.000% | 0.000% |
| AAV9-P3_Kidney_128 | 0.000% | 0.070% | 0.000% | 0.014% | 0.014% | 0.028% | 0.014% |
| AAV9-P3_Kidney_129 | 0.000% | 0.000% | 0.013% | 0.039% | 0.026% | 0.000% | 0.026% |
| AAV9-P14_Kidney_195 | 0.000% | 0.000% | 0.000% | 0.040% | 0.000% | 0.020% | 0.040% |
| AAV9-P14_Kidney_197 | 0.000% | 0.016% | 0.033% | 0.000% | 0.000% | 0.016% | 0.016% |
| AAV9-P14_Kidney_198 | 0.000% | 0.031% | 0.031% | 0.000% | 0.016% | 0.016% | 0.016% |
| AAV9-P14_Kidney_199 | 0.000% | 0.044% | 0.000% | 0.000% | 0.000% | 0.022% | 0.022% |
| AAV9-P14_Kidney_215 | 0.000% | 0.108% | 0.031% | 0.000% | 0.000% | 0.031% | 0.046% |
| AAV9-P14_Kidney_218 | 1.906% | 0.024% | 0.048% | 0.000% | 0.000% | 0.000% | 0.048% |
| AAV9-P3_Blad_122 | 0.000% | 0.000% | 0.000% | 0.051% | 0.000% | 0.000% | 0.000% |
| AAV9-P3_Blad_123 | 0.156% | 0.020% | 0.098% | 0.039% | 0.020% | 0.020% | 0.000% |
| AAV9-P3_Blad_124 | 0.000% | 0.018% | 0.018% | 0.000% | 0.000% | 0.018% | 0.018% |
| AAV9-P3_Blad_125 | 0.000% | 0.049% | 0.000% | 0.049% | 0.000% | 0.000% | 0.016% |
| AAV9-P3_Blad_126 | 0.000% | 0.015% | 0.015% | 0.044% | 0.044% | 0.044% | 0.029% |
| AAV9-P3_Blad_127 | 0.000% | 0.016% | 0.000% | 0.032% | 0.016% | 0.048% | 0.048% |
| AAV9-P3_Blad_128 | 0.000% | 0.039% | 0.000% | 0.026% | 0.052% | 0.000% | 0.013% |
| AAV9-P3_Blad_129 | 0.000% | 0.064% | 0.000% | 0.064% | 0.000% | 0.000% | 0.021% |
| AAV9-P14_Blad_195 | 0.000% | 0.019% | 0.000% | 0.019% | 0.000% | 0.038% | 0.057% |
| AAV9-P14_Blad_197 | 0.000% | 0.042% | 0.000% | 0.021% | 0.021% | 0.021% | 0.000% |
| AAV9-P14_Blad_198 | 0.000% | 0.000% | 0.000% | 0.019% | 0.000% | 0.000% | 0.037% |
| AAV9-P14_Blad_199 | 0.000% | 0.060% | 0.000% | 0.000% | 0.020% | 0.000% | 0.000% |
| AAV9-P14_Blad_215 | 0.000% | 0.016% | 0.000% | 0.000% | 0.000% | 0.016% | 0.032% |
| AAV9-P14_Blad_218 | 0.000% | 0.000% | 0.000% | 0.000% | 0.000% | 0.021% | 0.000% |
| AAV9-P3_SmaInt_122 | 0.000% | 0.015% | 0.000% | 0.030% | 0.000% | 0.000% | 0.030% |
| AAV9-P3_SmaInt_123 | 0.000% | 0.027% | 0.000% | 0.000% | 0.013% | 0.000% | 0.040% |
| AAV9-P3_SmaInt_124 | 0.000% | 0.000% | 0.014% | 0.014% | 0.000% | 0.000% | 0.000% |
| AAV9-P3_SmaInt_125 | 0.000% | 0.027% | 0.027% | 0.027% | 0.000% | 0.000% | 0.041% |
| AAV9-P3_SmaInt_126 | 0.000% | 0.010% | 0.020% | 0.030% | 0.040% | 0.000% | 0.030% |
| AAV9-P3_SmaInt_127 | 0.000% | 0.000% | 0.046% | 0.015% | 0.000% | 0.031% | 0.000% |
| AAV9-P3_SmaInt_128 | 0.000% | 0.014% | 0.028% | 0.014% | 0.000% | 0.014% | 0.000% |
| AAV9-P3_SmaInt_129 | 0.000% | 0.000% | 0.000% | 0.045% | 0.000% | 0.000% | 0.067% |
| AAV9-P14_SmaInt_195 | 0.000% | 0.016% | 0.000% | 0.000% | 0.000% | 0.000% | 0.032% |
| AAV9-P14_SmaInt_197 | 0.327% | 0.022% | 0.000% | 0.000% | 0.000% | 0.044% | 0.000% |
| AAV9-P14_SmaInt_198 | 0.000% | 0.018% | 0.018% | 0.000% | 0.000% | 0.000% | 0.000% |
| AAV9-P14_SmaInt_199 | 0.000% | 0.032% | 0.000% | 0.000% | 0.000% | 0.049% | 0.016% |
| AAV9-P14_SmaInt_215 | 0.000% | 0.020% | 0.020% | 0.000% | 0.000% | 0.020% | 0.020% |
| AAV9-P14_SmaInt_218 | 0.000% | 0.037% | 0.037% | 0.000% | 0.000% | 0.018% | 0.000% |
| AAV9-P3_LrgInt_122 | 0.000% | 0.000% | 0.000% | 0.019% | 0.019% | 0.000% | 0.019% |
| AAV9-P3_LrgInt_123 | 0.000% | 0.037% | 0.019% | 0.019% | 0.000% | 0.000% | 0.037% |
| AAV9-P3_LrgInt_124 | 0.000% | 0.020% | 0.060% | 0.020% | 0.000% | 0.020% | 0.000% |
| AAV9-P3_LrgInt_125 | 0.000% | 0.020% | 0.000% | 0.020% | 0.020% | 0.020% | 0.041% |
| AAV9-P3_LrgInt_126 | 3.094% | 0.035% | 0.012% | 0.024% | 0.000% | 0.059% | 0.035% |
| AAV9-P3_LrgInt_127 | 0.000% | 0.000% | 0.024% | 0.037% | 0.000% | 0.012% | 0.000% |
| AAV9-P3_LrgInt_128 | 0.154% | 0.024% | 0.036% | 0.012% | 0.000% | 0.000% | 0.071% |
| AAV9-P3_LrgInt_129 | 0.000% | 0.000% | 0.000% | 0.000% | 0.133% | 0.000% | 0.000% |
| AAV9-P14_LrgInt_195 | 0.000% | 0.034% | 0.000% | 0.000% | 0.000% | 0.000% | 0.017% |
| AAV9-P14_LrgInt_197 | 0.000% | 0.073% | 0.018% | 0.000% | 0.000% | 0.000% | 0.000% |
| AAV9-P14_LrgInt_198 | 0.340% | 0.000% | 0.000% | 0.060% | 0.000% | 0.040% | 0.000% |
| AAV9-P14_LrgInt_199 | 0.902% | 0.022% | 0.000% | 0.000% | 0.000% | 0.022% | 0.000% |
| AAV9-P14_LrgInt_215 | 3.771% | 0.017% | 0.000% | 0.000% | 0.000% | 0.017% | 0.035% |
| AAV9-P14_LrgInt_218 | 1.871% | 0.049% | 0.000% | 0.000% | 0.000% | 0.016% | 0.033% |
| AAVPR-P3_Liver_96 | 21.203% | 2.831% | 0.826% | 0.147% | 0.442% | 0.826% | 0.796% |
| AAVPR-P3_Liver_97 | 58.728% | 9.213% | 1.832% | 0.485% | 1.401% | 1.616% | 0.916% |
| AAVPR-P3_Liver_98 | 50.899% | 5.766% | 1.484% | 0.000% | 0.714% | 1.827% | 1.370% |
| AAVPR-P3_Liver_99 | 27.814% | 2.889% | 0.798% | 0.216% | 0.323% | 0.906% | 0.410% |
| AAVPR-P3_Liver_100 | 36.316% | 5.297% | 1.224% | 0.241% | 0.783% | 1.144% | 1.003% |
| AAVPR-P3_Liver_101 | 46.517% | 6.064% | 1.175% | 0.328% | 0.601% | 1.311% | 1.011% |
| AAVPR-P3_Liver_102 | 43.230% | 5.065% | 1.179% | 0.319% | 0.924% | 1.338% | 1.019% |
| AAVPR-P3_Liver_103 | 15.745% | 1.915% | 0.426% | 0.213% | 0.213% | 0.319% | 0.585% |
| AAVPR-P3_Liver_104 | 61.601% | 9.982% | 2.788% | 0.809% | 2.068% | 2.608% | 0.899% |
| AAVPR-P3_Liver_105 | 41.976% | 5.974% | 1.493% | 0.159% | 0.922% | 1.779% | 0.667% |
| AAVPR-P3_Liver_175 | * | * | * | * | * | * | * |
| AAVPR-P3_Liver_177 | 55.351% | 10.640% | 2.394% | 0.552% | 1.125% | 2.578% | 1.514% |
| AAVPR-P3_Liver_178 | 56.845% | 10.763% | 2.342% | 0.407% | 1.568% | 2.652% | 1.820% |
| AAVPR-P3_Liver_179 | 46.437% | 5.272% | 0.881% | 0.258% | 0.760% | 1.291% | 0.653% |
| AAVPR-P3_BrVasc_96 | 2.970% | 0.000% | 0.052% | 0.000% | 0.000% | 0.000% | 0.052% |
| AAVPR-P3_BrVasc_97 | 12.589% | 1.108% | 0.158% | 0.040% | 0.396% | 0.119% | 0.317% |
| AAVPR-P3_BrVasc_98 | 0.000% | 0.668% | 0.000% | 0.000% | 0.178% | 0.045% | 0.000% |
| AAVPR-P3_BrVasc_99 | 0.000% | 0.169% | 0.028% | 0.028% | 0.056% | 0.000% | 0.141% |
| AAVPR-P3_BrVasc_100 | 0.266% | 0.148% | 0.059% | 0.000% | 0.118% | 0.118% | 0.089% |
| AAVPR-P3_BrVasc_101 | 2.521% | 0.752% | 0.265% | 0.000% | 0.044% | 0.088% | 0.088% |
| AAVPR-P3_BrVasc_102 | 5.135% | 0.999% | 0.278% | 0.000% | 0.389% | 0.167% | 0.167% |
| AAVPR-P3_BrVasc_103 | 0.000% | 0.548% | 0.157% | 0.000% | 0.078% | 0.039% | 0.039% |
| AAVPR-P3_BrVasc_104 | 7.025% | 1.233% | 0.262% | 0.037% | 0.075% | 0.187% | 0.224% |
| AAVPR-P3_BrVasc_105 | 0.989% | 0.430% | 0.172% | 0.043% | 0.086% | 0.043% | 0.129% |
| AAVPR-P3_BrVasc_175 | 8.623% | 1.959% | 0.507% | 0.070% | 0.490% | 0.455% | 0.332% |
| AAVPR-P3_BrVasc_177 | 22.458% | 3.616% | 1.006% | 0.082% | 0.897% | 0.761% | 0.952% |
| AAVPR-P3_BrVasc_178 | 5.253% | 0.651% | 0.210% | 0.000% | 0.084% | 0.210% | 0.105% |
| AAVPR-P3_BrVasc_179 | 1.104% | 0.109% | 0.018% | 0.000% | 0.018% | 0.000% | 0.036% |
| AAVPR-P3_AsAort_96 | 0.799% | 0.000% | 0.000% | 0.000% | 0.000% | 0.032% | 0.000% |
| AAVPR-P3_AsAort_97 | 0.000% | 0.000% | 0.000% | 0.000% | 0.000% | 0.038% | 0.038% |
| AAVPR-P3_AsAort_98 | 0.000% | 0.064% | 0.096% | 0.000% | 0.000% | 0.000% | 0.064% |
| AAVPR-P3_AsAort_99 | 0.523% | 0.000% | 0.000% | 0.000% | 0.000% | 0.000% | 0.000% |
| AAVPR-P3_AsAort_100 | 0.000% | 0.000% | 0.030% | 0.000% | 0.000% | 0.030% | 0.000% |
| AAVPR-P3_AsAort_101 | 2.615% | 0.045% | 0.000% | 0.000% | 0.000% | 0.000% | 0.045% |
| AAVPR-P3_AsAort_102 | 0.000% | 0.000% | 0.000% | 0.000% | 0.000% | 0.000% | 0.000% |
| AAVPR-P3_AsAort_103 | 0.000% | 0.000% | 0.031% | 0.000% | 0.000% | 0.031% | 0.000% |
| AAVPR-P3_AsAort_104 | 0.000% | 0.028% | 0.000% | 0.000% | 0.000% | 0.000% | 0.028% |
| AAVPR-P3_AsAort_105 | 0.000% | 0.000% | 0.000% | 0.049% | 0.000% | 0.000% | 0.000% |
| AAVPR-P3_AsAort_175 | * | * | * | * | * | * | * |
| AAVPR-P3_AsAort_177 | * | * | * | * | * | * | * |
| AAVPR-P3_AsAort_178 | * | * | * | * | * | * | * |
| AAVPR-P3_AsAort_179 | 1.339% | 0.044% | 0.015% | 0.000% | 0.030% | 0.015% | 0.103% |
| AAVPR-P3_Heart_96 | 2.813% | 0.284% | 0.000% | 0.085% | 0.142% | 0.057% | 0.085% |
| AAVPR-P3_Heart_97 | 17.531% | 2.015% | 0.171% | 0.043% | 0.171% | 0.214% | 0.471% |
| AAVPR-P3_Heart_98 | 0.638% | 0.447% | 0.096% | 0.032% | 0.064% | 0.096% | 0.351% |
| AAVPR-P3_Heart_99 | 8.583% | 0.369% | 0.026% | 0.000% | 0.158% | 0.079% | 0.053% |
| AAVPR-P3_Heart_100 | 9.069% | 0.795% | 0.177% | 0.000% | 0.147% | 0.147% | 0.000% |
| AAVPR-P3_Heart_101 | 11.624% | 1.155% | 0.269% | 0.000% | 0.154% | 0.154% | 0.231% |
| AAVPR-P3_Heart_102 | 13.140% | 1.141% | 0.154% | 0.031% | 0.000% | 0.247% | 0.154% |
| AAVPR-P3_Heart_103 | 0.142% | 0.256% | 0.000% | 0.000% | 0.000% | 0.057% | 0.114% |
| AAVPR-P3_Heart_104 | 21.405% | 2.011% | 0.510% | 0.030% | 0.450% | 0.540% | 0.090% |
| AAVPR-P3_Heart_105 | 8.621% | 0.862% | 0.276% | 0.034% | 0.172% | 0.172% | 0.000% |
| AAVPR-P3_Heart_175 | 21.037% | 3.718% | 0.635% | 0.073% | 0.272% | 0.508% | 0.544% |
| AAVPR-P3_Heart_177 | 20.087% | 3.037% | 0.542% | 0.043% | 0.282% | 0.369% | 0.412% |
| AAVPR-P3_Heart_178 | 14.695% | 1.783% | 0.255% | 0.023% | 0.255% | 0.370% | 0.232% |
| AAVPR-P3_Heart_179 | 20.573% | 2.866% | 0.226% | 0.025% | 0.251% | 0.453% | 0.528% |
| AAVPR-P3_Lungs_96 | 0.000% | 0.000% | 0.000% | 0.000% | 0.029% | 0.000% | 0.029% |
| AAVPR-P3_Lungs_97 | 0.000% | 0.000% | 0.000% | 0.000% | 0.000% | 0.000% | 0.000% |
| AAVPR-P3_Lungs_98 | 0.000% | 0.058% | 0.029% | 0.000% | 0.000% | 0.058% | 0.058% |
| AAVPR-P3_Lungs_99 | 0.000% | 0.059% | 0.000% | 0.000% | 0.000% | 0.000% | 0.030% |
| AAVPR-P3_Lungs_100 | 0.878% | 0.000% | 0.063% | 0.000% | 0.000% | 0.000% | 0.000% |
| AAVPR-P3_Lungs_101 | 0.000% | 0.000% | 0.034% | 0.000% | 0.000% | 0.034% | 0.000% |
| AAVPR-P3_Lungs_102 | 0.000% | 0.000% | 0.000% | 0.000% | 0.000% | 0.036% | 0.000% |
| AAVPR-P3_Lungs_103 | 0.000% | 0.032% | 0.000% | 0.000% | 0.000% | 0.000% | 0.000% |
| AAVPR-P3_Lungs_104 | 0.821% | 0.063% | 0.032% | 0.000% | 0.000% | 0.000% | 0.000% |
| AAVPR-P3_Lungs_105 | 0.000% | 0.000% | 0.000% | 0.000% | 0.000% | 0.000% | 0.000% |
| AAVPR-P3_Lungs_175 | 0.000% | 0.119% | 0.051% | 0.000% | 0.000% | 0.000% | 0.068% |
| AAVPR-P3_Lungs_177 | 0.000% | 0.033% | 0.099% | 0.000% | 0.033% | 0.000% | 0.000% |
| AAVPR-P3_Lungs_178 | 2.065% | 0.128% | 0.000% | 0.000% | 0.000% | 0.064% | 0.021% |
| AAVPR-P3_Lungs_179 | 2.631% | 0.030% | 0.000% | 0.000% | 0.030% | 0.000% | 0.000% |
| AAVPR-P3_Kidney_96 | 0.000% | 0.000% | 0.000% | 0.000% | 0.000% | 0.000% | 0.057% |
| AAVPR-P3_Kidney_97 | 0.000% | 0.033% | 0.033% | 0.000% | 0.000% | 0.033% | 0.000% |
| AAVPR-P3_Kidney_98 | 0.000% | 0.025% | 0.000% | 0.000% | 0.000% | 0.000% | 0.000% |
| AAVPR-P3_Kidney_99 | 0.000% | 0.000% | 0.000% | 0.000% | 0.033% | 0.033% | 0.000% |
| AAVPR-P3_Kidney_100 | 0.000% | 0.029% | 0.000% | 0.000% | 0.000% | 0.029% | 0.000% |
| AAVPR-P3_Kidney_101 | 0.000% | 0.040% | 0.000% | 0.000% | 0.040% | 0.000% | 0.081% |
| AAVPR-P3_Kidney_102 | 0.000% | 0.032% | 0.000% | 0.032% | 0.000% | 0.032% | 0.063% |
| AAVPR-P3_Kidney_103 | 0.000% | 0.000% | 0.000% | 0.030% | 0.000% | 0.030% | 0.030% |
| AAVPR-P3_Kidney_104 | 2.327% | 0.000% | 0.000% | 0.000% | 0.000% | 0.000% | 0.036% |
| AAVPR-P3_Kidney_105 | 0.000% | 0.000% | 0.031% | 0.000% | 0.000% | 0.000% | 0.000% |
| AAVPR-P3_Kidney_175 | 0.000% | 0.044% | 0.044% | 0.000% | 0.000% | 0.000% | 0.044% |
| AAVPR-P3_Kidney_177 | 0.000% | 0.000% | 0.022% | 0.000% | 0.000% | 0.000% | 0.000% |
| AAVPR-P3_Kidney_178 | 0.000% | 0.025% | 0.000% | 0.000% | 0.025% | 0.000% | 0.000% |
| AAVPR-P3_Kidney_179 | 0.000% | 0.020% | 0.020% | 0.020% | 0.000% | 0.020% | 0.020% |
| AAVPR-P3_Blad_96 | 0.000% | 0.062% | 0.000% | 0.000% | 0.000% | 0.000% | 0.062% |
| AAVPR-P3_Blad_97 | 0.852% | 0.055% | 0.000% | 0.000% | 0.000% | 0.000% | 0.027% |
| AAVPR-P3_Blad_98 | 0.000% | 0.028% | 0.028% | 0.028% | 0.000% | 0.055% | 0.000% |
| AAVPR-P3_Blad_99 | 0.000% | 0.000% | 0.000% | 0.000% | 0.000% | 0.000% | 0.030% |
| AAVPR-P3_Blad_100 | 0.000% | 0.000% | 0.000% | 0.000% | 0.000% | 0.030% | .000% |
| AAVPR-P3_Blad_101 | 0.000% | 0.031% | 0.000% | 0.000% | 0.000% | 0.031% | 0.000% |
| AAVPR-P3_Blad_102 | 1.972% | 0.064% | 0.000% | 0.000% | 0.000% | 0.000% | 0.000% |
| AAVPR-P3_Blad_103 | 0.000% | 0.038% | 0.000% | 0.038% | 0.000% | 0.000% | 0.038% |
| AAVPR-P3_Blad_104 | 0.000% | 0.000% | 0.000% | 0.000% | 0.000% | 0.068% | 0.000% |
| AAVPR-P3_Blad_105 | 0.000% | 0.029% | 0.000% | 0.000% | 0.000% | 0.000% | 0.058% |
| AAVPR-P3_Blad_175 | 0.000% | 0.000% | 0.000% | 0.000% | 0.000% | 0.000% | 0.019% |
| AAVPR-P3_Blad_177 | 0.000% | 0.043% | 0.043% | 0.000% | 0.000% | 0.000% | 0.022% |
| AAVPR-P3_Blad_178 | 0.000% | 0.000% | 0.018% | 0.018% | 0.000% | 0.054% | 0.054% |
| AAVPR-P3_Blad_179 | 2.902% | 0.031% | 0.000% | 0.000% | 0.000% | 0.016% | 0.063% |
| AAVPR-P3_SmaInt_96 | 0.000% | 0.000% | 0.014% | 0.014% | 0.014% | 0.028% | 0.000% |
| AAVPR-P3_SmaInt_97 | 0.000% | 0.015% | 0.000% | 0.030% | 0.030% | 0.000% | 0.000% |
| AAVPR-P3_SmaInt_98 | 0.000% | 0.037% | 0.025% | 0.012% | 0.025% | 0.000% | 0.100% |
| AAVPR-P3_SmaInt_99 | 0.831% | 0.015% | 0.000% | 0.031% | 0.000% | 0.000% | 0.092% |
| AAVPR-P3_SmaInt_100 | 0.000% | 0.030% | 0.030% | 0.015% | 0.015% | 0.015% | 0.015% |
| AAVPR-P3_SmaInt_101 | 0.000% | 0.041% | 0.055% | 0.069% | 0.027% | 0.000% | 0.014% |
| AAVPR-P3_SmaInt_102 | 0.000% | 0.000% | 0.000% | 0.046% | 0.068% | 0.000% | 0.000% |
| AAVPR-P3_SmaInt_103 | 0.361% | 0.000% | 0.000% | 0.052% | 0.000% | 0.000% | 0.000% |
| AAVPR-P3_SmaInt_104 | 0.000% | 0.122% | 0.000% | 0.049% | 0.000% | 0.073% | 0.049% |
| AAVPR-P3_SmaInt_105 | 0.519% | 0.000% | 0.069% | 0.035% | 0.000% | 0.000% | 0.000% |
| AAVPR-P3_SmaInt_175 | 0.000% | 0.016% | 0.016% | 0.000% | 0.016% | 0.016% | 0.032% |
| AAVPR-P3_SmaInt_177 | 0.950% | 0.000% | 0.000% | 0.000% | 0.000% | 0.019% | 0.038% |
| AAVPR-P3_SmaInt_178 | 0.000% | 0.016% | 0.016% | 0.000% | 0.000% | 0.000% | 0.032% |
| AAVPR-P3_SmaInt_179 | 0.000% | 0.000% | 0.000% | 0.000% | 0.000% | 0.000% | 0.018% |
| AAVPR-P3_LrgInt_96 | 1.737% | 0.117% | 0.047% | 0.047% | 0.023% | 0.047% | 0.047% |
| AAVPR-P3_LrgInt_97 | 0.000% | 0.047% | 0.031% | 0.016% | 0.016% | 0.000% | 0.000% |
| AAVPR-P3_LrgInt_98 | 0.000% | 0.000% | 0.000% | 0.000% | 0.056% | 0.000% | 0.019% |
| AAVPR-P3_LrgInt_99 | 0.000% | 0.016% | 0.016% | 0.033% | 0.016% | 0.000% | 0.049% |
| AAVPR-P3_LrgInt_100 | 3.068% | 0.673% | 0.279% | 0.033% | 0.279% | 0.000% | 0.230% |
| AAVPR-P3_LrgInt_101 | 0.000% | 0.000% | 0.028% | 0.000% | 0.014% | 0.014% | 0.028% |
| AAVPR-P3_LrgInt_102 | 4.810% | 0.015% | 0.015% | 0.061% | 0.030% | 0.030% | 0.015% |
| AAVPR-P3_LrgInt_103 | 0.000% | 0.000% | 0.000% | 0.114% | 0.000% | 0.000% | 0.092% |
| AAVPR-P3_LrgInt_104 | 0.000% | 0.050% | 0.000% | 0.000% | 0.000% | 0.000% | 0.069% |
| AAVPR-P3_LrgInt_105 | 0.000% | 0.026% | 0.013% | 0.039% | 0.013% | 0.013% | 0.013% |
| AAVPR-P3_LrgInt_175 | 3.409% | 0.073% | 0.000% | 0.000% | 0.018% | 0.018% | 0.018% |
| AAVPR-P3_LrgInt_177 | 0.000% | 0.000% | 0.000% | 0.000% | 0.019% | 0.000% | 0.058% |
| AAVPR-P3_LrgInt_178 | 0.000% | 0.000% | 0.018% | 0.018% | 0.000% | 0.000% | 0.018% |
| AAVPR-P3_LrgInt_179 | 0.901% | 0.000% | 0.000% | 0.000% | 0.018% | 0.018% | 0.018% |
| Uninject_Liver_196 | 0.000% | 0.033% | 0.000% | 0.017% | 0.000% | 0.017% | 0.017% |
| Uninject_Liver_224 | 0.000% | 0.038% | 0.019% | 0.019% | 0.019% | 0.000% | 0.000% |
| Uninject_BrVasc_196 | 0.000% | 0.000% | 0.000% | 0.000% | 0.000% | 0.000% | 0.000% |
| Uninject_BrVasc_224 | 0.000% | 0.040% | 0.020% | 0.000% | 0.000% | 0.000% | 0.000% |
| Uninject_AsAort_196 | 0.000% | 0.000% | 0.000% | 0.000% | 0.000% | 0.000% | 0.000% |
| Uninject_AsAort_224 | 0.000% | 0.020% | 0.000% | 0.000% | 0.020% | 0.041% | 0.000% |
| Uninject_Heart_196 | 0.000% | 0.026% | 0.000% | 0.000% | 0.052% | 0.000% | 0.026% |
| Uninject_Heart_224 | 0.000% | 0.057% | 0.000% | 0.000% | 0.000% | 0.000% | 0.000% |
| Uninject_Lungs_196 | 0.000% | 0.000% | 0.000% | 0.000% | 0.000% | 0.020% | 0.000% |
| Uninject_Lungs_224 | 0.000% | 0.018% | 0.000% | 0.000% | 0.000% | 0.000% | 0.035% |
| Uninject_Kidney_196 | 0.000% | 0.000% | 0.036% | 0.000% | 0.018% | 0.000% | 0.036% |
| Uninject_Kidney_224 | 0.000% | 0.025% | 0.025% | 0.000% | 0.000% | 0.000% | 0.050% |
| Uninject_Blad_196 | 0.000% | 0.018% | 0.018% | 0.000% | 0.000% | 0.018% | 0.018% |
| Uninject_Blad_224 | 0.000% | 0.000% | 0.000% | 0.000% | 0.000% | 0.022% | 0.022% |
| Uninject_SmaInt_196 | 0.000% | 0.018% | 0.000% | 0.000% | 0.000% | 0.000% | 0.000% |
| Uninject_SmaInt_224 | 0.000% | 0.038% | 0.000% | 0.000% | 0.019% | 0.000% | 0.000% |
| Uninject_LrgInt_196 | 0.000% | 0.020% | 0.020% | 0.000% | 0.000% | 0.000% | 0.000% |
| Uninject_LrgInt_224 | 0.000% | 0.000% | 0.030% | 0.000% | 0.015% | 0.015% | 0.000% |

* Sample not available
LrgInt, Large intestine; SmaInt, Small intestine; Blad, Bladder; BrVasc, Brain vasculature; AsAort, Ascending aorta; DeAort, Descending aorta; Uninject, Uninjected animals.

### Example 10. Generation and Testing of HDAC9 Mini Promoters

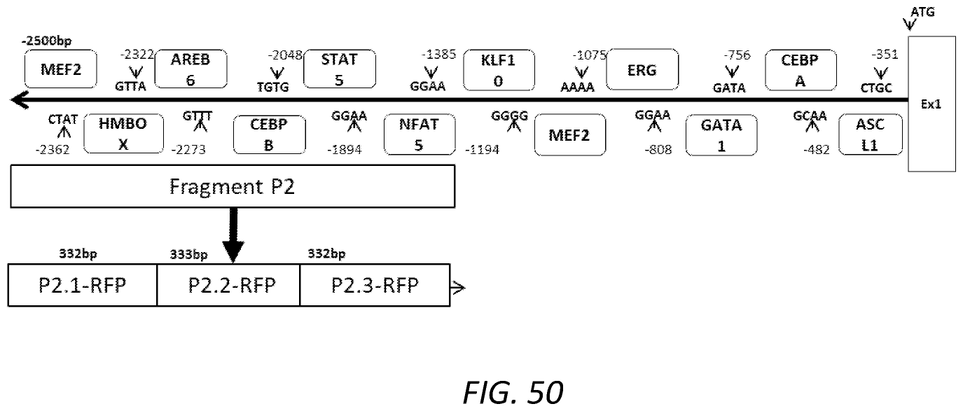

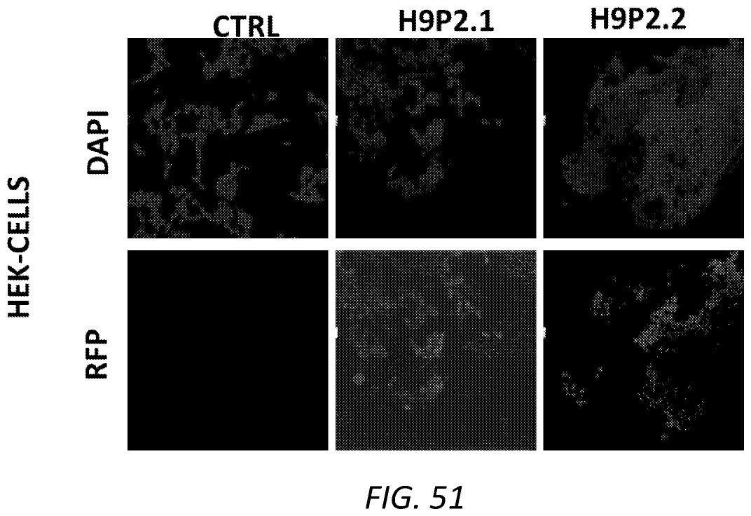

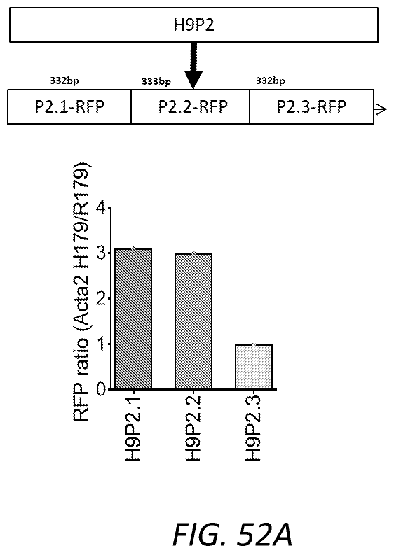

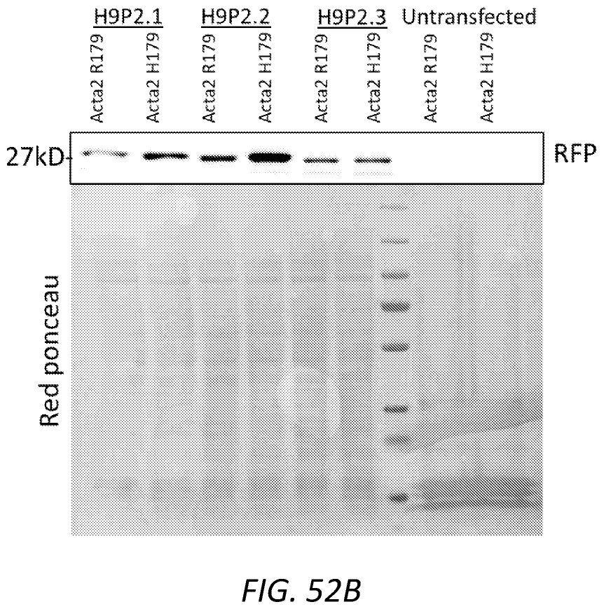

The HDAC9 fragment 2 (P2) identified above containing ˜1200 bp was further divided into 3 new fragments of about ˜330 bp (P2.1, P2.2 and P2.3; FIG. 50). Mini promoters were subcloned into a DNA plasmid to drive the expression of the RFP as an indicator of promoter activity. 2 ug each of plasmids containing P2.1, P2.2 and P2.3 driving expression of red fluorescent protein (RFP) were transfected in wild-type HEK cells. 72 hrs. post transfection, cells were fixed and analyzed on a fluorescent microscope. Results show promoter activity for P2.1 and P2.2. as observed by the red color intensity (FIG. 51). P2.3 showed lower activity. Western blotting confirmed these results (FIGS. 52A-B).

### REFERENCES

1. Jinek, M. et al. A Programmable Dual-RNA-Guided DNA Endonuclease in Adaptive Bacterial Immunity. Science 337, 816-821 (2012).
2. Doudna, J. A. The promise and challenge of therapeutic genome editing. Nature 578, 229-236 (2020).
3. Sternberg, S. H., Redding, S., Jinek, M., Greene, E. C. & Doudna, J. A. DNA interrogation by the CRISPR RNA-guided endonuclease Cas9. Nature 507, 62-67 (2014).
4. Kleinstiver, B. P. et al. Engineered CRISPR-Cas9 nucleases with altered PAM specificities. Nature 523, 481-485 (2015).
5. Kleinstiver, B. P. et al. Broadening the targeting range of Staphylococcus aureus CRISPR-Cas9 by modifying PAM recognition. Nat. Biotechnol. 33, 1293-1298 (2015).
6. Kleinstiver, B. P. et al. High-fidelity CRISPR-Cas9 nucleases with no detectable genome-wide off-target effects. Nature 529, 490-495 (2016).
7. Kleinstiver, B. P. et al. Engineered CRISPR-Cas12a variants with increased activities and improved targeting ranges for gene, epigenetic and base editing. Nat. Biotechnol. 37, 276-282 (2019).
8. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
9. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
10. Gaudelli, N. M. et al. Programmable base editing of A·T to G·C in genomic DNA without DNA cleavage. Nature 551, 464-471 (2017).
11. Rees, H. A. & Liu, D. R. Base editing: precision chemistry on the genome and transcriptome of living cells. Nat. Rev. Genet. 19, 770-788 (2018).
12. Porto, E. M., Komor, A. C., Slaymaker, I. M. & Yeo, G. W. Base editing: advances and therapeutic opportunities. Nat. Rev. Drug Discov. 19, 839-859 (2020).
13. Anzalone, A. V. et al. Search-and-replace genome editing without double-strand breaks or donor DNA. Nature 576, 149-157 (2019).
14. Anzalone, A. V., Koblan, L. W. & Liu, D. R. Genome editing with CRISPR-Cas nucleases, base editors, transposases and prime editors. Nat. Biotechnol. 38, 824-844 (2020).
15. Gaudelli, N. M. et al. Directed evolution of adenine base editors with increased activity and therapeutic application. Nat. Biotechnol. 38, 892-900 (2020).
16. Richter, M. F. et al. Phage-assisted evolution of an adenine base editor with improved Cas domain compatibility and activity. Nat. Biotechnol. 38, 883-891 (2020).
17. Kleinstiver, B. & Walton, R. T. Crispr-cas enzymes with enhanced on-target activity. US2021/0261932
18. Miller, S. M. et al. Continuous evolution of SpCas9 variants compatible with non-G PAMs. Nat. Biotechnol. 38, 471-481 (2020).
19. Levy, J. M. et al. Cytosine and adenine base editing of the brain, liver, retina, heart and skeletal muscle of mice via adeno-associated viruses. Nat. Biomed. Eng. 4, 97-110 (2020).
20. Wu, H., Hu, Z. & Liu, X.-Q. Protein trans-splicing by a split intein encoded in a split DnaE gene of Synechocystis sp. PCC6803. Proc. Natl. Acad. Sci. 95, 9226-9231 (1998).
21. Zettler, J., Schütz, V. & Mootz, H. D. The naturally split Npu DnaE intein exhibits an extraordinarily high rate in the protein trans-splicing reaction. FEBS Lett. 583, 909-914 (2009).
22. Stevens, A. J. et al. Design of a Split Intein with Exceptional Protein Splicing Activity. J. Am. Chem. Soc. 138, 2162-2165 (2016).
23. Koblan, L. W. et al. Improving cytidine and adenine base editors by expression optimization and ancestral reconstruction. Nat. Biotechnol. 36, 843-846 (2018).
24. Rohland, N. & Reich, D. Cost-effective, high-throughput DNA sequencing libraries for multiplexed target capture. Genome Res. 22, 939-946 (2012).
25. Clement, K. et al. CRISPResso2 provides accurate and rapid genome editing sequence analysis. Nat. Biotechnol. 37, 224-226 (2019).
26. Munot, P., et al. A novel distinctive cerebrovascular phenotype is associated with heterozygous Arg179 ACTA2 mutations. Brain 135, 2506-2514 (2012).
27. Regalado, E. S., et al. Clinical history and management recommendations of the smooth muscle dysfunction syndrome due to ACTA2 arginine 179 alterations. Genet Med 20, 1206-1215 (2018).
28. Etchevers, H. C., Vincent, C., Le Douarin, N. M. & Couly, G. F. The cephalic neural crest provides pericytes and smooth muscle cells to all blood vessels of the face and forebrain. Development 128, 1059-1068 (2001).
29. Jiang, X., et al. Normal fate and altered function of the cardiac neural crest cell lineage in retinoic acid receptor mutant embryos. Mech Dev 117, 115-122 (2002).
30. Li, Y., et al. Intrastriatal transplantation of bone marrow nonhematopoietic cells improves functional recovery after stroke in adult mice. J Cereb Blood Flow Metab 20, 1311-1319 (2000).
31. Chung, D. Y., et al. Determinants of Optogenetic Cortical Spreading Depolarizations. Cereb Cortex (2018).
32. Xie, H., et al. Differential effects of anesthetics on resting state functional connectivity in the mouse. J Cereb Blood Flow Metab 40, 875-884 (2020).
33. Ash, J. A., et al. Functional connectivity with the retrosplenial cortex predicts cognitive aging in rats. Proc Natl Acad Sci USA 113, 12286-12291 (2016).
34. Kitagawa, K., et al. Cerebral ischemia after bilateral carotid artery occlusion and intraluminal suture occlusion in mice: evaluation of the patency of the posterior communicating artery. J Cereb Blood Flow Metab 18, 570-579 (1998).
35. Hattori, Y., Kitamura, A., Nagatsuka, K. & Ihara, M. A novel mouse model of ischemic carotid artery disease. PloS one 9, e100257 (2014).
36. Papke, C. L., et al. Smooth muscle hyperplasia due to loss of smooth muscle alpha-actin is driven by activation of focal adhesion kinase, altered p53 localization and increased levels of platelet-derived growth factor receptor-beta. Hum Mol Genet 22, 3123-3137 (2013).
37. Lino Cardenas, C. L., et al. An HDAC9-MALAT1-BRG1 complex mediates smooth muscle dysfunction in thoracic aortic aneurysm. Nat Commun 9, 1009 (2018).
38. Schiffner, R., et al. Redistribution of Cerebral Blood Flow during Severe Hypovolemia and Reperfusion in a Sheep Model: Critical Role of alpha1-Adrenergic Signaling. Int J Mol Sci 18 (2017).
39. Grunstein, M. Histone acetylation in chromatin structure and transcription. Nature 389, 349-352 (1997).
40. Haberland, M., Montgomery, R. L. & Olson, E. N. The many roles of histone deacetylases in development and physiology: implications for disease and therapy. Nat Rev Genet 10, 32-42 (2009).
41. Kato, N., et al. Trans-ancestry genome-wide association study identifies 12 genetic loci influencing blood pressure and implicates a role for DNA methylation. Nat Genet 47, 1282-1293 (2015).
42. Foroud, T., et al. Genome-wide association study of intracranial aneurysm identifies a new association on chromosome 7. Stroke: a journal of cerebral circulation 45, 3194-3199 (2014).
43. International Stroke Genetics, C., et al. Genome-wide association study identifies a variant in HDAC9 associated with large vessel ischemic stroke. Nat Genet 44, 328-333 (2012).
44. Myocardial Infarction Genetics, C., et al. Genome-wide association of early-onset myocardial infarction with single nucleotide polymorphisms and copy number variants. Nat Genet 41, 334-341 (2009).
45. Lino Cardenas, C. L., et al. An HDAC9-MALATI-BRG1 complex mediates smooth muscle dysfunction in thoracic aortic aneurysm. Nat Commun 9, 1009 (2018).
46. Gallo, E. M., et al. Angiotensin II-dependent TGF-beta signaling contributes to Loeys-Dietz syndrome vascular pathogenesis. J Clin Invest 124, 448-460 (2014).
47. Regalado, E. S., et al. Aortic Disease Presentation and Outcome Associated with ACTA2 Mutations. Circulation. Cardiovascular genetics 8, 457-464 (2015).
48. Lino Cardenas, C. L., et al. HDAC9 complex inhibition improves smooth muscle-dependent stenotic vascular disease. JCI Insight 4 (2019).
49. Haberland, M., et al. Regulation of HDAC9 gene expression by MEF2 establishes a negative-feedback loop in the transcriptional circuitry of muscle differentiation. Mol Cell Biol 27, 518-525 (2007).
50. Judge, D. P., et al. Evidence for a critical contribution of haploinsufficiency in the complex pathogenesis of Marfan syndrome. J Clin Invest 114, 172-181 (2004).
51. Silasi, G., Xiao, D., Vanni, M. P., Chen, A. C. & Murphy, T. H. Intact skull chronic windows for mesoscopic wide-field imaging in awake mice. J Neurosci Methods 267, 141-149 (2016).
52. Xie, H., et al. Differential effects of anesthetics on resting state functional connectivity in the mouse. J Cereb Blood Flow Metab 40, 875-884 (2020).
53. White, B. R., et al. Imaging of functional connectivity in the mouse brain. PloS one 6, e16322 (2011).
54. Kura, S., et al. Intrinsic optical signal imaging of the blood volume changes is sufficient for mapping the resting state functional connectivity in the rodent cortex. J Neural Eng 15, 035003 (2018).
55. Edelstein, A. D., et al. Advanced methods of microscope control using muManager software. J Biol Methods 1 (2014).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated nucleic acid comprising a promoter sequence that has at least 90% identity to SEQ ID NO: 4 and optionally contains at least one substitution modification relative to SEQ ID NO: 4, or a promoter sequence that has at least 90% identity to SEQ ID NO: 5 and optionally contains at least one substitution modification relative to SEQ ID NO: 5, or a promoter sequence that has at least 90% identity to SEQ ID NO: 6 and optionally contains at least one substitution modification relative to SEQ ID NO: 6, or a promoter sequence that has at least 90% identity to SEQ ID NO: 85 and optionally contains at least one substitution modification relative to SEQ ID NO: 85, or a promoter sequence that has at least 90% identity to SEQ ID NO: 86 and optionally contains at least one substitution modification relative to SEQ ID NO: 86, or a promoter sequence that has at least 90% identity to SEQ ID NO: 87 and optionally contains at least one substitution modification relative to SEQ ID NO: 87.

2. An isolated nucleic acid comprising a heterologous transgene and a promoter sequence that has at least 90% identity to SEQ ID NO: 4, 5, 6, 85, 86, or 87, preferably wherein the promoter sequence is operably linked to the heterologous coding sequence.

3. The isolated nucleic acid of claim 1 or 2, wherein the promoter drives transcription of the heterologous coding sequence in vascular smooth muscle cells.

4. A vector comprising the nucleic acid of claim 1, and a heterologous nucleic acid sequence, optionally a transgene operably linked to the promoter sequence, optionally wherein the vector is encapsulated by an adeno-associated virus (AAV) capsid.

5. An adeno-associated virus (AAV) vector comprising (i) one or more of peptides selected from the group consisting of PRPPSTH (SEQ ID NO:1), MAEPGAR (SEQ ID NO:25), MLYADNT (SEQ ID NO: 26), and SQDPSTL (SEQ ID NO: 27), in its capsid, and (ii) a nucleic acid sequence comprising a smooth muscle cell-specific promoter or active portion thereof encapsulated by the capsid.

6. The AAV vector of claim 5, wherein the smooth muscle cell-specific promoter is an HDAC9-derived promoter.

7. The AAV vector of claim 6, wherein the HDAC9-derived promoter comprises a sequence that has at least 90% identity to SEQ ID NO: 4, 5, 6, 85, 86, or 87.

8. The AAV vector of any of claims 5-7, wherein the smooth muscle promoter is operably linked to a heterologous transgene.

9. The AAV vector of claim 8, wherein the transgene encodes a genome editor or an intein-split construct thereof, optionally selected from CRISPR nucleases, cytosine base editors (CBEs), adenine base editors (ABEs), and CRISPR prime editors (PEs).

10. The AAV vector of claim 9, wherein the transgene encodes:
(a) a CRISPR/Cas base editor or an intein-split construct thereof, and/or an Arg179His mutant-allele specific guide RNA directing a base editor to the mutation, and/or an Arg179Cys mutant-allele specific guide RNA directing a base editor to the mutation, or
(b) a CRISPR/Cas genome editor or an intein-split construct thereof, and/or an Arg179His mutant-allele specific guide RNA directing a genome editor to the mutation, and/or an Arg179Cys mutant-allele specific guide RNA directing a base editor to the mutation.

11. The AAV vector of claim 5, wherein the sequence PRPPSTH (SEQ ID NO: 1) is inserted into VP1 protein of the AAV vector.

12. The AAV vector of claim 11, wherein the sequence PRPPSTH (SEQ ID NO: 1) is inserted into the VP1 protein in a position corresponding to amino acids 588 and 589 of SEQ ID NO: 2.

13. The AAV vector of claims 5-12, wherein the AAV serotype is AAV-9 or AAV-2.

14. A composition comprising the vector of claim 4 or the AAV vectors of claims 5-13.

15. A recombinant episome comprising a nucleic acid sequence that encodes an adeno-associated virus (AAV) capsid having one or more of peptides selected from the group consisting of PRPPSTH (SEQ ID NO: 1), MAEPGAR (SEQ ID NO:25), MLYADNT (SEQ ID NO: 26), and SQDPSTL (SEQ ID NO: 27) in its capsid, and a nucleic acid sequence comprising a smooth muscle specific promoter or active portion thereof encapsulated by the capsid.

16. The recombinant episome of claim 15, wherein at least two copies of the one or more of peptides selected from the group consisting of PRPPSTH (SEQ ID NO:1), MAEPGAR (SEQ ID NO:25), MLYADNT (SEQ ID NO:26), and SQDPSTL (SEQ ID NO:27) are inserted into the capsid of the AAV vector.

17. The recombinant episome of claim 16, wherein the at least two copies are of a same peptide.

18. The recombinant episome of claim 16, wherein the at least two copies are of a different peptide.

19. The recombinant episome of claim 15, wherein the one or more of peptides selected from the group consisting of PRPPSTH (SEQ ID NO:1), MAEPGAR (SEQ ID NO:25), MLYADNT (SEQ ID NO:26), and SQDPSTL (SEQ ID NO:27) are inserted into the VP1 protein of the AAV vector.

20. The recombinant episome of claim 19, wherein the one or more of peptides selected from the group consisting of PRPPSTH (SEQ ID NO:1), MAEPGAR (SEQ ID NO:25), MLYADNT (SEQ ID NO: 26), and SQDPSTL (SEQ ID NO: 27) are inserted into the VP1 protein of the AAV vector in a position corresponding to amino acids 588 and 589 of SEQ ID NO: 2.

21. The recombinant episome of claims 15 to 19, wherein the AAV serotype is AAV-9 or AAV-2.

22. A composition comprising the AAV vector encoded by the recombinant episome of claims 15 to 21.

23. A method of correcting an Arg179His mutation or an Arg179Cys mutation in the genome of a cell, the method comprising delivering to the cell:
(i) a CRISPR/Cas base editor or an intein-split construct thereof, and an Arg179His or an Arg179Cys mutant-allele specific guide RNA directing the base editor to the mutation, or
(ii) a CRISPR/Cas genome editor or an intein-split construct thereof, and an Arg179His or an Arg179Cys mutant-allele specific guide RNA directing the genome editor to the mutation.

24. The method of claim 23, wherein the base editor and/or guide RNA are listed in Table 1 and/or Table 2 (comprising SEQ ID NOs: 55-79), optionally comprising SEQ ID NO:60.

25. The method of claim 23, wherein the Arg179His or the Arg179Cys mutant-allele specific CRISPR/Cas base editor is an adenine base editor or an intein-split construct thereof comprising the wild-type SpCas9, A61R/L1111R/D1135L/S1136W/G1218K/E1219Q/N1317R/A1322R/R1333P/R1335 Q/T1337R (SpRY), D10T(optional)/1322V/S409I/E427G/R654L/R753G/R1114G/D1135N/V1139A/D1180G/E1219V/Q1221H/A1320V/R1333K (SpCas9-NRRH), D10T(optional)/1322V/S409I/E427G/R654L/R753G/R1114G/D1135N/E1219V/D1332N/R1335Q/T1337N/S1338T/H1349R (SpCas9-NRCH), D1135M/S1136Q/G1218K/E1219S/R1335E/T1337R (MQSKER), D1135L/S1136W/G1218K/E1219Q/R1335Q/T1337R (SpG) or D1135V/G1218R/R1335Q/T1337R (VRQR) or S55R/D1135V/G1218R/R1335Q/T1337R (VRQR(S55R)) variants of Streptococcus pyogenes Cas9 protein (SpCas9).

26. The method of any of claims 23 to 25, wherein the spacer sequence of the guide RNA targets the sequence TGCATCTGGATCTGGCTGGC (SEQ ID NO:28: HES1208-A4 gRNA), optionally in combination with ABE8e-SpCas9-VRQR, ABE8e-SpCas9-VRQR(S55R), ABE8e-SpG, ABE8e-SpRY, or ABE8e-SpCas9-NRRH, or an intein-split construct thereof; the spacer sequence of the guide RNA targets the sequence TCATGCATCTGGATCTGGCT (SEQ ID NO: 24, HES1210-A7 gRNA), optionally in combination with ABE8e-SpG, ABE8e-SpRY, ABE8e-SpCas9-NRCH, ABE8e-MQSKER or an intein-split construct thereof; or the spacer sequence of the guide RNA targets the sequence ATCATGCATCTGGATCTGGC (SEQ ID NO:23, HES1212-A8), optionally in combination with ABE8e-SpCas9, ABE8e-SpG, ABE8e-SpRY, or ABE8e-SpCas9-NRRH or an intein-split construct thereof.

27. The method of claim 23 or 24, wherein the genome editor is a wild-type SpCas9 or an intein-split construct thereof.

28. The method of claim 27, wherein the spacer sequence of the guide RNA comprises the sequence TGCCATCATGCATCTGGATC (HES1235, SEQ ID NO:83) or AGCCAGATCCAGATGCATGA (HES1236, SEQ ID NO:84).

29. The method of any of claims 23 to 28, wherein the base editor or intein-split construct thereof and guide RNA, or the genome editor or intein-split construct thereof and guide RNA, are delivered to the cell using one or more adeno-associated virus (AAV) vectors, preferably AAV comprising the sequence PRPPSTH (SEQ ID NO: 1) inserted into the VP1 protein, in a position corresponding to between amino acids 588 and 589 of SEQ ID NO: 2, and preferably comprising an HDAC9 promoter or active portion thereof.

30. The method of claim 29, wherein the HDAC9 promoter comprises a sequence that has at least 90% identity to SEQ ID NO: 4, 5, 6, 85, 86, or 87.

31. The method of any of claims 23 to 30, wherein the cell is in a living subject.

32. The method of claim 31, wherein the living subject is a human with:
(i) an Arg179His mutation in an allele of ACTA2, and the method comprises delivering a base editor or intein-split construct thereof and/or guide RNA, or
(ii) an Arg179Cys mutation in an allele of ACTA2, and the method comprises delivering a genome editor or intein-split construct thereof and/or guide RNA.

33. A method of treating a vasculopathy, optionally multisystem smooth muscle dysfunction syndrome (MSMDS), in a subject who has an ACTA2 R179H mutation, the method comprising delivering to the vasculature of the subject a therapeutically effective amount of a gene therapy agent comprising:
(a) a CRISPR/Cas base editor or an intein-split construct thereof, and a guide RNA directing the base editor to the Arg179His mutation, or
(b) a CRISPR/Cas genome editor or an intein-split construct thereof, and a guide RNA directing the genome editor to the Arg179His mutation.

34. The method of claim 33, wherein the base editor or intein-split construct thereof, genome editor or intein-split construct thereof, and/or guide RNA are listed in Table 1 and/or Table 2(comprising SEQ ID NOs: 55-79), optionally comprising SEQ ID NO:60.

35. The method of claim 33 or 34, wherein the Arg179His mutant-allele specific CRISPR/Cas base editor is an adenine base editor or an intein-split construct thereof comprising the wild type Cas9, A61R/L1111R/D1135L/S1136W/G1218K/E1219Q/N1317R/A1322R/R1333P/R1335 Q/T1337R (SpRY), D10T(optional)/1322V/S409I/E427G/R654L/R753G/R1114G/D1135N/V1139A/D1180G/E1219V/Q1221H/A1320V/R1333K (SpCas9-NRRH), D10T(optional)/1322V/S409I/E427G/R654L/R753G/R1114G/D1135N/E1219V/D1332N/R1335Q/T1337N/S1338T/H1349R (SpCas9-NRCH), D1135M/S1136Q/G1218K/E1219S/R1335E/T1337R (MQSKER), D1135L/S1136W/G1218K/E1219Q/R1335Q/T1337R (SpG) or D1135V/G1218R/R1335Q/T1337R (VRQR) or S55R/D1135V/G1218R/R1335Q/T1337R (VRQR(S55R)) variants of Streptococcus pyogenes Cas9 protein (SpCas9).

36. The method of any of claims 33 to 35, wherein the spacer sequence of the guide RNA directing the base editor to the mutation comprises the sequence TGCATCTGGATCTGGCTGGC (SEQ ID NO:28, HES1208-A4 gRNA), optionally in combination with ABE8e-SpCas9-VRQR, ABE8e-SpCas9-VRQR(S55R), ABE8e-SpG, ABE8e-SpRY, or ABE8e-SpCas9-NRRH or an intein-split construct thereof: the spacer sequence of the guide RNA targets the sequence TCATGCATCTGGATCTGGCT (SEQ ID NO: 24, HES1210-A7 gRNA), optionally in combination with ABE8e-SpG, ABE8e-SpRY, ABE8e-SpCas9-NRCH, ABE8e-MQSKER or an intein-split construct thereof; or the spacer sequence of the guide RNA targets the sequence ATCATGCATCTGGATCTGGC (SEQ ID NO:23, HES1212-A8), optionally in combination with ABE8e-SpCas9, ABE8e-SpG, ABE8e-SpRY, or ABE8e-SpCas9-NRRH or an intein-split construct thereof.

37. The method of claim 33, wherein the genome editor and/or guide RNA are listed in Table 1 and/or Table 2 (comprising SEQ ID NOs: 55-79), optionally comprising SEQ ID NO:60.

38. The method of claim 37, wherein the genome editor is a wild-type SpCas9 or an intein-split construct thereof.

39. The method of any of claim 33, 37, or 38, wherein the spacer sequence of the guide RNA directing the genome editor to the mutation comprises the sequence TGCCATCATGCATCTGGATC (HES1235, SEQ ID NO:83) or AGCCAGATCCAGATGCATGA (HES1236, SEQ ID NO:84).

40. The method of any of claims 33 to 39, wherein the base editor or intein-split construct thereof and guide RNA are delivered to the cell using adeno-associated virus (AAV) vectors, preferably AAV comprising the sequence PRPPSTH (SEQ ID NO: 1) inserted into the VP1 protein, in a position corresponding to between amino acids 588 and 589 of SEQ ID NO:2, and preferably comprising an HDAC9-derived promoter.

41. The method of claim 40, wherein the HDAC9-derived promoter comprises a sequence that has at least 90% identity to SEQ ID NO: 4, 5, 6, 85, 86, or 87.

42. The method of any of claims 33 to 41, wherein the gene therapy agent is administered to the subject by local or systemic delivery.

43. The method of claim 42, wherein the gene therapy agent is administered systemically by intravenous or intra-arterial delivery.


SEQUENCE LISTING (87 sequences)

SEQ ID NO: 1 - protein, 7 aa, synthetic construct
PRPPSTH

SEQ ID NO: 2 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQPRPPSTHAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - DNA, 2500 bp, synthetic construct
  source 1..2500: mol_type = other DNA
ataaatgttttgtagaataaaaaaaaaaaaagttcttcaaaagaaatctcaaatctccaaatggaaacaggtaaaagtggagctcccctggttccacggagaaccttttttgaggaaacttaggcaactcgcaggtaccttatgtcatgagacagagtttgaaaactacaattgactatctctaaatttcctcccaggtctaaaatgtgatgatagttactacttcagtacatcatccttaaggaaaattattaggtccacactgtttctatcctttgaattttacacataaattttgtaatcaaaagtttatttgtaatatcagatggaatcagataattgctttttgttttttccactgacaggaacataagattttgttgtgtagcttaagtcaaacgcagtttggaatatatattttttaaaaattgtaacttacatatccaaatacaatttttcaagaagtagagtattcagtagaaattaatctgtgaaagaagaggaattcagcagtggcctatttgatgaatgatttaacgtgcttatttcttccctttcatcaaaactctgtgtccccttgtttgccccctctgacttcatactctggagttgaccaagatccctcttccatcggattgttctgggaattttgaaataatctgctttttcctctctttcccctgttgcttctgatgccttagaattacattttcctcgctgatttagtttagaaaagagaaaagagcttccatgactagtagattatcacttttgggtttgctcttggaagtgacaagatgctaggatccctctttggaatgtaaaatttatctcttatatagaaaggatataaatgtagcaccagagactataaaactctgatactatctactgtactgtatagctgaacgccacaatgtgtctggtaatctattgactatcataaatgctatttctacagaaaagttaggaggtccatatttcgggcaaccaatgtatagctgaatgcagaacagtcatagttgggtactaaccatatatatgatttatccatcaacaggtgcatatgctcagaaattctgtatccataagaaatcagactactttcttttccttttgcaagtaaattgaatttagcctgagaggctgaggggaaattttcacatataagccacggttttgtgttttgtgttttgttttgtttatagatatagtactaactggatggatgcgataaaattcataggtggtactaagatacaataggatttgtgaaatggacaattgtcttgcataaatagcaagtaaaaaatcaagcctgtccttcataaaaattttattctggggtgtgcttgttttccaaaagtacctctgctaaatctcctgttagtcctgaaactagaaggcagaaaagcttctagtgctacagccaactgcagtgtagcctgagaaacaggcaacaaaaatagaacaccaggattgctgtgcgtgggtgaggcagaaaccacattatgagcaaaagcttccagtattattttagaaccaatacagagctctgtacttctctcctctctcttccaaaaacacatactacaaaaataaaatgaaatgaaatgtatgtgcatttgccctcttagaattatgattcttaattttttttcttgccttcctttctttggaagcgaatcgccagtatggaaacacagtgtgtaaagcaagcttcgagagaggaaagagttaattggttttaaggccctgcgatagagaattatggttggaaagatagaggctggacagctgggtttgctggggtatttttaaatgcattaatgcaggctccaatcactcggccatgcttgacctatttttggctcaggccgaccattgttctatttctgtgcctgtgggccatgctgttgttgattcatatgcaaatggattatcactcgctttagccaacttgagctgagagagactgagaaagggggaagagaggcacagacacagataggagaagggcaccggctggagccacttgcaggactgagggtttttgcaacaaaaccctagcagcctgaagaactctaagccaggtttaattggtttctttttctcgtgggtagacttaataattttctacgtattctgacaaagaaataaccccgaagcacgttcctatttcccacctgcttgtagtttccgggataacctaaactccagagagctatagcatccactctgtcctttctgctttgcacacaggttggtaacatgggaaaagtgtccaggtctttttaaaagtggatgcccatttgagcagaaaggaaatcattgtcgaagttgatcctctgctgcttctcctcagggaggagggagaaccagcgagggtagctcctggggccggtgcactgagcagtgatgaatgtttcatgtagctgaagtaagagtgactggaatatgctgcagacaatttacgagagtgactcctgtttttcctcag

SEQ ID NO: 5 - DNA, 1350 bp, synthetic construct
  source 1..1350: mol_type = other DNA
ataaatgttttgtagaataaaaaaaaaaaaagttcttcaaaagaaatctcaaatctccaaatggaaacaggtaaaagtggagctcccctggttccacggagaaccttttttgaggaaacttaggcaactcgcaggtaccttatgtcatgagacagagtttgaaaactacaattgactatctctaaatttcctcccaggtctaaaatgtgatgatagttactacttcagtacatcatccttaaggaaaattattaggtccacactgtttctatcctttgaattttacacataaattttgtaatcaaaagtttatttgtaatatcagatggaatcagataattgctttttgttttttccactgacaggaacataagattttgttgtgtagcttaagtcaaacgcagtttggaatatatattttttaaaaattgtaacttacatatccaaatacaatttttcaagaagtagagtattcagtagaaattaatctgtgaaagaagaggaattcagcagtggcctatttgatgaatgatttaacgtgcttatttcttccctttcatcaaaactctgtgtccccttgtttgccccctctgacttcatactctggagttgaccaagatccctcttccatcggattgttctgggaattttgaaataatctgctttttcctctctttcccctgttgcttctgatgccttagaattacattttcctcgctgatttagtttagaaaagagaaaagagcttccatgactagtagattatcacttttgggtttgctcttggaagtgacaagatgctaggatccctctttggaatgtaaaatttatctcttatatagaaaggatataaatgtagcaccagagactataaaactctgatactatctactgtactgtatagctgaacgccacaatgtgtctggtaatctattgactatcataaatgctatttctacagaaaagttaggaggtccatatttcgggcaaccaatgtatagctgaatgcagaacagtcatagttgggtactaaccatatatatgatttatccatcaacaggtgcatatgctcagaaattctgtatccataagaaatcagactactttcttttccttttgcaagtaaattgaatttagcctgagaggctgaggggaaattttcacatataagccacggttttgtgttttgtgttttgttttgtttatagatatagtactaactggatggatgcgataaaattcataggtggtactaagatacaataggatttgtgaaatggacaattgtcttgcataaatagcaagtaaaaaatcaagcctgtccttcataaaaattttattctggggtgt

SEQ ID NO: 6 - DNA, 1150 bp, synthetic construct
  source 1..1150: mol_type = other DNA
gcttgttttccaaaagtacctctgctaaatctcctgttagtcctgaaactagaaggcagaaaagcttctagtgctacagccaactgcagtgtagcctgagaaacaggcaacaaaaatagaacaccaggattgctgtgcgtgggtgaggcagaaaccacattatgagcaaaagcttccagtattattttagaaccaatacagagctctgtacttctctcctctctcttccaaaaacacatactacaaaaataaaatgaaatgaaatgtatgtgcatttgccctcttagaattatgattcttaattttttttcttgccttcctttctttggaagcgaatcgccagtatggaaacacagtgtgtaaagcaagcttcgagagaggaaagagttaattggttttaaggccctgcgatagagaattatggttggaaagatagaggctggacagctgggtttgctggggtatttttaaatgcattaatgcaggctccaatcactcggccatgcttgacctatttttggctcaggccgaccattgttctatttctgtgcctgtgggccatgctgttgttgattcatatgcaaatggattatcactcgctttagccaacttgagctgagagagactgagaaagggggaagagaggcacagacacagataggagaagggcaccggctggagccacttgcaggactgagggtttttgcaacaaaaccctagcagcctgaagaactctaagccaggtttaattggtttctttttctcgtgggtagacttaataattttctacgtattctgacaaagaaataaccccgaagcacgttcctatttcccacctgcttgtagtttccgggataacctaaactccagagagctatagcatccactctgtcctttctgctttgcacacaggttggtaacatgggaaaagtgtccaggtctttttaaaagtggatgcccatttgagcagaaaggaaatcattgtcgaagttgatcctctgctgcttctcctcagggaggagggagaaccagcgagggtagctcctggggccggtgcactgagcagtgatgaatgtttcatgtagctgaagtaagagtgactggaatatgctgcagacaatttacgagagtgactcctgtttttcctcag

SEQ ID NO: 7 - DNA, 56 bp, synthetic construct
  source 1..56: mol_type = other DNA
acactctttccctacacgacgctcttccgatctgtgctggactctggagatggtgt

SEQ ID NO: 8 - DNA, 57 bp, synthetic construct
  source 1..57: mol_type = other DNA
gactggagttcagacgtgtgctcttccgatctggatggtcctggaagttactgagca

SEQ ID NO: 9 - DNA, 56 bp, synthetic construct
  source 1..56: mol_type = other DNA
acactctttccctacacgacgctcttccgatctgatggtgtgactcacaacgtgcc

SEQ ID NO: 10 - DNA, 57 bp, synthetic construct
  source 1..57: mol_type = other DNA
gactggagttcagacgtgtgctcttccgatctgccctccttctgataggttacagtg

SEQ ID NO: 11 - DNA, 20 bp, synthetic construct
  source 1..20: mol_type = other DNA
aatcaacctctggattacaa

SEQ ID NO: 12 - DNA, 20 bp, synthetic construct
  source 1..20: mol_type = other DNA
atagcatgatacaaaggcat

SEQ ID NO: 13 - DNA, 20 bp, synthetic construct
  source 1..20: mol_type = other DNA
gatgtccttggtgcacactc

SEQ ID NO: 14 - DNA, 18 bp, synthetic construct
  source 1..18: mol_type = other DNA
gagctgagattgccccgc

SEQ ID NO: 15 - DNA, 24 bp, synthetic construct
  source 1..24: mol_type = other DNA
cgagaagaagaatggcctgttcgg

SEQ ID NO: 16 - DNA, 23 bp, synthetic construct
  source 1..23: mol_type = other DNA
cgacatcctgagagtgaacaccg

SEQ ID NO: 17 - DNA, 25 bp, synthetic construct
  source 1..25: mol_type = other DNA
gatcaccctgaagtccaagctggtg

SEQ ID NO: 18 - DNA, 22 bp, synthetic construct
  source 1..22: mol_type = other DNA
ggcgatcatcttccgcacgtcg

SEQ ID NO: 19 - DNA, 21 bp, synthetic construct
  source 1..21: mol_type = other DNA
ggaaaaccccatcaacgccag

SEQ ID NO: 20 - DNA, 21 bp, synthetic construct
  source 1..21: mol_type = other DNA
gctcttgaagttgggggtcag

SEQ ID NO: 21 - DNA, 21 bp, synthetic construct
  source 1..21: mol_type = other DNA
gacggctggaaaatctgatcg

SEQ ID NO: 22 - DNA, 22 bp, synthetic construct
  source 1..22: mol_type = other DNA
ccgccctgatcaaaaagtaccc

SEQ ID NO: 23 - DNA, 20 bp, synthetic construct
  source 1..20: mol_type = other DNA
atcatgcatctggatctggc

SEQ ID NO: 24 - DNA, 20 bp, synthetic construct
  source 1..20: mol_type = other DNA
tcatgcatctggatctggct

SEQ ID NO: 25 - protein, 7 aa, synthetic construct
MAEPGAR

SEQ ID NO: 26 - protein, 7 aa, synthetic construct
MLYADNT

SEQ ID NO: 27 - protein, 7 aa, synthetic construct
SQDPSTL

SEQ ID NO: 28 - DNA, 20 bp, synthetic construct
  source 1..20: mol_type = other DNA
tgcatctggatctggctggc

SEQ ID NO: 29 - DNA, 21 bp, synthetic construct
  source 1..21: mol_type = other DNA
gccatcatgcgtctggatctg

SEQ ID NO: 30 - DNA, 21 bp, synthetic construct
  source 1..21: mol_type = other DNA
gccatcatgcatctggatctg

SEQ ID NO: 31 - DNA, 40 bp, synthetic construct
  source 1..40: mol_type = other DNA
cttgccccatgccatcatgcgtctggatctggctggccga

SEQ ID NO: 32 - DNA, 40 bp, synthetic construct
  source 1..40: mol_type = other DNA
cttgccccatgccatcatgcatctggatctggctggccga

SEQ ID NO: 33 - DNA, 40 bp, synthetic construct
  source 1..40: mol_type = other DNA
atgccttgccccatgccatcatgcgtctggatctggctgg

SEQ ID NO: 34 - DNA, 40 bp, synthetic construct
  source 1..40: mol_type = other DNA
atgccttgccccatgccatcatgcatctggatctggctgg

SEQ ID NO: 35 - DNA, 25 bp, synthetic construct
  source 1..25: mol_type = other DNA
atgccatgcgtctggatctggctgg

SEQ ID NO: 36 - DNA, 41 bp, synthetic construct
  source 1..41: mol_type = other DNA
atgccttgccccatgccatccatcatatgcgtctggatgtg

SEQ ID NO: 37 - protein, 11 aa, synthetic construct
AIMHLDLAGRD

SEQ ID NO: 38 - DNA, 23 bp, synthetic construct
  source 1..23: mol_type = other DNA
tgcatctggatctggctggccga

SEQ ID NO: 39 - DNA, 23 bp, synthetic construct
  source 1..23: mol_type = other DNA
tcatgcatctggatctggctggc

SEQ ID NO: 40 - DNA, 23 bp, synthetic construct
  source 1..23: mol_type = other DNA
atcatgcatctggatctggctgg

SEQ ID NO: 41 - DNA, 15 bp, synthetic construct
  source 1..15: mol_type = other DNA
atcatgcatctggat

SEQ ID NO: 42 - protein, 5 aa, synthetic construct
IMHLD

SEQ ID NO: 43 - DNA, 15 bp, synthetic construct
  source 1..15: mol_type = other DNA
atcgtgcatctggat

SEQ ID NO: 44 - protein, 5 aa, synthetic construct
IVHLD

SEQ ID NO: 45 - DNA, 11 bp, synthetic construct
  source 1..11: mol_type = other DNA
gcatctggatc

SEQ ID NO: 46 - DNA, 11 bp, synthetic construct
  source 1..11: mol_type = other DNA
gcatatggatc

SEQ ID NO: 47 - DNA, 11 bp, synthetic construct
  source 1..11: mol_type = other DNA
gcatgtggatc

SEQ ID NO: 48 - DNA, 11 bp, synthetic construct
  source 1..11: mol_type = other DNA
gcatttggatc

SEQ ID NO: 49 - DNA, 12 bp, synthetic construct
  source 1..12: mol_type = other DNA
catctggatctg

SEQ ID NO: 50 - DNA, 12 bp, synthetic construct
  source 1..12: mol_type = other DNA
catctgggtctg

SEQ ID NO: 51 - protein, 7 aa, synthetic construct
AIMRLDL

SEQ ID NO: 52 - protein, 7 aa, synthetic construct
AIMHLDL

SEQ ID NO: 53 - DNA, 27 bp, synthetic construct
  source 1..27: mol_type = other DNA
atcatgcatctggatctggctggccga

SEQ ID NO: 54 - protein, 9 aa, synthetic construct
IMHLDLAGR

SEQ ID NO: 55 - DNA, 23 bp, synthetic construct
  source 1..23: mol_type = other DNA
catcatgcatctggatctggctg

SEQ ID NO: 56 - DNA, 23 bp, synthetic construct
  source 1..23: mol_type = other DNA
atcatgcatctggatctggctgg

SEQ ID NO: 57 - DNA, 23 bp, synthetic construct
  source 1..23: mol_type = other DNA
tcatgcatctggatctggctggc

SEQ ID NO: 58 - DNA, 23 bp, synthetic construct
  source 1..23: mol_type = other DNA
catgcatctggatctggctggcc

SEQ ID NO: 59 - DNA, 23 bp, synthetic construct
  source 1..23: mol_type = other DNA
atgcatctggatctggctggccg

SEQ ID NO: 60 - DNA, 23 bp, synthetic construct
  source 1..23: mol_type = other DNA
tgcatctggatctggctggccga

SEQ ID NO: 61 - DNA, 23 bp, synthetic construct
  source 1..23: mol_type = other DNA
tgcatctggacttggctggccga

SEQ ID NO: 62 - DNA, 29 bp, synthetic construct
  source 1..29: mol_type = other DNA
tcatgcatctggatctggctggccgagat

SEQ ID NO: 63 - DNA, 28 bp, synthetic construct
  source 1..28: mol_type = other DNA
catgcatctggatctggctggccgagat

SEQ ID NO: 64 - DNA, 27 bp, synthetic construct
  source 1..27: mol_type = other DNA
atgcatctggatctggctggccgagat

SEQ ID NO: 65 - DNA, 26 bp, synthetic construct
  source 1..26: mol_type = other DNA
tgcatctggatctggctggccgagat

SEQ ID NO: 66 - DNA, 23 bp, synthetic construct
  source 1..23: mol_type = other DNA
gccccatgccatcatgcatctgg

SEQ ID NO: 67 - DNA, 23 bp, synthetic construct
  source 1..23: mol_type = other DNA
tgccatcatgcatctggatctgg

SEQ ID NO: 68 - DNA, 23 bp, synthetic construct
  source 1..23: mol_type = other DNA
agccagatccagatgcatgatgg

SEQ ID NO: 69 - DNA, 23 bp, synthetic construct
  source 1..23: mol_type = other DNA
ccccatgccatcatgcatctgga

SEQ ID NO: 70 - DNA, 23 bp, synthetic construct
  source 1..23: mol_type = other DNA
gccagccagatccagatgcatga

SEQ ID NO: 71 - DNA, 24 bp, synthetic construct
  source 1..24: mol_type = other DNA
gatctcggccagccagatccagat

SEQ ID NO: 72 - DNA, 24 bp, synthetic construct
  source 1..24: mol_type = other DNA
tgccttgccccatgccatcatgca

SEQ ID NO: 73 - DNA, 23 bp, synthetic construct
  source 1..23: mol_type = other DNA
gccatcatgcatctggatctggc

SEQ ID NO: 74 - DNA, 23 bp, synthetic construct
  source 1..23: mol_type = other DNA
gccagatccagatgcatgatggc

SEQ ID NO: 75 - DNA, 28 bp, synthetic construct
  source 1..28: mol_type = other DNA
cttgccccatgccatcatgcatctggat

SEQ ID NO: 76 - DNA, 28 bp, synthetic construct
  source 1..28: mol_type = other DNA
ctcggccagccagatccagatgcatgat

SEQ ID NO: 77 - DNA, 28 bp, synthetic construct
  source 1..28: mol_type = other DNA
gtgagatctcggccagccagatccagat

SEQ ID NO: 78 - DNA, 24 bp, synthetic construct
  source 1..24: mol_type = other DNA
tccagatgcatgatggcatggggc

SEQ ID NO: 79 - DNA, 24 bp, synthetic construct
  source 1..24: mol_type = other DNA
tccagatgcatgatggcatggggc

SEQ ID NO: 80 - DNA, 20 bp, synthetic construct
  source 1..20: mol_type = other DNA
gccccatgccatcatgcgtc

SEQ ID NO: 81 - DNA, 24 bp, synthetic construct
  source 1..24: mol_type = other DNA
gccagatccagatgcatgatggca

SEQ ID NO: 82 - DNA, 20 bp, synthetic construct
  source 1..20: mol_type = other DNA
ggcctcaccagtagtaacga

SEQ ID NO: 83 - DNA, 20 bp, synthetic construct
  source 1..20: mol_type = other DNA
tgccatcatgcatctggatc

SEQ ID NO: 84 - DNA, 20 bp, synthetic construct
  source 1..20: mol_type = other DNA
agccagatccagatgcatga

SEQ ID NO: 85 - DNA, 434 bp, synthetic construct
  source 1..434: mol_type = other DNA
ataaatgttttgtagaataaaaaaaaaaaaagttcttcaaaagaaatctcaaatctccaaatggaaacaggtaaaagtggagctcccctggttccacggagaaccttttttgaggaaacttaggcaactcgcaggtaccttatgtcatgagacagagtttgaaaactacaattgactatctctaaatttcctcccaggtctaaaatgtgatgatagttactacttcagtacatcatccttaaggaaaattattaggtccacactgtttctatcctttgaattttacacataaattttgtaatcaaaagtttatttgtaatatcagatggaatcagataattgctttttgttttttccactgacaggaacataagattttgttgtgtagcttaagtcaaacgcagtttggaatatatattttttaaaaattgtaa

SEQ ID NO: 86 - DNA, 482 bp, synthetic construct
  source 1..482: mol_type = other DNA
cttacatatccaaatacaatttttcaagaagtagagtattcagtagaaattaatctgtgaaagaagaggaattcagcagtggcctatttgatgaatgatttaacgtgcttatttcttccctttcatcaaaactctgtgtccccttgtttgccccctctgacttcatactctggagttgaccaagatccctcttccatcggattgttctgggaattttgaaataatctgctttttcctctctttcccctgttgcttctgatgccttagaattacattttcctcgctgatttagtttagaaaagagaaaagagcttccatgactagtagattatcacttttgggtttgctcttggaagtgacaagatgctaggatccctctttggaatgtaaaatttatctcttatatagaaaggatataaatgtagcaccagagactataaaactctgatactatctactgtactgtatagctgaacgccaca

SEQ ID NO: 87 - DNA, 434 bp, synthetic construct
  source 1..434: mol_type = other DNA
atgtgtctggtaatctattgactatcataaatgctatttctacagaaaagttaggaggtccatatttcgggcaaccaatgtatagctgaatgcagaacagtcatagttgggtactaaccatatatatgatttatccatcaacaggtgcatatgctcagaaattctgtatccataagaaatcagactactttcttttccttttgcaagtaaattgaatttagcctgagaggctgaggggaaattttcacatataagccacggttttgtgttttgtgttttgttttgtttatagatatagtactaactggatggatgcgataaaattcataggtggtactaagatacaataggatttgtgaaatggacaattgtcttgcataaatagcaagtaaaaaatcaagcctgtccttcataaaaattttattctggggtgt